SEQUENCE LISTING (17 sequences)

SEQ ID NO: 1 - protein, 21 aa, synthetic construct
  source 1..21: note = Ciltacabtagene autoleucel CAR CD8alpha signal peptide, CD8alpha SP amino acid sequence
MALPVTALLLPLALLLHAARP

SEQ ID NO: 2 - protein, 119 aa, synthetic construct
  source 1..119: note = Ciltacabtagene autoleucel CAR BCMA binding domain, VHH1 amino acid sequence
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSS

SEQ ID NO: 3 - protein, 5 aa, synthetic construct
  source 1..5: note = Ciltacabtagene autoleucel CAR BCMA binding domain, G4S linker amino acid sequence
GGGGS

SEQ ID NO: 4 - protein, 118 aa, synthetic construct
  source 1..118: note = Ciltacabtagene autoleucel CAR BCMA binding domain, VHH2 amino acid sequence
EVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSS

SEQ ID NO: 5 - protein, 45 aa, synthetic construct
  source 1..45: note = Ciltacabtagene autoleucel CAR CD8alpha hinge amino acid sequence
TTTPAPRPPTPAPTIASQPLSLRPEACRPAAGGAVHTRGLDFACD

SEQ ID NO: 6 - protein, 24 aa, synthetic construct
  source 1..24: note = Ciltacabtagene autoleucel CAR CD8alpha transmembrane amino acid sequence
IYIWAPLAGTCGVLLLSLVITLYC

SEQ ID NO: 7 - protein, 42 aa, synthetic construct
  source 1..42: note = Ciltacabtagene autoleucel CAR CD137 Cytoplasmic amino acid sequence
KRGRKKLLYIFKQPFMRPVQTTQEEDGCSCRFPEEEEGGCEL

SEQ ID NO: 8 - protein, 112 aa, synthetic construct
  source 1..112: note = Ciltacabtagene autoleucel CAR CD3z Cytoplasmic amino acid sequence
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - DNA, 63 bp, synthetic construct
  source 1..63: mol_type = other DNA; note = Ciltacabtagene autoleucel CAR CD8alpha signal peptide CD8alpha SP nucleic acid sequence
atggctctgcccgtcaccgctctgctgctgcctctggctctgctgctgcacgctgctcgccct

SEQ ID NO: 10 - DNA, 357 bp, synthetic construct
  source 1..357: mol_type = other DNA; note = Ciltacabtagene autoleucel CAR BCMA binding domain, VHH1 nucleic acid sequence
caggtcaaactggaagaatctggcggaggcctggtgcaggcaggacggagcctgcgcctgagctgcgcagcatccgagcacaccttcagctcccacgtgatgggctggtttcggcaggccccaggcaaggagagagagagcgtggccgtgatcggctggagggacatctccacatcttacgccgattccgtgaagggccggttcaccatcagccgggacaacgccaagaagacactgtatctgcagatgaacagcctgaagcccgaggacaccgccgtgtactattgcgcagcaaggagaatcgacgcagcagactttgattcctggggccagggcacccaggtgacagtgtctagc

SEQ ID NO: 11 - DNA, 15 bp, synthetic construct
  source 1..15: mol_type = other DNA; note = Ciltacabtagene autoleucel CAR BCMA binding domain, G4S linker (SEQ ID NO: 3) nucleic acid sequence
ggaggaggaggatct

SEQ ID NO: 12 - DNA, 354 bp, synthetic construct
  source 1..354: mol_type = other DNA; note = Ciltacabtagene autoleucel CAR BCMA binding domain, VHH2 nucleic acid sequence
gaggtgcagctggtggagagcggaggcggcctggtgcaggccggaggctctctgaggctgagctgtgcagcatccggaagaaccttcacaatgggctggtttaggcaggcaccaggaaaggagagggagttcgtggcagcaatcagcctgtcccctaccctggcctactatgccgagagcgtgaagggcaggtttaccatctcccgcgataacgccaagaatacagtggtgctgcagatgaactccctgaaacctgaggacacagccctgtactattgtgccgccgatcggaagagcgtgatgagcattagaccagactattgggggcagggaacacaggtgaccgtgagcagc

SEQ ID NO: 13 - DNA, 135 bp, synthetic construct
  source 1..135: mol_type = other DNA; note = Ciltacabtagene autoleucel CAR CD8alpha hinge nucleic acid sequence
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 14 - DNA, 72 bp, synthetic construct
  source 1..72: mol_type = other DNA; note = Ciltacabtagene autoleucel CAR CD8alpha transmembrane nucleic acid sequence
atctacatctgggcgcccttggccgggacttgtggggtccttctcctgtcactggttatcaccctttactgc

SEQ ID NO: 15 - DNA, 126 bp, synthetic construct
  source 1..126: mol_type = other DNA; note = Ciltacabtagene autoleucel CAR CD137 Cytoplasmic nucleic acid sequence
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 16 - DNA, 339 bp, synthetic construct
  source 1..339: mol_type = other DNA; note = Ciltacabtagene autoleucel CAR CD3z Cytoplasmic nucleic acid sequence
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 17 - protein, 488 aa, synthetic construct
  source 1..488: note = Ciltacabtagene autoleucel CAR amino acid sequence
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2023/0270786 A1
(43) Pub. Date: Aug. 31, 2023

(54) BCMA-TARGETED CAR-T CELL THERAPY FOR MULTIPLE MYELOMA

(71) Applicants: JANSSEN BIOTECH, INC., Horsham, PA (US); NANJING LEGEND BIOTECH CO., LTD., Nanjing (CN); LEGEND BIOTECH USA INC., Somerset, NJ (US)

(72) Inventors: Xiaohu FAN, Edmonton (CA); Jordan SCHECTER, Raritan, NJ (US); Lida PACAUD, Somerset, NJ (US); Michael HIRSCHMANN, Somerset, NJ (US)

(73) Assignees: JANSSEN BIOTECH, INC., Horsham, PA (US); NANJING LEGEND BIOTECH CO., LTD., Nanjing, Jiangsu, PA (CN); LEGEND BIOTECH USA INC., Somerset, NJ (US)

(21) Appl. No.: 18/175,347

(22) Filed: Feb. 27, 2023

Related U.S. Application Data
(60) Provisional application No. 63/314,968, filed on Feb. 28, 2022.

(51) Int. Cl. A61K 35/17; A61K 31/675; A61K 31/52; A61K 45/06; A61P 35/00

(52) U.S. Cl. CPC A61K 35/17; A61K 31/675; A61K 31/52; A61K 45/06; A61P 35/00

(57) ABSTRACT

Provided herein are methods of treating a subject who has multiple myeloma with a ciltacabtagene autoleucel suspension. Also provided are pharmaceutical products containing ciltacabtagene autoleucel suspensions, instructions for use of the ciltacabtagene autoleucel suspensions, and methods for selling a drug product containing ciltacabtagene autoleucel suspensions.

### CROSS-REFERENCE TO RELATED APPLICATION

This application claims benefit of priority of U.S. Provisional Patent Application No. 63/314,968 filed on Feb. 28, 2022, the disclosure of which is incorporated by reference herein in its entirety.

### SEQUENCE LISTING

This application contains a computer readable Sequence Listing which has been submitted in XML file format via Patent Center, the entire content of which is incorporated by reference herein in its entirety. The Sequence Listing XML file submitted via Patent Center is entitled “14651-054-999_SEQ_LISTING.xml”, was created on Feb. 23, 2023, and is 21,916 bytes in size.

### BACKGROUND

Multiple myeloma is a neoplasm of plasma cells that is aggressive. Multiple myeloma is considered to be a B-cell neoplasm that proliferates uncontrollably in the bone marrow. Symptoms include one or more of hypercalcemia, renal insufficiency, anemia, bony lesions, bacterial infections, hyperviscosity and amyloidosis. Multiple myeloma is still considered to be an incurable disease, despite availability of new therapies that include proteasome inhibitors, immunomodulatory drugs, and monoclonal antibodies that have significantly improved patient outcomes. Because most patients will either relapse or become refractory to treatment, there is an ongoing need for new therapies for multiple myeloma.

### SUMMARY OF THE DISCLOSURE

In one aspect is provided a method of treating a subject who has multiple myeloma, the method comprising administering to the subject via a single intravenous infusion a composition comprising T cells comprising a chimeric antigen receptor (CAR) comprising:
a) an extracellular antigen binding domain comprising a first anti-BCMA binding moiety and a second BCMA binding moiety;
b) a transmembrane domain; and
c) an intracellular signaling domain,
   to deliver to the subject a dose of CAR expressing T cells (CAR-T cells),
   wherein the first BCMA binding moiety comprises the amino acid sequence set forth in SEQ ID NO: 2 and the second BCMA binding moiety comprises the amino acid sequence set forth in SEQ ID NO: 4.

In some embodiments, the dose comprises 1.0×10⁵ to 5.0×10⁶ of said CAR-T cells per kilogram of the mass of the subject. In some embodiments, the dose comprises 5.0×10⁵ to 1.0×10⁶ of said CAR-T cells per kilogram of the mass of the subject. In some embodiments, the dose comprises approximately 0.75×10⁶ of said CAR-T cells per kilogram of the mass of the subject. In some embodiments, the dose comprises less than 1.0×10⁸ of said CAR-T cells per subject.

In some embodiments, said single intravenous infusion is administered using a single bag of said CAR-T cells. In some embodiments, said administration of said single bag of said CAR-T cells is completed no later than three hours following the thawing of said single bag of CAR-T cells.

In some embodiments, said single intravenous infusion is administered using two bags of said CAR-T cells. In some embodiments, said administration of each of said two bags of said CAR-T cells is completed no later than three hours following the thawing of said each of said two bags of CAR-T cells.

In some embodiments, said method is effective in obtaining minimal residual disease (MRD) negative status in said subject assessed in the bone marrow after said infusion of said CAR-T cells. In some embodiments, said MRD negative status is obtained at a first follow-up time of between approximately 28 days and approximately 179 days after said infusion of said CAR-T cells.

In some embodiments, a lymphodepleting regimen precedes said infusion of CAR-T cells. In some embodiments, said lymphodepleting regimen comprises administration of cyclophosphamide, or administration of fludarabine. In some embodiments, the lymphodepleting regimen is administered intravenously. In some embodiments, said lymphodepleting regimen precedes said infusion of CAR-T cells by 5 to 7 days. In some embodiments, said lymphodepleting regimen comprises intravenous administration of cyclophosphamide and fludarabine 5 to 7 days prior to said infusion of CAR-T cells. In some embodiments, said cyclophosphamide is administered intravenously at 300 mg/m². In some embodiments, said fludarabine is administered intravenously at 30 mg/m².

In some embodiments, the method further comprises treating said subject for cytokine release syndrome (CRS) more than 3 days following the infusion without significantly reducing CAR-T cell expansion in vivo. In some embodiments, said treatment of CRS comprises administering to the subject an IL-6R inhibitor. In some embodiments, said IL-6R inhibitor is an antibody. In some embodiments, said antibody inhibits IL-6R by binding its extracellular domain. In some embodiments, said IL-6R inhibitor prevents the binding of IL-6 to IL-6R. In some embodiments, the IL-6R inhibitor is tocilizumab.

In some embodiments, the subject is treated with pre-infusion medication comprising an antipyretic and an antihistamine up to 1 hour prior to the infusion comprising CAR-T cells. In some embodiments, said antipyretic comprises either paracetamol or acetaminophen. In some embodiments, said antipyretic is administered to the subject either orally or intravenously. In some embodiments, said antipyretic is administered to the subject at a dosage of between 650 mg and 1000 mg. In some embodiments, said antihistamine comprises diphenhydramine. In some embodiments, said antihistamine is administered to the subject either orally or intravenously. In some embodiments, said antihistamine is administered at a dosage of between 25 mg and 50 mg, or its equivalent.

In some embodiments, the infusion comprising CAR-T cells further comprises an excipient selected from dimethyl sulfoxide or dextran-40.

In some embodiments, the subject received prior treatment with at least three prior lines of treatment. In some embodiments, said at least three prior lines of treatment comprises treatment with at least one medicament, said at least one medicament comprising of at least one of a proteasome inhibitor (PI), an immunomodulatory agent (IMiD), or an anti-CD38 antibody.

In some embodiments, the subject has relapsed after said at least three prior lines of treatment. In some embodiments, the multiple myeloma is refractory to at least two medicaments following said at least three prior lines of treatment. In some embodiments, said at least two medicaments to which the subject is refractory comprise a PI and an IMiD. In some embodiments, the subject is refractory to at least three medicaments. In some embodiments, the subject is refractory to at least four medicaments. In some embodiments, the subject is refractory to at least five medicaments.

In some embodiments, said method is effective in obtaining an overall response rate of greater than 91%. In some embodiments, said method is effective in obtaining an overall response rate of greater than 93%. In some embodiments, said method is effective in obtaining an overall response rate of greater than 95%. In some embodiments, said method is effective in obtaining an overall response rate of greater than 97%. In some embodiments, said method is effective in obtaining an overall response rate of greater than 99%. In some embodiments, the overall response rate is assessed at a median follow-up time of at least 12 months following said infusion of said CAR-T cells.

In some embodiments, said method is effective in obtaining a median time to first response of less than 1.15 months. In some embodiments, said method is effective in obtaining a median time to first response of less than 1.10 months. In some embodiments, said method is effective in obtaining a median time to first response of less than 1.05 months. In some embodiments, said method is effective in obtaining a median time to first response of less than 1.00 months. In some embodiments, said method is effective in obtaining a median time to first response of less than 0.95 months.

In some embodiments, said method is effective in obtaining a median time to best response of less than 2.96 months. In some embodiments, said method is effective in obtaining a median time to best response of less than 2.86 months. In some embodiments, said method is effective in obtaining a median time to best response of less than 2.76 months. In some embodiments, said method is effective in obtaining a median time to best response of less than 2.66 months. In some embodiments, said method is effective in obtaining a median time to best response of less than 2.56 months.

In some embodiments, said method is effective in obtaining minimal residual disease (MRD) negative status in said subject assessed in the bone marrow at a follow-up time of approximately 28 days or greater following said infusion of said CAR-T cells. In some embodiments, said method is effective in maintaining said MRD negative status in said subject assessed in the bone marrow at a follow-up time of approximately 12 months or greater following said infusion of said CAR-T cells.

In some embodiments, the first BCMA binding moiety and/or the second BCMA binding moiety is an anti-BCMA VHH. In some embodiments, the first BCMA binding moiety is a first anti-BCMA VHH and the second BCMA binding moiety is a second anti-BCMA VHH. In some embodiments, the first BCMA binding moiety comprises a polypeptide encoded by the nucleic acid sequence of SEQ ID NO: 10. In some embodiments, the second BCMA binding moiety comprises a polypeptide encoded by the nucleic acid sequence of SEQ ID NO: 12. In some embodiments, the first BCMA binding moiety and the second BCMA binding moiety are connected to each other via a peptide linker.

In some embodiments, the peptide linker comprises the amino acid sequence of SEQ ID NO: 3. In some embodiments, the peptide linker comprises a polypeptide encoded by the nucleic acid sequence of SEQ ID NO: 11.

In some embodiments, the CAR polypeptide further comprises a signal peptide located at the N-terminus of the polypeptide. In some embodiments, the signal peptide is derived from CD8-alpha. In some embodiments, the signal peptide comprises the amino acid sequence of SEQ ID NO: 1. In some embodiments, the signal peptide comprises a polypeptide encoded by the nucleic acid sequence of SEQ ID NO: 9.

In some embodiments, the transmembrane domain comprises the amino acid sequence of SEQ ID NO: 6. In some embodiments, the transmembrane domain comprises a polypeptide encoded by the nucleic acid sequence of SEQ ID NO: 14.

In some embodiments, the intracellular signaling domain comprises a primary intracellular signaling domain of an immune effector cell. In some embodiments, the intracellular signaling domain is derived from CD3ζ. In some embodiments, the intracellular signaling domain comprises one or more co-stimulatory signaling domains. In some embodiments, the intracellular signaling domain comprises the amino acid sequence of SEQ ID NO: 8. In some embodiments, the intracellular signaling domain comprises a polypeptide encoded by the nucleic acid sequence of SEQ ID NO: 16. In some embodiments, the intracellular signaling domain comprises the amino acid sequence of SEQ ID NO: 7. In some embodiments, the intracellular signaling domain comprises a polypeptide encoded by the nucleic acid sequence of SEQ ID NO: 15.

In some embodiments, the CAR polypeptide further comprises a hinge domain located between the C-terminus of the extracellular antigen binding domain and the N-terminus of the transmembrane domain. In some embodiments, the hinge domain comprises the amino acid sequence of SEQ ID NO: 5. In some embodiments, the hinge domain comprises a polypeptide encoded by the nucleic acid sequence of SEQ ID NO: 13.

In some embodiments, the T cells are autologous T cells. In some embodiments, the T cells are allogeneic T cells.

In some embodiments, the subject is human.

In one aspect is provided a method of treating a subject who has multiple myeloma and received at least three prior lines of treatment, the method comprising administering to the subject via a single intravenous infusion a composition comprising T cells comprising a chimeric antigen receptor (CAR) comprising the amino acid sequence of SEQ ID NO: 17 to deliver to the subject a dose of approximately 0.75×10⁶ CAR expressing T cells (CAR-T cells) per kilogram of the mass of the subject, wherein said method is effective in obtaining minimal residual disease (MRD) negative status in said subject assessed in the bone marrow at a follow-up time of greater than or equal to 28 days following said infusion of said CAR-T cells.

In some embodiments, said method is effective in obtaining said MRD negative status at a rate of between approximately 44% and approximately 65% at a sensitivity threshold level of 10⁻⁵ at a follow-up time of approximately 12 months after said infusion of said CAR-T cells, a rate of between approximately 57% and approximately 76% at a sensitivity threshold level of 10⁻⁴ at a follow-up time of approximately 18 months after said infusion of said CAR-T cells, a rate of between approximately 47% and approximately 68% at a sensitivity threshold level of 10⁻⁵ at a follow-up time of approximately 18 months after said infusion of said CAR-T cells, or a rate of between approximately 29% and approximately 50% at a sensitivity threshold level of 10⁻⁶ at a follow-up time of approximately 18 months after said infusion of said CAR-T cells.

In some embodiments, said method is effective in obtaining said MRD negative status at a rate of approximately 55% at a sensitivity threshold level of 10⁻⁵ at a follow-up time of approximately 12 months after said administration of said CAR-T cells, a rate of approximately 67% at a sensitivity threshold level of 10⁻⁴ at a follow-up time of approximately 18 months after said infusion of said CAR-T cells, a rate of approximately 58% at a sensitivity threshold level of 10⁻⁵ at a follow-up time of approximately 18 months after said infusion of said CAR-T cells, or a rate of approximately 39% at a sensitivity threshold level of 10⁻⁶ at a follow-up time of approximately 18 months after said infusion of said CAR-T cells.

In some embodiments, said method is effective in obtaining said minimal residual disease (MRD) negative status at a rate of between approximately 83% and approximately 98% in subjects with evaluable samples at a sensitivity threshold level of 10⁻⁵ at a follow-up time of approximately 12 months after said infusion of said CAR-T cells, or at a rate of between approximately 82% and approximately 97% in subjects with evaluable samples at a sensitivity threshold level of 10⁻⁵ at a follow-up time of approximately 18 months after said infusion of said CAR-T cells.

In some embodiments, said method is effective in obtaining said minimal residual disease (MRD) negative status at a rate of approximately 93% in subjects with evaluable samples at a sensitivity threshold level of 10⁻⁵ at a follow-up time of approximately 12 months after said infusion of said CAR-T cells, or at a rate of approximately 92% in subjects with evaluable samples at a sensitivity threshold level of 10⁻⁵ at a follow-up time of approximately 18 months after said infusion of said CAR-T cells.

In some embodiments, said method is effective in obtaining at least one response in the subject after said infusion of said CAR-T cells, wherein said at least one response comprises, in order from better to worse, a stringent complete response, a complete response, a very good partial response, a partial response, or a minimal response.

In some embodiments, said method is effective in obtaining a first response before a time of between approximately 27 days and approximately 321 days after said infusion of said CAR-T cells. In some embodiments, said method is effective in obtaining a first response before a time of between approximately 27 days and approximately 89 days after said infusion of said CAR-T cells. In some embodiments, said method is effective in obtaining a first response before approximately 42 days after said infusion of said CAR-T cells. In some embodiments, said method is effective in obtaining a first response before approximately 29 days after said infusion of said CAR-T cells.

In some embodiments, said method is effective in obtaining a best response of any one of minimal response, partial response, very good partial response, complete response or stringent complete response. In some embodiments, said method is effective in obtaining said best response of any one of minimal response, partial response, very good partial response, complete response or stringent complete response at a rate of between approximately 91% and approximately 99% at a follow-up time of approximately 12 months after said infusion of said CAR-T cells or at a rate of between approximately 93% and approximately 100% at a follow-up time of approximately 18 months after said infusion of said CAR-T cells. In some embodiments, said method is effective in obtaining said best response of any one of minimal response, partial response, very good partial response, complete response or stringent complete response at a rate of approximately 97% at a follow-up time of approximately 12 months after said infusion of said CAR-T cells or at a rate of approximately 98% at a follow-up time of approximately 18 months after said infusion of said CAR-T cells.

In some embodiments, said method is effective in obtaining a best response of any one of partial response, very good partial response, complete response or stringent complete response. In some embodiments, said method is effective in obtaining said best response of any one of partial response, very good partial response, complete response or stringent complete response at a rate of between approximately 91% and approximately 99% at a follow-up time of approximately 12 months after said infusion of said CAR-T cells or at a rate of between approximately 93% and approximately 100% at a follow-up time of approximately 18 months after said infusion of said CAR-T cells. In some embodiments, said method is effective in obtaining said best response of any one of partial response, very good partial response, complete response or stringent complete response at a rate of approximately 97% at a follow-up time of approximately 12 months after said infusion of said CAR-T cells or at a rate of approximately 97% at a follow-up time of approximately 18 months after said infusion of said CAR-T cells.

In some embodiments, said method is effective in obtaining a best response of any one of very good partial response, complete response or stringent complete response. In some embodiments, said method is effective in obtaining said best response of any one of very good partial response, complete response or stringent complete response at a rate of between approximately 86% and approximately 97% at a follow-up time of approximately 12 months after said infusion of said CAR-T cells or at a rate of between approximately 88% and approximately 98% at a follow-up time of approximately 18 months after said infusion of said CAR-T cells. In some embodiments, said method is effective in obtaining said best response of any one of very good partial response, complete response or stringent complete response at a rate of approximately 93% at a follow-up time of approximately 12 months after said infusion of said CAR-T cells or at a rate of approximately 95% at a follow-up time of approximately 18 months after said infusion of said CAR-T cells.

In some embodiments, said method is effective in obtaining a best response of complete response or stringent complete response. In some embodiments, said method is effective in obtaining said best response of complete response or stringent complete response at a rate of between approximately 57% and approximately 76% at a follow-up time of approximately 12 months after said infusion of said CAR-T cells or at a rate of between approximately 73% and approximately 89% at a follow-up time of approximately 18 months after said infusion of said CAR-T cells. In some embodiments, said method is effective in obtaining said best response of complete response or stringent complete response at a rate of approximately 67% at a follow-up time of approximately 12 months after said infusion of said CAR-T cells or at a rate of approximately 83% at a follow-up time of approximately 18 months after said infusion of said CAR-T cells.

In some embodiments, said method is effective in obtaining a best response of stringent complete response. In some embodiments, said method is effective in obtaining said best response of stringent complete response at a rate of between approximately 57% and approximately 76% at a follow-up time of approximately 12 months after said infusion of said CAR-T cells or at a rate of between approximately 73% and approximately 89% at a follow-up time of approximately 18 months after said infusion of said CAR-T cells. In some embodiments, said method is effective in obtaining said best response of stringent complete response at a rate of approximately 67% at a follow-up time of approximately 12 months after said infusion of said CAR-T cells or at a rate of approximately 83% at a follow-up time of approximately 18 months after said infusion of said CAR-T cells.

In some embodiments, said method is effective in obtaining progression-free survival of the subject. In some embodiments, said method is effective in obtaining said progression-free survival of the subject at a time between said infusion of said CAR-T cells and approximately 209 days after said infusion of said CAR-T cells, approximately 386 days after said infusion of said CAR-T cells, approximately 632 days after said infusion of said CAR-T cells, or approximately 684 days after said infusion of said CAR-T cells.

In some embodiments, said method is effective in obtaining said progression-free survival at a rate of between approximately 79% and approximately 93% at a follow-up time of approximately 6 months after said infusion of said CAR-T cells, a rate of between approximately 67% and approximately 84% at a follow-up time of approximately 12 months after said infusion of said CAR-T cells, a rate of between approximately 57% and approximately 75% at a follow-up time of approximately 18 months after said infusion of said CAR-T cells, a rate of between approximately 57% and approximately 75% at a follow-up time of approximately 21 months after said infusion of said CAR-T cells, or a rate of between approximately 49% and approximately 70% at a follow-up time of approximately 24 months after said infusion of said CAR-T cells.

In some embodiments, said method is effective in obtaining said progression-free survival at a rate of approximately 88% at a follow-up time of approximately 6 months after said infusion of said CAR-T cells, a rate of approximately 76% at a follow-up time of approximately 12 months after said infusion of said CAR-T cells, a rate of approximately 67% at a follow-up time of approximately 18 months after said infusion of said CAR-T cells, a rate of approximately 67% at a follow-up time of approximately 21 months after said infusion of said CAR-T cells, or a rate of approximately 61% at a follow-up time of approximately 24 months after said infusion of said CAR-T cells.

In some embodiments, said method further comprises treating said subject for cytokine release syndrome more than approximately 1 day after said infusion of said CAR-T cells. In some embodiments, said method is effective in obtaining a rate of recovery from said cytokine release syndrome of between approximately 1% and approximately 99% at a time of approximately 1, 3, 4, 6, 16 or 97 days after first observance of said cytokine release syndrome.

In some embodiments, said method further comprises treating said subject for immune effector cell-associated neurotoxicity more than approximately 3 days after said infusion of said CAR-T cells. In some embodiments, said method is effective in obtaining a rate of recovery from said immune effector cell-associated neurotoxicity of between approximately 1% and approximately 17% at a time of approximately 1, 4, 5, 8, 12 or 16 days after first observance of said immune effector cell-associated neurotoxicity.

In some embodiments, said method is effective in obtaining said best response before a time of between approximately 27 days and approximately 534 days after said infusion of said CAR-T cells. In some embodiments, said method is effective in obtaining said best response before a time of between approximately 27 days and approximately 293 days after said infusion of said CAR-T cells. In some embodiments, said method is effective in obtaining said best response before approximately 153 days after said infusion of said CAR-T cells. In some embodiments, said method is effective in obtaining said best response before approximately 78 days after said infusion of said CAR-T cells.

In some embodiments, said method is effective in maintaining a response in the subject at a follow-up time between the time of said first response and approximately 180 days after said infusion of said CAR-T cells, approximately 357 days after said infusion of said CAR-T cells, approximately 606 days after said infusion of said CAR-T cells, or approximately 654 days after said infusion of said CAR-T cells. In some embodiments, said method is effective in maintaining a response at a rate of between approximately 77% and approximately 91% at a follow-up time of approximately 6 months after said infusion of said CAR-T cells, a rate of between approximately 63% and approximately 81% at a follow-up time of approximately 12 months after said infusion of said CAR-T cells, a rate of between approximately 56% and approximately 75% at a follow-up time of approximately 18 months after said infusion of said CAR-T cells, a rate of between approximately 52% and approximately 72% at a follow-up time of approximately 21 months after said infusion of said CAR-T cells, or a rate of between approximately 48% and approximately 70% at a follow-up time of approximately 24 months after said infusion of said CAR-T cells. In some embodiments, said method is effective in maintaining a response at a rate of approximately 85% at a follow-up time of approximately 6 months after said infusion of said CAR-T cells, a rate of approximately 74% at a follow-up time of approximately 12 months after said infusion of said CAR-T cells, a rate of approximately 67% at a follow-up time of approximately 18 months after said infusion of said CAR-T cells, a rate of approximately 63% at a follow-up time of approximately 21 months after said infusion of said CAR-T cells, or a rate of approximately 60% at a follow-up time of approximately 24 months after said infusion of said CAR-T cells.

In some embodiments, said method is effective in obtaining minimal residual disease (MRD) negative status in said subject assessed in the bone marrow at a sensitivity threshold level of 10⁻⁵ between the time of said administration of said CAR-T cells and approximately 3 months after said administration of said CAR-T cells. In some embodiments, said method is effective in obtaining either minimal residual disease (MRD) negative complete response or minimal residual disease (MRD) negative stringent complete response at a rate of between approximately 25% and approximately 44% at a follow-up time of approximately 12 months after said infusion of said CAR-T cells or at a rate of between approximately 33% and approximately 54% at a follow-up time of approximately 18 months after said infusion of said CAR-T cells. In some embodiments, said method is effective in obtaining either minimal residual disease (MRD) negative complete response or minimal residual disease (MRD) negative stringent complete response at a rate of approximately 34% at a follow-up time of approximately 12 months after said infusion of said CAR-T cells or at a rate of approximately 43% at a follow-up time of approximately 18 months after said infusion of said CAR-T cells.

The disclosure further relates to a pharmaceutical product comprising a ciltacabtagene autoleucel suspension for intravenous infusion, wherein the pharmaceutical product is packaged, and wherein the package includes a label that identifies the ciltacabtagene autoleucel suspension as an approved drug product for the treatment of adult patients with relapsed or refractory multiple myeloma after four or more prior lines of therapy, including a proteasome inhibitor, an immunomodulatory agent and an anti-CD38 monoclonal antibody.

The disclosure provides methods for treating relapsed or refractory multiple myeloma in an adult patient in need thereof, comprising administering an approved drug product comprising the ciltacabtagene autoleucel suspension in an amount and manner that is described in a drug product label for the approved drug product and/or in a treatment regimen described herein.

The disclosure also provides methods of selling an approved drug product comprising the ciltacabtagene autoleucel suspension, said method comprising selling such drug product, wherein a drug product label for a reference product for such drug product includes instructions for treating a patient with relapsed or refractory multiple myeloma after four or more prior lines of therapy, including a proteasome inhibitor, an immunomodulatory agent and an anti-CD38 monoclonal antibody.

The disclosure further provides methods of offering for sale a drug product comprising the ciltacabtagene autoleucel suspension, said method comprising offering for sale such drug product, wherein a drug product label for a reference product for such drug product includes instructions for treating a patient with relapsed or refractory multiple myeloma after four or more prior lines of therapy, including a proteasome inhibitor, an immunomodulatory agent and an anti-CD38 monoclonal antibody.

### BRIEF DESCRIPTION OF THE DRAWINGS

FIG. 1 shows the expression of BCMA antigen on the surface of GC, memory and plasmablast cells in the lymph node, long-lived plasma cells in the bone marrow LN and MALT, and on multiple myeloma cells. BAFF-R antigen is not expressed on plasmablast cells, long-lived plasma cells, or multiple myeloma cells. TACI is expressed on memory and plasmablast cells, long-lived plasma cells, and multiple myeloma cells. CD138 is expressed only on long-lived plasma cells and multiple myeloma cells.

FIG. 2 shows the design of the ciltacabtagene autoleucel CAR. Ciltacabtagene autoleucel comprises two VHH domains, as opposed to a single VL domain and a single VH domain found on various other CARs. Ciltacabtagene autoleucel comprises intracellular CD137 and human CD3 zeta domains.

FIG. 3 shows a schematic for preparing virus encoding ciltacabtagene autoleucel CAR, transduction of the virus into a T cell from the patient, and then preparation of CAR T cells expressing ciltacabtagene autoleucel.

FIG. 4 shows a schematic of study design for ciltacabtagene autoleucel CAR T-cells. The patient population includes those with relapsed or Refractory Multiple Myeloma, with 3 prior lines or double refractory to PI/IMiD and prior PI, IMiD, and anti-CD38 exposure. A primary objective is safety and establishment of RP2D, such as studying incidence and severity of adverse events (Phase 1b). Another primary objective is efficacy: ORR— PR or better as defined by IMWG (Phase 2). The following are secondary objectives: Incidence and severity of adverse events (Phase 2), and any further efficacy characterization.

FIG. 5A is a graph showing, at a median follow-up time of 12.4 months, the response and duration of response (DOR), based on an Independent Review Committee (IRC) assessment, for the 50 most followed up responders in the all treated analysis set, ranked along the vertical axis in order of the length of duration of follow-up with the responders. FIG. 5B is a graph showing, at a median follow-up time of 12.4 months, the response and duration of response (DOR), based on an Independent Review Committee (IRC) assessment, for the 44 least followed up responders in the all treated analysis set, ranked along the vertical axis in order of the length of duration of follow-up with the responders.

FIG. 6 shows a Kaplan-Meier plot for Duration of Response (DOR) based on an Independent Review Committee (IRC) assessment for responders in the all treated analysis set at a median follow-up time of 12.4 months, showing that the probabilities of the responders remaining in response at 9 months and 12 months were approximately 80.2% and 68.2%, respectively.

FIG. 7 shows a Kaplan-Meier plot for Overall Survival (OS) for all subjects in the all treated analysis set at a median follow-up time of 12.4 months, showing that 9-month and 12-month survival rates were approximately 90.7% and 88.5%, respectively.

FIG. 8, FIG. 9, FIG. 10, FIG. 11, FIG. 12, FIG. 13, FIG. 14, and FIG. 15 show a description of the protocol and data obtained at a median follow-up time of 12.4 months in the Phase 1b-2 clinical trial described herein, in which patients having relapsed, refractory multiple myeloma were treated with ciltacabtagene autoleucel.

FIG. 16, FIG. 17, FIG. 18, and FIG. 19 show a description of the evaluation and assessment of cytokine release syndrome (CRS) in the Phase 1b-2 clinical trial described herein, in which patients having relapsed, refractory multiple myeloma were treated with ciltacabtagene autoleucel.

FIG. 20 is a graph showing, at a median follow-up time of 18 months, the response and duration of response (DOR), based on an Independent Review Committee (IRC) assessment for responders in the all treated analysis set.

FIG. 21 shows a Kaplan-Meier plot for Duration of Response (DOR) based on an Independent Review Committee (IRC) assessment for responders in the all treated analysis set at a median follow-up time of 18 months.

FIG. 22 shows a Kaplan-Meier plot for Progression-Free Survival (PFS) based on an Independent Review Committee (IRC) assessment for responders in the all treated analysis set at a median follow-up time of 18 months.

FIG. 23 shows a Kaplan-Meier plot for Overall Survival (OS) for all subjects in the all treated analysis set at a median follow-up time of 18 months.

FIG. 24A, FIG. 24B, and FIG. 24C show Forest plots for the subgroup analysis of the Overall Response Rate (ORR) based on Independent Review Committee (IRC) assessment in the all treated analysis set at a median follow-up time of 18 months.

FIG. 25A, FIG. 25B, and FIG. 25C show Forest plots for the subgroup analysis of the Duration of Response (DOR) based on Independent Review Committee (IRC) assessment in the all treated analysis set at a median follow-up time of 18 months.

FIG. 26A, FIG. 26B, and FIG. 26C show Forest plots for the subgroup analysis of the overall MRD negativity rate at 10⁻⁵ in the bone marrow in the all treated analysis set at a median follow-up time of 18 months.

FIG. 27A, FIG. 27B, and FIG. 27C show Forest plots for the subgroup analysis of the overall MRD negativity rate at 10⁻⁵ in the bone marrow for subjects with evaluable sample in the all treated analysis set at a median follow-up time of 18 months.

FIG. 28 summarizes the selection of Comparator Arms for indirect treatment comparisons (ITC) Analyses.

FIG. 29A and FIG. 29B show a meta-analysis comparison, using all index dates, of the overall survival of patients treated with ciltacabtagene autoleucel and patients treated with physician's choice of treatment.

FIG. 30 shows a meta-analysis comparison, using all index dates, of the progression-free survival of patients treated with ciltacabtagene autoleucel and patients treated with physician's choice of treatment.

FIG. 31 shows a meta-analysis comparison, using first index dates, of the overall survival of patients treated with ciltacabtagene autoleucel and patients treated with physician's choice of treatment.

FIG. 32 shows a meta-analysis comparison, using first index dates, of the progression-free survival of patients treated with ciltacabtagene autoleucel and patients treated with physician's choice of treatment.

### DETAILED DESCRIPTION

The disclosure also provides related nucleic acids, recombinant expression vectors, host cells, populations of cells, antibodies, or antigen binding portions thereof, and pharmaceutical compositions relating to the immune cells and CAR-expressing T cells of the disclosure. Dosage regimens and dosage forms, and methods of treatment with the CAR-T cells are also provided.

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines and animals. The present disclosure is not limited by the illustrated ordering of acts or events, as some acts may occur in different orders and/or concurrently with other acts or events. Furthermore, not all illustrated acts, steps or events are required to implement a methodology in accordance with the present disclosure.

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The term “antibody” includes monoclonal antibodies (including full length 4-chain antibodies or full length heavy-chain only antibodies which have an immunoglobulin Fc region), antibody compositions with polyepitopic specificity, multi specific antibodies (e.g., bispecific antibodies, diabodies, and single-chain molecules), as well as antibody fragments (e.g., Fab, F(ab′)2, and Fv). The term “immunoglobulin” (Ig) is used interchangeably with “antibody” herein. Antibodies contemplated herein include single-domain antibodies, such as heavy chain only antibodies.

The term “heavy chain-only antibody” or “HCAb” refers to a functional antibody, which comprises heavy chains, but lacks the light chains usually found in 4-chain antibodies. Camelid animals (such as camels, llamas, or alpacas) are known to produce HCAbs.

The term “single-domain antibody” or “sdAb” refers to a single antigen-binding polypeptide having three complementary determining regions (CDRs). The sdAb alone is capable of binding to the antigen without pairing with a corresponding CDR-containing polypeptide. In some cases, single-domain antibodies are engineered from camelid HCAbs, and their heavy chain variable domains are referred herein as “VHHs”. Some VHHs may also be known as “Nanobodies”. A camelid sdAb is one of the smallest known antigen-binding antibody fragments (see, e.g., Hamers-Casterman et al., Nature 363:446-8 (1993); Greenberg et al., Nature 374:168-73 (1995); Hassanzadeh-Ghassabeh et al., Nanomedicine (Lond), 8:1013-26 (2013)). A basic VHH has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites. Heavy-chain only antibodies from the Camelid species have a single heavy chain variable region, which is referred to as “VHH” domain. VHH is thus a special type of variable region.

The term “variable” refers to the fact that certain segments of the variable domains differ extensively in sequence among antibodies. The V domain (i.e., variable domain) mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a β-sheet configuration, connected by three HVRs, which form loops connecting, and in some cases forming part of, the β-sheet structure. The HVRs in each chain are held together in close proximity by the FR regions and contribute to the formation of the antigen binding site of antibodies (with the HVRs from the other chain, if the antibody is not a sdAb or HCAb) (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The terms “fragment of an antibody”, “antibody fragment”, “functional fragment of an antibody”, and “antigen-binding portion” are used interchangeably herein to mean one or more fragments or portions of an antibody that retain the ability to specifically bind to an antigen (see, generally, Holliger et al., Nat. Biotech., 23(9): 1 126-1129 (2005)). The antigen recognition moiety of the CARs encoded by the nucleic acid sequences disclosed herein can contain any BCMA-binding antibody fragment. The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the VL, VH, CL, and CHI domains; (ii) a F(ab′)2 fragment, which is a bivalent fragment comprising two Fab fragments linked by a disulfide bridge at the hinge region; (iii) a Fv fragment consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al., Science, 242: 423-426 (1988); Huston et al., Proc. Natl. Acad. Sci. USA, 85: 5879-5883 (1988); and Osbourn et al., Nat. Biotechnol, 16: 778 (1998)) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites. Antibody fragments are known in the art and are described in more detail in, e.g., U.S. Patent Application Publication 2009/0093024 A1.

As used herein, the terms “specifically binds”, “specifically recognizes”, or “specific for” refer to measurable and reproducible interactions such as binding between a target and an antigen binding protein (such as a CAR or a VHH), which is determinative of the presence of the target in the presence of a heterogeneous population of molecules including biological molecules.

The term “specificity” refers to selective recognition of an antigen binding protein (such as a CAR or a VHH) for a particular epitope of an antigen. Natural antibodies, for example, are monospecific.

A “chimeric antigen receptor” or “CAR” is an artificially constructed hybrid protein or polypeptide containing the antigen binding domains of an antibody (or antibody fragment) linked to T-cell signaling domains. Characteristics of CARs can include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigens independent of antigen processing, thus bypassing a major mechanism of tumor evasion. Moreover, when expressed in T-cells, advantageously, CARs do not dimerize with endogenous T cell receptor (TCR) α- and β-chains. T cells expressing a CAR are referred to herein as CAR T cells, CAR-T cells or CAR modified T cells, and these terms are used interchangeably herein. The cell can be genetically modified to stably express an antibody binding domain on its surface, conferring novel antigen specificity that is MHC independent. “BCMA CAR” refers to a CAR having an extracellular binding domain specific for BCMA. “Bi-epitope CAR” refers to a CAR having an extracellular binding domain specific for two different epitopes on BCMA.

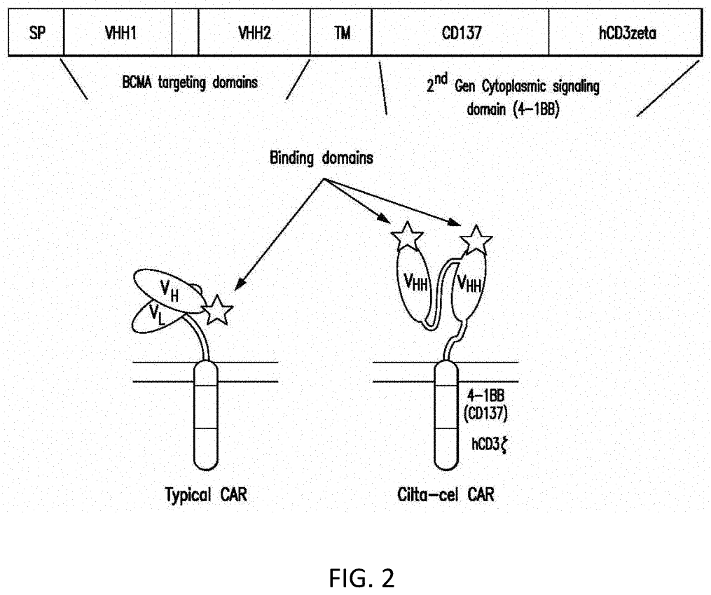

“Ciltacabtagene autoleucel” (“cilta-cel”) is a chimeric antigen receptor T cell (CAR-T) therapy comprising two B-cell maturation antigen (BCMA)-targeting VHH domains designed to confer avidity for BCMA. Cilta-cel can comprise T lymphocytes transduced with the ciltacabtagene autoleucel CAR, a CAR encoded by a lentiviral vector. The CAR targets the human B cell maturation antigen (BCMA CAR). A diagram of the lentiviral vector encoding cilta-cel CAR is provided in FIG. 2. The amino acid sequence of the cilta-cel CAR is the amino acid sequence of SEQ ID NO: 17.

The terms “express” and “expression” mean allowing for or causing the information in a gene or DNA sequence to become produced. For example, expression can take the form of producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or transmembrane.

The terms “treat” or “treatment” refer to therapeutic treatment wherein the object is to slow down or lessen an undesired physiological change or disease, or provide a beneficial or desired clinical outcome during treatment. Beneficial or desired clinical outcomes include alleviation of symptoms, diminishment of extent of disease, stabilized (i.e., not worsening) state of disease, delay or slowing of disease progression, amelioration or palliation of the disease state, and/or remission (whether partial or total), whether detectable or undetectable. “Treatment” can also mean prolonging survival as compared to expected survival if a subject was not receiving treatment. Those in need of treatment include those subjects already with the undesired physiological change or disease as well as those subjects prone to having the physiological change or disease. Treatment may involve a treatment agent, also referred to herein as a “medicament” or “medication,” that may be intended to help achieve the beneficial or desired clinical outcome of interest by its action. Treatment agents or medicaments may be administered to a subject by many routes, including at least intravenous and oral routes. The term “intravenous,” in connection to the administration of treatment agents or medicaments, refers to the administration of said treatment agents or medicaments within one or more veins. The term “oral,” in connection to the administration of treatment agents or medicaments, refers to the administration of said treatment agents or medicaments via an oral passage such as the mouth.

As used herein, the term “subject” refers to an animal. The terms “subject” and “patient” may be used interchangeably herein in reference to a subject. As such, a “subject” includes a human that is being treated for a disease, or prevention of a disease, as a patient. The methods described herein may be used to treat an animal subject belonging to any classification. Examples of such animals include mammals. Mammals, include, but are not limited to, mammals of the order Rodentia, such as mice and hamsters, and mammals of the order Logomorpha, such as rabbits. The mammals may be of the order Carnivora, including felines (cats) and canines (dogs). The mammals may be of the order Artiodactyla, including bovine (cows) and swine (pigs) or of the order Perssodactyla, including equines (horses). The mammals may be of the order Primates, Ceboids, or Simoids (monkeys) or of the order Anthropoids (humans and apes). In some embodiments, the mammal is a human.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “line of therapy,” as used in connection with methods of treatment herein, refers to one or more cycles of a planned treatment program, which may have consisted of one or more planned cycles of single-agent therapy or combination therapy, as well as a sequence of treatments administered in a planned manner. For example, a planned treatment approach of induction therapy followed by autologous stem cell transplantation followed by maintenance is one line of therapy. A new line of therapy is considered to have started when a planned course of therapy has been modified to include other treatment agents or medicaments (alone or in combination) as a result of disease progression, relapse, or toxicity. A new line of therapy is also considered to have started when a planned period of observation off therapy had been interrupted by a need for additional treatment for the disease.

The term “refractory,” as used in connection to treatment with a particular treatment agent or medicament herein, refers to diseases or disease subjects that fail to respond to said treatment agent or medicament. The phrase “refractory myeloma” refers to disease that is nonresponsive while on primary or salvage therapy, or progressed within 60 days of last therapy.

The phrase “nonresponsive disease” refers to either failure to achieve minimal response or development of progressive disease while on therapy.

The terminology used herein is for the purpose of describing particular embodiments only and is not intended to be limiting. As used herein, the indefinite articles “a”, “an” and “the” should be understood to include plural reference unless the context clearly indicates otherwise.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

### Vectors

Polynucleotide sequences encoding the CARs described in the present application can be obtained using standard recombinant techniques. Desired polynucleotide sequences may be isolated and sequenced from antibody producing cells such as hybridoma cells. Alternatively, polynucleotides can be synthesized using nucleotide synthesizers or PCR techniques.

The disclosure also provides a vector comprising the nucleic acid sequence encoding the CARs disclosed herein. The vector can be, for example, a plasmid, a cosmid, a viral vector (e.g., retroviral or adenoviral), or a phage. Suitable vectors and methods of vector preparation are well known in the art (see, e.g., Sambrook et al. and Ausubel et al.).

In addition to the nucleic acid sequences encoding the CARs disclosed herein, the vector preferably comprises expression control sequences, such as promoters, enhancers, polyadenylation signals, transcription terminators, internal ribosome entry sites (IRES), and the like, that provide for the expression of the nucleic acid sequence in a host cell. Exemplary expression control sequences are known in the art and described in, for example, Goeddel, Gene Expression Technology: Methods in Enzymology, Vol. 185, Academic Press, San Diego, Calif. (1990).

In some embodiments, the vector comprises a promoter. A large number of promoters recognized by a variety of potential host cells are well known. The selected promoter can be operably linked to cistron DNA encoding the CARs disclosed herein by removing the promoter from the source DNA via restriction enzyme digestion and inserting the isolated promoter sequence into the vector of the present application. A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, and the RSV promoter. Inducible promoters include, for example, the Tet system (U.S. Pat. Nos. 5,464,758 and 5,814,618), the Ecdysone inducible system (No et al., Proc. Natl. Acad. Sci., 93: 3346-3351 (1996)), the T-REX™ system (Invitrogen, Carlsbad, Calif.), LACSWITCH™ System (Stratagene, San Diego, Calif.), and the Cre-ERT tamoxifen inducible recombinase system (Indra et al., Nuc. Acid. Res., 27: 4324-4327 (1999); Nuc. Acid. Res., 28: e99 (2000); U.S. Pat. No. 7,112,715; and Kramer & Fussenegger, Methods Mol. Biol, 308: 123-144 (2005)).

In some embodiments, the vector comprises an “enhancer”. The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (e.g., from depositories such as the ATCC as well as other commercial or individual sources). A number of polynucleotides comprising promoters (such as the commonly used CMV promoter) also comprise enhancer sequences. Enhancers can be located upstream, within, or downstream of coding sequences. The term “Ig enhancers” refers to enhancer elements derived from enhancer regions mapped within the immunoglobulin (Ig) locus. Such Ig enhancers include for example, the heavy chain (mu) 5′ enhancers, light chain (kappa) 5′ enhancers, kappa and mu intronic enhancers, and 3′ enhancers (see generally Paul W. E. (ed), Fundamental Immunology, 3rd Edition, Raven Press, New York (1993), pages 353-363; and U.S. Pat. No. 5,885,827).

In some embodiments, the vector comprises a “selectable marker gene.” The term “selectable marker gene”, as used herein, refers to a nucleic acid sequence that allows cells expressing the nucleic acid sequence to be specifically selected for or against, in the presence of a corresponding selective agent. Suitable selectable marker genes are known in the art and described in, e.g., International Patent Application Publications WO 1992/08796 and WO 1994/28143; Wigler et al., Proc. Natl. Acad. Sci. USA, 77: 3567 (1980); O'Hare et al., Proc. Natl. Acad. Sci. USA, 78: 1527 (1981); Mulligan & Berg, Proc. Natl. Acad. Sci. USA, 78: 2072 (1981); Colberre-Garapin et al., J. Mol. Biol., 150: 1 (1981); Santerre et al., Gene, 30: 147 (1984); Kent et al., Science, 237: 901-903 (1987); Wigler et al., Cell, IP. 223 (1977); Szybalska & Szybalski, Proc. Natl. Acad. Sci. USA, 48: 2026 (1962); Lowy et al., Cell, 22: 817 (1980); and U.S. Pat. Nos. 5,122,464 and 5,770,359.

In some embodiments, the vector is an “episomal expression vector” or “episome,” which is able to replicate in a host cell, and persists as an extrachromosomal segment of DNA within the host cell in the presence of appropriate selective pressure (see, e.g., Conese et al., Gene Therapy, 11: 1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, Calif.) and pB-CMV from Stratagene (La Jolla, Calif.) represent non-limiting examples of an episomal vector that uses T-antigen and the SV40 origin of replication in lieu of EBNA1 and oriP.

In some embodiments, the vector is an “integrating expression vector,” which may randomly integrate into the host cell's DNA or may include a recombination site to enable recombination between the expression vector and a specific site in the host cell's chromosomal DNA. Such integrating expression vectors may utilize the endogenous expression control sequences of the host cell's chromosomes to effect expression of the desired protein. Examples of vectors that integrate in a site specific manner include, for example, components of the flp-in system from Invitrogen (Carlsbad, Calif.) (e.g., pcDNA™5/FRT), or the cre-lox system, such as can be found in the pExchange-6 Core Vectors from Stratagene (La Jolla, Calif.). Examples of vectors that randomly integrate into host cell chromosomes include, for example, pcDNA3.1 (when introduced in the absence of T-antigen) from Invitrogen (Carlsbad, Calif.), and pCI or pFNI OA (ACT) FLEXI™ from Promega (Madison, Wis.).

In some embodiments, the vector is a viral vector. Representative viral expression vectors include, but are not limited to, the adenovirus-based vectors (e.g., the adenovirus-based Per.C6 system available from Crucell, Inc. (Leiden, The Netherlands)), lentivirus-based vectors (e.g., the lentiviral-based pLP1 from Life Technologies (Carlsbad, Calif.)), and retroviral vectors (e.g., the pFB-ERV plus pCFB-EGSH from Stratagene (La Jolla, Calif.)). In a preferred embodiment, the viral vector is a lentivirus vector.

The vector comprising the inventive nucleic acid encoding the CAR can be introduced into a host cell that is capable of expressing the CAR encoded thereby, including any suitable prokaryotic or eukaryotic cell. Preferred host cells are those that can be easily and reliably grown, have reasonably fast growth rates, have well characterized expression systems, and can be transformed or transfected easily and efficiently.

As used herein, the term “host cell” refers to any type of cell that can contain the expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK 293 cells, and the like. In a preferred embodiment, the host cells are HEK 293 cells. In some embodiments, the HEK 293 cells are derived from the ATCC SD-3515 line. In some embodiments, the HEK 293 cells are derived from, the IU-VPF MCB line. In some embodiments, the HEK 293 cells are derived from the IU-VPF MWCB line. In some embodiments, the host cell can be a peripheral blood lymphocyte (PBL), a peripheral blood mononuclear cell (PBMC), or a natural killer (NK). Preferably, the host cell is a natural killer (NK) cell. More preferably, the host cell is a T-cell.

For purposes of amplifying or replicating the recombinant expression vector, the host cell may be a prokaryotic cell, e.g., a DH5a cell. For purposes of producing a virus from a viral expression vector, the host cell may be a eukaryotic cell, e.g., a HEK 293 cell. For purposes of producing a recombinant CAR, the host cell can be a mammalian cell. The host cell preferably is a human cell. The host cell can be of any cell type, can originate from any type of tissue, and can be of any developmental stage. Methods for selecting suitable mammalian host cells and methods for transformation, culture, amplification, screening, and purification of cells are known in the art.

In some embodiments, the disclosure provides an isolated host cell which expresses the nucleic acid sequence encoding the CARs described herein.

In some embodiments, the host cell is a T-cell. The T-cell of the disclosure can be any T-cell, such as a cultured T-cell, e.g., a primary T-cell, or a T-cell from a cultured T-cell line, or a T-cell obtained from a mammal. If obtained from a mammal, the T-cell can be obtained from numerous sources, including but not limited to blood, bone marrow, lymph node, the thymus, or other tissues or fluids. T-cells can also be enriched for or purified. The T-cell preferably is a human T-cell (e.g., isolated from a human). The T-cell can be of any developmental stage, including but not limited to, a CD4+/CD8+ double positive T-cell, a CD4+ helper T-cell, e.g., Th, and Th2 cells, a CD8+ T-cell (e.g., a cytotoxic T-cell), a tumor infiltrating cell, a memory T-cell, a naive T-cell, and the like. In one embodiment, the T-cell is a CD8+ T-cell or a CD4+ T-cell. T-cell lines are available from, e.g., the American Type Culture Collection (ATCC, Manassas, Va.), and the German Collection of Microorganisms and Cell Cultures (DSMZ) and include, for example, Jurkat cells (ATCC TM-152), Sup-Tl cells (ATCC CRL-1942), RPMI 8402 cells (DSMZ ACC-290), Karpas 45 cells (DSMZ ACC-545), and derivatives thereof.

In some embodiments, the host cell is a natural killer (NK) cell. NK cells are a type of cytotoxic lymphocyte that plays a role in the innate immune system. NK cells are defined as large granular lymphocytes and constitute a third kind of cells differentiated from the common lymphoid progenitor which also gives rise to B and T lymphocytes (see, e.g., Immunobiology, 5th ed., Janeway et al., eds., Garland Publishing, New York, N.Y. (2001)). NK cells differentiate and mature in the bone marrow, lymph node, spleen, tonsils, and thymus. Following maturation, NK cells enter into the circulation as large lymphocytes with distinctive cytotoxic granules. NK cells are able to recognize and kill some abnormal cells, such as, for example, some tumor cells and virus-infected cells, and are thought to be important in the innate immune defense against intracellular pathogens. As described above with respect to T-cells, the NK cell can be any NK cell, such as a cultured NK cell, e.g., a primary NK cell, or an NK cell from a cultured NK cell line, or an NK cell obtained from a mammal. If obtained from a mammal, the NK cell can be obtained from numerous sources, including but not limited to blood, bone marrow, lymph node, the thymus, or other tissues or fluids. NK cells can also be enriched for or purified. The NK cell preferably is a human NK cell (e.g., isolated from a human). NK cell lines are available from, e.g., the American Type Culture Collection (ATCC, Manassas, Va.) and include, for example, NK-92 cells (ATCC CRL-2407), NK92MI cells (ATCC CRL-2408), and derivatives thereof.

In some embodiments, the nucleic acid sequences encoding a CAR may be introduced into a cell by “transfection”, “transformation”, or “transduction”. “Transfection”, “transformation”, or transduction”, as used herein, refer to the introduction of one or more exogenous polynucleotides into a host cell by using physical or chemical methods.

Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)). Phage or viral vectors can be introduced into host cells, after growth of infectious particles in suitable packaging cells, many of which are commercially available.

### Chimeric Antigen Receptors

International Patent Publication No. WO 2018/028647 is incorporated by reference herein in its entirety. US Patent Publication No. 2018/0230225 is incorporated by reference herein in its entirety.

The disclosure provides for methods of treating a subject with cells expressing a chimeric antigen receptor (CAR). The CAR comprises an extracellular antigen binding domain comprising one or more single-domain antibodies. In various embodiments, there is provided a CAR targeting BCMA (also referred herein as “BCMA CAR”) comprising a polypeptide comprising: (a) an extracellular antigen binding domain comprising an anti-BCMA binding moiety; (b) a transmembrane domain; and (c) an intracellular signaling domain. In some embodiments, the anti-BCMA binding moiety is camelid, chimeric, human, or humanized. In some embodiments, the intracellular signaling domain comprises a primary intracellular signaling domain of an immune effector cell (such as T cell). In some embodiments, the primary intracellular signaling domain is derived from CD4. In some embodiments, the primary intracellular signaling domain is derived from CD3-zeta. In some embodiments, the intracellular signaling domain comprises a co-stimulatory signaling domain. In some embodiments, the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and combinations thereof. In certain embodiments, the transmembrane domain is derived from CD137.

In some embodiments, the BCMA CAR further comprises a hinge domain (such as a CD8-alpha hinge domain) located between the C-terminus of the extracellular antigen binding domain and the N-terminus of the transmembrane domain. In some embodiments, the BCMA CAR further comprises a signal peptide (such as a CD8-alpha signal peptide) located at the N-terminus of the polypeptide. In some embodiments, the polypeptide comprises from the N-terminus to the C-terminus: a CD8-alpha signal peptide, the extracellular antigen-binding domain, a CD8-alpha hinge domain, a CD28 transmembrane domain, a first co-stimulatory signaling domain derived from CD28, a second co-stimulatory signaling domain derived from CD137, and a primary intracellular signaling domain derived from CD4. In some embodiments, the polypeptide comprises from the N-terminus to the C-terminus: a CD8-alpha signal peptide, the extracellular antigen-binding domain, a CD8-alpha hinge domain, a CD8-alpha transmembrane domain, a second co-stimulatory signaling domain derived from CD137, and a primary intracellular signaling domain derived from CD3-zeta. In some embodiments, the BCMA CAR is monospecific. In some embodiments, the BCMA CAR is monovalent.

The present application also provides CARs that have two or more (including, but not limited to, any one of 2, 3, 4, 5, 6, or more) binding moieties that specifically bind to an antigen, such as BCMA. In some embodiments, one or more of the binding moieties are antigen binding fragments. In some embodiments, one or more of the binding moieties comprise single-domain antibodies. In some embodiments, one or more of the binding moieties comprise a VHH.

In some embodiments, the CAR is a multivalent (such as bivalent, trivalent, or of higher number of valencies) CAR comprising a polypeptide comprising: (a) an extracellular antigen binding domain comprising a plurality (such as at least about any one of 2, 3, 4, 5, 6, or more) of binding moieties specifically binding to an antigen (such as a tumor antigen); (b) a transmembrane domain; and (c) an intracellular signaling domain.

In some embodiments, the binding moieties, such as VHHs (including the plurality of VHHs, or the first VHH and/or the second VHH) are camelid, chimeric, human, or humanized. In some embodiments, the binding moieties or VHHs are connected to each other via peptide bonds or peptide linkers. In some embodiments, each peptide linker is no more than about 50 (such as no more than about any one of 35, 25, 20, 15, 10, or 5) amino acids long.

In some embodiments, the first BCMA binding moiety and/or the second BCMA binding moiety is an anti-BCMA VHH. In some embodiments, the first BCMA binding moiety is a first anti-BCMA VHH and the second BCMA binding moiety is a second anti-BCMA VHH.

In some embodiments, the first BCMA binding moiety and the second BCMA binding moiety are connected to each other via a peptide linker. In some embodiments, the peptide linker comprises the amino acid sequence of SEQ ID NO: 3. In some embodiments, the peptide linker comprises a polypeptide encoded by the nucleic acid sequence of SEQ ID NO: 11.

In some embodiments, the CAR further comprises a hinge domain (such as a CD8-alpha hinge domain) located between the C-terminus of the extracellular antigen binding domain and the N-terminus of the transmembrane domain. In some embodiments, the CAR further comprises a signal peptide (such as a CD8-alpha signal peptide) located at the N-terminus of the polypeptide.

Without wishing to be bound by theory, the CARs that are multivalent, or those CARs comprising an extracellular antigen binding domain comprising a first BCMA binding moiety and a second BCMA binding moiety, may be specially suitable for targeting multimeric antigens via synergistic binding by the different antigen binding sites, or for enhancing binding affinity or avidity to the antigen. Improved avidity may allow for a substantial reduction in the dose of CAR-T cells needed to achieve a therapeutic effect, such as a dose ranging from 4.0×104 to 1.0×106 CAR-T cells per kilogram of the mass of the subject, or 3.0×106 to 1.0×108 total CAR-T expressing cells. Monovalent CARs, such as bb2121, may need to be dosed at 5 to 10 times these amounts to achieve a comparable effect. In various embodiments, reduced dosage ranges may provide for substantial reduction in cytokine release syndrome (CRS) and other potentially dangerous side-effects of CAR-T therapy.

The various binding moieties (e.g., an extracellular antigen binding domain comprising a first BCMA binding moiety and a second BCMA binding moiety) in the CARs described herein may be connected to each other via peptide linkers. The peptide linkers connecting different binding moieties (such as VHHs) may be the same or different. Different domains of the CARs may also be connected to each other via peptide linkers. In some embodiments, the binding moieties (such as VHHs) are directly connected to each other without any peptide linkers.

The peptide linker in the CARs described herein can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acids to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

The CARs of the present application comprise a transmembrane domain that can be directly or indirectly connected to the extracellular antigen binding domain.

The CAR may comprise a T-cell activation moiety. The T-cell activation moiety can be any suitable moiety derived or obtained from any suitable molecule. In one embodiment, for example, the T-cell activation moiety comprises a transmembrane domain. The transmembrane domain can be any transmembrane domain derived or obtained from any molecule known in the art. For example, the transmembrane domain can be obtained or derived from a CD8a molecule or a CD28 molecule. Without wishing to be bound by theory, CD8 is a transmembrane glycoprotein that serves as a co-receptor for the T-cell receptor (TCR), and is expressed primarily on the surface of cytotoxic T-cells. The most common form of CD8 exists as a dimer composed of a CD8 alpha (CD8α) and CD8 beta (CD8β) chain. CD28 is expressed on T-cells and provides co-stimulatory signals required for T-cell activation. CD28 is the receptor for CD80 (B7.1) and CD86 (B7.2). In a preferred embodiment, the CD8α and CD28 are human.

In addition to the transmembrane domain, the T-cell activation moiety may further comprise an intracellular (i.e., cytoplasmic) T-cell signaling domain. The intercellular T-cell signaling domain can be obtained or derived from a CD28 molecule, a CD3 zeta (ζ) molecule or modified versions thereof, a human Fc receptor gamma (FcRγ) chain, a CD27 molecule, an OX40 molecule, a 4-1BB molecule, or other intracellular signaling molecules known in the art. Without wishing to be bound by theory: (1) CD28 is a T-cell marker important in T-cell co-stimulation; (2) CD3 associates with TCRs to produce a signal and contains immunoreceptor tyrosine-based activation motifs (ITAMs); and (3) 4-1BB, also known as CD137, transmits a potent costimulatory signal to T-cells, promoting differentiation and enhancing long-term survival of T lymphocytes. In a preferred embodiment, the CD28, CD3 zeta, 4-1BB, OX40, and CD27 are human.

The T-cell activation domain of a CAR encoded by the nucleic acid sequences disclosed herein can comprise any one of aforementioned transmembrane domains and any one or more of the aforementioned intercellular T-cell signaling domains in any combination. For example, the nucleic acid sequences disclosed herein can encode a CAR comprising a CD28 transmembrane domain and intracellular T-cell signaling domains of CD28 and CD3 zeta. Alternatively, for example, the nucleic acid sequences disclosed herein can encode a CAR comprising a CD8α transmembrane domain and intracellular T-cell signaling domains of CD28, CD3 zeta, the Fc receptor gamma (FcRγ) chain, and/or 4-1BB.

In some embodiments, the CAR polypeptide further comprises a signal peptide located at the N-terminus of the polypeptide. In some embodiments, the signal peptide is derived from CD8-alpha. In some embodiments, the signal peptide comprises the amino acid sequence of SEQ ID NO: 1. In some embodiments, signal peptide comprises a polypeptide encoded by the nucleic acid sequence of SEQ ID NO: 9.

In certain embodiments, the transmembrane domain comprises the amino acid sequence of SEQ ID NO: 6. In certain embodiments, the transmembrane domain comprises a polypeptide encoded by the nucleic acid sequence of SEQ ID NO: 14.

In some embodiments, the intracellular signaling domain comprises a primary intracellular signaling domain of an immune effector cell. In some embodiments, the intracellular signaling domain is derived from CD3ζ. In some embodiments, the intracellular signaling domain comprises at least one co-stimulatory signaling domain. In some embodiments, the intracellular signaling domain comprises the amino acid sequence of SEQ ID NO: 8. In some embodiments, the intracellular signaling domain comprises a polypeptide encoded by the nucleic acid sequence of SEQ ID NO: 16. In some embodiments, the intracellular signaling domain comprises the amino acid sequence of SEQ ID NO: 7. In some embodiments, the intracellular signaling domain comprises a polypeptide encoded by the nucleic acid sequence of SEQ ID NO: 15.

In some embodiments, the CAR polypeptide further comprises a hinge domain located between the C-terminus of the extracellular antigen binding domain and the N-terminus of the transmembrane domain. In some embodiments, the hinge domain comprises the amino acid sequence of SEQ ID NO: 5. In some embodiments, the hinge domain comprises a polypeptide encoded by the nucleic acid sequence of SEQ ID NO: 13.

In some embodiments, the CAR comprises one or more of, or all of, SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. In one embodiment, the CAR comprises SEQ ID NO: 17. In some embodiments, the CAR comprises a polypeptide encoded by the nucleic acid sequence of one or more of, or all of, SEQ ID NO: 9, SEQ ID NO: 10, SEQ ID NO: 11, SEQ ID NO: 12, SEQ ID NO: 13, SEQ ID NO: 14, SEQ ID NO: 15 and SEQ ID NO: 16.

### Immune Effector Cell Compositions

“Immune effector cells” are immune cells that can perform immune effector functions. In some embodiments, the immune effector cells express at least FcγRIII and perform ADCC effector function. Examples of immune effector cells which mediate ADCC include peripheral blood mononuclear cells (PBMC), natural killer (NK) cells, monocytes, cytotoxic T cells, neutrophils, and eosinophils. In some embodiments, the immune effector cells are T cells. In some embodiments, the T cells are autologous T cells. In some embodiments, the T cells are allogeneic T cells. In some embodiments, the T cells are CD4+/CD8−, CD4−/CD8+, CD4+/CD8+, CD4−/CD8−, or combinations thereof. In some embodiments, the T cells produce IL-2, TFN, and/or TNF upon expressing the CAR and binding to the target cells, such as CD20+ or CD19+ tumor cells. In some embodiments, the CD8+ T cells lyse antigen-specific target cells upon expressing the CAR and binding to the target cells.

Biological methods for introducing the vector into an immune effector cell include the use of DNA and RNA vectors. Viral vectors have become the most widely used method for inserting genes into mammalian, e.g., human cells. Chemical means for introducing the vector into an immune effector cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

Provided herein are dosage forms comprising 3.0×107 to 1.0×108 CAR-T cells comprising a CAR comprising a polypeptide comprising: (a) an extracellular antigen binding domain comprising a first BCMA binding moiety specifically binding to a first epitope of BCMA, and a second BCMA binding moiety specifically binding to a second epitope of BCMA; (b) a transmembrane domain; and (c) an intracellular signaling domain, wherein the first epitope and the second epitope are different. In certain embodiments, the dosage form comprises 3.0×107 to 4.0×107 of the CAR-T cells. In certain embodiments, the dosage form comprises 3.5×107 to 4.5×107 of the CAR-T cells. In certain embodiments, the dosage form comprises 4.0×107 to 5.0×107 of the CAR-T cells. In certain embodiments, the dosage form comprises 4.5×107 to 5.5×107 of the CAR-T cells. In certain embodiments, the dosage form comprises 5.0×107 to 6.0×107 of the CAR-T cells. In certain embodiments, the dosage form comprises 5.5×107 to 6.5×107 of the CAR-T cells. In certain embodiments, the dosage form comprises 6.0×107 to 7.0×107 of the CAR-T cells. In certain embodiments, the dosage form comprises 6.5×107 to 7.5×107 of the CAR-T cells. In certain embodiments, the dosage form comprises 7.0×107 to 8.0×107 of the CAR-T cells. In certain embodiments, the dosage form comprises 7.5×107 to 8.5×107 of the CAR-T cells. In certain embodiments, the dosage form comprises 8.0×107 to 9.0×107 of the CAR-T cells. In certain embodiments, the dosage form comprises 8.5×107 to 9.5×107 of the CAR-T cells. In certain embodiments, the dosage form comprises 9.0×107 to 1.0×108 of the CAR-T cells.

In some embodiments, there are provided dosage forms comprising 3.0×107 to 1.0×108 engineered immune effector cells (such as T-cells) comprising a CAR comprising a polypeptide comprising: (a) an extracellular antigen binding domain comprising a first anti-BCMA VHH specifically binding to a first epitope of BCMA, and a second anti-BCMA VHH specifically binding to a second epitope of BCMA; (b) a transmembrane domain; and (c) an intracellular signaling domain, wherein the first epitope and the second epitope are different. In certain embodiments, the dosage form comprises 3.0×107 to 4.0×107 of the CAR-T cells. In certain embodiments, the dosage form comprises 3.5×107 to 4.5×107 of the CAR-T cells. In certain embodiments, the dosage form comprises 4.0×107 to 5.0×107 of the CAR-T cells. In certain embodiments, the dosage form comprises 4.5×107 to 5.5×107 of the CAR-T cells. In certain embodiments, the dosage form comprises 5.0×107 to 6.0×107 of the CAR-T cells. In certain embodiments, the dosage form comprises 5.5×107 to 6.5×107 of the CAR-T cells. In certain embodiments, the dosage form comprises 6.0×107 to 7.0×107 of the CAR-T cells. In certain embodiments, the dosage form comprises 6.5×107 to 7.5×107 of the CAR-T cells. In certain embodiments, the dosage form comprises 7.0×107 to 8.0×107 of the CAR-T cells. In certain embodiments, the dosage form comprises 7.5×107 to 8.5×107 of the CAR-T cells. In certain embodiments, the dosage form comprises 8.0×107 to 9.0×107 of the CAR-T cells. In certain embodiments, the dosage form comprises 8.5×107 to 9.5×107 of the CAR-T cells. In certain embodiments, the dosage form comprises 9.0×107 to 1.0×108 of the CAR-T cells.

In some embodiments, the cell population of the CAR-T dosage forms described herein comprise a T cell or population of T cells, e.g., at various stages of differentiation. Stages of T cell differentiation include naïve T cells, stem central memory T cells, central memory T cells, effector memory T cells, and terminal effector T cells, from least to most differentiated. After antigen exposure, naïve T cells proliferate and differentiate into memory T cells, e.g., stem central memory T cells and central memory T cells, which then differentiate into effector memory T cells. Upon receiving appropriate T cell receptor, costimulatory, and inflammatory signals, memory T cells further differentiate into terminal effector T cells. See, e.g., Restifo. Blood. 124.4(2014):476-77; and Joshi et al. J. Immunol. 180.3(2008):1309-15.

Naïve T cells can have the following expression pattern of cell surface markers: CCR7+, CD62L+, CD45RO−, CD95−. Stem central memory T cells (Tscm) can have the following expression pattern of cell surface markers: CCR7+, CD62L+, CD45RO−, CD95+. Central memory T cells (Tcm) can have the following expression pattern of cell surface markers: CCR7+, CD62L+, CD45RO+, CD95+. Effector memory T cells (Tem) can have the following expression pattern of cell surface markers: CCR7−, CD62L−, CD45RO+, CD95+. Terminal effector T cells (Teff) can have the following expression pattern of cell surface markers: CCR7−, CD62L−, CD45RO−, CD95+. See, e.g., Gattinoni et al. Nat. Med. 17(2011):1290-7; and Flynn et al. Clin. Translat. Immunol. 3(2014):e20.

### Pharmaceutical Compositions and Formulations

Further provided by the present application are pharmaceutical compositions comprising any one of the anti-BCMA antibodies of the disclosure, or any one of the engineered immune effector cells comprising any one of the CARs (such as BCMA CARs) as described herein, and a pharmaceutically acceptable carrier. Pharmaceutical compositions can be prepared by mixing any of the immune effector cells described herein, having the desired degree of purity, with optional pharmaceutically acceptable carriers, excipients or stabilizers (Remington's Pharmaceutical Sciences 16th edition, Osol, A. Ed. (1980)), in the form of lyophilized formulations or aqueous solutions. In certain embodiments, a pharmaceutical composition of CAR-T cells further comprises an excipient selected from dimethyl sulfoxide or dextran-40.

The compositions described herein may be administered as part of a pharmaceutical composition comprising one or more carriers. The choice of carrier will be determined in part by the particular nucleic acid sequence, vector, or host cells expressing the CARs disclosed herein, as well as by the particular method used to administer the nucleic acid sequence, vector, or host cells expressing the CARs disclosed herein. Accordingly, there are a variety of suitable formulations of the pharmaceutical compositions of the disclosure.

For example, the pharmaceutical compositions can contain preservatives. Suitable preservatives may include, for example, methylparaben, propylparaben, sodium benzoate, and benzalkonium chloride. A mixture of two or more preservatives optionally may be used. The preservative or mixtures thereof are typically present in an amount of about 0.0001% to about 2% by weight of the total composition.

In addition, buffering agents may be used in the compositions. Suitable buffering agents include, for example, citric acid, sodium citrate, phosphoric acid, potassium phosphate, and various other acids and salts. A mixture of two or more buffering agents optionally may be used. The buffering agent or mixtures thereof are typically present in an amount of about 0.001% to about 4% by weight of the total composition.

The compositions comprising the nucleic acid sequence encoding the CARs disclosed herein, or host cells expressing the CARs disclosed herein, can be formulated as an inclusion complex, such as cyclodextrin inclusion complex, or as a liposome. Liposomes can serve to target the host cells (e.g., T-cells or NK cells) or the nucleic acid sequences disclosed herein to a particular tissue. Liposomes also can be used to increase the half-life of the nucleic acid sequences disclosed herein. Many methods are available for preparing liposomes, such as those described in, for example, Szoka et al., Ann. Rev. Biophys. Bioeng., 9: 467 (1980), and U.S. Pat. Nos. 4,235,871; 4,501,728; 4,837,028; and 5,019,369. The compositions can employ time-released, delayed release, and sustained release delivery systems such that the delivery of the compositions disclosed herein occurs prior to, and with sufficient time to cause, sensitization of the site to be treated. Many types of release delivery systems are available and known to those of ordinary skill in the art. Such systems can avoid repeated administrations of the composition, thereby increasing convenience to the subject and the physician, and may be particularly suitable for certain composition embodiments of the disclosure.

In certain embodiments, the CAR-T cells are formulated at a dose of about 1.0×105 to 2.0×105 cells/kg, 1.5×105 to 2.5×105 cells/kg, 2.0×105 to 3.0×105 cells/kg, 2.5×105 to 3.5×105 cells/kg, 3.0×105 to 4.0×105 cells/kg, 3.5×105 to 4.5×105 cells/kg, 4.0×105 to 5.0×105 cells/kg, 4.5×105 to 5.5×10⁵ cells/kg, 5.0×10⁵ to 6.0×10⁵ cells/kg, 5.5×10⁵ to 6.5×10⁵ cells/kg, 6.0×10⁵ to 7.0×10⁵ cells/kg, 6.5×10⁵ to 7.5×10⁵ cells/kg, 7.0×10⁵ to 8.0×10⁵ cells/kg, 7.5×10⁵ to 8.5×10⁵ cells/kg, 8.0×10⁵ to 9.0×10⁵ cells/kg, 8.5×105 to 9.5×10⁵ cells/kg, 9.0×10⁵ to 1.0×10⁶ cells/kg. In a preferred embodiment, the dose is formulated at approximately 0.75×10⁶ cells/kg. In certain embodiments, the CAR-T cells are formulated at a dose of less than 1.0×10⁸ cells per subject.

### Methods of Treatment

The present application further relates to methods and compositions for use in cell immunotherapy. In some embodiments, the cell immunotherapy is for treating cancer in a subject, including but not limited to hematological malignancies and solid tumors. In some embodiments, the subject is human. In some embodiments, the methods are suitable for treatment of adults and pediatric population, including all subsets of age, and can be used as any line of treatment, including first line or subsequent lines.

Any of the anti-BCMA VHHs, CARs, and engineered immune effector cells (such as CAR-T cells) described herein may be used in the method of treating cancer. In some embodiments, the immune effector cells are autologous. In some embodiments, the immune effector cells are allogeneic.

In certain embodiments, the CAR-T cells are administered at a dose of about 1.0×105 to 2.0×105 cells/kg, 1.5×105 to 2.5×105 cells/kg, 2.0×105 to 3.0×105 cells/kg, 2.5×105 to 3.5×105 cells/kg, 3.0×105 to 4.0×105 cells/kg, 3.5×105 to 4.5×105 cells/kg, 4.0×105 to 5.0×105 cells/kg, 4.5×105 to 5.5×105 cells/kg, 5.0×105 to 6.0×105 cells/kg, 5.5×105 to 6.5×105 cells/kg, 6.0×105 to 7.0×105 cells/kg, 6.5×105 to 7.5×105 cells/kg, 7.0×105 to 8.0×105 cells/kg, 7.5×105 to 8.5×105 cells/kg, 8.0×105 to 9.0×105 cells/kg, 8.5×105 to 9.5×105 cells/kg, 9.0×105 to 1.0×106 cells/kg, 1.0×106 to 2.0×106 cells/kg, 1.5×106 to 2.5×106 cells/kg, 2.0×106 to 3.0×106 cells/kg, 2.5×106 to 3.5×106 cells/kg, 3.0×106 to 4.0×106 cells/kg, 3.5×106 to 4.5×106 cells/kg, 4.0×106 to 5.0×106 cells/kg, 4.5×106 to 5.5×106 cells/kg, or 5.0×106 to 6.0×106 cells/kg. In a preferred embodiment, the dose comprises approximately 0.75×106 cells/kg. In certain embodiments, the CAR-T cells are administered at a dose of about 1.0×108 cells per subject.

In certain embodiments, the CAR-T cells are administered at a dose of less than 1.0×108 cells per subject. In certain embodiments, the CAR-T cells are administered at a dose of about 3.0 to 4.0×107 cells. In certain embodiments, the CAR-T cells are administered at a dose of about 3.5 to 4.5×107 cells. In certain embodiments, the CAR-T cells are administered at a dose of about 4.0 to 5.0×107 cells. In certain embodiments, the CAR-T cells are administered at a dose of about 4.5 to 5.5×107 cells. In certain embodiments, the CAR-T cells are administered at a dose of about 5.0 to 6.0×107 cells. In certain embodiments, the CAR-T cells are administered at a dose of about 5.5 to 6.5×107 cells. In certain embodiments, the CAR-T cells are administered at a dose of about 6.0 to 7.0×107 cells. In certain embodiments, the CAR-T cells are administered at a dose of about 6.5 to 7.5×107 cells. In certain embodiments, the CAR-T cells are administered at a dose of about 7.0 to 8.0×107 cells. In certain embodiments, the CAR-T cells are administered at a dose of about 7.5 to 8.5×107 cells. In certain embodiments, the CAR-T cells are administered at a dose of about 8.0 to 9.0×107 cells. In certain embodiments, the CAR-T cells are administered at a dose of about 8.5 to 9,5×107 cells. In certain embodiments, the CAR-T cells are administered at a dose of about 9.0×107 to 1.0×108 cells.

In certain embodiments, the CAR-T cells are administered at a dose of about 0.693×106 CAR-positive viable T-cells/kg. In certain embodiments, the CAR-T cells are administered at a dose of about 0.52×106 CAR-positive viable T-cells/kg. In certain embodiments, the CAR-T cells are administered at a dose of about 0.94×106 CAR-positive viable T-cells/kg. In certain embodiments, the CAR-T cells are administered at a dose of about 0.709×106 CAR-positive viable T-cells/kg. In certain embodiments, the CAR-T cells are administered at a dose of about 0.51×106 CAR-positive viable T-cells/kg. In certain embodiments, the CAR-T cells are administered at a dose of about 0.95×106 CAR-positive viable T-cells/kg. In certain embodiments, the CAR-T cells are administered in an outpatient setting.

In some embodiments, the composition comprising CAR-T cells administered to the subject further comprises an excipient selected from dimethyl sulfoxide or dextran-40.

In certain embodiments, the CAR-T cells (e.g., at any of the foregoing doses) are administered in one or more intravenous infusions. In certain embodiments, said administration of said CAR-T cells is via a single intravenous infusion. In certain embodiments, said single intravenous infusion is administered using a single bag of said CAR-T cells. In certain embodiments, said administration of said single bag of said CAR-T cells is completed between the time at which said single bag of CAR-T cells is thawed and three hours after said single bag of CAR-T cells is thawed. In certain embodiments, single intravenous administration is administered using two bags of said CAR-T cells. In certain embodiments, said administration of each of said two bags of said CAR-T cells is completed between the time at which a first bag of said two bags of CAR-T cells is thawed and three hours after said first bag of CAR-T cells is thawed.

In certain embodiments, the time since the initial apheresis to the administration of CAR-T cells is less than 41, 47, 54, 61, 68, 75, 82, 89, 96, 103, 110, 117, 124, 131, 138, 145, 152, 159, 166 or 167 days. In certain embodiments, the time since the initial apheresis to the administration of CAR-T cells is greater than 41, 47, 54, 61, 68, 75, 82, 89, 96, 103, 110, 117, 124, 131, 138, 145, 152, 159, 166 or 167 days.

In certain embodiments, a lymphodepleting regimen precedes the administration of CAR-T cells. In certain embodiments, the lymphodepleting regimen comprises administration of cyclophosphamide and/or administration of fludarabine. In certain embodiments, the lymphodepleting regimen is administered intravenously. In certain embodiments, the lymphodepleting regimen precedes the administration of CAR-T cells by 5 to 7 days. In certain embodiments, the lymphodepleting regimen precedes the administration of CAR-T cells by 2 to 4 days. In certain embodiments, the lymphodepleting regimen comprises intravenous administration of cyclophosphamide and fludarabine 5 to 7 days prior to the administration of CAR-T cells. In certain embodiments, the lymphodepleting regimen comprises intravenous administration of cyclophosphamide and fludarabine 2 to 4 days prior to the administration of CAR-T cells. In certain embodiments, the lymphodepleting regimen comprises cyclophosphamide administered intravenously at 300 mg/m2. In certain embodiments, the lymphodepleting regimen comprises fludarabine administered intravenously at 30 mg/m2. In some embodiments, the lymphodepleting regimen is performed daily for 3 days. In situations wherein the administration of the CAR-T cells is delayed by more than 14 days, the lymphodepleting regimen may be repeated.

In certain embodiments, the method of treatment with CAR-T cells further comprises treating the subject for cytokine release syndrome (CRS) within 3 days of CAR-T cell administration without significantly reducing CAR-T cell expansion in vivo. In certain embodiments, the treatment of CRS comprises administering the subject with an IL-6R inhibitor. In certain embodiments, the IL-6R inhibitor is an antibody. In certain embodiments, the IL-6 inhibitor inhibits IL-6R by binding its extracellular domain. In certain embodiments, the IL-6R inhibitor prevents the binding of IL-6 to IL-6R. In certain embodiments, the IL-6R inhibitor is tocilizumab. CRS can be identified based on clinical presentation [see Approved Label provided herein]. In some embodiments, other causes of fever, hypoxia and hypotension are evaluated and treated. Laboratory testing to monitor for disseminated intravascular coagulation, hematology parameters, as well as pulmonary, cardiac, renal, and hepatic function can be used. CRS can be managed according to the recommendations in Table 1 of the herein disclosed Approved Label. The methods can comprise administering anti-seizure prophylaxis with levetiracetam in patients who experience CRS. In some embodiments, the methods comprises monitoring patients who experience Grade 2 or higher CRS (e.g., hypotension not responsive to fluids, or hypoxia requiring supplemental oxygenation) with continuous cardiac telemetry and pulse oximetry. In some embodiments, intensive care unit level monitoring and supportive therapy can be used for severe or life-threatening CRS. For CRS refractory to first line interventions such as tocilizumab or tocilizumab and corticosteroids, the methods comprise alternate treatment options (i.e., higher corticosteroid dose, alternative anti-cytokine agents, e.g. anti-IL1 and/or anti-TNFα, anti-T cell therapies). Refractory CRS is characterized by fevers, end-organ toxicity (e.g., hypoxia, hypotension) not improving within 12 hours of first line interventions or development of HLH/MAS.

In certain embodiments, the method of treatment with CAR-T cells further comprises treating the subject with pre-infusion medication comprising an antipyretic and an antihistamine up to 1 hour prior to the administration of CAR-T cells. In certain embodiments, the antipyretic comprises either paracetamol or acetaminophen. In certain embodiments, the antipyretic is administered to the subject either orally or intravenously. In certain embodiments, the antipyretic is administered to the subject at a dosage of between 650 mg and 1000 mg. In certain embodiments, the antihistamine comprises diphenhydramine. In certain embodiments, the antihistamine is administered to the subject either orally or intravenously. In certain embodiments, the antihistamine is administered at a dosage of between 25 mg and 50 mg, or its equivalent. The composition comprising the host cells expressing the CAR-encoding nucleic acid sequences disclosed herein, or a vector comprising the CAR-encoding nucleic acid sequences disclosed herein, can be administered to a mammal using standard administration techniques, including oral, intravenous, intraperitoneal, subcutaneous, pulmonary, transdermal, intramuscular, intranasal, buccal, sublingual, or suppository administration. The composition preferably is suitable for parenteral administration. The term “parenteral”, as used herein, includes intravenous, intramuscular, subcutaneous, rectal, vaginal, and intraperitoneal administration. More preferably, the composition is administered to a mammal using peripheral systemic delivery by intravenous, intraperitoneal, or subcutaneous injection. Most preferably, the composition is administered by intravenous infusion.

The composition comprising the host cells expressing the CAR-encoding nucleic acid sequences disclosed herein, or a vector comprising the CAR-encoding nucleic acid sequences disclosed herein, can be administered with one or more additional therapeutic agents, which can be coadministered to the mammal. By “coadministering” is meant administering one or more additional therapeutic agents and the composition comprising the host cells disclosed herein or the vectors disclosed herein sufficiently close in time such that the CARs disclosed herein can enhance the effect of one or more additional therapeutic agents, or vice versa. In this regard, the composition comprising the host cells disclosed herein or the vectors disclosed herein can be administered first, and the one or more additional therapeutic agents can be administered second, or vice versa.

A CAR-expressing cell described herein and the at least one additional therapeutic agent can be administered simultaneously, in the same or in separate compositions, or sequentially. For sequential administration, the CAR-expressing cell described herein can be administered first, and the additional agent can be administered second, or the order of administration can be reversed.

In certain embodiments, a lymphodepleting regimen precedes the administration of CAR-T cells. In certain embodiments, the lymphodepleting regimen precedes said administration of CAR-T cells by approximately 2 days to approximately 7 days. In certain embodiments, lymphodepleting regimen is administered intravenously. In certain embodiments, said lymphodepleting regimen comprises administration of cyclophosphamide or administration of fludarabine. In certain embodiments, said cyclophosphamide is administered intravenously at 300 mg/m2. In certain embodiments, said fludarabine is administered intravenously at 30 mg/m2.

In certain embodiments, a lymphodepleting regimen comprising cyclophosphamide administered intravenously at 300 mg/m2 and fludarabine administered intravenously at 30 mg/m2 precedes said administration of CAR-T cells by approximately 2 days to approximately 7 days.

In certain embodiments, the subject further receives bridging therapy, wherein said bridging therapy comprises short-term treatment with at least one bridging medicament between apheresis and said lymphodepleting regimen, and wherein said at least one bridging medicament had previously obtained an outcome of stable disease, minimal response, partial response, very good partial response, complete response or stringent complete response for the subject. In certain embodiments, the subject had an increase in tumor burden despite said bridging therapy. In certain embodiments, the subject had an increase in tumor burden of approximately 25% or greater despite said bridging therapy. Suitable bridging therapies include, for example, dexamethasone, bortezomib, cyclophosphamide, and pomalidomide. In some embodiments, the bridging therapy comprises dexamethasone. In some embodiments, the bridging therapy comprises bortezomib. In some embodiments, the bridging therapy comprises cyclophosphamide. In some embodiments, the bridging therapy comprises pomalidomide.

In certain embodiments, the subject is treated with pre-administration medication comprising an antipyretic and an antihistamine up to approximately 1 hour before said administration of said CAR-T cells. In certain embodiments, said antipyretic comprises either paracetamol or acetaminophen. In certain embodiments, said antipyretic is administered to the subject either orally or intravenously. In certain embodiments, said antipyretic is administered to the subject at a dosage of between 650 mg and 1000 mg. In certain embodiments, said antihistamine comprises diphenhydramine. In certain embodiments, said antihistamine is administered to the subject either orally or intravenously. In certain embodiments, said antihistamine is administered at a dosage of between 25 mg and 50 mg, or its equivalent. In certain embodiments, said antipyretic comprises either paracetamol or acetaminophen and said antipyretic is administered to the subject either orally or intravenously at a dosage of between 650 mg and 1000 mg, and wherein said antihistamine comprises diphenhydramine and said antihistamine is administered to the subject either orally or intravenously at a dosage of between 25 mg and 50 mg, or its equivalent.

In some embodiments, the methods comprise, prior to administration of the CAR-T cells, administering a lymphodepleting chemotherapy regimen comprising cyclophosphamide 300 mg/m2 intravenously (IV) and fludarabine 30 mg/m2 IV daily for 3 days, and administering pre-infusion medications comprising an antipyretic (such as oral or intravenous acetaminophen 650 to 1000 mg) and antihistamine (such as oral or intravenous diphenhydramine 25 to 50 mg or equivalent), wherein:
the CAR-T cells are administered 2-4 days after completion of the lymphodepleting chemotherapy and
the CAR-T cells are administered 30-60 minutes after the administration of the pre-infusion medications.

In some embodiments, the CAR-T cells are not administered, or the administration of the CAR-T cells is delayed, if the patient has any of the following conditions: clinically significant active infection or inflammatory disorder; or Grade ≥3 non-hematologic toxicities of cyclophosphamide and fludarabine conditioning, except for Grade 3 nausea, vomiting, diarrhea, or constipation. Administration of the CAR-T cells should be delayed until resolution of these events to Grade ≤1. In some embodiments, prophylactic systemic corticosteroids are not administered.

In some embodiments, the method further comprises diagnosing said subject for cytokine release syndrome (CRS). In preferred embodiments, the diagnosis is made according to the American Society of Transplantation and Cellular Therapy (ASTCT), formerly the American Society for Blood and Marrow Transplantation (ASBMT) consensus grading. A non-limiting summary of the ASTCT consensus grading for CRS diagnosis is provided in Table 13. In some embodiments, the CRS is assessed by evaluating the levels of one or more of, or all of, IL-6, IL-10, IFN-□, C-reactive protein (CRP) and ferritin.

In some embodiments, the method further comprises treating said subject for cytokine release syndrome (CRS). In some embodiments, the treatment of CRS is with an antipyretic. In some examples, the treatment of CRS is with anticytokine therapy. In some embodiments, the treatment of CRS occurs more than approximately 3 days following the infusion. In some embodiments, the treatment of CRS occurs without significantly reducing CAR-T cell expansion in vivo. In certain embodiments, said method further comprises treating said subject for cytokine release syndrome more than approximately 3 days following said administration of said CAR-T cells without significantly reducing expansion of said CAR-T cells in vivo. In some embodiments, the treatment of CRS comprises administering to the subject an IL-6R inhibitor. In some embodiments, the IL-6R inhibitor is an antibody. In some embodiments, the antibody inhibits IL-6R by binding its extracellular domain. In some embodiments, the IL-6R inhibitor prevents the binding of IL-6 to IL-6R. In some embodiments, the IL-6R inhibitor is tocilizumab. In some embodiments, the anticytokine therapy comprises administration of tocilizumab. In some embodiments, the anticytokine therapy comprises administration of steroids. In some embodiments, treatment for CRS comprises treatment with monoclonal antibodies other than tocilizumab. In some embodiments, the antibodies other than tocilizumab target cytokines. In some embodiments, the cytokine that the antibodies other than tocilizumab target is IL-1. In some embodiments, the IL-1 targeting antibody is Anakinra. In some embodiments, the cytokine that the antibodies other than tocilizumab target is TNFα. In some embodiments, the treatment of CRS comprises administering to the subject a corticosteroid. In some embodiments, the treatment of CRS comprises using a vasopressor. In some embodiments, the treatment of CRS comprises intubation or mechanical ventilation. In some embodiments, the treatment of CRS comprises administering to the subject cyclophosphamide. In some embodiments, the treatment of CRS comprises administering to the subject etanercept. In some embodiments, the treatment of CRS comprises administering to the subject levetiracetam. In some embodiments, the treatment of CRS comprises supportive care.

In some embodiments, the method further comprises diagnosing said subject for immune cell effector-associated neurotoxicity (ICANS). In some embodiments, the diagnosis is made according to the National Cancer Institute Common Terminology Criteria for Adverse Events (NCI CTCAE) criteria. In some embodiments, the diagnosis is made according to the NCI CTCAE criteria, Version 5.0. In some embodiments, the diagnosis is made according to the American Society of Transplantation and Cellular Therapy (ASTCT) consensus grading system. In some embodiments, the embodiments, there is neurotoxicity consistent with ICAN. A non-limiting summary of the ASTCT consensus grading system for ICANS diagnosis is provided in Table 14. In some embodiments, the treatment of ICANS comprises administering to the subject an IL-6R inhibitor. In some embodiments, the IL-6R inhibitor is an antibody. In some embodiments, the antibody inhibits IL-6R by binding its extracellular domain. In some embodiments, the IL-6R inhibitor prevents the binding of IL-6 to IL-6R. In some embodiments, the IL-6R inhibitor is tocilizumab. In some embodiments, the treatment of ICANS comprises administering to the subject an IL-1 inhibitor. In some embodiments the IL-1 inhibitor is an antibody. In a preferred embodiment, the IL-1 inhibiting antibody is Anakinra. In some embodiments, the treatment of ICANS comprises administering to the subject a corticosteroid. In some embodiments, the treatment of ICANS comprises administering to the subject levetiracetam. In some embodiments, the treatment of ICANS comprises administering to the subject dexamethasone. In some embodiments, the treatment of ICANS comprises administering to the subject methylprednisone sodium succinate. In some embodiments, the treatment of ICANS comprises administering to the subject pethidine. In some embodiments, the treatment of ICANS comprises administering to the subject one or more of, or all of, tocilizumab, Anakinra, a corticosteroid, levetiracetam, dexamethasone, methylprednisone sodium succinate or pethidine.

If concurrent neurologic toxicity is suspected during CRS or vice versa, the methods can comprise administering:
Corticosteroids according to the more aggressive intervention based on the CRS and neurologic toxicity grades in Tables 1 and 2 of the herein disclosed Approved Label
Tocilizumab according to the CRS grade in Table 1 of the herein disclosed Approved Label
Anti-seizure medication according to the neurologic toxicity in Table 2 of the herein disclosed Approved Label

In some embodiments, the method further comprises diagnosing said subject for cytopenias. In some embodiments, the cytopenias comprise one or more of, or all of, lymphopenia, neutropenia, and thrombocytopenia. Without being bound by theory, a Grade 3 or Grade 4 but not a Grade 2 or lower lymphopenia is characterized by to a lymphocyte count less than 0.5×109 cells per liter of a subject's blood sample, a Grade 3 or Grade 4 but not a Grade 2 or lower neutropenia is characterized by a neutrophil count less than 1000 cells per microliter of a subject's blood sample, and a Grade 3 or Grade 4 but not a Grade 2 or lower thrombocytopenia is characterized by a platelet count less than 50,000 cells per microliter of a subject's blood sample. In some embodiments, greater than 75% of subjects with Grade 3 or Grade 4 lymphopenia following CAR-T cell administration recover to Grade 2 or lower lymphopenia 60 days following CAR-T cell administration. In some embodiments, greater than 80% of subjects with Grade 3 or Grade 4 lymphopenia following CAR-T cell administration recover to Grade 2 or lower lymphopenia 60 days following CAR-T cell administration. In some embodiments, greater than 85% of subjects with Grade 3 or Grade 4 lymphopenia following CAR-T cell administration recover to Grade 2 or lower lymphopenia 60 days following CAR-T cell administration. In some embodiments, greater than 90% of subjects with Grade 3 or Grade 4 lymphopenia following CAR-T cell administration recover to Grade 2 or lower lymphopenia 60 days following CAR-T cell administration. In some embodiments, greater than 70% of subjects with Grade 3 or Grade 4 neutropenia following CAR-T cell administration recover to Grade 2 or lower neutropenia 60 days following CAR-T cell administration. In some embodiments, greater than 75% of subjects with Grade 3 or Grade 4 neutropenia following CAR-T cell administration recover to Grade 2 or lower neutropenia 60 days following CAR-T cell administration. In some embodiments, greater than 80% of subjects with Grade 3 or Grade 4 neutropenia following CAR-T cell administration recover to Grade 2 or lower neutropenia 60 days following CAR-T cell administration. In some embodiments, greater than 85% of subjects with Grade 3 or Grade 4 neutropenia following CAR-T cell administration recover to Grade 2 or lower neutropenia 60 days following CAR-T cell administration. In some embodiments, greater than 30% of subjects with Grade 3 or Grade 4 thrombocytopenia following CAR-T cell administration recover to Grade 2 or lower thrombocytopenia 60 days following CAR-T cell administration. In some embodiments, greater than 34% of subjects with Grade 3 or Grade 4 thrombocytopenia following CAR-T cell administration recover to Grade 2 or lower thrombocytopenia 60 days following CAR-T cell administration. In some embodiments, greater than 38% of subjects with Grade 3 or Grade 4 thrombocytopenia following CAR-T cell administration recover to Grade 2 or lower thrombocytopenia 60 days following CAR-T cell administration. In some embodiments, greater than 42% of subjects with Grade 3 or Grade 4 thrombocytopenia following CAR-T cell administration recover to Grade 2 or lower thrombocytopenia 60 days following CAR-T cell administration.

Once the composition comprising host cells expressing the CAR-encoding nucleic acid sequences disclosed herein, or a vector comprising the CAR-encoding nucleic acid sequences disclosed herein, is administered to a mammal (e.g., a human), the biological activity of the CAR can be measured by any suitable method known in the art. In accordance with the methods disclosed herein, the CAR binds to BCMA on the multiple myeloma cells, and the multiple myeloma cells are destroyed. Binding of the CAR to BCMA on the surface of multiple myeloma cells can be assayed using any suitable method known in the art, including, for example, ELISA and flow cytometry. The ability of the CAR to destroy multiple myeloma cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). The biological activity of the CAR also can be measured by assaying expression of certain cytokines, such as CD 107a, IFNγ, IL-2, and TNF.

The methods described herein may be used for treating various cancers, including both solid cancer and liquid cancer. In certain embodiments, the methods are used to treat multiple myeloma. The methods described herein may be used as a first therapy, second therapy, third therapy, or combination therapy with other types of cancer therapies known in the art, such as chemotherapy, surgery, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neoadjuvant setting.

In certain embodiments, the cancer is multiple myeloma. In certain embodiments, the cancer is stage I, stage II or stage III, and/or stage A or stage B multiple myeloma based on the Durie-Salmon staging system. In certain embodiments, the cancer is stage I, stage II or stage III multiple myeloma based on the International staging system published by the International Myeloma Working Group (IMWG). In some embodiments, the multiple myeloma is progressive.

In certain embodiments, the subject received prior treatment with at least three prior lines of therapy. In certain embodiments, the median number of lines of prior therapy is 6. In certain embodiments, prior lines of therapy include surgery, radiotherapy, or autologous or allogeneic transplant, or any combination of such treatments. In certain embodiments, the at least three prior lines of therapy comprise treatment with a medicament that is a proteasomal inhibitor (PI). Non-limiting examples of a PI include bortezomib, carfilzomib and ixazomib. In certain embodiments, the at least three prior lines of therapy comprise treatment with a medicament that is an immunomodulatory drug (IMiD). Non-limiting examples of an IMiD include lenalidomide, pomalidomide and thalidomide. In certain embodiments, the at least three prior lines of therapy comprise treatment with a medicament that is a corticosteroid. Non-limiting examples of a corticosteroid include dexamethasone and prednisone. In certain embodiments, the at least three prior lines of therapy comprise treatment with a medicament that is an alkylating agent. In certain embodiments, the at least three prior lines of therapy comprise treatment with a medicament that is an anthracycline. In certain embodiments, the at least three prior lines of therapy comprise treatment with a medicament that is an anti-CD38 antibody. Non-limiting examples of an anti-CD38 antibody include daratumumab, isatuximab and the investigational antibody TAK-079. In certain embodiments, the at least three prior lines of therapy comprise treatment with a medicament that is elotuzumab. In certain embodiments, the at least three prior lines of therapy comprise treatment with a medicament that is panobinostat. In certain embodiments, the at least three prior lines of therapy comprise treatment with at least one medicament, said at least one medicament comprising of at least one of PI, an IMiD, and an anti-CD38 antibody. In certain embodiments, the at least three prior lines of therapy comprise treatment with at least one medicament, said at least one medicament comprising of at least one of PI, an IMiD, and an alkylating agent. In certain embodiments, the subject has relapsed after said at least three prior lines of therapy. In certain embodiments, the multiple myeloma is refractory to one or more of, or all of, bortezomib, carfilzomib, ixazomib, lenalidomide, pomalidomide, thalidomide, dexamethasone, prednisone, alkylating agents, daratumumab, isatuximab, TAK-079, elotuzumab and panobinostat. In certain embodiments, the multiple myeloma is refractory to at least two medicaments following said at least three prior lines of therapy. In certain embodiments, the at least two medicaments to which the multiple myeloma is refractory comprise PI and an IMiD. In certain embodiments, the multiple myeloma is refractory to at least three medicaments following said at least three prior lines of therapy. In certain embodiments, the multiple myeloma is refractory to at least four medicaments following said at least three prior lines of therapy. In certain embodiments, the at least four prior lines of therapy comprise treatment with at least one medicament, said at least one medicament comprising of at least one of PI, an IMiD, anti-CD38 antibody, and an alkylating agent. In certain embodiments, the multiple myeloma is refractory to at least five medicaments following said at least three prior lines of therapy.

In some embodiments, the subject has bone marrow plasma cells of between approximately 10% and approximately 30% before said administration of said CAR-T cells.

In certain embodiments, bone marrow aspirate or biopsy may be performed for clinical assessments or bone marrow aspirate may be performed for biomarker evaluations. In certain embodiments, clinical staging (morphology, cytogenetics, and immunohistochemistry or immunofluorescence or flow cytometry) may be done. In certain embodiments, a portion of the bone marrow aspirate may be immunophenotyped and monitored for BCMA, checkpoint ligand expression in CD138-positive multiple myeloma cells, and checkpoint expression on T cells. In certain embodiments, minimal residual disease (MRD) may be monitored in subjects using next generation sequencing (NGS) of bone marrow aspirate DNA. The NGS of bone marrow aspirate DNA is known to one of ordinary skill in the art. In certain embodiments, the NGS is performed via clonoSeq. In certain embodiments, baseline bone marrow aspirates may be used to define the myeloma clones, and post-treatment samples may be used to evaluate MRD negativity. In certain embodiments, the MRD negativity status may be based on samples that are evaluable. In certain embodiments, evaluable samples are those that passed one or more of, or all of, calibration, quality control, and sufficiency of cells evaluable at a particular sensitivity level. In some embodiments, the sensitivity level is 10-6. In certain embodiments, the sensitivity level is 10-6, the sensitivity level is 10-5. In certain embodiments, the sensitivity level is 10-4. In certain embodiments, the sensitivity level is 10-3.

In certain embodiments, a subject's response to the method of treatment is assessed using the International Myeloma Working Group (IMWG)-based response criteria, which are summarized in Table 6. In certain embodiments, the response may be classified as a stringent complete response (sCR). In certain embodiments, the response may be classified as a complete response (CR), which is worse than a stringent complete response (sCR). In certain embodiments, the response may be classified as a very good partial response (VGPR), which is worse than a complete response (CR). In certain embodiments, the response may be classified as a partial response (PR), which is worse than a very good partial response (VGPR). In certain embodiments, the response may be classified as a minimal response (MR), which is worse than a partial response (PR). In certain embodiments, the response may be classified as a stable disease (SD), which is worse than a minimal response (MR). In certain embodiments, the response may be classified as a progressive disease (PD), which is worse than a stable disease.

In certain embodiments, the tests used to assess International Myeloma Working Group (IMWG)-based response criteria are Myeloma protein (M-protein) measurements in serum and urine, serum calcium corrected for albumin, bone marrow examination, skeletal survey and documentation of extramedullary plasmacytomas.

Non-limiting examples of tests for M-protein measurement in blood and urine are known to one of ordinary skill in the art and comprise serum quantitative Ig, serum protein electrophoresis (SPEP), serum immunofixation electrophoresis, serum FLC assay, 24-hour urine M-protein quantitation by electrophoresis (UPEP), urine immunofixation electrophoresis, and serum β2-microglobulin.

Calculating serum calcium corrected for albumin in blood samples for detection of hypercalcemia is known to one of ordinary skill in the art. Without wishing to be bound by theory, calcium binds to albumin and only the unbound (free) calcium is biologically active; therefore, the serum calcium level must be adjusted for abnormal albumin levels (“corrected serum calcium”).

In certain embodiments, a skeletal survey of any one of, or all of, the skull, the entire vertebral column, the pelvis, the chest, the humeri, the femora, and any other bones, my be performed and evaluated by either roentgenography (“X-rays”) or low-dose computed tomography (CT) diagnostic quality scans without the use of IV contras, both of which are known to one of ordinary skill in the art. In certain embodiments, following T cell administration and before disease progression is confirmed, X-rays or CT scans may be performed locally, whenever clinically indicated based on symptoms, to document response or progression. In certain embodiments, magnetic resonance imaging (MM) may be used for evaluating bone disease but does not replace a skeletal survey. MM is known to one of ordinary skill in the art. In certain embodiments, if a radionuclide bone scan is used at screening, in addition to the complete skeletal survey, both methods may be used to document disease status. Radionuclide bone scans are known to one of ordinary skill in the art. In certain embodiments, the radionuclide bone scan and complete skeletal survey may be performed at the same time. In certain embodiments, a radionuclide bone scan may not replace a complete skeletal survey. In certain embodiments, if a subject presents with disease progression manifested by symptoms of pain due to bone changes, then disease progression may be documented by skeletal survey or other radiographs, depending on the symptoms that the subject experiences.

In certain embodiments, extramedullary plasmacytomas may be documented by clinical examination or MRI. In certain embodiments, if there was no contraindication to the use of IV contrast, extramedullary plasmacytomas may be documented by CT scan. In certain embodiments, extramedullary plasmacytomas may be documented by a fusion of positron emission tomography (PET) and CT scans if the CT component is of sufficient diagnostic quality. In certain embodiments, assessment of measurable sites of extramedullary disease may be performed, measured, or evaluated locally every 4 weeks for subjects until development of confirmed CR or confirmed disease progression. In certain embodiments, evaluation of extramedullary plasmacytomas may be done every 12 weeks.

In certain embodiments, to qualify for VGPR or PR or MR, the sum of products of the perpendicular diameters of the existing extramedullary plasmacytomas may have decreased by over 90% or at least 50%, respectively. In certain embodiments, to qualify for disease progression, either the sum of products of the perpendicular diameters of the existing extramedullary plasmacytomas must have increased by at least 50%, or the longest diameter of previous lesion >1 cm in short axis must have increased at least 50%, or a new plasmacytoma must have developed. In certain embodiments, to qualify for disease progression when not all existing extramedullary plasmacytomas are reported, the sum of products of the perpendicular diameters of the reported plasmacytomas had increased by at least 50%. In certain embodiments, if the study treatment interferes with the immunofixation assay, CR may be defined as the disappearance of the original M-protein associated with multiple myeloma on immunofixation.

In certain embodiments, a subject's response to the method of treatment is assessed in terms of change in disease burden or tumor burden. Disease burden or tumor burden represents the type of measurable disease in the subject. In some embodiments, the change in tumor burden may be assessed in terms of paraprotein level changes upon treatment. In some embodiments, the paraprotein is an M-protein in the serum. In some embodiments, the paraprotein is an M-protein in the serum. In some embodiments, the change in tumor burden is assessed in terms of the difference between involved and uninvolved free light chain (dFLC). In some embodiments, the change in tumor burden is assessed in terms of the maximum paraprotein reduction from baseline, i.e., from prior to the administration of the CAR-T cells. In some embodiments, the change in tumor burden is assessed at a median follow-up time of greater than or equal to 28 days following the administration of CAR-T cells. In some embodiments, the change in tumor burden is assessed at a median follow-up time of greater than or equal to 1 month following the administration of CAR-T cells. In some embodiments, the change in tumor burden is assessed at a median follow-up time of greater than or equal to 3 months following the administration of CAR-T cells. In some embodiments, the change in tumor burden is assessed at a median follow-up time of greater than or equal to 6 months following the administration of CAR-T cells. In some embodiments, the change in tumor burden is assessed at a median follow-up time of greater than or equal to 9 months following the administration of CAR-T cells. In some embodiments, the change in tumor burden is assessed at a median follow-up time of greater than or equal to 12 months following the administration of CAR-T cells.

In certain embodiments, the subject is re-treated by administration via a second intravenous infusion of a second dose of CAR-T cells. In certain embodiments, the re-treatment dose comprises 1.0×105 to 5.0×106 of CAR-T cells per kilogram of the mass of the subject. In certain embodiments, the re-treatment dose comprises approximately 0.75×105 of CAR-T cells per kilogram of the mass of the subject. In certain embodiments, the subject is re-treated upon exhibiting progressive disease after a best response of minimal response or better following the first infusion of CAR-T cells. In certain embodiments, the time between the first infusion of CAR-T cells and the detection of the progressive disease comprises at least six months.

In one aspect is provided a method of treating a subject who has multiple myeloma, said method comprising administering to the subject via a single intravenous infusion a composition comprising a therapeutically effective number of T cells comprising a chimeric antigen receptor (CAR) to deliver to the subject a dose of CAR expressing T cells (CAR-T cells).

In some embodiments, the subject received prior treatment with at least three prior lines of therapy. In some embodiments, said at least three prior lines of therapy comprises treatment with at least one medicament, said at least one medicament comprising of at least one of a PI, an IMiD, and an anti-CD38 antibody. In some embodiments, the subject has relapsed after said at least three prior lines of therapy.

In some embodiments, the multiple myeloma is refractory to at least two medicaments following said at least three prior lines of therapy. In some embodiments, said at least two medicaments to which the subject is refractory comprise PI and an IMiD. In some embodiments, the multiple myeloma is refractory to at least three medicaments following said at least three prior lines of therapy. In some embodiments, the multiple myeloma is refractory to at least four medicaments following said at least three prior lines of therapy. In some embodiments, the multiple myeloma is refractory to at least five medicaments following said at least three prior lines of therapy.

In some embodiments, the subject is greater than 65 years of age. In some embodiments, the subject is Black or African American. In some embodiments, the subject has received 3 prior lines of therapy. In some embodiments, the subject has received at least 4 prior lines of therapy. In some embodiments, the multiple myeloma or the subject is refractory to three classes of medicaments, i.e., the multiple myeloma or the subject is triple-class refractory. In some embodiments, the multiple myeloma or the subject is refractory to five medicaments or drugs, i.e., the multiple myeloma or the subject is penta-drug refractory. In some embodiments, the subject has standard-risk cytogenetics. In some embodiments, the subject has high-risk cytogenetics. In some embodiments, the subject or multiple myeloma has been characterized as stage III per the International Staging System. In some embodiments, the subject has bone marrow plasma cells of between approximately 10% and approximately 30% before said administration of said CAR-T cells. In some embodiments, the subject has bone marrow plasma cells of between approximately 31% and approximately 59% before said administration of said CAR-T cells. In some embodiments, the subject has bone marrow plasma cells of between approximately 60% and approximately 100% before said administration of said CAR-T cells. In some embodiments, the subject has BCMA expression in the tumor less than the median in a population of multiple myeloma patients, or in any randomly selected population. In some embodiments, the subject has BCMA expression in the tumor greater than or equal to the median in a population of multiple myeloma patients, or in any randomly selected population. In some embodiments, plasmacytomas are present in the subject. In some embodiments, the plasmacytomas are bone based. In some embodiments, the plasmacytomas are extramedullary. In some embodiments, the plasmacytomas are both bone based and extramedullary.

In certain embodiments, the method of treatment is effective in obtaining in the subject a reduction in tumor burden. In certain embodiments, the method of treatment is effective in obtaining in the subject a reduction in tumor burden of between approximately between approximately 1% and approximately 100%, between approximately 60% and approximately 100%, between approximately 65% and approximately 100%, between approximately 70% and approximately 100%, between approximately 75% and approximately 100%, between approximately 80% and approximately 100%, between approximately 85% and approximately 100%, between approximately 90% and approximately 100%, between approximately 92% and approximately 100%, between approximately 95% and approximately 100%, between approximately 96% and approximately 100%, between approximately 97% and approximately 100%, between approximately 98% and approximately 100%, or between approximately 99% and approximately 100%. In certain embodiments, the method of treatment is effective in obtaining in the subject a reduction in tumor burden of approximately 100%. In certain embodiments, the method of treatment is effective in obtaining in the subject a reduction in tumor burden of between approximately 1% and approximately 100% at a rate of between approximately 1% and approximately 100%. In certain embodiments, the method of treatment is effective in obtaining in the subject a reduction in tumor burden of between approximately 60% and approximately 100% at a rate of between approximately 1% and approximately 100%. In certain embodiments, the method of treatment is effective in obtaining in the subject a reduction in tumor burden of between approximately 65% and approximately 100% at a rate of between approximately 1% and approximately 92%. In certain embodiments, the method of treatment is effective in obtaining in the subject a reduction in tumor burden of between approximately 70% and approximately 100% at a rate of between approximately 1% and approximately 88%. In certain embodiments, the method of treatment is effective in obtaining in the subject a reduction in tumor burden of between approximately 90% and approximately 100% at a rate of between approximately 1% and approximately 88%. In certain embodiments, the method of treatment is effective in obtaining in the subject a reduction in tumor burden of between approximately 95% and approximately 100% at a rate of between approximately 1% and approximately 88%. In certain embodiments, the method of treatment is effective in obtaining in the subject a reduction in tumor burden of between approximately 99% and approximately 100% at a rate of between approximately 1% and approximately 88%. In certain embodiments, the method of treatment is effective in obtaining in the subject a reduction in tumor burden of approximately 100% at a rate of between approximately 1% and approximately 83%.

In certain embodiments, the method of treatment is effective in obtaining in the subject minimal residual disease (MRD) negative status or maintaining said minimal residual disease (MRD) status. In certain embodiments, the method of treatment is effective in obtaining in the subject a minimal residual disease (MRD) negative status at a sensitivity level of 10-6. In certain embodiments, the method of treatment is effective in obtaining in the subject minimal residual disease (MRD) negative status at a sensitivity level of 10-5. In certain embodiments, the method of treatment is effective in obtaining in the subject minimal residual disease (MRD) negative status at a sensitivity level of 10-4. In certain embodiments, the method of treatment is effective in obtaining in the subject minimal residual disease (MRD) negative status at a sensitivity level of 10-3. In certain embodiments, the method of treatment is effective in obtaining MRD negative status when assessed in the bone marrow. In certain embodiments, the method of treatment is effective in maintaining the MRD negative status when assessed using a bone marrow sample that is evaluable. In certain embodiments, the method of treatment is effective in obtaining MRD negative status when assessed using bone marrow DNA. In some embodiments, said method is effective in obtaining minimal residual disease (MRD) negative status in said subject assessed in the bone marrow at a follow-up time of approximately 28 days or later after said administration of said CAR-T cells, approximately 2 months or later after said administration of said CAR-T cells, approximately 3 months or later after said administration of said CAR-T cells, approximately 6 months or later after said administration of said CAR-T cells, approximately 9 months or later after said administration of said CAR-T cells, or approximately 12 months or later after said administration of said CAR-T cells. In some embodiments, said minimal residual disease (MRD) negative status is obtained at a first follow-up time of between approximately 28 days and approximately 179 days after said infusion of said CAR-T cells.

In certain embodiments, the method of treatment is effective in maintaining in the subject a first obtained minimal residual disease (MRD) negative status. In certain embodiments, the method of treatment is effective in maintaining MRD negative status at a sensitivity level of 10-5. In certain embodiments, the method of treatment is effective in obtaining in the subject minimal residual disease (MRD) negative status at a sensitivity level of 10-6. In certain embodiments, the method of treatment is effective in maintaining MRD negative status at a sensitivity level of 10-4. In certain embodiments, the method of treatment is effective in maintaining MRD negative status at a sensitivity level of 10-3. In certain embodiments, the method of treatment is effective in maintaining the MRD negative status when assessed using a bone marrow sample. In certain embodiments, the method of treatment is effective in maintaining the MRD negative status when assessed using a bone marrow sample that is evaluable. In certain embodiments, the method of treatment is effective in maintaining MRD negative status is maintained when assessed using bone marrow DNA. In some embodiments, said method is effective in maintaining said minimal residual disease (MRD) negative status in said subject assessed in the bone marrow at a second follow-up time of between approximately 29 days and approximately 359 days after said administration of said CAR-T cells, between approximately 29 days and approximately 9 months after said administration of said CAR-T cells, between approximately 29 days and approximately 6 months after said administration of said CAR-T cells, between approximately 29 days and approximately 3 months after said administration of said CAR-T cells, or between approximately 29 days and approximately 2 months after said administration of said CAR-T cells. In some embodiments, said method is effective in maintaining said minimal residual disease (MRD) negative status in said subject assessed in the bone marrow at a second follow-up time of between approximately 180 days and approximately 359 days after said infusion of said CAR-T cells. In some embodiments, said method is effective in maintaining said minimal residual disease (MRD) negative status in said subject assessed in the bone marrow at a second follow-up time of between approximately 360 days and approximately 539 days after said infusion of said CAR-T cells.

In certain embodiments, the efficacy of the method of treatment is assessed by evaluating the proportion of subjects with MRD negative status. In certain embodiments, the efficacy of the method of treatment is assessed by evaluating the proportion of subjects with MRD negative status at a sensitivity level of 10-6. In certain embodiments, the efficacy of the method of treatment is assessed by evaluating the proportion of subjects with MRD negative status at a sensitivity level of 10-5. In certain embodiments, the efficacy of the method of treatment is assessed by evaluating the proportion of subjects with MRD negative status at a sensitivity level of 10-4. In certain embodiments, the efficacy of the method of treatment is assessed by evaluating the proportion of subjects with MRD negative status at a sensitivity level of 10-3. In certain embodiments, the efficacy of the method of treatment is assessed by evaluating the proportion of subjects with MRD negative status at a median follow-up time between the administration of the CAR-T cells and approximately 359 days after the administration of the CAR-T cells, between the administration of the CAR-T cells and approximately 9 months after the administration of the CAR-T cells, between the administration of the CAR-T cells and approximately 6 months after the administration of the CAR-T cells, between the administration of the CAR-T cells and approximately 3 months after the administration of the CAR-T cells, between the administration of the CAR-T cells and approximately 2 months after the administration of the CAR-T cells, or between the administration of the CAR-T cells and approximately 29 days after the administration of the CAR-T cells. In some embodiments, said method is effective in obtaining said minimal residual disease (MRD) negative status at a rate of approximately 44% or less at a sensitivity threshold level of 10-5 at a follow-up time of approximately 12 months after said infusion of said CAR-T cells, a rate of approximately 55% at a sensitivity threshold level of 10-5 at a follow-up time of approximately 12 months after said administration of said CAR-T cells, a rate of approximately 65% or less at a sensitivity threshold level of 10-5 at a follow-up time of approximately 12 months after said infusion of said CAR-T cells, a rate of approximately 57% or less at a sensitivity threshold level of 10-4 at a follow-up time of approximately 18 months after said infusion of said CAR-T cells, a rate of approximately 67% at a sensitivity threshold level of 10-4 at a follow-up time of approximately 18 months after said infusion of said CAR-T cells, a rate of between approximately 76% or less at a sensitivity threshold level of 10-4 at a follow-up time of approximately 18 months after said infusion of said CAR-T cells, a rate of approximately 47% or less at a sensitivity threshold level of 10-5 at a follow-up time of approximately 18 months after said infusion of said CAR-T cells, a rate of approximately 58% at a sensitivity threshold level of 10-5 at a follow-up time of approximately 18 months after said infusion of said CAR-T cells, a rate of approximately 68% or less at a sensitivity threshold level of 10-5 at a follow-up time of approximately 18 months after said infusion of said CAR-T cells, a rate of approximately 29% or less at a sensitivity threshold level of 10-6 at a follow-up time of approximately 18 months after said infusion of said CAR-T cells, a rate of approximately 39% at a sensitivity threshold level of 10-6 at a follow-up time of approximately 18 months after said infusion of said CAR-T cells or a rate of approximately 50% or less at a sensitivity threshold level of 10-6 at a follow-up time of approximately 18 months after said infusion of said CAR-T cells. In some embodiments, said method is effective in obtaining said minimal residual disease (MRD) negative status at a rate of between approximately 44% and approximately 65% at a sensitivity threshold level of 10-5 at a follow-up time of approximately 12 months after said infusion of said CAR-T cells, a rate of between approximately 57% and approximately 76% at a sensitivity threshold level of 10-4 at a follow-up time of approximately 18 months after said infusion of said CAR-T cells, a rate of between approximately 47% and approximately 68% at a sensitivity threshold level of 10-5 at a follow-up time of approximately 18 months after said infusion of said CAR-T cells, or a rate of between approximately 29% and approximately 50% at a sensitivity threshold level of 10-6 at a follow-up time of approximately 18 months after said infusion of said CAR-T cells. In some embodiments, said method is effective in obtaining said minimal residual disease (MRD) negative status at a rate of approximately 55% at a sensitivity threshold level of 10-5 at a follow-up time of approximately 12 months after said administration of said CAR-T cells, a rate of approximately 67% at a sensitivity threshold level of 10-4 at a follow-up time of approximately 18 months after said infusion of said CAR-T cells, a rate of approximately 58% at a sensitivity threshold level of 10-5 at a follow-up time of approximately 18 months after said infusion of said CAR-T cells, or a rate of approximately 39% at a sensitivity threshold level of 10-6 at a follow-up time of approximately 18 months after said infusion of said CAR-T cells.

In certain embodiments, the efficacy of the method of treatment is assessed by evaluating the proportion of subjects with evaluable bone marrow and MRD negative status. In certain embodiments, the efficacy of the method of treatment is assessed by evaluating the proportion of subjects with evaluable bone marrow and MRD negative status at a sensitivity level of 10-6. In certain embodiments, the efficacy of the method of treatment is assessed by evaluating the proportion of subjects with evaluable bone marrow and MRD negative status at a sensitivity level of 10-5. In certain embodiments, the efficacy of the method of treatment is assessed by evaluating the proportion of subjects with evaluable bone marrow and MRD negative status at a sensitivity level of 10-4. In certain embodiments, the efficacy of the method of treatment is assessed by evaluating the proportion of subjects with evaluable bone marrow and MRD negative status at a sensitivity level of 10-3. In certain embodiments, the efficacy of the method of treatment is assessed by evaluating the proportion of subjects with evaluable bone marrow and MRD negative status at a median follow-up time between the administration of the CAR-T cells and approximately 359 days after the administration of the CAR-T cells, between the administration of the CAR-T cells and approximately 9 months after the administration of the CAR-T cells, between the administration of the CAR-T cells and approximately 6 months after the administration of the CAR-T cells, between the administration of the CAR-T cells and approximately 3 months after the administration of the CAR-T cells, between the administration of the CAR-T cells and approximately 2 months after the administration of the CAR-T cells, or between the administration of the CAR-T cells and approximately 29 days after the administration of the CAR-T cells. In some embodiments, said method is effective in obtaining said minimal residual disease (MRD) negative status at a rate of approximately 83% or less in subjects with evaluable samples at a sensitivity threshold level of 10-5 at a follow-up time of approximately 12 months after said infusion of said CAR-T cells, a rate of approximately 93% in subjects with evaluable samples at a sensitivity threshold level of 10-5 at a follow-up time of approximately 12 months after said infusion of said CAR-T cells, a rate of approximately 98% or less in subjects with evaluable samples at a sensitivity threshold level of 10-5 at a follow-up time of approximately 12 months after said infusion of said CAR-T cells, a rate of approximately 82% or less in subjects with evaluable samples at a sensitivity threshold level of 10-5 at a follow-up time of approximately 18 months after said infusion of said CAR-T cells, a rate of approximately 92% in subjects with evaluable samples at a sensitivity threshold level of 10-5 at a follow-up time of approximately 18 months after said infusion of said CAR-T cells, or a rate of approximately 97% or less in subjects with evaluable samples at a sensitivity threshold level of 10-5 at a follow-up time of approximately 18 months after said infusion of said CAR-T cells. In some embodiments, said method is effective in obtaining said minimal residual disease (MRD) negative status at a rate of between approximately 83% and approximately 98% in subjects with evaluable samples at a sensitivity threshold level of 10-5 at a follow-up time of approximately 12 months after said infusion of said CAR-T cells, or at a rate of between approximately 82% and approximately 97% in subjects with evaluable samples at a sensitivity threshold level of 10-5 at a follow-up time of approximately 18 months after said infusion of said CAR-T cells. In some embodiments, said method is effective in obtaining said minimal residual disease (MRD) negative status at a rate of approximately 93% in subjects with evaluable samples at a sensitivity threshold level of 10-5 at a follow-up time of approximately 12 months after said infusion of said CAR-T cells, or at a rate of approximately 92% in subjects with evaluable samples at a sensitivity threshold level of 10-5 at a follow-up time of approximately 18 months after said infusion of said CAR-T cells.

In some embodiments, said method is effective in obtaining at least one response in the subject after said infusion of said CAR-T cells, wherein said at least one response comprises, in order from better to worse, a stringent complete response, a complete response, a very good partial response, a partial response, or a minimal response.

In some embodiments, said method is effective in obtaining a first response within approximately 27 days or later, approximately 29 days or later, approximately 42 days or later, approximately 89 days or later, or approximately 321 days or later after said infusion of said CAR-T cells. In some embodiments, said method is effective in obtaining a first response before a time of between approximately 27 days and approximately 321 days after said infusion of said CAR-T cells. In some embodiments, said method is effective in obtaining a first response before a time of between approximately 27 days and approximately 89 days after said infusion of said CAR-T cells. In some embodiments, said method is effective in obtaining a first response before approximately 42 days after said infusion of said CAR-T cells. In some embodiments, said method is effective in obtaining a first response before approximately 29 days after said infusion of said CAR-T cells.

In certain embodiments, the efficacy of the method of treatment is assessed by evaluating the proportion of subjects with a stringent complete response. In certain embodiments, the efficacy of the method of treatment is assessed by evaluating the proportion of subjects with a complete response or better. In certain embodiments, the efficacy of the method of treatment is assessed by evaluating the proportion of subjects with a very good partial response or better. In certain embodiments, the efficacy of the method of treatment is assessed by evaluating the proportion of subjects with a partial response or better. In certain embodiments, the efficacy of the method of treatment is assessed by evaluating the proportion of subjects with a minimal response or better.

In some embodiments, said method is effective in obtaining a best response of any one of minimal response, partial response, very good partial response, complete response or stringent complete response, i.e., a best response of minimal response or better. In some embodiments, the rate at which said method is effective in obtaining a best response of minimal response or better is called the clinical benefit rate. In some embodiments, said method is effective in obtaining said best response of minimal response or better at a rate of approximately 91% or less at a follow-up time of approximately 12 months after said infusion of said CAR-T cells, a rate of approximately 97% or less at a follow-up time of approximately 12 months after said infusion of said CAR-T cells, a rate of between approximately 99% or less at a follow-up time of approximately 12 months after said infusion of said CAR-T cells, at a rate of approximately 93% or less at a follow-up time of approximately 18 months after said infusion of said CAR-T cells, a rate of approximately 98% or less at a follow-up time of approximately 18 months after said infusion of said CAR-T cells, or a rate of between approximately 100% or less at a follow-up time of approximately 18 months after said infusion of said CAR-T cells. In some embodiments, said method is effective in obtaining said best response of any one of minimal response, partial response, very good partial response, complete response or stringent complete response at a rate of between approximately 91% and approximately 99% at a follow-up time of approximately 12 months after said infusion of said CAR-T cells or at a rate of between approximately 93% and approximately 100% at a follow-up time of approximately 18 months after said infusion of said CAR-T cells. In some embodiments, said method is effective in obtaining said best response of any one of minimal response, partial response, very good partial response, complete response or stringent complete response at a rate of approximately 97% at a follow-up time of approximately 12 months after said infusion of said CAR-T cells or at a rate of approximately 98% at a follow-up time of approximately 18 months after said infusion of said CAR-T cells.

In some embodiments, said method is effective in obtaining a best response of any one of partial response, very good partial response, complete response or stringent complete response, i.e., a best response of partial response or better. In some embodiments, the rate at which said method is effective in obtaining a best response of partial response or better is called the overall survival rate or the overall response rate. In some embodiments, said method is effective in obtaining a best response of partial response or better at a rate of approximately 91% or less at a follow-up time of approximately 12 months after said infusion of said CAR-T cells, a rate of approximately 97% or less at a follow-up time of approximately 12 months after said infusion of said CAR-T cells, a rate of approximately 99% or less at a follow-up time of approximately 12 months after said infusion of said CAR-T cells, a rate of approximately 93% or less at a follow-up time of approximately 18 months after said infusion of said CAR-T cells, a rate of approximately 97% or less at a follow-up time of approximately 18 months after said infusion of said CAR-T cells, or a rate of approximately 100% or less at a follow-up time of approximately 18 months after said infusion of said CAR-T cells. In some embodiments, said method is effective in obtaining said best response of any one of partial response, very good partial response, complete response or stringent complete response at a rate of between approximately 91% and approximately 99% at a follow-up time of approximately 12 months after said infusion of said CAR-T cells or at a rate of between approximately 93% and approximately 100% at a follow-up time of approximately 18 months after said infusion of said CAR-T cells. In some embodiments, said method is effective in obtaining said best response of any one of partial response, very good partial response, complete response or stringent complete response at a rate of approximately 97% at a follow-up time of approximately 12 months after said infusion of said CAR-T cells or at a rate of approximately 97% at a follow-up time of approximately 18 months after said infusion of said CAR-T cells.

In some embodiments, said method is effective in obtaining a best response of any one of very good partial response, complete response or stringent complete response, i.e., a best response of very good partial response or better. In some embodiments, said method is effective in obtaining said best response of very good partial response or better at a rate of approximately 86% or less at a follow-up time of approximately 12 months after said infusion of said CAR-T cells, a rate of approximately 93% or less at a follow-up time of approximately 12 months after said infusion of said CAR-T cells, a rate of approximately 97% or less at a follow-up time of approximately 12 months after said infusion of said CAR-T cells, a rate of approximately 88% or less at a follow-up time of approximately 18 months after said infusion of said CAR-T cells, a rate of approximately 95% or less at a follow-up time of approximately 18 months after said infusion of said CAR-T cells, or a rate of approximately 98% or less at a follow-up time of approximately 18 months after said infusion of said CAR-T cells. In some embodiments, said method is effective in obtaining said best response of any one of very good partial response, complete response or stringent complete response at a rate of between approximately 86% and approximately 97% at a follow-up time of approximately 12 months after said infusion of said CAR-T cells or at a rate of between approximately 88% and approximately 98% at a follow-up time of approximately 18 months after said infusion of said CAR-T cells. In some embodiments, said method is effective in obtaining said best response of any one of very good partial response, complete response or stringent complete response at a rate of approximately 93% at a follow-up time of approximately 12 months after said infusion of said CAR-T cells or at a rate of approximately 95% at a follow-up time of approximately 18 months after said infusion of said CAR-T cells.

In some embodiments, said method is effective in obtaining a best response of complete response or stringent complete response, i.e., a best response of complete response or better. In some embodiments, said method is effective in obtaining said best response of complete response or better at a rate of approximately 57% or less at a follow-up time of approximately 12 months after said infusion of said CAR-T cells, a rate of approximately 67% or less at a follow-up time of approximately 12 months after said infusion of said CAR-T cells, a rate of approximately 76% or less at a follow-up time of approximately 12 months after said infusion of said CAR-T cells, a rate of approximately 73% or less at a follow-up time of approximately 18 months after said infusion of said CAR-T cells, a rate of approximately 83% or less at a follow-up time of approximately 18 months after said infusion of said CAR-T cells, or a rate of approximately 89% or less at a follow-up time of approximately 18 months after said infusion of said CAR-T cells. In some embodiments, said method is effective in obtaining said best response of complete response or stringent complete response at a rate of between approximately 57% and approximately 76% at a follow-up time of approximately 12 months after said infusion of said CAR-T cells or at a rate of between approximately 73% and approximately 89% at a follow-up time of approximately 18 months after said infusion of said CAR-T cells. In some embodiments, said method is effective in obtaining said best response of complete response or stringent complete response at a rate of approximately 67% at a follow-up time of approximately 12 months after said infusion of said CAR-T cells or at a rate of approximately 83% at a follow-up time of approximately 18 months after said infusion of said CAR-T cells.

In some embodiments, said method is effective in obtaining a best response of stringent complete response. In some embodiments, said method is effective in obtaining said best response of stringent complete response at a rate of approximately 57% or less at a follow-up time of approximately 12 months after said infusion of said CAR-T cells, a rate of approximately 67% or less at a follow-up time of approximately 12 months after said infusion of said CAR-T cells, a rate of approximately 76% or less at a follow-up time of approximately 12 months after said infusion of said CAR-T cells, a rate of approximately 73% or less at a follow-up time of approximately 18 months after said infusion of said CAR-T cells, a rate of approximately 83% or less at a follow-up time of approximately 18 months after said infusion of said CAR-T cells, or a rate of approximately 89% or less at a follow-up time of approximately 18 months after said infusion of said CAR-T cells. In some embodiments, said method is effective in obtaining said best response of stringent complete response at a rate of between approximately 57% and approximately 76% at a follow-up time of approximately 12 months after said infusion of said CAR-T cells or at a rate of between approximately 73% and approximately 89% at a follow-up time of approximately 18 months after said infusion of said CAR-T cells. In some embodiments, said method is effective in obtaining said best response of stringent complete response at a rate of approximately 67% at a follow-up time of approximately 12 months after said infusion of said CAR-T cells or at a rate of approximately 83% at a follow-up time of approximately 18 months after said infusion of said CAR-T cells.

In some embodiments, said method is effective in obtaining said best response within approximately 27 days or later, 78 days or later, 153 days or later, 293 days or later, or approximately 534 days or later after said infusion of said CAR-T cells. In some embodiments, said method is effective in obtaining said best response before a time of between approximately 27 days and approximately 534 days after said infusion of said CAR-T cells. In some embodiments, said method is effective in obtaining said best response before a time of between approximately 27 days and approximately 293 days after said infusion of said CAR-T cells. In some embodiments, said method is effective in obtaining said best response before approximately 153 days after said infusion of said CAR-T cells. In some embodiments, said method is effective in obtaining said best response before approximately 78 days after said infusion of said CAR-T cells.

In some embodiments, said method is effective in maintaining a response in the subject at a follow-up time between the time of said first response and approximately 180 days after said infusion of said CAR-T cells, between the time of said first response and approximately 357 days after said infusion of said CAR-T cells, between the time of said first response and approximately 606 days after said infusion of said CAR-T cells, or between the time of said first response and approximately 654 days after said infusion of said CAR-T cells. In some embodiments, said method is effective in maintaining a response at a rate of approximately 77% or less at a follow-up time of approximately 6 months after said infusion of said CAR-T cells, a rate of approximately 85% or less at a follow-up time of approximately 6 months after said infusion of said CAR-T cells, a rate of approximately 91% or less at a follow-up time of approximately 6 months after said infusion of said CAR-T cells, a rate of approximately 63% or less at a follow-up time of approximately 12 months after said infusion of said CAR-T cells, a rate of approximately 74% or less at a follow-up time of approximately 12 months after said infusion of said CAR-T cells, a rate of approximately 81% or less at a follow-up time of approximately 12 months after said infusion of said CAR-T cells, a rate of approximately 56% or less at a follow-up time of approximately 18 months after said infusion of said CAR-T cells, a rate of approximately 67% or less at a follow-up time of approximately 18 months after said infusion of said CAR-T cells, a rate of approximately 75% or less at a follow-up time of approximately 18 months after said infusion of said CAR-T cells, a rate of approximately 52% or less at a follow-up time of approximately 21 months after said infusion of said CAR-T cells, a rate of approximately 63% or less at a follow-up time of approximately 21 months after said infusion of said CAR-T cells, a rate of approximately 72% or less at a follow-up time of approximately 21 months after said infusion of said CAR-T cells, a rate of approximately 48% or less at a follow-up time of approximately 24 months after said infusion of said CAR-T cells, a rate of approximately 60% or less at a follow-up time of approximately 24 months after said infusion of said CAR-T cells, or a rate of approximately 70% or less at a follow-up time of approximately 24 months after said infusion of said CAR-T cells. In some embodiments, said method is effective in maintaining a response at a rate of between approximately 77% and approximately 91% at a follow-up time of approximately 6 months after said infusion of said CAR-T cells, a rate of between approximately 63% and approximately 81% at a follow-up time of approximately 12 months after said infusion of said CAR-T cells, a rate of between approximately 56% and approximately 75% at a follow-up time of approximately 18 months after said infusion of said CAR-T cells, a rate of between approximately 52% and approximately 72% at a follow-up time of approximately 21 months after said infusion of said CAR-T cells, or a rate of between approximately 48% and approximately 70% at a follow-up time of approximately 24 months after said infusion of said CAR-T cells. In some embodiments, said method is effective in maintaining a response at a rate of approximately 85% at a follow-up time of approximately 6 months after said infusion of said CAR-T cells, a rate of approximately 74% at a follow-up time of approximately 12 months after said infusion of said CAR-T cells, a rate of approximately 67% at a follow-up time of approximately 18 months after said infusion of said CAR-T cells, a rate of approximately 63% at a follow-up time of approximately 21 months after said infusion of said CAR-T cells, or a rate of approximately 60% at a follow-up time of approximately 24 months after said infusion of said CAR-T cells.

In some embodiments, wherein said method is effective in obtaining minimal residual disease (MRD) negative status in said subject assessed in the bone marrow at a sensitivity threshold level of 10⁻⁵ between the time of said administration of said CAR-T cells and approximately 3 months after said administration of said CAR-T cells. In some embodiments, said method is effective in obtaining either minimal residual disease (MRD) negative complete response or minimal residual disease (MRD) negative stringent complete response at a rate of approximately 25% or less at a follow-up time of approximately 12 months after said infusion of said CAR-T cells, a rate of approximately 34% or less at a follow-up time of approximately 12 months after said infusion of said CAR-T cells, a rate of approximately 44% or less at a follow-up time of approximately 12 months after said infusion of said CAR-T cells, a rate of approximately 33% or less at a follow-up time of approximately 18 months after said infusion of said CAR-T cells, a rate of approximately 43% or less at a follow-up time of approximately 18 months after said infusion of said CAR-T cells, or a rate of approximately 54% or less at a follow-up time of approximately 18 months after said infusion of said CAR-T cells. In some embodiments, said method is effective in obtaining either minimal residual disease (MRD) negative complete response or minimal residual disease (MRD) negative stringent complete response at a rate of between approximately 25% and approximately 44% at a follow-up time of approximately 12 months after said infusion of said CAR-T cells or at a rate of between approximately 33% and approximately 54% at a follow-up time of approximately 18 months after said infusion of said CAR-T cells. In some embodiments, said method is effective in obtaining either minimal residual disease (MRD) negative complete response or minimal residual disease (MRD) negative stringent complete response at a rate of approximately 34% at a follow-up time of approximately 12 months after said infusion of said CAR-T cells or at a rate of approximately 43% at a follow-up time of approximately 18 months after said infusion of said CAR-T cells.

In some embodiments, said method is effective in obtaining progression-free survival of the subject. In some embodiments, said method is effective in obtaining said progression-free survival of the subject at a time between said infusion of said CAR-T cells and approximately 209 days after said infusion of said CAR-T cells, between said infusion of said CAR-T cells and approximately 386 days after said infusion of said CAR-T cells, between said infusion of said CAR-T cells and approximately 632 days after said infusion of said CAR-T cells, or between said infusion of said CAR-T cells and approximately 684 days after said infusion of said CAR-T cells. In some embodiments, said method is effective in obtaining said progression-free survival at a rate of approximately 79% or less at a follow-up time of approximately 6 months after said infusion of said CAR-T cells, a rate of approximately 88% or less at a follow-up time of approximately 6 months after said infusion of said CAR-T cells, a rate of approximately 93% or less at a follow-up time of approximately 6 months after said infusion of said CAR-T cells, a rate of approximately 67% or less at a follow-up time of approximately 12 months after said infusion of said CAR-T cells, a rate of approximately 76% or less at a follow-up time of approximately 12 months after said infusion of said CAR-T cells, a rate of approximately 84% or less at a follow-up time of approximately 12 months after said infusion of said CAR-T cells, a rate of approximately 57% or less at a follow-up time of approximately 18 months after said infusion of said CAR-T cells, a rate of approximately 67% or less at a follow-up time of approximately 18 months after said infusion of said CAR-T cells, a rate of approximately 75% or less at a follow-up time of approximately 18 months after said infusion of said CAR-T cells, a rate of approximately 57% or less at a follow-up time of approximately 21 months after said infusion of said CAR-T cells, a rate of approximately 67% or less at a follow-up time of approximately 21 months after said infusion of said CAR-T cells, a rate of approximately 75% or less at a follow-up time of approximately 21 months after said infusion of said CAR-T cells, a rate of approximately 49% or less at a follow-up time of approximately 24 months after said infusion of said CAR-T cells, a rate of approximately 61% or less at a follow-up time of approximately 24 months after said infusion of said CAR-T cells, or a rate of approximately 70% or less at a follow-up time of approximately 24 months after said infusion of said CAR-T cells. In some embodiments, said method is effective in obtaining said progression-free survival at a rate of between approximately 79% and approximately 93% at a follow-up time of approximately 6 months after said infusion of said CAR-T cells, a rate of between approximately 67% and approximately 84% at a follow-up time of approximately 12 months after said infusion of said CAR-T cells, a rate of between approximately 57% and approximately 75% at a follow-up time of approximately 18 months after said infusion of said CAR-T cells, a rate of between approximately 57% and approximately 75% at a follow-up time of approximately 21 months after said infusion of said CAR-T cells, or a rate of between approximately 49% and approximately 70% at a follow-up time of approximately 24 months after said infusion of said CAR-T cells. In some embodiments, said method is effective in obtaining said progression-free survival at a rate of approximately 88% at a follow-up time of approximately 6 months after said infusion of said CAR-T cells, a rate of approximately 76% at a follow-up time of approximately 12 months after said infusion of said CAR-T cells, a rate of approximately 67% at a follow-up time of approximately 18 months after said infusion of said CAR-T cells, a rate of approximately 67% at a follow-up time of approximately 21 months after said infusion of said CAR-T cells, or a rate of approximately 61% at a follow-up time of approximately 24 months after said infusion of said CAR-T cells.

In some embodiments, said method further comprises treating said subject for cytokine release syndrome more than approximately 1 day after said infusion of said CAR-T cells. In some embodiments, said method is effective in obtaining a rate of recovery from said cytokine release syndrome of between approximately 1% and approximately 99% at a time of approximately 1, 3, 4, 6, 16 or 97 days after first observance of said cytokine release syndrome.

In some embodiments, said method further comprises treating said subject for immune effector cell-associated neurotoxicity more than approximately 3 days after said infusion of said CAR-T cells. In some embodiments, said method is effective in obtaining a rate of recovery from said immune effector cell-associated neurotoxicity of between approximately 1% and approximately 17% at a time of approximately 1, 4, 5, 8, 12 or 16 days after first observance of said immune effector cell-associated neurotoxicity.

In certain embodiments, the method of treatment is effective in obtaining in the subject a reduction in tumor burden. In certain embodiments, the method of treatment is effective in obtaining a reduction in tumor burden in greater than 90% of the subjects. In certain embodiments, the method of treatment is effective in obtaining a reduction in tumor burden in greater than 91% of the subjects. In certain embodiments, the method of treatment is effective in obtaining a reduction in tumor burden in greater than 92% of the subjects. In certain embodiments, the method of treatment is effective in obtaining a reduction in tumor burden in greater than 93% of the subjects. In certain embodiments, the method of treatment is effective in obtaining a reduction in tumor burden in greater than 94% of the subjects. In certain embodiments, the method of treatment is effective in obtaining a reduction in tumor burden in greater than 95% of the subjects. In certain embodiments, the method of treatment is effective in obtaining a reduction in tumor burden in greater than 96% of the subjects. In certain embodiments, the method of treatment is effective in obtaining a reduction in tumor burden in greater than 97% of the subjects. In certain embodiments, the method of treatment is effective in obtaining a reduction in tumor burden in greater than 98% of the subjects. In certain embodiments, the method of treatment is effective in obtaining a reduction in tumor burden in greater than 99% of the subjects. In some embodiments, the method of treatment is effective in obtaining a reduction in tumor burden in 100% of the subjects.

In certain embodiments, the method of treatment is effective in obtaining in the subject minimal residual disease (MRD) negative status or maintaining said minimal residual disease (MRD) status. In certain embodiments, the method of treatment is effective in obtaining in the subject minimal residual disease (MRD) negative status. In certain embodiments, the method of treatment is effective in obtaining in the subject a minimal residual disease (MRD) negative status at a sensitivity level of 10-6. In certain embodiments, the method of treatment is effective in obtaining in the subject minimal residual disease (MRD) negative status at a sensitivity level of 10-5. In certain embodiments, the method of treatment is effective in obtaining in the subject minimal residual disease (MRD) negative status at a sensitivity level of 10-4. In certain embodiments, the method of treatment is effective in obtaining in the subject minimal residual disease (MRD) negative status at a sensitivity level of 10-3. In certain embodiments, the method of treatment is effective in obtaining MRD negative status when assessed in the bone marrow. In certain embodiments, the method of treatment is effective in maintaining the MRD negative status when assessed using a bone marrow sample that is evaluable. In certain embodiments, the method of treatment is effective in obtaining MRD negative status when assessed using bone marrow DNA. In certain embodiments, the method of treatment is effective in obtaining MRD negative status when assessed at a follow-up time of greater than or equal to 28 days following the administration of CAR-T cells. In certain embodiments, the method of treatment is effective in obtaining MRD negative status when assessed at a follow-up time of greater than or equal to 1 month following the administration of CAR-T cells. In certain embodiments, the method of treatment is effective in obtaining MRD negative status when assessed at a follow-up time of greater than or equal to 3 months following the administration of CAR-T cells. In certain embodiments, the method of treatment is effective in obtaining MRD negative status when assessed at a follow-up time of greater than or equal to 6 months following the administration of CAR-T cells. In certain embodiments, the method of treatment is effective in obtaining MRD negative status when assessed at a follow-up time of greater than or equal to 9 months following the administration of CAR-T cells. In certain embodiments, the method of treatment is effective in obtaining MRD negative status when assessed at a follow-up time of greater than or equal to 12 months following the administration of CAR-T cells.

In certain embodiments, the method of treatment is effective in maintaining in the subject a first obtained minimal residual disease (MRD) negative status. In certain embodiments, the method of treatment is effective in maintaining MRD negative status at a sensitivity level of 10-5. In certain embodiments, the method of treatment is effective in obtaining in the subject minimal residual disease (MRD) negative status at a sensitivity level of 10-6. In certain embodiments, the method of treatment is effective in maintaining MRD negative status at a sensitivity level of 10-4. In certain embodiments, the method of treatment is effective in maintaining MRD negative status at a sensitivity level of 10-3. In certain embodiments, the method of treatment is effective in maintaining the MRD negative status when assessed using a bone marrow sample. In certain embodiments, the method of treatment is effective in maintaining the MRD negative status when assessed using a bone marrow sample that is evaluable. In certain embodiments, the method of treatment is effective in maintaining MRD negative status is maintained when assessed using bone marrow DNA. In certain embodiments, the method of treatment is effective in maintaining MRD negative status when assessed at a follow-up time of greater than or equal to 1 month following the administration of CAR-T cells. In certain embodiments, the method of treatment is effective in maintaining MRD negative status when assessed at a follow-up time of greater than or equal to 3 months following the administration of CAR-T cells. In certain embodiments, the method of treatment is effective in maintaining MRD negative status when assessed at a follow-up time of greater than or equal to 6 months following the administration of CAR-T cells. In certain embodiments, the method of treatment is effective in maintaining MRD negative status when assessed at a follow-up time of greater than or equal to 9 months following the administration of CAR-T cells. In certain embodiments, the method of treatment is effective in maintaining MRD negative status when assessed at a follow-up time of greater than or equal to 12 months following the administration of CAR-T cells.

In certain embodiments, the efficacy of the method of treatment is assessed by evaluating the proportion of subjects with MRD negative status. In certain embodiments, the efficacy of the method of treatment is assessed by evaluating the proportion of subjects with MRD negative status at a sensitivity level of 10-6. In certain embodiments, the efficacy of the method of treatment is assessed by evaluating the proportion of subjects with MRD negative status at a sensitivity level of 10-5. In certain embodiments, the efficacy of the method of treatment is assessed by evaluating the proportion of subjects with MRD negative status at a sensitivity level of 10-4. In certain embodiments, the efficacy of the method of treatment is assessed by evaluating the proportion of subjects with MRD negative status at a sensitivity level of 10-3. In certain embodiments, the efficacy of the method of treatment is assessed by evaluating the proportion of subjects with MRD negative status at a median follow-up time of greater than or equal to 28 days following the administration of CAR-T cells. In certain embodiments, the efficacy of the method of treatment is assessed by evaluating the proportion of subjects with MRD negative status at a median follow-up time of greater than or equal to 1 month following the administration of CAR-T cells. In certain embodiments, the efficacy of the method of treatment is assessed by evaluating the proportion of subjects with MRD negative status at a median follow-up time of greater than or equal to 3 months following the administration of CAR-T cells. In certain embodiments, the efficacy of the method of treatment is assessed by evaluating the proportion of subjects with MRD negative status at a median follow-up time of greater than or equal to 6 months following the administration of CAR-T cells. In certain embodiments, the efficacy of the method of treatment is assessed by evaluating the proportion of subjects with MRD negative status at a median follow-up time of greater than or equal to 9 months following the administration of CAR-T cells. In certain embodiments, the efficacy of the method of treatment is assessed by evaluating the proportion of subjects with MRD negative status at a median follow-up time of greater than or equal to 12 months following the administration of CAR-T cells.

In certain embodiments, the efficacy of the method of treatment is assessed by evaluating the proportion of subjects with evaluable bone marrow and MRD negative status. In certain embodiments, the efficacy of the method of treatment is assessed by evaluating the proportion of subjects with evaluable bone marrow and MRD negative status at a sensitivity level of 10-6. In certain embodiments, the efficacy of the method of treatment is assessed by evaluating the proportion of subjects with evaluable bone marrow and MRD negative status at a sensitivity level of 10-5. In certain embodiments, the efficacy of the method of treatment is assessed by evaluating the proportion of subjects with evaluable bone marrow and MRD negative status at a sensitivity level of 10-4. In certain embodiments, the efficacy of the method of treatment is assessed by evaluating the proportion of subjects with evaluable bone marrow and MRD negative status at a sensitivity level of 10-3. In certain embodiments, the efficacy of the method of treatment is assessed by evaluating the proportion of subjects with evaluable bone marrow and MRD negative status at a median follow-up time of greater than or equal to 28 days following the administration of CAR-T cells. In certain embodiments, the efficacy of the method of treatment is assessed by evaluating the proportion of subjects with evaluable bone marrow and MRD negative status at a median follow-up time of greater than or equal to 1 month following the administration of CAR-T cells. In certain embodiments, the efficacy of the method of treatment is assessed by evaluating the proportion of subjects with evaluable bone marrow and MRD negative status at a median follow-up time of greater than or equal to 3 months following the administration of CAR-T cells. In certain embodiments, the efficacy of the method of treatment is assessed by evaluating the proportion of subjects with evaluable bone marrow and MRD negative status at a median follow-up time of greater than or equal to 6 months following the administration of CAR-T cells. In certain embodiments, the efficacy of the method of treatment is assessed by evaluating the proportion of subjects with evaluable bone marrow and MRD negative status at a median follow-up time of greater than or equal to 9 months following the administration of CAR-T cells. In certain embodiments, the efficacy of the method of treatment is assessed by evaluating the proportion of subjects with evaluable bone marrow and MRD negative status at a median follow-up time of greater than or equal to 12 months following the administration of CAR-T cells.

In certain embodiments, the efficacy of the method of treatment is assessed by evaluating the proportion of subjects with a stringent complete response. In certain embodiments, the efficacy of the method of treatment is assessed by evaluating the proportion of subjects with a complete response or better. In certain embodiments, the efficacy of the method of treatment is assessed by evaluating the proportion of subjects with a very good partial response or better. In certain embodiments, the efficacy of the method of treatment is assessed by evaluating the proportion of subjects with a partial response or better. In certain embodiments, the efficacy of the method of treatment is assessed using an overall response rate. In some embodiments, the overall response rate is the proportion of subjects with a partial response or better.

In certain embodiments, the method is effective in obtaining a minimal residual disease (MRD) negativity rate of greater than 39% at a sensitivity threshold level of 10-5. In certain embodiments, the method is effective in obtaining a minimal residual disease (MRD) negativity rate of greater than 44% at a sensitivity threshold level of 10-5. In certain embodiments, the method is effective in obtaining a minimal residual disease (MRD) negativity rate of greater than 49% at a sensitivity threshold level of 10-5. In certain embodiments, the method is effective in obtaining a minimal residual disease (MRD) negativity rate of greater than 54% at a sensitivity threshold level of 10-5. In certain embodiments, the method is effective in obtaining a minimal residual disease (MRD) negativity rate of greater than 59% at a sensitivity threshold level of 10-5. In certain embodiments, the method is effective in obtaining a minimal residual disease (MRD) negativity rate of greater than 64% at a sensitivity threshold level of 10-5. In certain embodiments, the method is effective in obtaining a minimal residual disease (MRD) negativity rate of greater than 69% at a sensitivity threshold level of 10-5. In certain embodiments, the method is effective in obtaining a minimal residual disease (MRD) negativity rate of greater than 74% at a sensitivity threshold level of 10-5. In certain embodiments, the method is effective in obtaining a minimal residual disease (MRD) negativity rate of greater than 70% at a sensitivity threshold level of 10-5. In certain embodiments, the method is effective in obtaining a minimal residual disease (MRD) negativity rate of greater than 75% at a sensitivity threshold level of 10-5 in evaluable bone marrow. In certain embodiments, the method is effective in obtaining a minimal residual disease (MRD) negativity rate of greater than 80% at a sensitivity threshold level of 10-5 in evaluable bone marrow. In certain embodiments, the method is effective in obtaining a minimal residual disease (MRD) negativity rate of greater than 85% at a sensitivity threshold level of 10-5 in evaluable bone marrow. In certain embodiments, the method is effective in obtaining a minimal residual disease (MRD) negativity rate of greater than 90% at a sensitivity threshold level of 10-5 in evaluable bone marrow. In certain embodiments, the method is effective in obtaining a minimal residual disease (MRD) negativity rate of greater than 95% at a sensitivity threshold level of 10-5 in evaluable bone marrow. In certain embodiments, the method is effective in obtaining a minimal residual disease (MRD) negativity rate of 100% at a sensitivity threshold level of 10-5 in evaluable bone marrow.

In certain embodiments, the method of treatment is effective in obtaining an overall response rate of greater than 90%. In certain embodiments, the method of treatment is effective in obtaining an overall response rate of greater than 91%. In certain embodiments, the method is effective in obtaining an overall response rate of greater than 93%. In certain embodiments, the method is effective in obtaining an overall response rate of greater than 95%. In certain embodiments, the method is effective in obtaining an overall response rate of greater than 97%. In certain embodiments, the method is effective in obtaining an overall response rate of greater than 99%. In some embodiments, the method is effective in obtaining an overall response rate of 100%. In certain embodiments, the overall response rate is assessed at a median follow-up time of at least 6 months following said infusion of said CAR-T cells. In certain embodiments, the overall response rate is assessed at a median follow-up time of at least 12 months following said infusion of said CAR-T cells.

In certain embodiments, more than 70% of subjects are responding to the method of treatment at 9 months after the administration of CAR-T cells. In certain embodiments, more than 72% of subjects are responding to the method of treatment at 9 months after the administration of CAR-T cells. In certain embodiments, more than 74% of subjects are responding to the method of treatment at 9 months after the administration of CAR-T cells. In certain embodiments, more than 76% of subjects are responding to the method of treatment at 9 months after the administration of CAR-T cells. In certain embodiments, more than 78% of subjects are responding to the method of treatment at 9 months after the administration of CAR-T cells. In certain embodiments, more than 80% of subjects are responding to the method of treatment at 9 months after the administration of CAR-T cells. In certain embodiments, more than 82% of subjects are responding to the method of treatment at 9 months after the administration of CAR-T cells. In certain embodiments, more than 84% of subjects are responding to the method of treatment at 9 months after the administration of CAR-T cells. In certain embodiments, more than 86% of subjects are responding to the method of treatment at 9 months after the administration of CAR-T cells.

In certain embodiments, more than 54% of responding subjects are responding to the method of treatment at 12 months after the administration of CAR-T cells. In certain embodiments, more than 58% of responding subjects are responding to the method of treatment at 12 months after the administration of CAR-T cells. In certain embodiments, more than 62% of responding subjects are responding to the method of treatment at 12 months after the administration of CAR-T cells. In certain embodiments, more than 66% of responding subjects are responding to the method of treatment at 12 months after the administration of CAR-T cells. In certain embodiments, more than 70% of responding subjects are responding to the method of treatment at 12 months after the administration of CAR-T cells. In certain embodiments, more than 74% of responding subjects are responding to the method of treatment at 12 months after the administration of CAR-T cells. In certain embodiments, more than 78% of responding subjects are responding to the method of treatment at 12 months after the administration of CAR-T cells.

In certain embodiments, the method of treatment is effective in obtaining a duration of response greater than 9 months, 10 months, 11 months, 12 months, 13 months, 14 months, or 15 months. In certain embodiments, the method of treatment is effective in obtaining a duration of response greater than 12.4 months. In certain embodiments, the method of treatment is effective in obtaining a duration of response greater than 15.9 months.

In certain embodiments, the method of treatment is effective in obtaining a median duration of response greater than 9 months, 10 months, 11 months, 12 months, 13 months, 14 months, or 15 months. In certain embodiments, the method of treatment is effective in obtaining a median duration of response greater than 12.4 months. In certain embodiments, the method of treatment is effective in obtaining a median duration of response greater than 15.9 months.

In certain embodiments, the method of treatment is effective in obtaining a complete response or better in greater than 60% of the subjects. In certain embodiments, the method of treatment is effective in obtaining a complete response or better in greater than 61% of the subjects. In certain embodiments, the method of treatment is effective in obtaining a complete response or better in greater than 62% of the subjects. In certain embodiments, the method of treatment is effective in obtaining a complete response or better in greater than 63% of the subjects. In certain embodiments, the method of treatment is effective in obtaining a complete response or better in greater than 64% of the subjects. In certain embodiments, the method of treatment is effective in obtaining a complete response or better in greater than 65% of the subjects. In certain embodiments, the method of treatment is effective in obtaining a complete response or better in greater than 66% of the subjects. In certain embodiments, the method of treatment is effective in obtaining a complete response or better in greater than 67% of the subjects. In certain embodiments, the complete response or better is assessed less than 1 month after the administration of CAR-T cells. In certain embodiments, the complete response or better is assessed less than 3 months after the administration of CAR-T cells. In certain embodiments, the complete response or better is assessed less than 6 months after the administration of CAR-T cells. In certain embodiments, the complete response or better is assessed less than 9 months after the administration of CAR-T cells. In certain embodiments, the complete response or better is assessed less than 12 months after the administration of CAR-T cells. In certain embodiments, the complete response or better is assessed less than 15 months after the administration of CAR-T cells. In certain embodiments, the complete response or better is assessed more than 15 months the administration of CAR-T cells.

In certain embodiments, the method of treatment is effective in obtaining a very good partial response or better in greater than 85% of the subjects. In certain embodiments, the method of treatment is effective in obtaining a very good partial response or better in greater than 86% of the subjects. In certain embodiments, the method of treatment is effective in obtaining a very good partial response or better in greater than 87% of the subjects. In certain embodiments, the method of treatment is effective in obtaining a very good partial response or better in greater than 88% of the subjects. In certain embodiments, the method of treatment is effective in obtaining a very good partial response or better in greater than 89% of the subjects. In certain embodiments, the method of treatment is effective in obtaining a very good partial response or better in greater than 90% of the subjects. In certain embodiments, the method of treatment is effective in obtaining a very good partial response or better in greater than 91% of the subjects. In certain embodiments, the method of treatment is effective in obtaining a very good partial response or better in greater than 92% of the subjects. In certain embodiments, the very good partial response or better is assessed less than 1 month after the administration of CAR-T cells. In certain embodiments, the very good partial response or better is assessed less than 3 months after the administration of CAR-T cells. In certain embodiments, the very good partial response or better is assessed less than 6 months after the administration of CAR-T cells. In certain embodiments, the very good partial response or better is assessed less than 9 months after the administration of CAR-T cells. In certain embodiments, the very good partial response or better is assessed less than 12 months after the administration of CAR-T cells. In certain embodiments, the very good partial response or better is assessed less than 15 months after the administration of CAR-T cells. In certain embodiments, the very good partial response or better is assessed more than 15 months the administration of CAR-T cells.

In certain embodiments, the method of treatment is effective in obtaining a median time to first response of less than 1.15 months. In certain embodiments, the method of treatment is effective in obtaining a median time to first response of less than 1.10 months. In certain embodiments, the method of treatment is effective in obtaining a median time to first response of less than 1.05 months. In certain embodiments, the method of treatment is effective in obtaining a median time to first response of less than 1.00 months. In certain embodiments, the method of treatment is effective in obtaining a median time to first response of less than 0.95 months.

In certain embodiments, the method of treatment is effective in obtaining a median time to best response of less than 2.96 months. In certain embodiments, the method of treatment is effective in obtaining a median time to best response of less than 2.86 months. In certain embodiments, the method of treatment is effective in obtaining a median time to best response of less than 2.76 months. In certain embodiments, the method of treatment is effective in obtaining a median time to best response of less than 2.66 months. In certain embodiments, the method of treatment is effective in obtaining a median time to best response of less than 2.56 months.

In certain embodiments, the method is effective in obtaining an overall survival rate of greater than 80% at 9 months after the administration of CAR-T cells. In certain embodiments, the method is effective in obtaining an overall survival rate of greater than 82% at 9 months after the administration of CAR-T cells. In certain embodiments, the method is effective in obtaining an overall survival rate of greater than 85% at 9 months after the administration of CAR-T cells. In certain embodiments, the method is effective in obtaining an overall survival rate of greater than 87% at 9 months after the administration of CAR-T cells. In certain embodiments, the method is effective in obtaining an overall survival rate of greater than 90% at 9 months after the administration of CAR-T cells. In certain embodiments, the method is effective in obtaining an overall survival rate of greater than 92% at 9 months after the administration of CAR-T cells. In certain embodiments, the method is effective in obtaining an overall survival rate of greater than 95% at 9 months after the administration of CAR-T cells.

In certain embodiments, the method is effective in obtaining an overall survival rate of greater than 80% at 12 months after the administration of CAR-T cells. In certain embodiments, the method is effective in obtaining an overall survival rate of greater than 83% at 12 months after the administration of CAR-T cells. In certain embodiments, the method is effective in obtaining an overall survival rate of greater than 86% at 12 months after the administration of CAR-T cells. In certain embodiments, the method is effective in obtaining an overall survival rate of greater than 89% at 12 months after the administration of CAR-T cells. In certain embodiments, the method is effective in obtaining an overall survival rate of greater than 92% at 12 months after the administration of CAR-T cells. In certain embodiments, the method is effective in obtaining an overall survival rate of greater than 93% at 12 months after the administration of CAR-T cells.

In certain embodiments, the method is effective in obtaining a progression free survival rate of greater than 70% at 9 months after the administration of CAR-T cells. In certain embodiments, the method is effective in obtaining a progression free survival rate of greater than 72% at 9 months after the administration of CAR-T cells. In certain embodiments, the method is effective in obtaining a progression free survival rate of greater than 75% at 9 months after the administration of CAR-T cells. In certain embodiments, the method is effective in obtaining a progression free survival rate of greater than 77% at 9 months after the administration of CAR-T cells. In certain embodiments, the method is effective in obtaining a progression free survival rate of greater than 80% at 9 months after the administration of CAR-T cells. In certain embodiments, the method is effective in obtaining a progression free survival rate of greater than 82% at 9 months after the administration of CAR-T cells. In certain embodiments, the method is effective in obtaining a progression free survival rate of greater than 85% at 9 months after the administration of CAR-T cells. In certain embodiments, the method is effective in obtaining a progression free survival rate of greater than or equal to 87% at 9 months after the administration of CAR-T cells.

In certain embodiments, the method is effective in obtaining a progression free survival rate of greater than 66% at 12 months after the administration of CAR-T cells. In certain embodiments, the method is effective in obtaining a progression free survival rate of greater than 69% at 12 months after the administration of CAR-T cells. In certain embodiments, the method is effective in obtaining a progression free survival rate of greater than 72% at 12 months after the administration of CAR-T cells. In certain embodiments, the method is effective in obtaining a progression free survival rate of greater than 76% at 12 months after the administration of CAR-T cells. In certain embodiments, the method is effective in obtaining a progression free survival rate of greater than 80% at 12 months after the administration of CAR-T cells. In certain embodiments, the method is effective in obtaining a progression free survival rate of greater than 84% at 12 months after the administration of the CAR-T cells.

In certain embodiments, the method of treatment is effective in obtaining that greater than 86% of subjects recover from cytokine release syndrome. In certain embodiments, the method of treatment is effective in obtaining that greater than 88% of subjects recover from cytokine release syndrome. In certain embodiments, the method of treatment is effective in obtaining that greater than 90% of subjects recover from cytokine release syndrome. In certain embodiments, the method of treatment is effective in obtaining that greater than 92% of subjects recover from cytokine release syndrome. In certain embodiments, the method of treatment is effective in obtaining that greater than 94% of subjects recover from cytokine release syndrome. In certain embodiments, the method of treatment is effective in obtaining that greater than 96% of subjects recover from cytokine release syndrome. In certain embodiments, the method of treatment is effective in obtaining that greater than 98% of subjects recover from cytokine release syndrome. In certain embodiments, the method of treatment is effective in obtaining that greater than 99% of subjects recover from cytokine release syndrome. In certain embodiments, the method of treatment is effective in obtaining that 100% of subjects recover from cytokine release syndrome.

In certain embodiments, the method of treatment is effective in obtaining that greater than 90% of subjects recover from immune effector cell-associated neurotoxicity, if any. In certain embodiments, the method of treatment is effective in obtaining that greater than 92% of subjects recover from immune effector cell-associated neurotoxicity, if any. In certain embodiments, the method of treatment is effective in obtaining that greater than 94% of subjects recover from immune effector cell-associated neurotoxicity, if any. In certain embodiments, the method of treatment is effective in obtaining that greater than 96% of subjects recover from immune effector cell-associated neurotoxicity, if any. In certain embodiments, the method of treatment is effective in obtaining that greater than 98% of subjects recover from immune effector cell-associated neurotoxicity, if any. In certain embodiments, the method of treatment is effective in obtaining that 100% of subjects recover from immune effector cell-associated neurotoxicity, if any.

In some embodiments, the method further comprises diagnosing said subject for cytopenias. In some embodiments, the cytopenias comprise one or more of, or all of, lymphopenia, neutropenia, and thrombocytopenia. Without being bound by theory, a Grade 3 or Grade 4 but not a Grade 2 or lower lymphopenia is characterized by to a lymphocyte count less than 0.5×109 cells per liter of a subject's blood sample, a Grade 3 or Grade 4 but not a Grade 2 or lower neutropenia is characterized by a neutrophil count less than 1000 cells per microliter of a subject's blood sample, and a Grade 3 or Grade 4 but not a Grade 2 or lower thrombocytopenia is characterized by a platelet count less than 50,000 cells per microliter of a subject's blood sample. In some embodiments, greater than 75% of subjects with Grade 3 or Grade 4 lymphopenia following CAR-T cell administration recover to Grade 2 or lower lymphopenia 60 days following CAR-T cell administration. In some embodiments, greater than 80% of subjects with Grade 3 or Grade 4 lymphopenia following CAR-T cell administration recover to Grade 2 or lower lymphopenia 60 days following CAR-T cell administration. In some embodiments, greater than 85% of subjects with Grade 3 or Grade 4 lymphopenia following CAR-T cell administration recover to Grade 2 or lower lymphopenia 60 days following CAR-T cell administration. In some embodiments, greater than 90% of subjects with Grade 3 or Grade 4 lymphopenia following CAR-T cell administration recover to Grade 2 or lower lymphopenia 60 days following CAR-T cell administration. In some embodiments, greater than 70% of subjects with Grade 3 or Grade 4 neutropenia following CAR-T cell administration recover to Grade 2 or lower neutropenia 60 days following CAR-T cell administration. In some embodiments, greater than 75% of subjects with Grade 3 or Grade 4 neutropenia following CAR-T cell administration recover to Grade 2 or lower neutropenia 60 days following CAR-T cell administration. In some embodiments, greater than 80% of subjects with Grade 3 or Grade 4 neutropenia following CAR-T cell administration recover to Grade 2 or lower neutropenia 60 days following CAR-T cell administration. In some embodiments, greater than 85% of subjects with Grade 3 or Grade 4 neutropenia following CAR-T cell administration recover to Grade 2 or lower neutropenia 60 days following CAR-T cell administration. In some embodiments, greater than 30% of subjects with Grade 3 or Grade 4 thrombocytopenia following CAR-T cell administration recover to Grade 2 or lower thrombocytopenia 60 days following CAR-T cell administration. In some embodiments, greater than 34% of subjects with Grade 3 or Grade 4 thrombocytopenia following CAR-T cell administration recover to Grade 2 or lower thrombocytopenia 60 days following CAR-T cell administration. In some embodiments, greater than 38% of subjects with Grade 3 or Grade 4 thrombocytopenia following CAR-T cell administration recover to Grade 2 or lower thrombocytopenia 60 days following CAR-T cell administration. In some embodiments, greater than 42% of subjects with Grade 3 or Grade 4 thrombocytopenia following CAR-T cell administration recover to Grade 2 or lower thrombocytopenia 60 days following CAR-T cell administration.

In certain embodiments, the subject is re-treated by administration via a second intravenous infusion of a second dose of CAR-T cells. In certain embodiments, the re-treatment dose comprises 1.0×105 to 5.0×106 of CAR-T cells per kilogram of the mass of the subject. In certain embodiments, the re-treatment dose comprises approximately 0.75×105 of CAR-T cells per kilogram of the mass of the subject. In certain embodiments, the subject is re-treated upon exhibiting progressive disease after a best response of minimal response or better following the first infusion of CAR-T cells. In certain embodiments, the time between the first infusion of CAR-T cells and the detection of the progressive disease comprises at least six months.

### Kits and Articles of Manufacture

Any of the compositions described herein may be comprised in a kit. In some embodiments, engineered immortalized CAR-T cells are provided in the kit, which also may include reagents suitable for expanding the cells, such as media.

In a non-limiting example, a chimeric receptor expression construct, one or more reagents to generate a chimeric receptor expression construct, cells for transfection of the expression construct, and/or one or more instruments to obtain immortalized T cells for transfection of the expression construct (such an instrument may be a syringe, pipette, forceps, and/or any such medically approved apparatus).

In some aspects, the kit comprises reagents or apparatuses for electroporation of cells.

In some embodiments, the kit comprises artificial antigen presenting cells.

The kits may comprise one or more suitably aliquoted compositions of the present disclosure or reagents to generate compositions of the disclosure. The components of the kits may be packaged either in aqueous media or in lyophilized form. The container means of the kits may include at least one vial, test tube, flask, bottle, syringe, or other container means, into which a component may be placed, and preferably, suitably aliquoted. Where there is more than one component in the kit, the kit also will generally contain a second, third, or other additional container into which the additional components may be separately placed. However, various combinations of components may be comprised in a vial. The kits of the present disclosure also will typically include a means for containing the chimeric receptor construct and any other reagent containers in close confinement for commercial sale. Such containers may include injection or blow molded plastic containers into which the desired vials are retained, for example.

### Approved Drug Product

The disclosure also provides methods for treating adult patients with relapsed or refractory multiple myeloma, comprising administering to the patient an approved drug product comprising a ciltacabtagene autoleucel suspension in an amount and manner that is described in a drug product label for the approved drug product and/or in an administration and/or treatment regimen described herein.

The disclosure further relates to a pharmaceutical product comprising a ciltacabtagene autoleucel suspension, wherein the pharmaceutical product is packaged, and wherein the package includes a label that identifies the ciltacabtagene autoleucel suspension as a an approved drug product for the treatment of adult patients with relapsed or refractory multiple myeloma after four or more prior lines of therapy, including a proteasome inhibitor, an immunomodulatory agent and an anti-CD38 monoclonal antibody.

The disclosure also provides methods of selling an approved drug product comprising the ciltacabtagene autoleucel suspension, said method comprising selling the approved drug product, wherein a drug product label for a reference product for the approved drug product includes instructions for treating a patient with relapsed or refractory multiple myeloma after four or more prior lines of therapy, including a proteasome inhibitor, an immunomodulatory agent and an anti-CD38 monoclonal antibody.

In some embodiments, the methods of selling comprise methods of selling a biosimilar of an approved drug product comprising a ciltacabtagene autoleucel suspension, said method comprising selling the biosimilar, wherein a drug product label for a reference product for the biosimilar includes instructions for treating a patient with relapsed or refractory multiple myeloma after four or more prior lines of therapy, including a proteasome inhibitor, an immunomodulatory agent and an anti-CD38 monoclonal antibody.

The disclosure further provides methods of offering for sale a drug product comprising the ciltacabtagene autoleucel suspension, said method comprising offering for sale such drug product, wherein a drug product label for a reference product for such drug product includes instructions for treating a patient with relapsed or refractory multiple myeloma after four or more prior lines of therapy, including a proteasome inhibitor, an immunomodulatory agent and an anti-CD38 monoclonal antibody.

In some embodiments, the methods of offering for sale comprise methods of offering for sale a biosimilar of an approved drug comprising a ciltacabtagene autoleucel suspension, the method comprising offering for sale the biosimilar, wherein a drug product label for a reference product for the biosimilar includes instructions for treating a patient with relapsed or refractory multiple myeloma after four or more prior lines of therapy, including a proteasome inhibitor, an immunomodulatory agent and an anti-CD38 monoclonal antibody.

The terms “sale” or “selling” as used herein refers to transferring a drug product, e.g., a pharmaceutical composition or a dosage form, from a seller to a buyer.

The term “offering for sale,” as used herein, refers to the proposal of a sale by a seller to a buyer for a drug product, e.g., a pharmaceutical composition or a dosage form. These methods comprise offering the drug product for sale.

The term “drug product” refers to a product that contains an active pharmaceutical ingredient that has been approved for marketing by a governmental authority, e.g., the Food and Drug Administration or the similar authority in other countries.

“Label” or “drug product label” refers to information provided to a patient which provides relevant information regarding the drug product. Such information includes, without limitation, one or more of: the description of the drug product, clinical pharmacology, indications (uses for the drug product), contraindication (who should not take the drug product), warnings, precautions, adverse events (side effects), drug abuse and dependence, dosage and administration, use in pregnancy, use in nursing mothers, use in children and older patients, how the drug product is supplied, safety information for the patient, or any combination thereof. In certain embodiments, the label or drug product label provides an instruction for use in a patient requiring BCMA CAR T cells. In further embodiments, the label or drug product label identifies the ciltacabtagene autoleucel suspension and provides instructions for its use in a patient requiring the BCMA CAR T cells.

The term “reference product” refers to an FDA approved biological product (approved drug product) against which a proposed biosimilar product is compared. A reference product is approved based on, among other things, a full complement of safety and effectiveness data. A proposed biosimilar product is compared to, and evaluated against, a reference product to ensure that the product is highly similar and has no clinically meaningful differences.

A “biosimilar” is a biological product that is highly similar to, and has no clinically meaningful differences from, an existing FDA-approved reference product (approved drug product). A biosimilar can be shown to be highly similar to the reference product by extensively analyzing (i.e., characterizing) the structure and function of both the reference product and the proposed biosimilar and comparing characteristics of the products, such as purity, chemical identity, and bioactivity. Minor differences between the reference product and the proposed biosimilar product in clinically inactive components (such as minor differences in the stabilizer or buffer compared to what is used in the reference product) are acceptable. Any differences between the proposed biosimilar product and the reference product are carefully evaluated by FDA to ensure the biosimilar meets FDA's high approval standards. Slight differences (i.e., acceptable within-product variations) are expected during the manufacturing process for biological products, regardless of whether the product is a biosimilar or a reference product. A manufacturer must also demonstrate that its proposed biosimilar product has no clinically meaningful differences from the reference product in terms of safety, purity, and potency (safety and effectiveness), which is generally demonstrated through human pharmacokinetic (exposure) and pharmacodynamic (response) studies, an assessment of clinical immunogenicity, and, if needed, additional clinical studies.

The manufacturer of a proposed biosimilar product generates data comparing the proposed product to the FDA-approved reference product in order to demonstrate biosimilarity. A biosimilar product application must include data demonstrating biosimilarity to the reference product, which includes data from:
Analytical studies demonstrating that the biological product is highly similar to the reference product, notwithstanding minor differences in clinically inactive components;
Animal studies, including an assessment of toxicity; and
A clinical study or studies sufficient to demonstrate safety, purity, and potency of the proposed biosimilar product in one or more of the indications for which the reference product is licensed, which typically includes assessing immunogenicity, pharmacokinetics (PK), and, in some cases, pharmacodynamics (PD) and may also include a comparative clinical study.

Rather than generating the same full profile of nonclinical and clinical data as the reference product, a manufacturer that shows its proposed biosimilar product is highly similar to and has no clinically meaningful differences from the FDA-approved reference product may rely in part on the FDA's previous determination of safety and effectiveness for the reference product for approval. Thus, the biosimilar manufacturer may not need to conduct as many clinical trials.

Also disclosed herein are methods of supplying, storing, and handling an approved drug product comprising a ciltacabtagene autoleucel suspension. The approved drug product comprising the ciltacabtagene autoleucel suspension is supplied in one infusion bag containing a frozen ciltacabtagene autoleucel suspension in 5% DMSO, either as a: 70 mL suspension in an infusion bag and metal cassette (NDC 57894-111-01) or 30 mL suspension in an infusion bag and metal cassette (NDC 57894-111-02). Each infusion bag is individually packed in an aluminum cryo-cassette. The infusion bags are stored and transported below −120° C., e.g., in a container for cryogenic storage in the vapor phase of liquid nitrogen, and the approved drug product is stored in the original packaging containing the cassette protecting the infusion bag. In some embodiments, the time from leukapheresis to product availability can be 27 to 66 days. In some embodiments, the time from leukapheresis to product availability can be 32 days.

The exemplary embodiments below are intended to be purely exemplary of the disclosure and should therefore not be considered to limit the disclosure in any way.

### EXEMPLARY EMBODIMENTS

1. A method of treating a subject, comprising administering to the subject a single infusion of a dose of a composition comprising T cells comprising a chimeric antigen receptor (CAR),
   wherein the CAR comprises the amino acid sequence of SEQ ID NO: 17;
   wherein the dose comprises 0.5×106 to 1.0×106 of the T cells/kg of body weight of the subject; and
   wherein the method comprises completing administering to the subject the dose of T cells within about 2.5 hours at a temperature of about 20° C. to 25° C.
2. The method of embodiment 1, wherein the subject has relapsed or refractory multiple myeloma, who has received multiple prior lines of therapy, and wherein optionally the subject has received three or more prior lines of therapy.
3. The method of embodiment 1 or 2, wherein the subject has received four or more prior lines of therapy.
4. The method of embodiment 2 or 3, wherein the prior lines of therapy comprise a proteasome inhibitor, an immunomodulatory agent, and an anti-CD38 monoclonal antibody.
5. The method of any one of embodiments 1-4, wherein the T cells are autologous T cells.
6. The method of any one of embodiments 1-5, wherein the method further comprises administering to the subject a lymphodepleting chemotherapy regimen prior to administering to the subject the T cells.
7. The method of embodiment 6, wherein the lymphodepleting chemotherapy regimen comprises administering cyclophosphamide and fludarabine to the subject.
8. The method of embodiment 7, wherein the lymphodepleting chemotherapy regimen comprises administering cyclophosphamide and fludarabine to the subject intravenously.
9. The method of embodiment 8, wherein the lymphodepleting chemotherapy regimen comprises administering to the subject intravenously cyclophosphamide at a dose of about 300 mg/m² and fludarabine at a dose of 30 mg/m² daily.
10. The method of any one of embodiments 6-9, wherein the lymphodepleting chemotherapy regimen is for about 3 days.
11. The method of any one of embodiments 6-10, wherein the method comprises administering to the subject the lymphodepleting chemotherapy regimen for at least about 2-4 days prior to administering to the subject the T cells.
12. The method of any one of embodiments 1-11, wherein the method further comprises administering to the subject a premedication for up to 60 minutes prior to administering to the subject the T cells and wherein the premedication comprises an antipyretics and an antihistamine.
13. The method of embodiment 12, wherein the method comprises administering to the subject the premedication for about 30-60 minutes prior to administering to the subject the T cells.
14. The method of embodiment 12 or 13, wherein the antipyretics comprises paracetamol or acetaminophen.
15. The method of embodiment 14, wherein the antipyretics comprises acetaminophen at a dose of about 650-1000 mg.
16. The method of any one of embodiments 12-15, wherein the antihistamine comprises diphenhydramine.
17. The method of embodiment 16, wherein the diphenhydramine is at a dose of about 25-50 mg or equivalent.
18. The method of any one of embodiments 12-17, wherein the premedication is administered orally or intravenously.
19. The method of any one of embodiments 12-18, wherein the premedication does not comprise a systemic corticosteroid.
20. The method of any one of embodiments 1-19, wherein the method comprises thawing the dose of the T cells prior to administration, wherein the thawing is completed in no more than about 15 minutes.
21. The method of embodiment 20, wherein thawing the dose of the T cells is at a temperature of about 37° C.±2° C.
22. The method of any one of embodiments 1-21, wherein the method further comprises treating the subject for cytokine release syndrome (CRS) after administering the dose of the T cells.
23. The method of embodiment 22, wherein CRS comprises fever, pyrexia, hypotension, increased aspartate aminotransferase, chills, increased alanine aminotransferase, sinus tachycardia, hyperbilirubinemia, hypoxia, respiratory failure, acute kidney injury, disseminated intravascular coagulation and hemorrhage (e.g., retroperitoneal, intracerebral or gastrointestinal hemorrhage), hemophagocytic lymphohistiocytosis (HLH), macrophage activation syndrome (MAS), angina pectoris, supraventricular and ventricular tachycardia, malaise, myalgias, increased-C-reactive protein, ferritin, blood alkaline phosphatase, gamma-glutamyl transferase, organ toxicity, or any combination thereof
24. The method of embodiment 22 or 23, wherein treating the subject for CRS comprises administering an anti-cytokine agent or a corticosteroid to the subject.
25. The method of embodiment 24, wherein the anti-cytokine agent comprises a monoclonal antibody targeting cytokines.
26. The method of embodiment 25, wherein the monoclonal antibody targeting cytokines is an IL-6R inhibitor.
27. The method of embodiment 26, wherein the IL-6R inhibitor is tocilizumab.
28. The method of embodiment 27, wherein the method comprises administering tocilizumab intravenously at a dose of about 8 mg/kg over about 1 hour.
29. The method of embodiment 28, wherein the dose of tocilizumab does not exceed about 800 mg.
30. The method of embodiment 28 or 29, wherein the dose of tocilizumab is no more than 3 doses in 24 hours.
31. The method of any one of embodiments 28-30, wherein the dose of tocilizumab is no more than 4 doses in total.
32. The method of embodiment 24, wherein the anti-cytokine agent further comprises an anti-cytokine agent other than tocilizumab.
33. The method of embodiment 24, wherein the anti-cytokine agent further comprises a monoclonal antibody targeting cytokines other than tocilizumab.
34. The method of any one of embodiments 24-33, wherein the corticosteroid comprises dexamethasone or methylprednisolone.
35. The method of embodiment 34, wherein the corticosteroid is dexamethasone.
36. The method of embodiment 35, wherein the method comprises administering to the subject a dose of about 10 mg of dexamethasone intravenously about every 12-24 hours.
37. The method of embodiment 36, wherein the method comprises administering to the subject a dose of about 10 mg of dexamethasone intravenously about every 12 hours.
38. The method of embodiment 35, wherein the method comprises administering to the subject a dose of about 20 mg of dexamethasone intravenously about every 6-12 hours.
39. The method of embodiment 38, wherein the method comprises administering to the subject a dose of about 20 mg of dexamethasone intravenously about every 6 hours.
40. The method of embodiment 34, wherein the corticosteroid is methylprednisolone.
41. The method of embodiment 40, wherein the method comprises administering to the subject a dose of about 2 mg/kg methylprednisolone intravenously about every 12 hours.
42. The method of embodiment 40, wherein the method comprises administering to the subject a dose of about 1-2 g of methylprednisolone intravenously about every 24 hours.
43. The method of embodiment 22 or 23, wherein the method comprises administering an immunosuppressant to the subject.
44. The method of any one of embodiments 1-43, wherein the method further comprises treating the subject for neurologic toxicity after administering the dose of the T cells.
45. The method of embodiment 44, wherein the neurologic toxicity comprises an immune effector cell-associated neurotoxicity syndrome (ICANS), parkinsonism, Guillain-Barré Syndrome, immune mediated myelitis, peripheral neuropathy, cranial nerve palsy or any combination thereof
46. The method of embodiment 45, wherein the neurologic toxicity comprises an ICANS and wherein the ICANS comprises encephalopathy, aphasia, headache, depressed level of consciousness, seizure, motor finding, raised intracranial pressure (ICP), celebral edema, or any combination thereof
47. The method of embodiment 46, wherein the ICANS comprises focal or generalized seizure, non-convulsive seizure on electroencephalogram (EEG), life-threatening prolonged seizure, repetitive clinical or electrical seizure, deep focal motor weakness, hemiparesis, paraparesis, focal or local edema on neuroimaging, stupor, coma, diffuse cerebral edema on neuroimaging, decerebrate or decorticate posturing, cranial nerve VI palsy, papilledema, Cushing's triad, or any combination thereof.
48. The method of embodiment 46 or 47, wherein the method comprises administering to the subject a dose of about 10 mg of dexamethasone intravenously about every 12-24 hours for about 2-3 days.
49. The method of embodiment 48, wherein the method comprises administering to the subject a dose of about 10 mg of dexamethasone intravenously about every 12 hours for about 2-3 days or longer.
50. The method of embodiment 46 or 47, wherein the method comprises administering to the subject a dose of about 10-20 mg of dexamethasone intravenously about every 6 hours.
51. The method of any one of embodiments 46-50, wherein the method comprises administering to the subject a dose of methylprednisolone at about 1-2 g/day about every 24 hours.
52. The method of any one of embodiments 46-50, wherein the ICANS comprises celebral edema.
53. The method of embodiment 52, wherein the method comprises administering to the subject hyperventilation and hyperosmolar therapy.
54. The method of any one of embodiments 46-53, wherein the method comprises administering to the subject a non-sedating anti-seizure medicine.
55. The method of embodiment 54, wherein the non-sedating anti-seizure medicine is levetiracetam.
56. The method of embodiment 45, wherein the neurologic toxicity comprises parkinsonism.
57. The method of embodiment 56, wherein the parkinsonism comprises a parkinsonian symptom or a non-parkinsonian symptom.
58. The method of embodiment 57, wherein the parkinsonian symptom or the non-parkinsonian symptom comprises tremor, bradykinesia, involuntary movements, stereotypy, loss of spontaneous movements, masked facies, apathy, flat affect, fatigue, rigidity, psychomotor retardation, micrographia, dysgraphia, apraxia, lethargy, confusion, somnolence, loss of consciousness, delayed reflexes, hyperreflexia, memory loss, difficulty swallowing, bowel incontinence, falls, stooped posture, shuffling gait, muscle weakness and wasting, motor dysfunction, motor and sensory loss, akinetic mutism, frontal lobe release signs, or any combination thereof
59. The method of any one of embodiments 56-58, wherein the method comprises administering a treatment to the subject to alleviate parkinsonism.
60. The method of embodiment 45, wherein the neurologic toxicity comprises Guillain-Barré Syndrome.
61. The method of embodiment 60, wherein Guillain-Barré Syndrome comprises a symptom consistent with Miller-Fisher variant of Guillain-Barré Syndrome, encephalopathy, motor weakness, speech disturbances, polyradiculoneuritis, or any combination thereof.
62. The method of embodiment 61, wherein the method comprises administering a treatment to the subject to alleviate Guillain-Barré Syndrome.
63. The method of embodiment 45, wherein the neurologic toxicity comprises immune mediated myelitis.
64. The method of embodiment 63, wherein a symptom of immune mediated myelitis comprises hypoesthesia of a lower extremity or lower abdomen with impaired sphincter control.
65. The method of embodiment 63 or 64, wherein the method comprises administering a treatment to the subject to alleviate immune mediated myelitis, wherein optionally the treatment comprises a corticosteroid or an immune globulin, and wherein optionally the method comprises administering the immune globulin intravenously.
66. The method of embodiment 45, wherein the neurologic toxicity comprises peripheral neuropathy.
67. The method of embodiment 66, wherein the peripheral neuropathy comprises sensory, motor, sensorimotor neuropathy, or any combination thereof.
68. The method of embodiment 66 or 67, wherein the method comprises administering a treatment to the subject to alleviate peripheral neuropathy.
69. The method of embodiment 45, wherein the neurologic toxicity comprises cranial nerve palsy.
70. The method of embodiment 69, wherein cranial nerve palsy comprises 3rd cranial nerve palsy, 6th cranial nerve palsy, 7th cranial nerve palsy, or bilateral 7th cranial nerve palsy.
71. The method of embodiment 69 or 70, wherein the method comprises administering a treatment to the subject to alleviate cranial nerve palsy.
72. The method of embodiment 23, wherein CRS comprises hemophagocytic lymphohistiocytosis (HLH) or macrophage activation syndrome (MAS) and wherein symptom of HLH or MAS comprises hypotension, hypoxia with diffuse alveolar damage, coagulopathy, cytopenia, multi-organ dysfunction including renal dysfunction, or any combination thereof
73. The method of embodiment 72, wherein the method comprises administering a treatment to the subject to alleviate HLH or MAS.
74. The method of any one of embodiments 1-73, wherein the method further comprises treating the subject for prolonged or recurrent cytopenia after administering to the subject a lymphodepleting chemotherapy regimen prior to administering to the subject the T cells comprising the CAR or after administering the dose of the T cells comprising the CAR.
75. The method of embodiment 74, wherein the prolonged recurrent cytopenia comprises prolonged neutropenia, prolonged thrombocytopenia, recurrent neutropenia, thrombocytopenia, lymphopenia, anemia, or any combination thereof
76. The method of embodiment 1-75, wherein the method further comprises treating the subject for an infection.
77. The method of embodiment 76, wherein the infection is viral, bacterial, fungal, or by an unspecified pathogen, wherein optionally the infection comprises lung abscess, sepsis, pneumonia, bronchopulmonary aspergillosis, Pneumocystis jirovecii pneumonia, CMV colitis (with HSV-1 hepatitis), mycotic aneurysm, cerebral aspergillosis or COVID-19 infection.
78. The method of embodiment 76, wherein the infection causes febrile neutropenia or subarachnoid hemorrhage.
79. The method of any one of embodiments 76-78, wherein the method comprises administering to the subject an antimicrobial.
80. The method of embodiment 79, wherein the antimicrobial is an antibiotic.
81. The method of embodiment 80, wherein the antibiotic is a broad-spectrum antibiotic.
82. The method of embodiment 77, wherein the infection is viral.
83. The method of embodiment 82, wherein the method comprises administering to the subject an antiviral therapy or a vaccine.
84. The method of embodiment 1-83, wherein the method further comprises treating the subject for hypogammaglobulinemia.
85. The method of embodiment 84, wherein hypogammaglobulinemia comprises a laboratory IgG level below about 500 mg/dL after administering the dose of the T cells comprising the CAR.
86. The method of embodiment 84 or 85, wherein the method comprises administering to the subject a dose of intravenous immunoglobulin (IVIG) after administering the dose of the T cells comprising the CAR.
87. The method of any one of embodiments 1-86, wherein the method further comprises treating the subject for a hypersensitivity reaction.
88. The method of embodiment 87, wherein the hypersensitivity reaction comprises flushing, chest discomfort, tachycardia, wheezing, tremor, burning sensation, anaphylaxis, or any combination thereof
89. The method of embodiment 87 or 88, wherein the method comprises administering to the subject a treatment to alleviate the hypersensitivity reaction.
90. The method of any one of embodiments 1-89, wherein the method further comprises treating the subject for a secondary malignancy.
91. The method of any one of embodiments 1-90, wherein the composition further comprises an excipient selected from dimethyl sulfoxide or dextran-40.
92. The method of embodiment 91, wherein the excipient is dimethyl sulfoxide.
93. The method of embodiment 92, wherein the excipient is about 1-10% of dimethyl sulfoxide.
94. The method of embodiment 93, wherein the excipient is about 5% of dimethyl sulfoxide.
95. A pharmaceutical product comprising a ciltacabtagene autoleucel suspension for intravenous infusion, wherein the pharmaceutical product is packaged, and wherein the package includes a label that identifies the ciltacabtagene autoleucel suspension as an approved drug product for the treatment of adult patients with relapsed or refractory multiple myeloma after four or more prior lines of therapy, including a proteasome inhibitor, an immunomodulatory agent and an anti-CD38 monoclonal antibody.
96. A method for treating relapsed or refractory multiple myeloma in a patient in need thereof, comprising administering an approved drug product comprising a ciltacabtagene autoleucel suspension in an amount and manner that is described in a drug product label for the approved drug product.
97. A method of selling an approved drug product comprising a ciltacabtagene autoleucel suspension, said method comprising selling such drug product, wherein a drug product label for a reference product for such drug product includes instructions for treating adult patients with relapsed or refractory multiple myeloma after four or more prior lines of therapy, including a proteasome inhibitor, an immunomodulatory agent and an anti-CD38 monoclonal antibody.
98. A method of offering for sale a drug product comprising a ciltacabtagene autoleucel suspension, said method comprising offering for sale such drug product, wherein a drug product label for a reference product for such drug product includes instructions for treating adult patients with relapsed or refractory multiple myeloma after four or more prior lines of therapy, including a proteasome inhibitor, an immunomodulatory agent and an anti-CD38 monoclonal antibody.

### EXAMPLES

The following examples are provided to further describe some of the embodiments disclosed herein. The examples are intended to illustrate, not to limit, the disclosed embodiments.

### Example 1: Ciltacabtagene Autoleucel

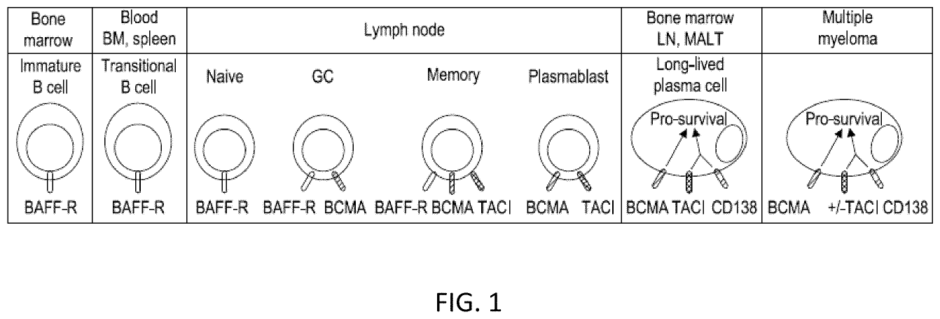

B cell maturation antigen (BCMA, also known as CD269 and TNFRSF17) is a 20 kilodalton, type III membrane protein that is part of the tumor necrosis receptor superfamily. BCMA is a cell surface antigen that is predominantly expressed in B-lineage cells at high levels. FIG. 1 shows the expression of BCMA on various immune-derived cells. Comparative studies have shown a lack of BCMA in most normal tissues and absence of expression on CD34-positive hematopoietic stem cells. BCMA binds 2 ligands that induce B cell proliferation, and plays a critical role in B cell maturation and subsequent differentiation into plasma cells. The selective expression and the biological importance for the proliferation and survival of myeloma cells makes BCMA a promising target for CAR-T based immunotherapy, ciltacabtagene autoleucel.

Ciltacabtagene autoleucel is an autologous chimeric antigen receptor T cell (CAR-T) therapy that targets BCMA. The ciltacabtagene autoleucel chimeric antigen receptor (CAR) comprises two B-cell maturation antigen (BCMA)-targeting VHH domains designed to confer avidity. A map of the construct is depicted in FIG. 2. Ciltacabtagene autoleucel includes a VHH domain comprising the amino acid sequence set forth in SEQ ID NO: 2 and a VHH domain comprising the amino acid sequence set forth in SEQ ID NO: 4.

### Example 2: Method of Treatment with Ciltacabtagene Autoleucel

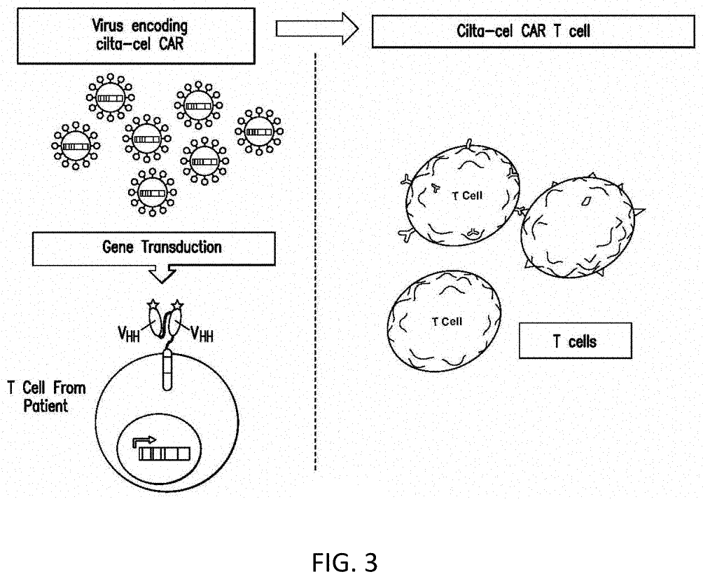

Herein, we describe a Phase 1b-2, open-label, multicenter study that was conducted to evaluate the safety and efficacy of ciltacabtagene autoleucel in adult subjects with relapsed or refractory multiple myeloma. In the Phase 1b portion of the study, a recommended Phase 2 dose (RP2D) of cilta-cel was confirmed. In Phase 2, subjects were treated at the RP2D established from Phase 1b. The objective of the phase 2 portion of the study was to further establish the safety and efficacy of cilta-cel. A schematic overview of the study flow chart, which consists of a lymphodepleting regimen prior to cilta-cel infusion, is depicted in FIG. 3.

The first analysis was conducted approximately 6 months after the last subject received their initial dose of cilta-cel. This report is generated from the protocol-specified first analysis. A summary of the subjects enrolled in the study is presented in Table 1, in which percentages were calculated with the number of subjects in the all enrolled analysis set as denominator. A total of 113 subjects (Phase 1b: 35; Phase 2: 78) were enrolled (apheresed) in the US, out of which 101 subjects (Phase 1b: 30; Phase 2: 71) received conditioning regimen and 97 subjects (Phase 1b: 29; Phase 2: 68) received cilta-cel infusion and received it at the targeted RP2D. These 97 subjects constituted the all treated analysis set, which is the basis for all efficacy and safety analyses presented below. At the clinical cutoff, the median duration of follow-up, based on Kaplan-Meier product limit estimate, for the all treated analysis set was 12.4 months. A summary of the study's duration of follow-up is presented in Table 2, which lists duration of follow up relative to the date of the initial cilta-cel infusion (Day 1).

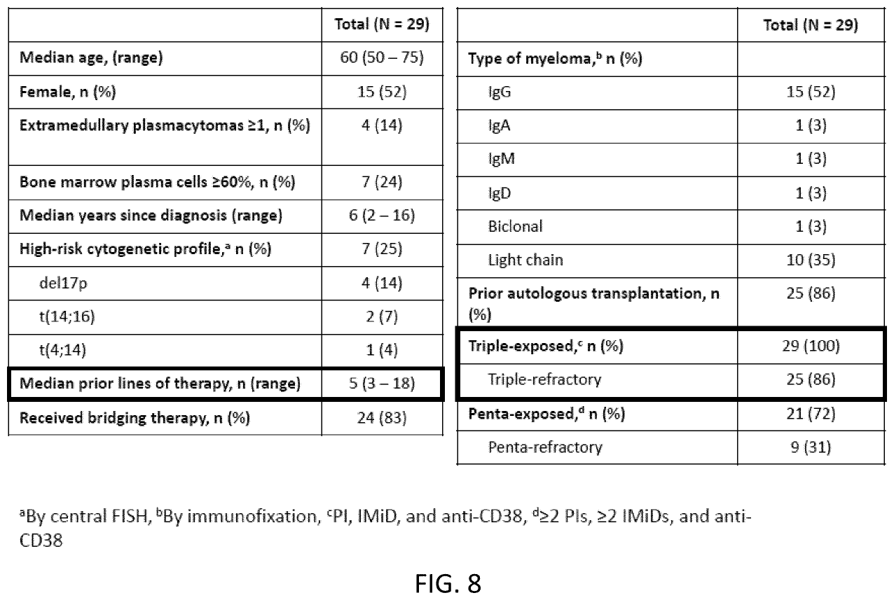

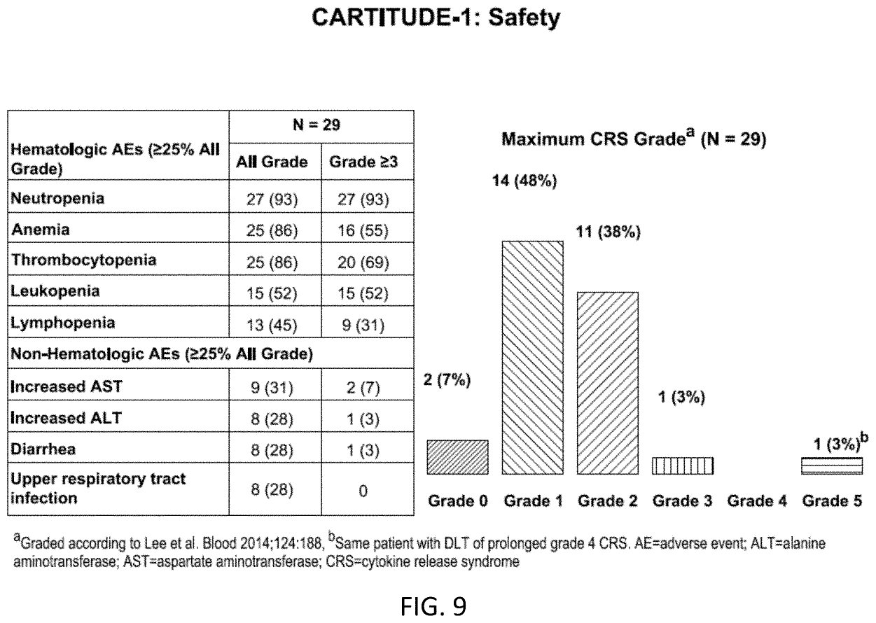

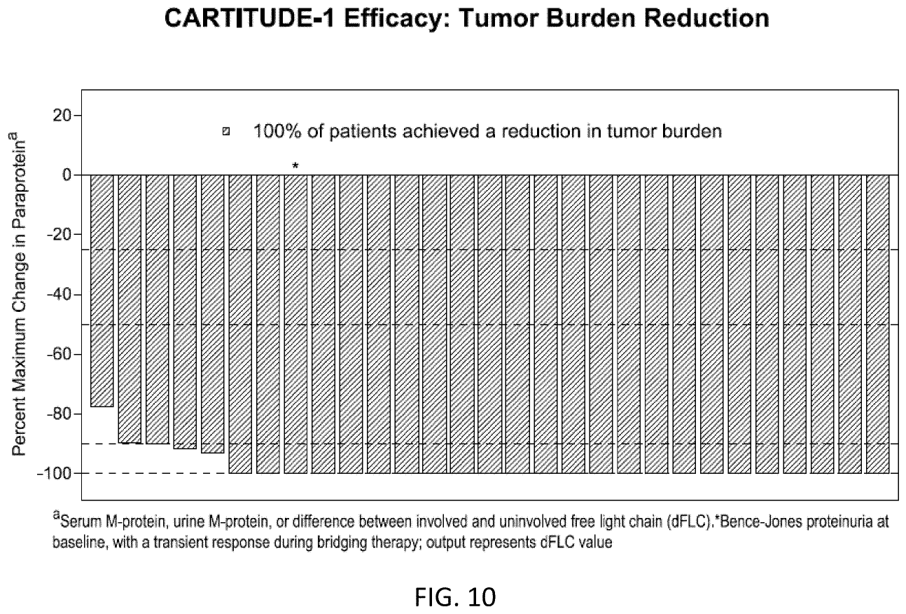

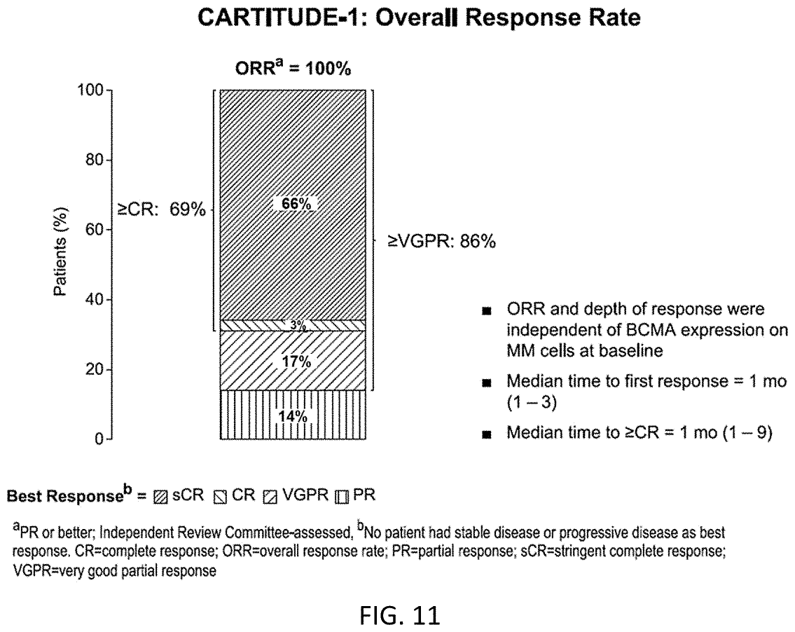

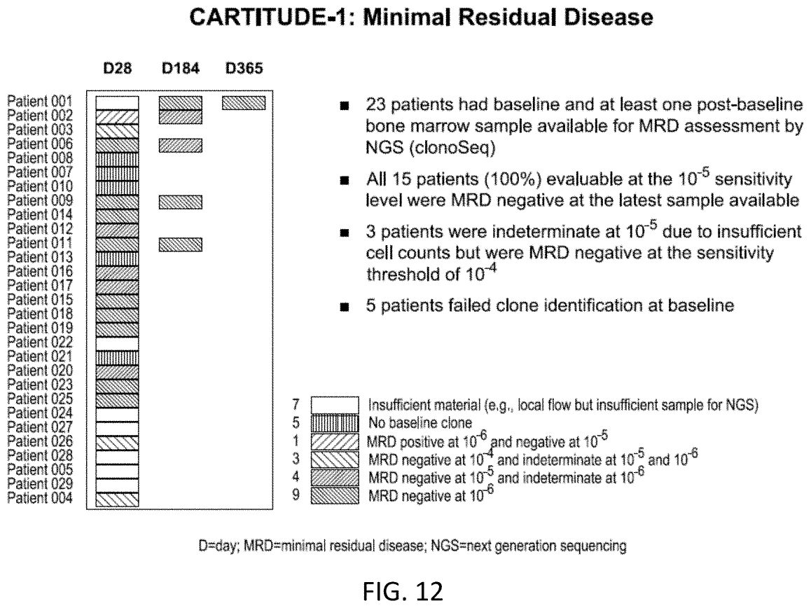

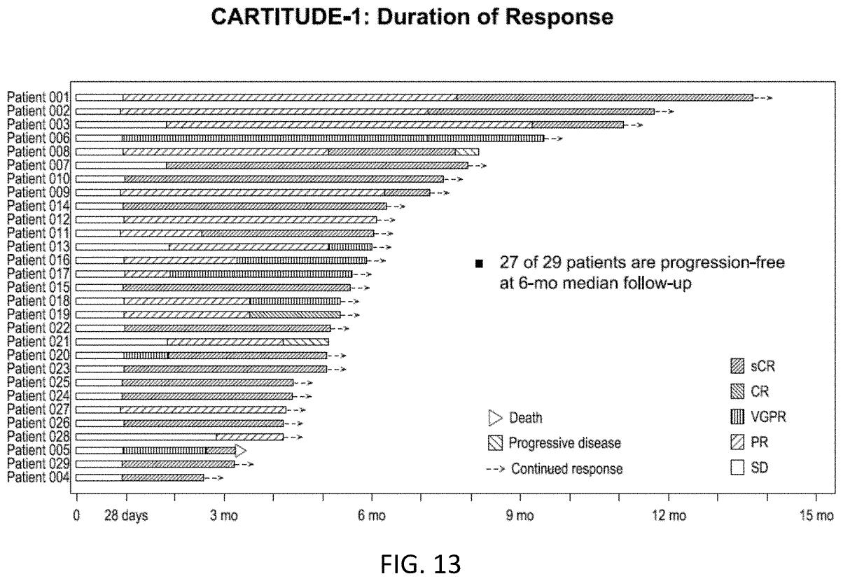

The patient population was screened to include those with relapsed or Refractory Multiple Myeloma, with 3 prior lines or double refractory to PI/IMiD and prior PI, IMiD, anti-CD38 exposure, where PI is a proteasomal inhibitor and IMiD is an immunomodulatory drug. Another possible medicament is an alkylating agent (ALKY). Eligible patients were ≥18 years of age, had a diagnosis of MINI per International Myeloma Working Group (IMWG) diagnostic criteria, measurable disease at baseline, and an Eastern Cooperative Oncology Group (ECOG) performance status score of 0, 1 or 2. Demographic and disease characteristics of the patient population in the Phase 1b portion of the study is shown in FIG. 8.

Eligible subjects underwent apheresis for collection of peripheral blood mononuclear cells (PBMC). Study enrollment was defined at the day of apheresis. The ciltacabtagene autoleucel drug product (DP) was generated from T cells selected from the apheresis. Subjects for whom apheresis or manufacturing failed were allowed a second attempt at apheresis.

Bridging therapy (anti-plasma cell directed treatment between apheresis and the first dose of the conditioning regimen) was allowed when clinically indicated (i.e., to maintain disease stability while waiting for manufacturing of ciltacabtagene autoleucel). Additional cycles of bridging therapy were considered based on the subject's clinical status and timing of availability of CAR-T product. A bridging therapy is defined as short-term treatment which had previously generated at least a response of stable disease for the subject.

After meeting safety criteria for treatment, subjects were administered a conditioning regimen to help achieve to lymphodepletion and promote CAR-T cell expansion in the subject. The lymphodepleting regimen comprised intravenous (IV) administration of cyclophosphamide 300 mg/m2 and fludarabine 30 mg/m2 daily for 3 days. Cyclophosphamide 300 mg/m2 and fludarabine 30 mg/m2 before cilta-cel infusion is consistent with the lymphodepletion regimen used in the marketed CAR-T products Kymriah and Yescarta.

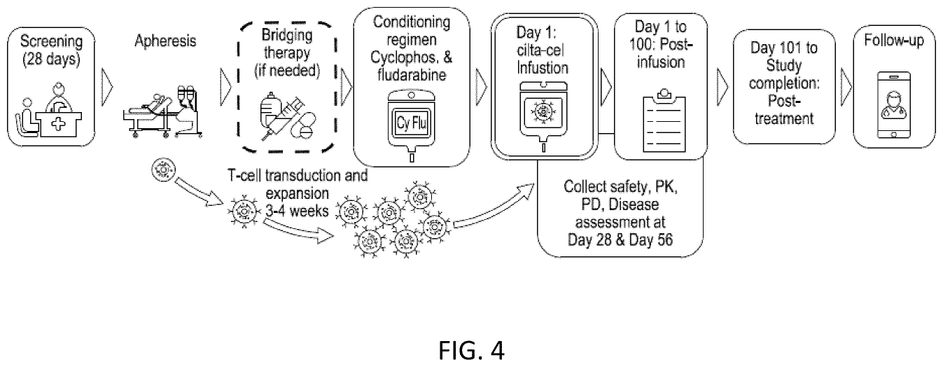

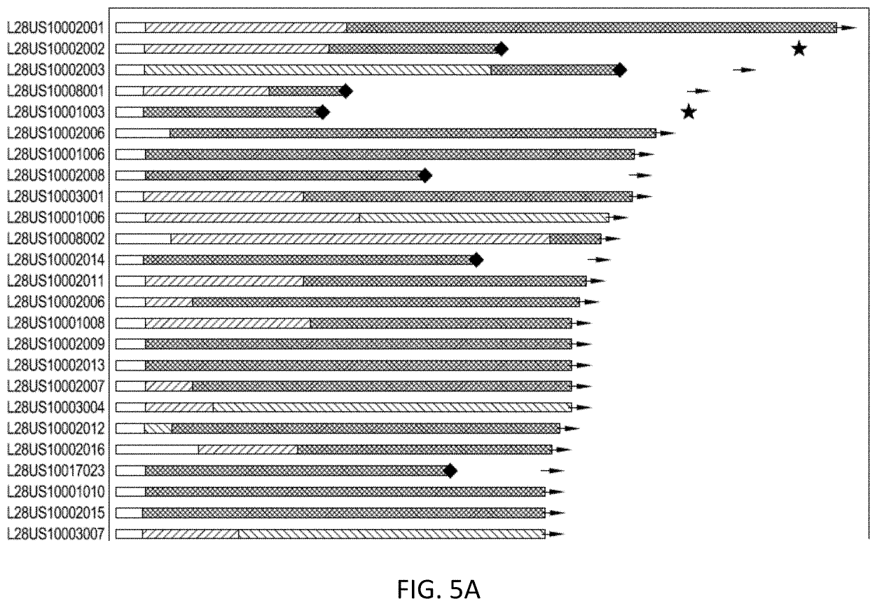

5 to 7 days after start of the conditioning regimen, cilta-cel, which had been prepared from apheresed material via viral transduction as shown in FIG. 4, was administered on a day defined as Day 1. Approximately one hour prior to cilta-cel infusion, subjects received premedication. Corticosteroids were not used during pre-infusion. Pre-infusion medication is listed in Table 5. Following treatment with the pre-infusion medication, cilta-cel administration was performed in a single infusion at a total targeted dose of 0.75×106 CAR-positive viable T cells/kg (range: 0.5-1.0×106 CAR-positive viable T cells/kg) with a maximum total dose of 1.0×108 CAR-positive viable T cells.

A dose of ciltacabtagene autoleucel was contained in either 1 or 2 cryopreserved patient-specific infusion bags. The timing of cilta-cel thaw was coordinated with the timing of the infusion. The infusion time was confirmed in advance, and the start time for thaw was adjusted so that cilta-cel was available for infusion when the patient would have been ready. If more than one bag was received for the treatment infusion, 1 bag was thawed at a time. The thawing/infusion of the next bag was made to wait until it was determined that the previous bag had been safely administered.

The post-infusion period started after the completion of cilta-cel infusion on Day 1 and lasted until Day 100. The post-treatment period started on Day 101 and lasted until study completion, defined as 2 years after the last subject had received his or her initial dose of cilta-cel. The expansion and persistence of cilta-cel as measured by blood concentration is summarized in FIG. 14.

### Example 3: Evaluation of Efficacy of Method of Treatment with Ciltacabtagene Autoleucel

Using the IMWG-based response criteria summarized in Table 6, this study classified a response, in order from better to worse, as either a stringent complete response (sCR), a complete response (CR), a very good partial response (VGPR), a partial response (PR), a minimal response (MR), a stable disease or a progressive disease. Disease progression was consistently documented across clinical study sites. The tests performed to assess IMWG-based response criteria are as follows:
Myeloma Protein Measurements in Serum and Urine: Myeloma protein (M-protein) measurements were made using the following tests from blood and 24-hour urine samples: serum quantitative Ig, serum protein electrophoresis (SPEP), serum immunofixation electrophoresis, serum FLC assay (for subject in suspected CR/sCR and every disease assessment for subjects with serum FLC only disease), 24-hour urine M-protein quantitation by electrophoresis (UPEP), urine immunofixation electrophoresis, serum β2-microglobulin. Disease progression based on one of the laboratory tests alone were confirmed by at least 1 repeat investigation. Disease evaluations continued beyond relapse from CR until disease progression was confirmed. Serum and urine immunofixation and serum free light chain (FLC) assays were performed at screening and thereafter when a CR was suspected (when serum or 24-hour urine M-protein electrophoresis [by SPEP or UPEP] were 0 or non-quantifiable). For subjects with light chain multiple myeloma, serum and urine immunofixation tests were performed routinely.
Serum Calcium Corrected for Albumin: Blood samples for calculating serum calcium corrected for albumin were collected and analyzed until the development of confirmed disease progression; development of hypercalcemia (corrected serum calcium >11.5 mg/dL [>2.9 mmol/L]) may indicate disease progression or relapse if it is not attributable to any other cause. Calcium binds to albumin and only the unbound (free) calcium is biologically active; therefore, the serum calcium level must be adjusted for abnormal albumin levels (“corrected serum calcium”).
Bone Marrow Examination: Bone marrow aspirate or biopsy was performed for clinical assessments. Bone marrow aspirate was performed for biomarker evaluations. Clinical staging (morphology, cytogenetics, and immunohistochemistry or immunofluorescence or flow cytometry) was done. A portion of the bone marrow aspirate was immunophenotyped and monitor for BCMA, checkpoint ligand expression in CD138-positive multiple myeloma cells, and checkpoint expression on T cells. If feasible, bone marrow aspirate also was performed to confirm CR and sCR and at disease progression. Additionally, since minimal residual disease (MRD) negativity was being evaluated as a potential surrogate for PFS and OS in multiple myeloma treatment, MRD was monitored in subjects using next generation sequencing (NGS) on bone marrow aspirate DNA. Baseline bone marrow aspirates were used to define the myeloma clones, and post-treatment samples were used to evaluate MRD negativity. A fresh bone marrow aspirate was collected prior to the first dose of conditioning regimen (≤7 days).
Skeletal Survey: A skeletal survey (including skull, entire vertebral column, pelvis, chest, humeri, femora, and any other bones for which the investigator suspects involvement by disease) was performed during the screening phase and evaluated by either roentgenography (“X-rays”) or low-dose computed tomography (CT) scans without the use of IV contrast. If a CT scan was used, it was of diagnostic quality. Following cilta-cel infusion, and before disease progression was confirmed, X-rays or CT scans were performed locally, whenever clinically indicated based on symptoms, to document response or progression. Magnetic resonance imaging (MRI) was an acceptable method for evaluation of bone disease, and was included at discretion; however, it did not replace the skeletal survey. If a radionuclide bone scan was used at screening, in addition to the complete skeletal survey, then both methods were used to document disease status. These tests were performed at the same time. A radionuclide bone scan did not replace a complete skeletal survey. If a subject presented with disease progression manifested by symptoms of pain due to bone changes, then disease progression was documented by skeletal survey or other radiographs, depending on the symptoms that the subject experiences. If the diagnosis of disease progression was obvious by radiographic investigations, then no repeat confirmatory X-rays were thought necessary to perform. If changes were equivocal, then a repeat X-ray was performed in 1 to 3 weeks.
Documentation of Extramedullary Plasmacytomas: Sites of known extramedullary plasmacytomas were documented ≤14 days prior to the first dose of the conditioning regimen. Clinical examination or MRI were used to document extramedullary sites of disease. CT scan evaluations were considered an acceptable alternative if there was no contraindication to the use of IV contrast. Positron emission tomography scan or ultrasound tests were not acceptable to document the size of extramedullary plasmacytomas. However, PET/CT fusion scans were optionally used to document extramedullary plasmacytomas if the CT component of the PET/CT fusion scan was of sufficient diagnostic quality. Extramedullary plasmacytomas were assessed for all subjects with a history of plasmacytomas or if clinically indicated at ≤14 days prior to the first dose of the conditioning regimen, by clinical examination or radiologic imaging. Assessment of measurable sites of extramedullary disease were performed, measured, and evaluated locally every 4 weeks (for physical examination) for subjects with a history of plasmacytomas or as clinically indicated during treatment for other subjects until development of confirmed CR or confirmed disease progression. If assessment could only be performed radiologically, then evaluation of extramedullary plasmacytomas was done every 12 weeks. Irradiated or excised lesions were considered not measurable and were monitored only for disease progression. To qualify for VGPR or PR/minimal response (MR), the sum of products of the perpendicular diameters of the existing extramedullary plasmacytomas must have decreased by over 90% or at least 50%, respectively, and new plasmacytomas must not have developed. To qualify for disease progression, either the sum of products of the perpendicular diameters of the existing extramedullary plasmacytomas must have increased by at least 50%, or the longest diameter of previous lesion >1 cm in short axis must have increased at least 50%, or a new plasmacytoma must have developed. When not all existing extramedullary plasmacytomas were reported, but the sum of products of the perpendicular diameters of the reported plasmacytomas had increased by at least 50%, then the criterion for disease progression was met.

If it was determined that the study treatment interfered with the immunofixation assay, CR was defined as the disappearance of the original M-protein associated with multiple myeloma on immunofixation, and the determination of CR was not affected by unrelated M-proteins secondary to the study treatment.

Study endpoints, as assessed by an independent review committee (IRC), were as follows:
MRD was assessed at baseline, day 28, and 6-, 12-, 18-, and 24-month follow-ups using next-generation sequencing (clonoSEQ version 2.0) (Adaptive Biotechnologies, Seattle, Wash., USA) in patients at the time of suspected complete response, and then every 12 months until disease progression for patients who remained on study. MRD negativity was assessed in samples that passed calibration or quality control and included sufficient cells for evaluation at the testing threshold of 10⁻⁵. Durability of MRD-negative status was evaluated by estimating MRD negativity rates at 6- and 12-month follow-ups.
Clinical benefit rate (CBR) was defined as the proportion of subjects who achieved a MR or better according to the IMWG criteria (sCR+CR+VGPR+PR+MR).
Overall response rate (ORR) was defined as the proportion of subjects who achieved a PR or better according to the IMWG criteria (sCR+CR+VGPR+PR).
VGPR or better response rate was defined as the proportion of subjects who achieve a VGPR or better response according to the IMWG criteria (sCR+CR+VGPR).
Duration of response (DOR) was calculated among responders (with a PR or better response) from the date of initial documentation of a response (PR or better) to the date of first documented evidence of progressive disease, as defined in the IMWG criteria. Relapse from CR by positive immunofixation or trace amount of M-protein was not considered as disease progression. Disease evaluations continued beyond relapse from CR until disease progression was confirmed.
Time to response (TTR) was defined as the time between date of the initial infusion of cilta-cel and the first efficacy evaluation at which the subject had met all criteria for PR or better.
Progression-free survival (PFS) was defined as the time from the date of the initial infusion of cilta-cel to the date of first documented disease progression, as defined in the IMWG criteria, or death due to any cause, whichever occurred first.
Overall survival (OS) was measured from the date of the initial infusion of cilta-cel to the date of the subject's death.

For ORR, the response rate and its 95% exact confidence interval (CI) was calculated based on binomial distribution, and the null hypothesis was rejected if the lower bound of the confidence interval exceeded 30%. Analysis of VGPR or better response rate, DOR, PFS, and OS was conducted at the same cutoff as the ORR. Time-to-event efficacy endpoints (DOR, PFS, and OS) were estimated using the Kaplan-Meier method. The distribution (median and Kaplan-Meier curves) of DOR was provided using Kaplan-Meier estimates. Similar analysis was performed for OS, PFS, and TTR.

### Example 4: Evaluation of Safety of Method of Treatment with Ciltacabtagene Autoleucel

Adverse events were followed, reported and graded according to the National Cancer Institute Common Terminology Criteria for Adverse Events (NCI-CTCAE Version 5.0), with the exception of CRS and CAR-T cell-related neurotoxicity (e.g., ICANS). CRS was evaluated according to the ASTCT consensus grading, summarized in Table 7. At the first sign of CRS (such as fever), subjects were immediately hospitalized for evaluation. Tocilizumab intervention was discretionally used to treat subjects presenting symptoms of fever when other sources of fever had been eliminated. Tocilizumab was discretionally used for early treatment in subjects at high risk of severe CRS (for example, high baseline tumor burden, early fever onset, or persistent fever after 24 hours of symptomatic treatment). Other monoclonal antibodies targeting cytokines (for example, anti-IL1 and/or anti-TNFα) were optionally used, especially for cases of CRS which did not respond to tocilizumab.

CAR-T cell-related neurotoxicity (e.g., ICANS) was graded using the ASTCT consensus grading, summarized in Table 8. Additionally, all individual symptoms of CRS (e.g., fever, hypotension) and ICANS (e.g., depressed level of consciousness, seizures) were captured as individual adverse events and graded by CTCAE criteria. Neurotoxicity that was not temporarily associated with CRS, or any other neurologic adverse events that did not qualify as ICANS, were graded by CTCAE criteria. Any adverse event or serious adverse event not listed in the NCI CTCAE Version 5.0 was graded according to investigator clinical judgment by using the standard grades as follows:
Grade 1: Mild; asymptomatic or mild symptoms; clinical or diagnostic observations only; intervention not indicated.
Grade 2: Moderate; minimal, local or noninvasive intervention indicated; limiting age-appropriate instrumental activities of daily living.
Grade 3: Severe or medically significant but not immediately life-threatening; hospitalization or prolongation of hospitalization indicated; disabling; limiting self-care activities of daily living.
Grade 4: Life-threatening consequences; urgent intervention indicated.
Grade 5: Death related to adverse event.

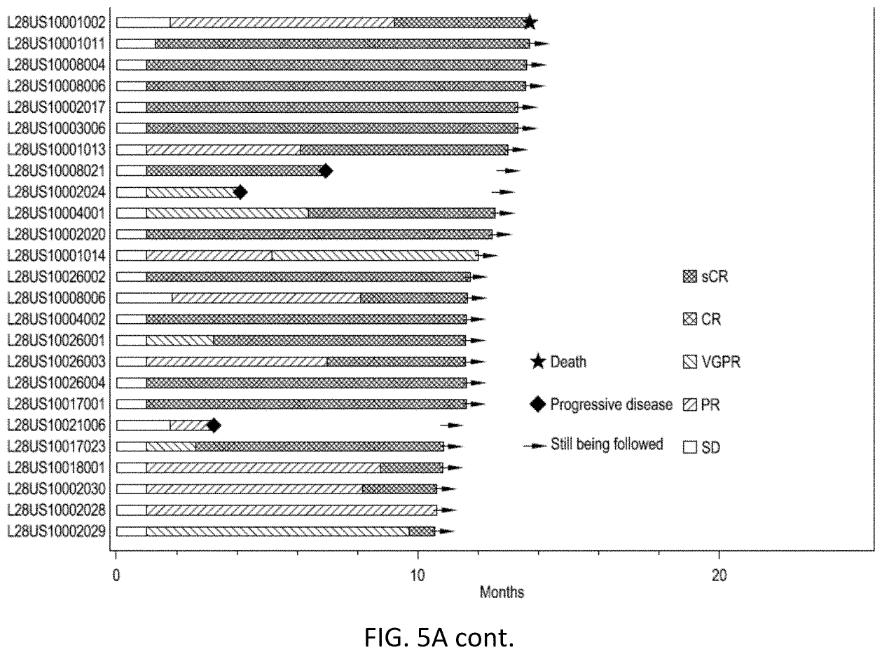

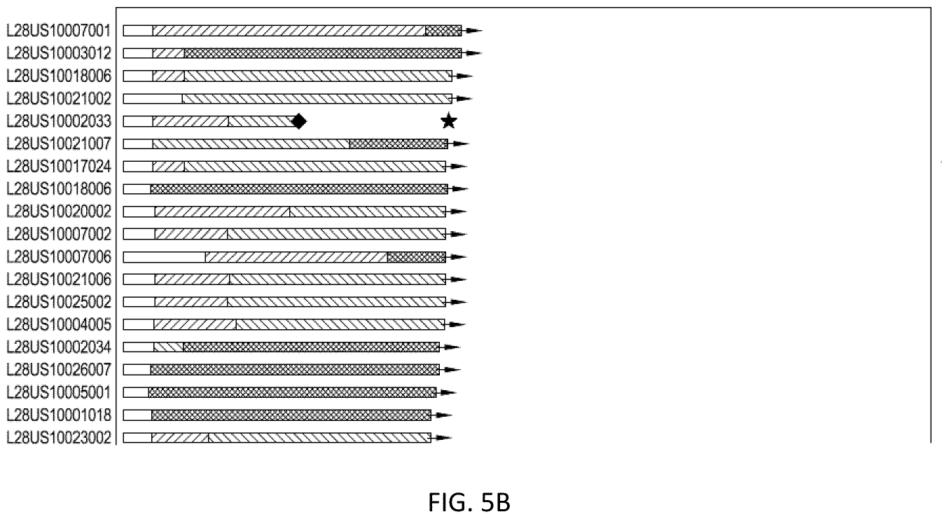

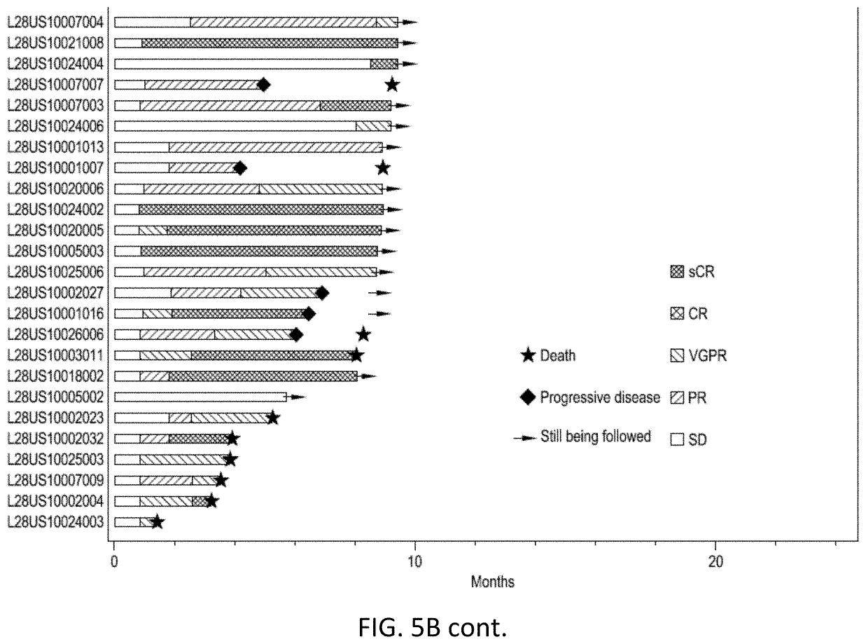

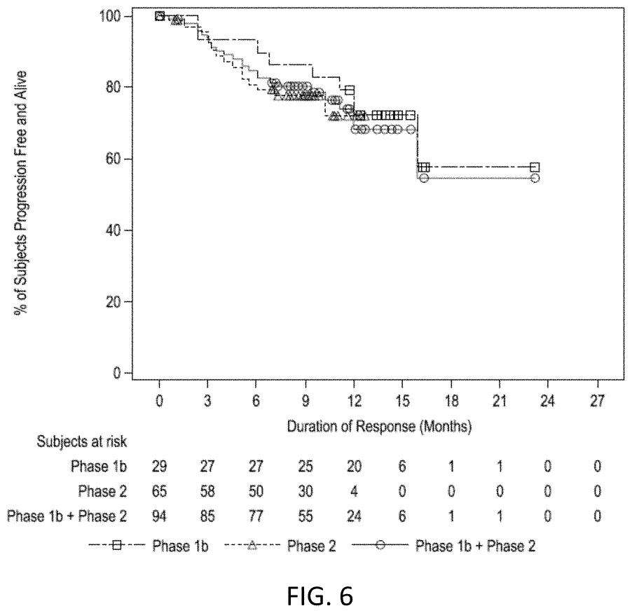

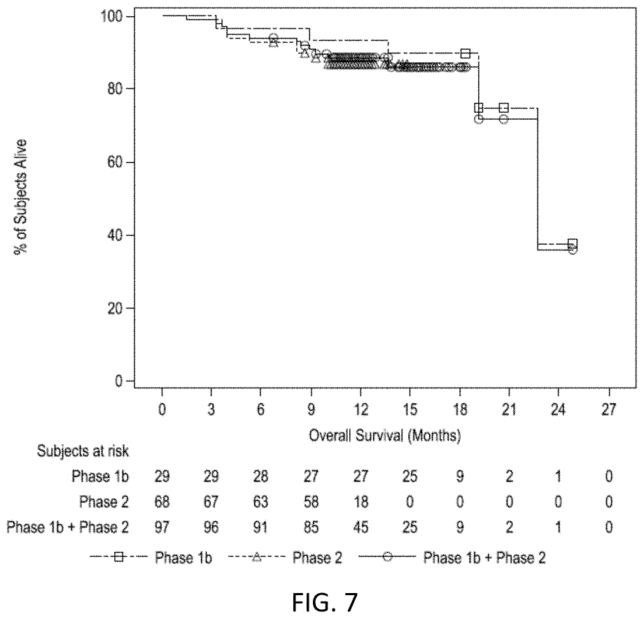

The response and duration of response of responders in the all treated analysis set at a median follow-up time of 12.4 months, based on Independent Review Committee (IRC) assessment, is presented in FIG. 5. The overall best response for subjects in the all treated analysis set is summarized in Table 9. In the all treated analysis set, based on IRC assessment 94 subjects (96.9%) achieved a response of PR or better, 65 subjects (67.0%) achieved complete response (CR) or better, CBR was 96.9%. The deep and durable response induced by ciltacabtagene autoleucel were demonstrated by a VGPR or better rate of 92.8% and a CR or better rate of 67.0%, and a median DOR not reached with a median follow-up of 12.4 months at the time of clinical cutoff. The metrics used to evaluate ciltacabtagene autoleucel efficacy are summarized below:
Tumor burden reduction: Tumor burden was reduced in 100% of subjects. A graph of the tumor burden reduction in patients in the phase 1b-2 study is presented in FIG. 10.
Overall Response Rate (ORR): 96.9% of subjects had overall responses, with 95% exact CI (91.2%, 99.4%). A summary of the ORR in patients in the phase 1b-2 study is presented in FIG. 11.
VGPR or better: 90 subjects (92.8% of subjects) achieved VGPR (very good partial response) or better.
Duration of Response (DOR): Median DOR was not reached with 95% CI (15.9, NE) months; the probabilities of the responders remaining in response at 9 months and 12 months were 80.2% (95% CI: 70.4%, 87.0%) and 68.2% (95% CI: 54.4%, 78.6%), respectively. A Kaplan-Meier plot for DOR for all responders in the all treated analysis set is presented in FIG. 6, and DOR for all responders in the all treated analysis set is summarized in Table 10. A graph of the DOR for patients in the phase 1b-2 study is presented in FIG. 13.
Time to Response (TTR): Median time to first response (PR or better) and median time to best response were 0.95 and 2.56 months, respectively.
Progression-Free Survival (PFS): Median PFS was not reached with 95% CI (16.79, NE) months; 9-month and 12-month PFS rates (95% CI) were 80.3% (70.9%, 87.0%) and 76.6% (66.0%, 84.3%), respectively. A summary of the PFS in the all treated analysis set is presented in Table 11.
Overall Survival (OS): Fourteen subjects (14.4%) had died at the time of clinical cutoff. Nine-month and 12-month overall survival rates (95% CI) were 90.7% (82.8%, 95.0%) and 88.5% (80.2%, 93.5%), respectively. A Kaplan-Meier plot for OS based on the all treated analysis set is presented in FIG. 7, and OS based on the all treated analysis set is summarized in Table 12.
Mean Residual Disease (MRD) negative rate (at 10⁻⁵ sensitivity level): MRD negative rate was 54.6% (95% CI: 44.2%, 64.8%), and 33 (34.0%) subjects achieved MRD-negative CR/sCR. Summaries of overall MDR negativity rate at 10⁻⁵ in the bone marrow are presented, for all subjects in the all treated analysis set in Table 13 and for subjects with evaluable sample at 10⁻⁵ in the all treated analysis set in Table 14. Evaluable samples were those that passed calibration and quality control, and included sufficient cells for evaluation at the respective testing threshold. A summary of the MRD in patients in the phase 1b-2 study is presented in FIG. 12 and FIG. 15.

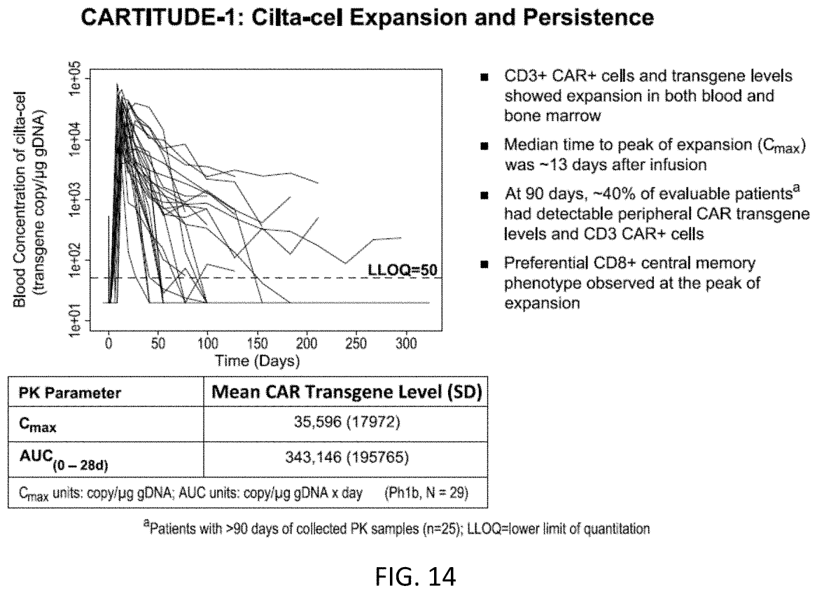

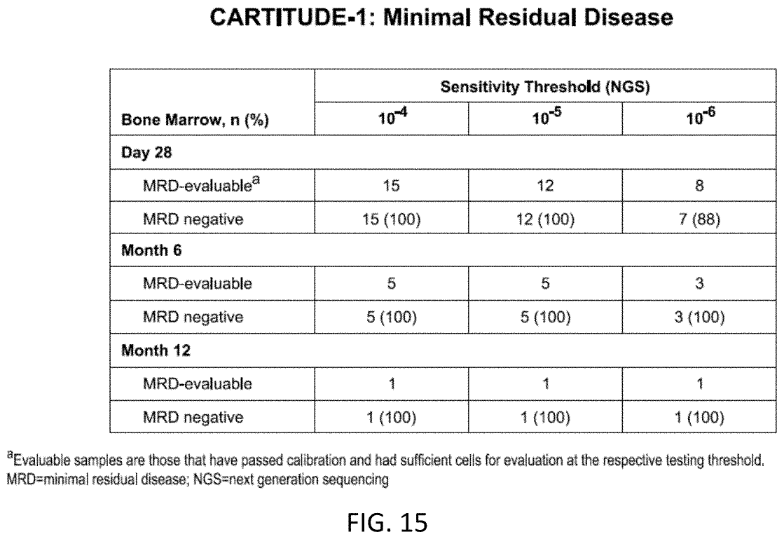

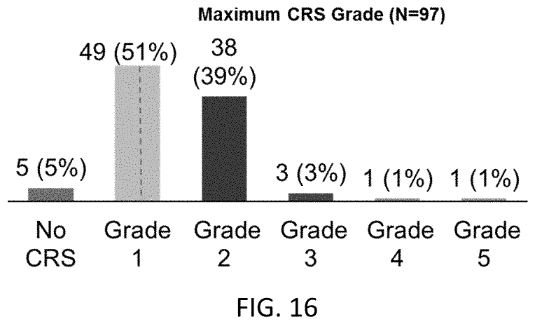

Ciltacabtagene autoleucel was determined to have a safety profile consistent with the mechanism of action of CAR-T therapy. A summary of adverse events in the Phase 1b-2 trial is shown in FIG. 9.
CRS: CAR-T cell-related adverse events of CRS were common (94.8%) but most were low grade. All-grade CRS was reported for 92 (94.8%) subjects, as evaluated by the ASTCT consensus grading system. All events of CRS had recovered, with the exception of 1 (1.1%) fatal event from a subject with a 97-day duration of CRS. A summary of treatment-emergent CRS events in the all treated analysis set is presented in Table 15 and FIG. 16. FIGS. 17-19 present graphs and summaries of various CRS protein markers observed in all patients.
Immune Effector Cell-Associated Neurotoxicity (ICANS): All-grade ICANS was reported for 16 (16.5%) subjects, as evaluated by the ASTCT consensus grading system. All events had recovered. A summary of ICANS, with onset after cilta-cel infusion, in the all treated analysis set is presented in Table 16.
Cytopenias: Grade 3 or 4 cytopenias were common in the post-infusion period, including lymphopenia, neutropenia, thrombocytopenia, but the majority of these events recovered by Day 60. 96 (99.0%), 95 (97.9%) and 60 (61.9%) subjects had Grade 3 or 4 lymphopenia, neutropenia, and thrombocytopenia, respectively, in the first 100 days after cilta-cel infusion. 88 (90.7%), 85 (87.6%), and 41 (42.3%) subjects had their initial Grade 3 or 4 events recovered to Grade 2 or lower by Day 60 for lymphopenia, neutropenia, and thrombocytopenia, respectively. A summary of cytopenias following treatment with cilta-cell in the all treated analysis set is presented in Table 17.

In conclusion, single-agent and one-time infusion of ciltacabtagene autoleucel demonstrated unprecedented clinical activity in a heavily pretreated patient population, including with an ORR of 96.9% and rapid onset of response in less than 1 month.

### Example 5: Results from Evaluation of Method of Treatment with Ciltacabtagene Autoleucel at a Median Follow-Up Time of 18 Months

As of this analysis, 97 patients received a cilta-cel infusion (median administered dose, 0.71×106; range, 0.51×106-0.95×106 CAR-positive viable T cells/kg). Of 91 patients with baseline cytogenetic data, 23 patients (23.7%) had a high-risk cytogenetic profile based on the presence of at least one chromosomal abnormality, including dell7p (19.6%), t(4;14) (3.1%) and/or t(14;16) (2.1%). Of 96 patients with evaluable bone marrow biopsy and/or aspirate samples, more than half of patients (60.4%) had low disease burden (≤30 plasma cells), 21.9% patients had high disease burden (≥60 plasma cells), and 17.7% patients had intermediate disease burden (>30 to <60 plasma cells). Plasmacytomas at screening were detected in 19.6% patients.

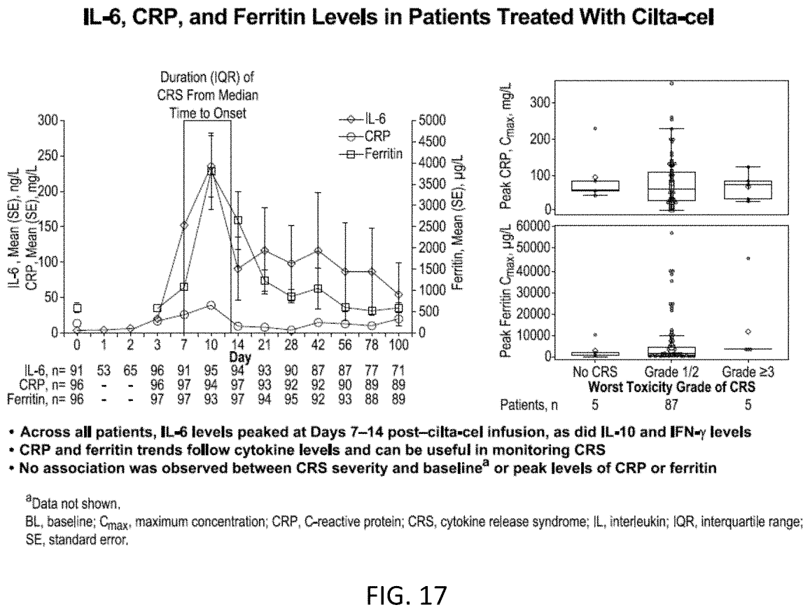

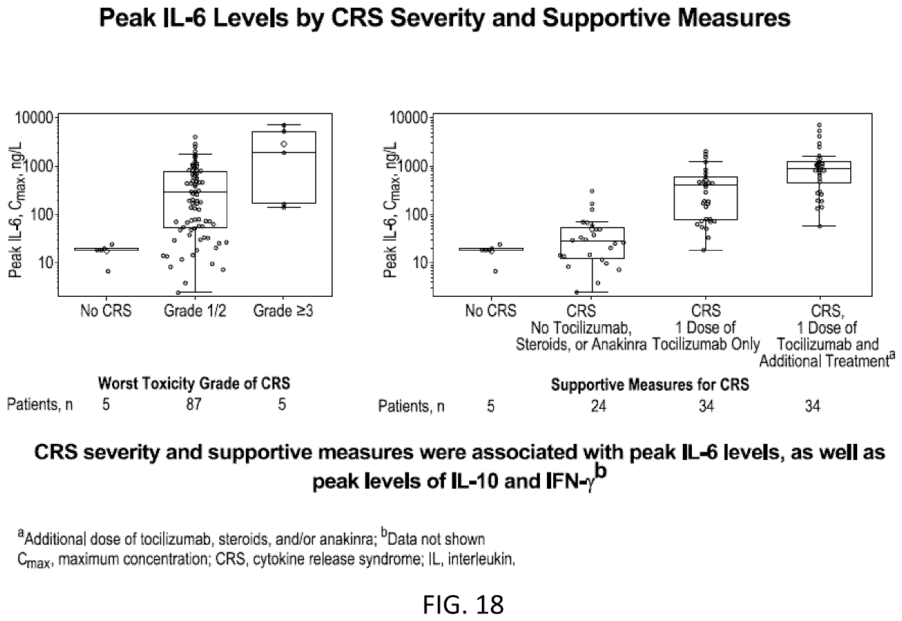

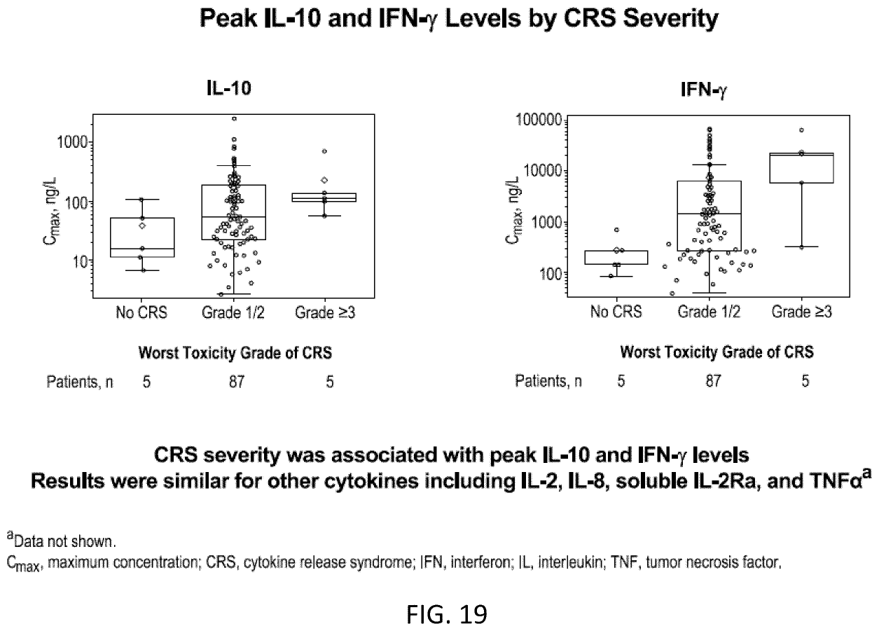

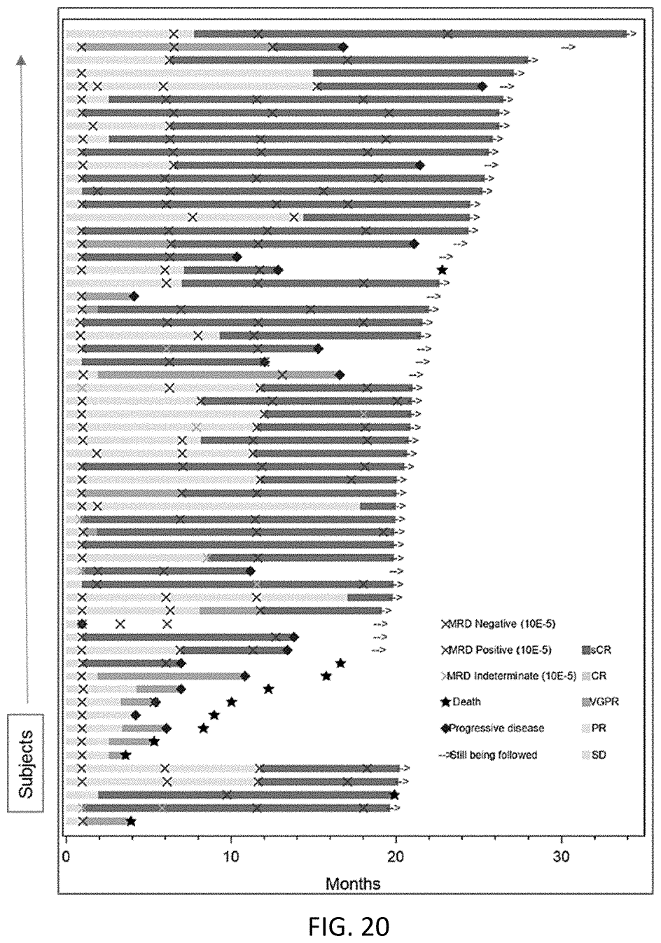

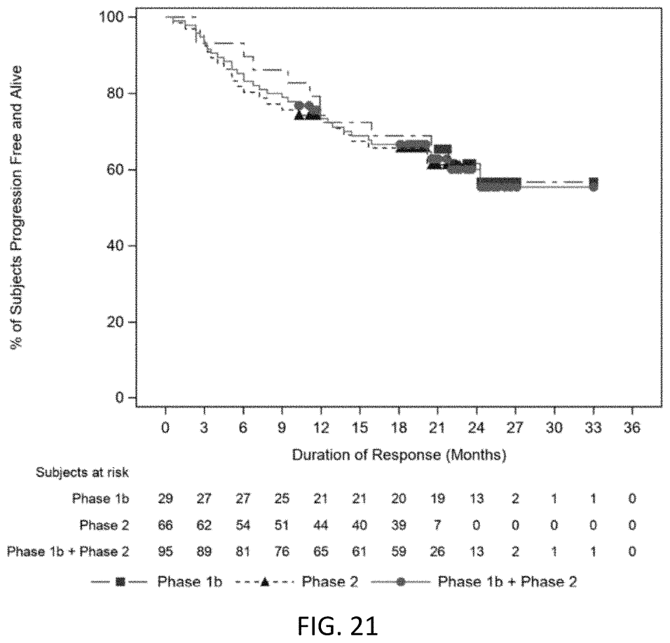

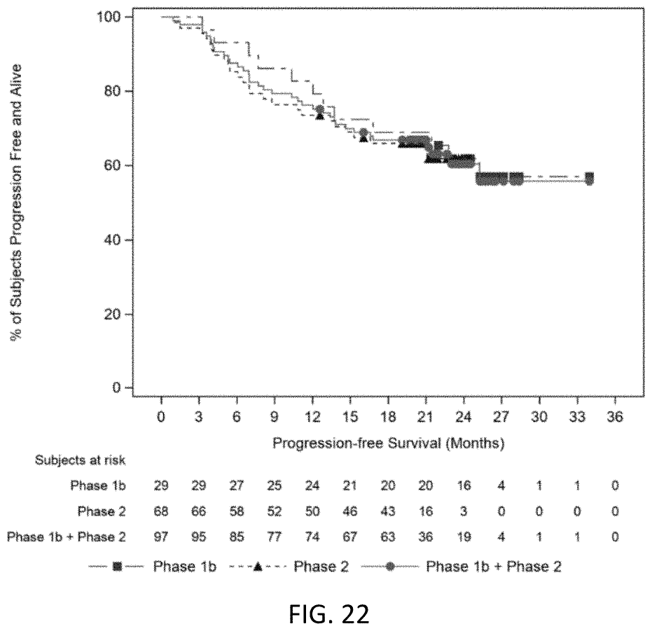

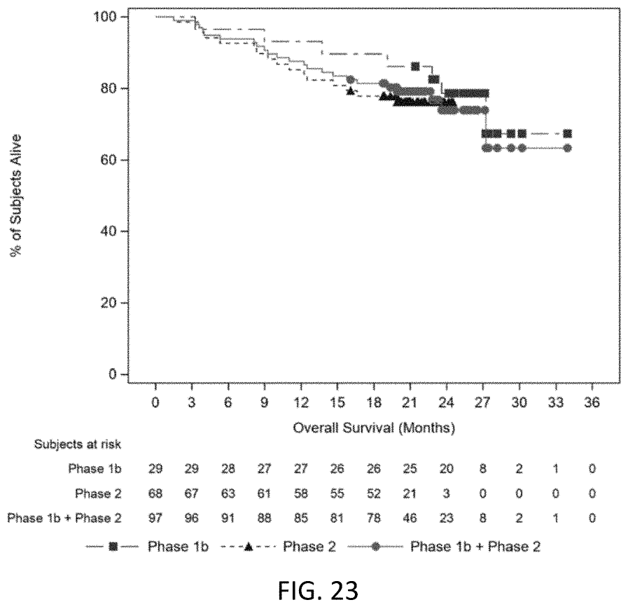

Tables 18-26 and FIGS. 20-23 detail various safety/efficacy parameters at a median follow-up time of 18 months, which are also summarized below.

### Efficacy

At the median follow-up of 18 months, ORR was 97.9% (95% CI, 92.7-99.7), sCR rate was 80.4%, VGPR rate was 14.4%, and PR rate was 3.1% (Table 1).
The median time to first response was 1 month (range, 0.9-10.7), median time to best response was 2.6 months (range, 0.9-15.2), and the median time to CR or better was 2.6 months (range, 0.9-15.2).
The median DOR was 21.8 months (95% CI, 21.8—not estimable) in the overall population, and was not reached in patients with sCR.
Median PFS was 22.8 months (95% CI, 22.8—not estimable) in all patients, and was not reached in patients with sCR (FIG. 20). The 18-month PFS rates were 66.0% (95% CI, 54.9-75.0) and 75.9% (95% CI, 63.6-84.5) in all patients and patients with sCR, respectively.
The 18-month OS rate in all patients was 80.9% (95% CI, 71.4-87.6) (FIG. 20).
Of 61 patients evaluable for MRD, 91.8% achieved MRD-negative status at the 10⁻⁵ threshold. MRD negativity was sustained for ≥6 months in 44.3% (27/61) and ≥12 months in 18% (11/61) of patients.
   The 18-month PFS rates in patients who achieved sustained MRD for ≥6 months and ≥12 months were 96.3% (95% CI, 76.5-99.5) and 100%, respectively.
ORRs were consistently high across all subgroups (range, 95.1-100%), including patients treated with 3 prior lines of therapy (100% [95% CI, 80.5-100]), with a high-risk cytogenetic profile (100% [95% CI, 85.2-100]), high disease burden (≥60% bone marrow plasma cells; 95.2% [95% CI, 76.2-99.9]), and in patients with plasmacytomas (100.0% [95% CI, 82.4-100]) (Table 2). MRD negativity rates (threshold, 10⁻⁵) were 80-100% across all subgroups of MRD-evaluable patients.
Despite consistent ORR and MRD negativity benefit, other efficacy outcomes were not maintained in particular subgroups.
Patients at ISS stage III had lower median DOR (13.8 months [95% CI, 5.1—not estimable]), 18-month PFS rate (34.3% [95% CI, 9.4-61.6]), and 18-month OS rate (48.2% [95% CI, 20.8-71.2]), while patients with presence of plasmacytomas at baseline had lower median DOR (6.8 months [4.0—not estimable]), 18-month PFS rate (46.8% [95% CI, 23.7-67.0]), and 18-month OS rate (64.5% [95% CI, 35.6-83.0]).
Patients with high tumor burden (≥60% bone marrow plasma cells) had lower 18-month PFS rate (50.6% [95% CI, 27.5-69.9]), and 18-month OS rate (71.4% [95% CI, 47.2-86.0]).

### Safety

No new safety signals were observed in patients treated with cilta-cel with longer duration of follow-up.
The most common (≥25%) grade 3/4 treatment-emergent AEs (TEAE) were neutropenia (94.8%), anemia (68.0%), leukopenia (60.8%), thrombocytopenia (59.8%), and lymphopenia (49.5%) (Table 3).
The most common grade 3/4 non-hematologic TEAEs were hypophosphatemia (7.2%), fatigue (5.2%) and increased aspartate aminotransferase (AST) (5.2%) (Table 3).
CRS was reported in 94.8% (n=92) of patients (94.6% were grade 1/2), with the median onset time of 7 days (range, 1-12) and the median duration of 4 days (range, 1-97). CRS resolved within 14 days in 91 of 92 patients; one patient with grade 5 CRS and hemophagocytic lymphohistiocytosis died on day 99 subsequent to sequelae of prolonged grade 4 CRS.
No new neurotoxicity events were reported with the extended follow-up, and no new cases of neurocognitive TEAEs were identified compared with the primary analysis.
One patient with progressive disease was retreated with cilta-cel; the patient had stable disease post-retreatment (per computerized algorithm) and no occurrence of treatment-related neurotoxicity.

This extended follow up of the cilta-cel trial described herein demonstrates that cilta-cel maintained clinical benefit in patients with RRMM with a median of 6 prior therapies for 18 months and such patients experienced a manageable side-effect profile with no new safety signals. ORR remained high (97.9%) over an additional 6 months of follow up. Deep and durable responses were observed, with 80.4% of patients showing sCR. MRD negativity was also maintained in 91.8% of evaluable patients and was sustained for 6-12 months. Assessment of durability of MRD-negative status was of particular interest given evidence showing the prognostic value of MRD negativity for improved long-term survival outcomes across multiple disease settings, including patients exposed to several lines of prior therapy.

After 18 months of follow-up for cilta-cel, no new safety signals were identified. The most frequently reported TEAEs were consistent with the side-effect profile of cilta-cel reported in the primary analysis. No new neurotoxicity events were reported, and no new cases of movement and neurocognitive TEAEs were observed.

In conclusion, data from the cilta-cel trial with longer follow-up described herein confirm substantial clinical benefits of cilta-cel in triple-class exposed patients with RRMM.

### Example 6: Results from Evaluation of Method of Treatment with Ciltacabtagene Autoleucel in Patient Subgroups

Next, we assessed the efficacy and safety of cilta-cel in various subgroups of patients in the Phase 1b-2 cilta-cel clinical trial. Eligible patients had multiple myeloma (MM) and had received at least 3 prior regimens or were double refractory to a proteasome inhibitor (PI) and immunomodulatory drug (IMiD), and had received a PI, IMiD, and anti-CD38 antibody. After apheresis, bridging therapy was permitted. Patients received a single cilta-cel infusion (target dose: 0.75×106 CAR+ viable T cells/kg; range 0.5-1.0×106) 5-7 days after lymphodepletion (300 mg/m2 cyclophosphamide, 30 mg/m2 fludarabine daily for 3 days). Primary objectives were to characterize cilta-cel safety, confirm the recommended phase 2 dose (phase 1b), and evaluate efficacy (phase 2). Cytokine release syndrome (CRS) was graded by Lee et al (Blood 2014) and neurotoxicity by Common Terminology Criteria for Adverse Events (CTCAE), v5.0 (in phase 1b). CRS and Immune effector cell-associated neurotoxicity (ICANS) were graded by American Society for Transplantation and Cellular Therapy (ASTCT) criteria (in phase 2). Here, Lee et al and CTCAE v5.0 were mapped to ASTCT for CRS and ICANS, respectively. Efficacy and safety were evaluated in the following subgroups by baseline (BL) characteristics: ≥65 years of age, Black/African American, 3 prior lines of therapy (LOT), ≥4 prior LOT, triple-class refractory, penta-drug refractory, standard- and high-risk cytogenetics, International Staging System stage III, bone marrow plasma cells (≤30%, >30 to <60%, and ≥60%), BCMA tumor expression (<median, ≥median), and presence of plasmacytomas (bone based and extramedullary).

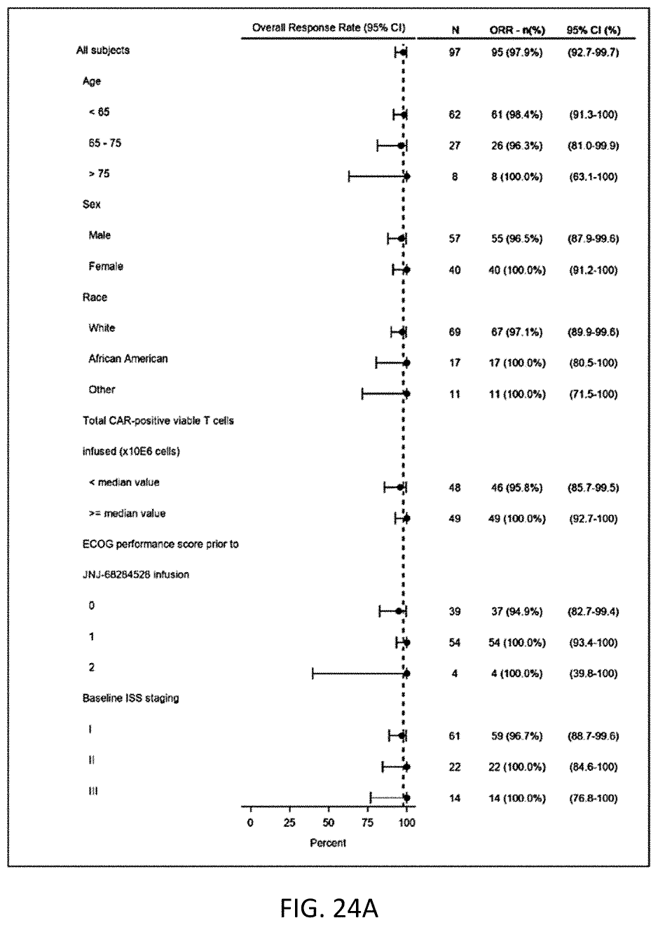

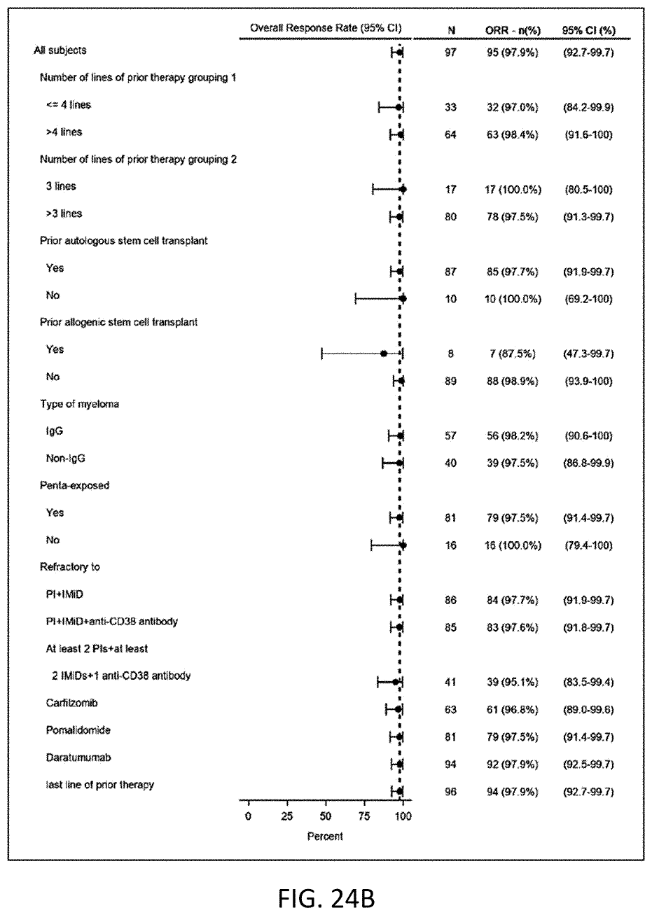

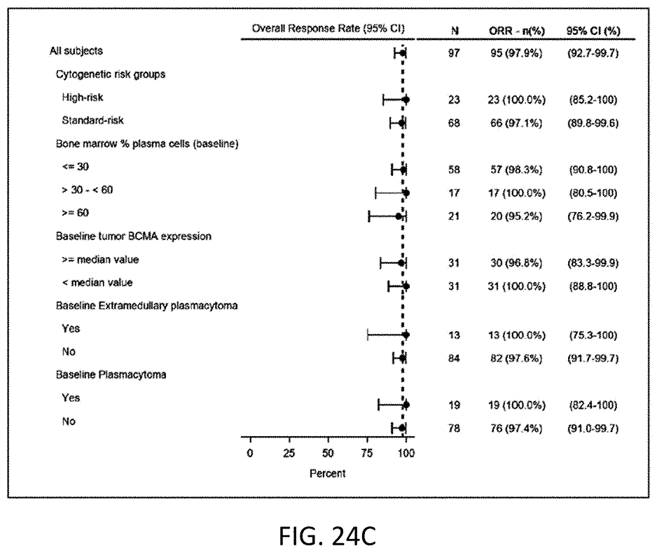

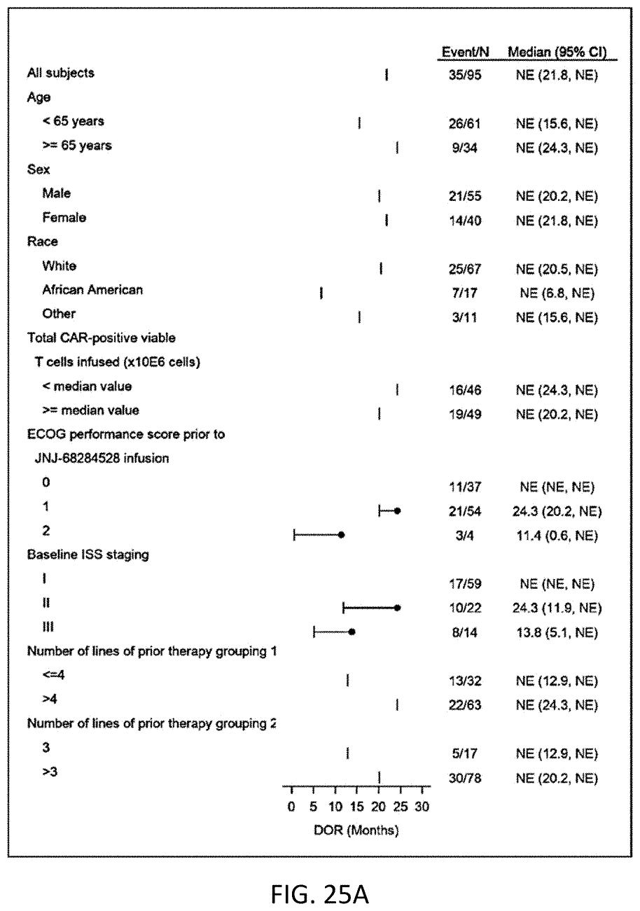

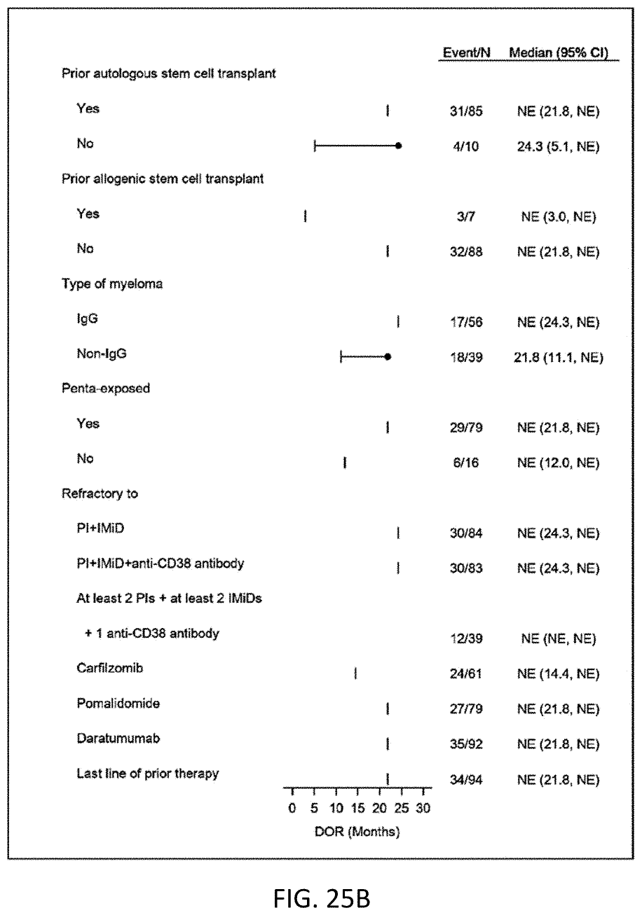

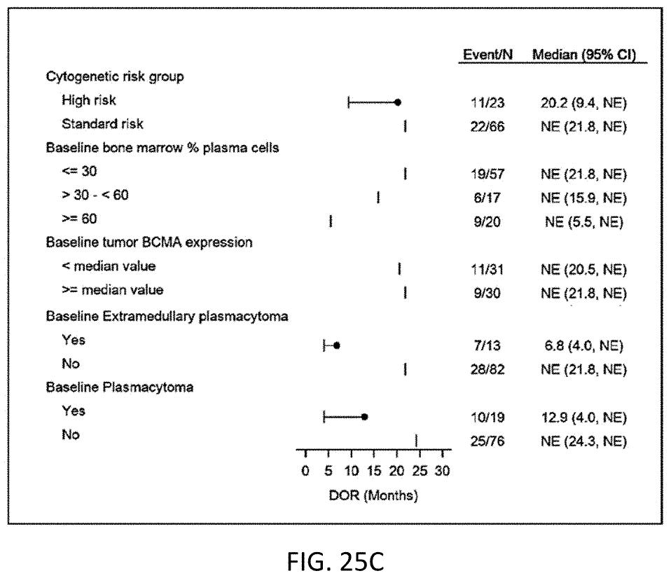

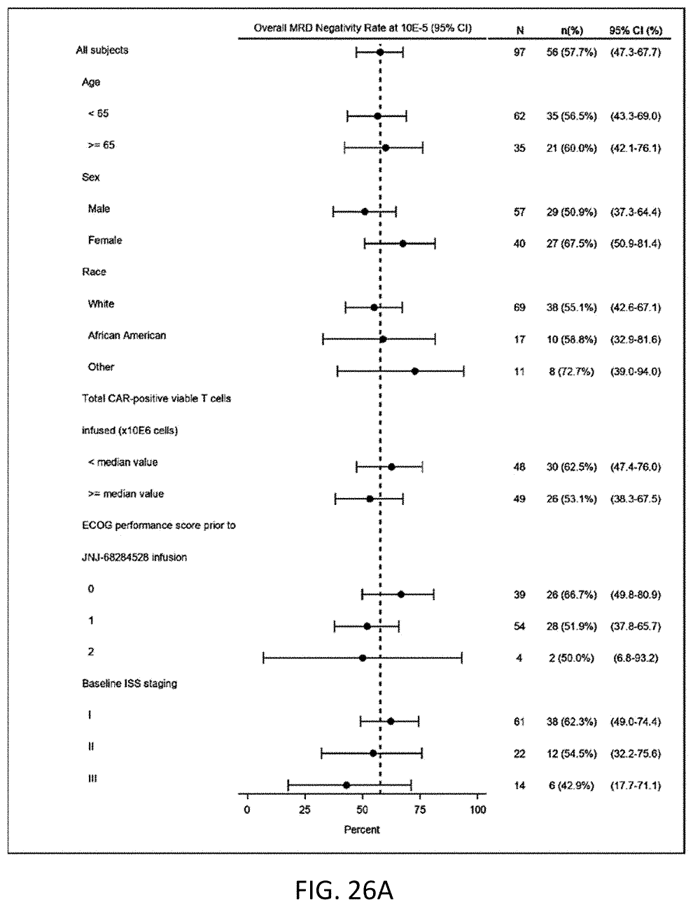

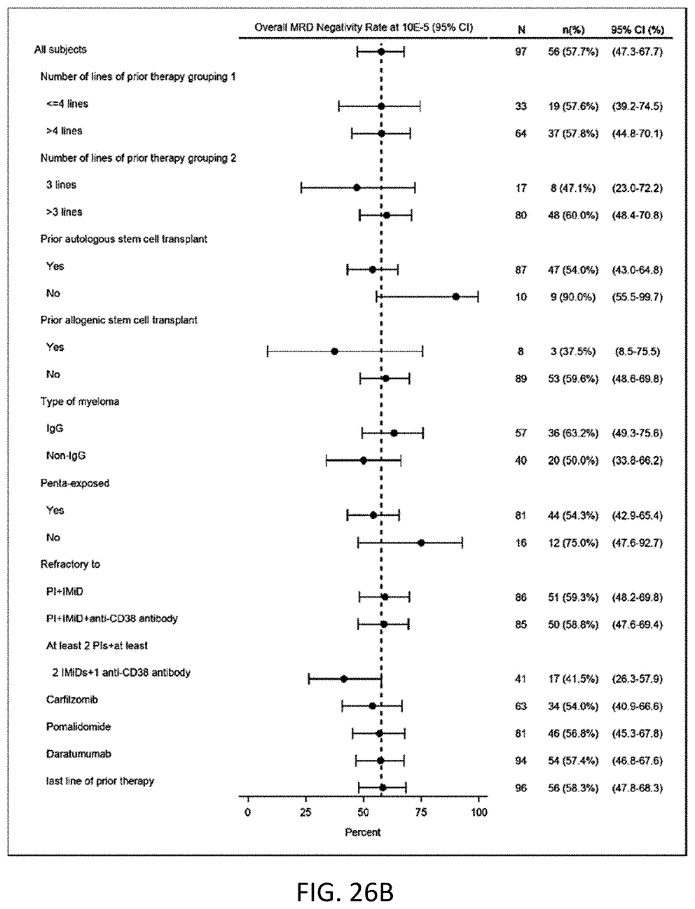

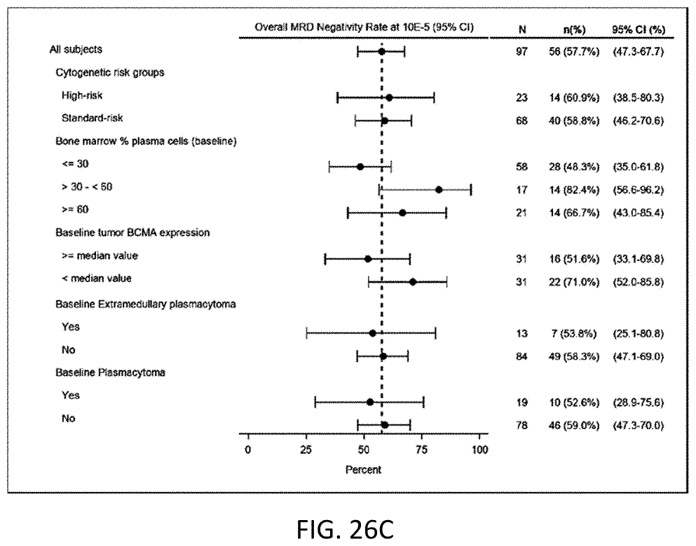

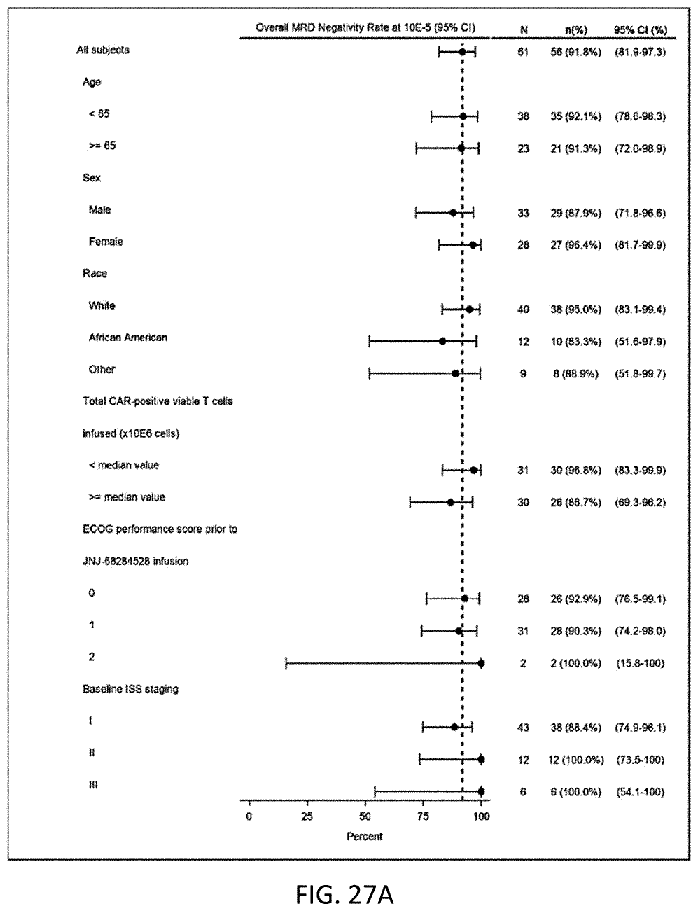

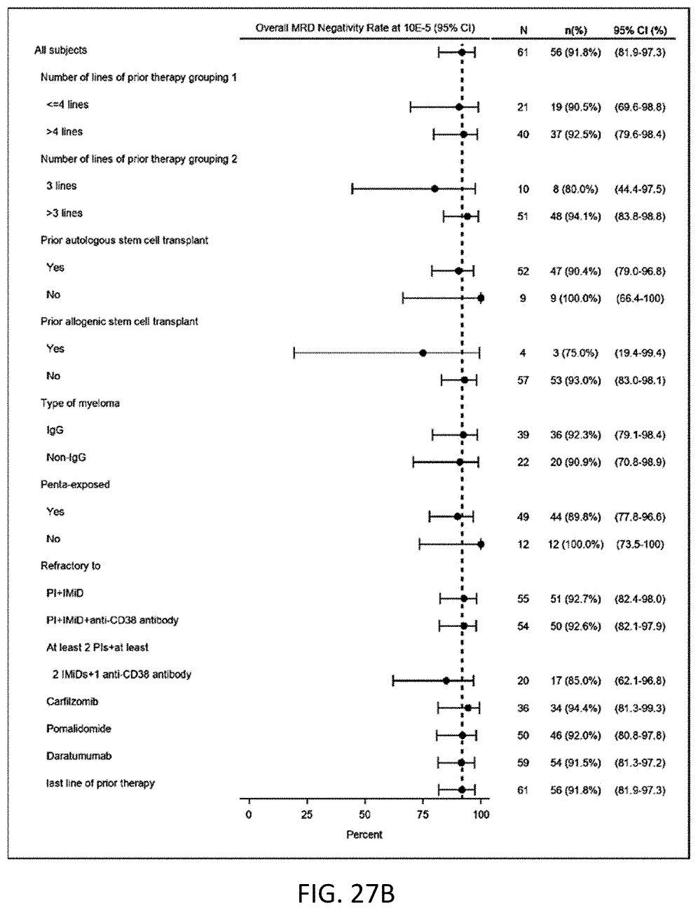

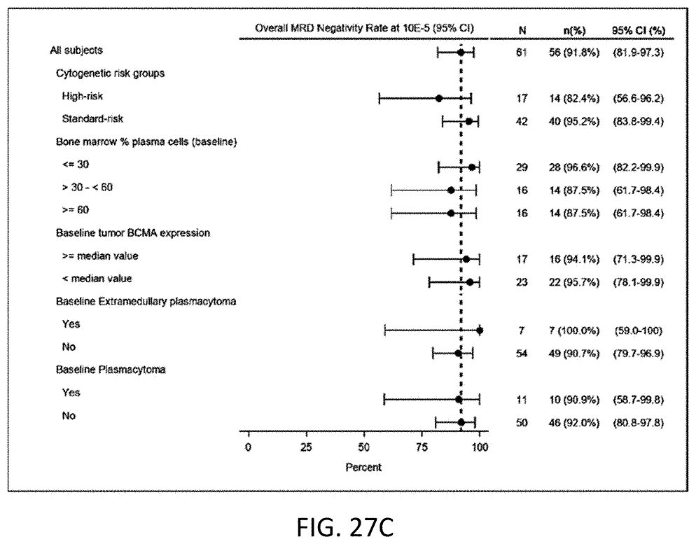

Tables 28-32 and FIGS. 24-27 detail various safety/efficacy parameters for the various subgroups analyzed at a median follow-up time of 18 months. These parameters are also summarized below.

As of this subgroup analysis, at a median follow-up time of 18 months, 97 patients in the overall population (58.8% male; median age, 61 years [range 43-78]; median time from diagnosis to enrollment was 5.9 years [1.6-18.2]) received cilta-cel. Efficacy outcomes were comparable to the overall population, with consistently high ORR (range 95.1-100%; Table) across all evaluated subgroups, including those with high-risk cytogenetics, ISS stage III MM, BL bone marrow cells ≥60%, and BL plasmacytomas. Median DOR and median PFS were consistent with the overall population or not reached for most subgroups, whereas it was lower in patients with high-risk disease such as ISS stage III and BL plasmacytomas (Table). Across all subgroups, the majority of patients (80%-100%) who were evaluable for MRD at the 10-5 threshold achieved MRD negativity. The 18-month PFS and OS rates were consistent with the overall population in most subgroups. These improvements were also achieved in patients with high-risk disease, albeit at lower rates (Table). Incidence of CRS, ICANS, and other CAR T-cell neurotoxicities (events not reported as ICANS [i.e., onset after a period of recovery from CRS and ICANS]) in various subgroups was consistent with the overall population, with no new safety signals.

At a median follow-up of 18 months, a single infusion of cilta-cel yielded deep, durable responses in all evaluated high-risk subgroups of patients with poor prognoses described herein. ORR was achieved in 90%-100% of patients across various subgroups, including those with high-risk cytogenetics, ISS stage III MM, BL bone marrow cells ≥60%, and BL plasmacytomas. Cilta-cel safety profile across the subgroups was consistent with the overall population, with no new safety signals.

### Example 7: Comparison of Method of Treatment with Ciltacabtagene Autoleucel with Physician's Choice Treatment

As described in Examples 2-4, supra, cilta-cel is effective and safe in patients with relapsed or refractory multiple myeloma (“RRMM”) who are triple-class exposed (to immunomodulatory drugs, proteasome inhibitors, and an anti-CD38 monoclonal antibody). As there is no clear standard of care for this indication, and due to an absence of direct head-to-head trials evaluating cilta-cel and other relevant treatments, we performed indirect treatment comparisons (ITCs) between cilta-cel and treatments used in current clinical practice, i.e., physician's choice of treatment (“PCT”). Briefly, meta-analyses were performed to derive single summary effect estimates for overall survival (“OS”) and progression-free survival (“PFS”) by pooling ITCs evaluating cilta-cel versus PCT in patients with triple-class exposed RRMM.

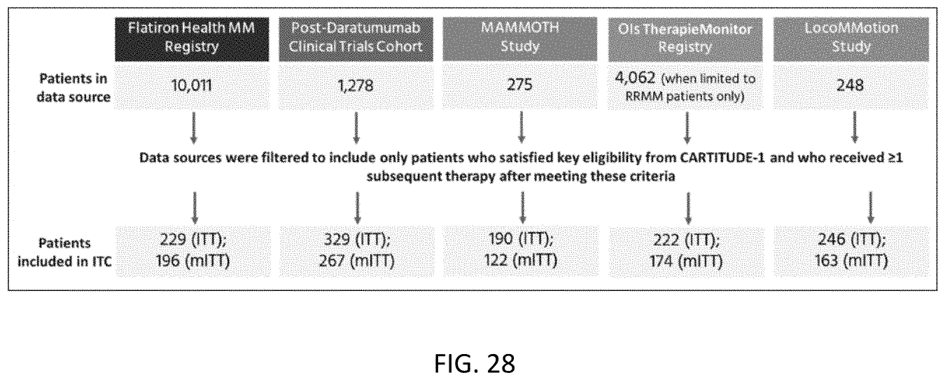

ITCs examining the comparative effectiveness of cilta-cel versus PCT on OS and PFS were included. The selection of comparator arms for ITC analyses is summarized in FIG. 28. Data on PCT was leveraged from the following sources: (i) the Flatiron database, a primarily US community-based multiple myeloma registry, (ii) the long-term follow-up results of three global RRMM daratumumab randomized clinical trials (POLLUX [NCT02076009], CASTOR [NCT02136134], and EQUULEUS [NCT01998971]), (iii) the US-based retrospective MAMMOTH study, (iv) a representative German patient registry maintained by OncologylnformationService (OIs), and (v) the LocoMMotion study [NCT04035226], a prospective, non-interventional, multinational study of the efficacy and safety of real-life standard-of-care in patients with RRMM who had received at least three prior lines of therapy comprising at least one PI, IMiD and an anti-CD38 antibody. In each ITC, the PCT group was comprised of patients who satisfied key eligibility criteria for the Phase 1b-2 clinical trial for cilta-cel described in Examples 2-4 (“the cilta-cel trial”) and was made comparable to the cilta-cel trial using inverse probability of treatment weighting. Hence, the ITCs were deemed appropriate to meta-analyze. The meta-analyses used a robust variance estimator to account for the use of the cilta-cel trial in each pairwise ITC.

We used two populations for the meta-analysis: (1) the “ITT” (intention-to-treat) population, which included all enrolled (apheresed) participants in the cilta-cel study, and all eligible patients in the PCT arms; and (2) the “mITT” (modified intention-to-treat) population, which included all participants infused with cilta-cel in the cilta-cel study, and all eligible patients in the PCT arms who had not progressed or died within 47 days (52 days for OIs and LocoMMotion) after initiating treatment; the median (mean) period between apheresis and infusion in the cilta-cel study. The meta-analysis was performed in two formats:
1. The main meta-analyses considered all participants treated with cilta-cel in the cilta-cel trial compared with PCT, using all index dates for both the modified intention-to-treat (“mITT”) and intention-to-treat (“ITT”) populations. For these meta-analyses, the start of each eligible LOT was used as an index date. Eligible patients in the PCT arms with multiple subsequent therapies contributed multiple LOTs to the analyses (as independent observations), if they remained eligible at the start of each LOT. ITCs with all index dates were not available for MAMMOTH and LocoMMotion.
2. Additional analyses were conducted with ITCs using first index date for both mITT and ITT populations. For these meta-analyses, the start of the first eligible LOT was used as the index date and each patient in the PCT arms contributed only their first eligible LOT to the analyses.

Sensitivity analyses considered ITC effect estimates based on all enrolled participants in the cilta-cel trial. Pooled summary effect estimates were presented as hazard ratios (HRs) with the corresponding 95% confidence intervals (CIs).

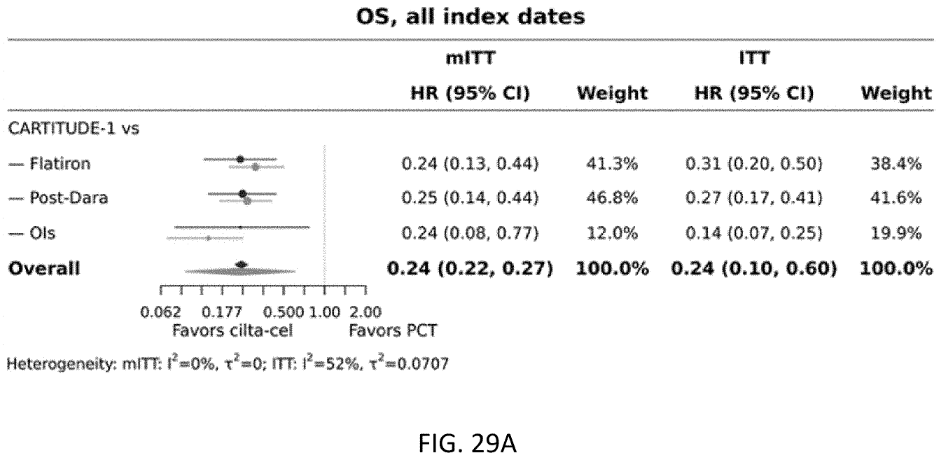

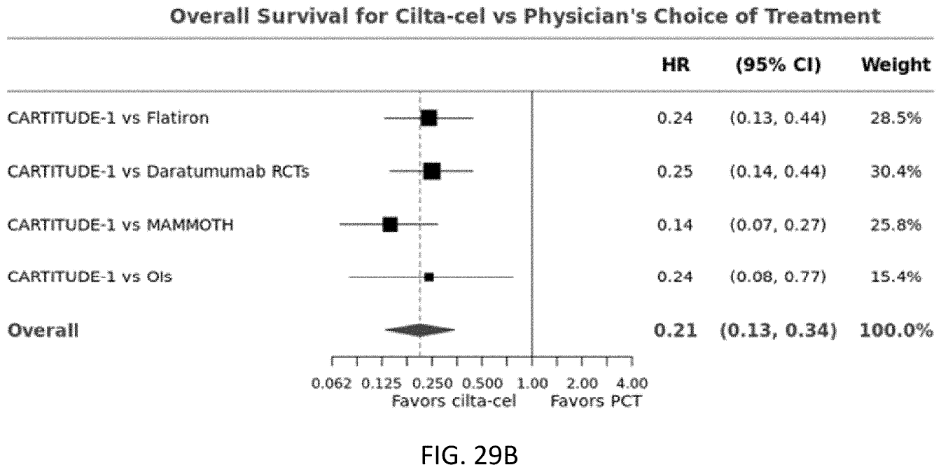

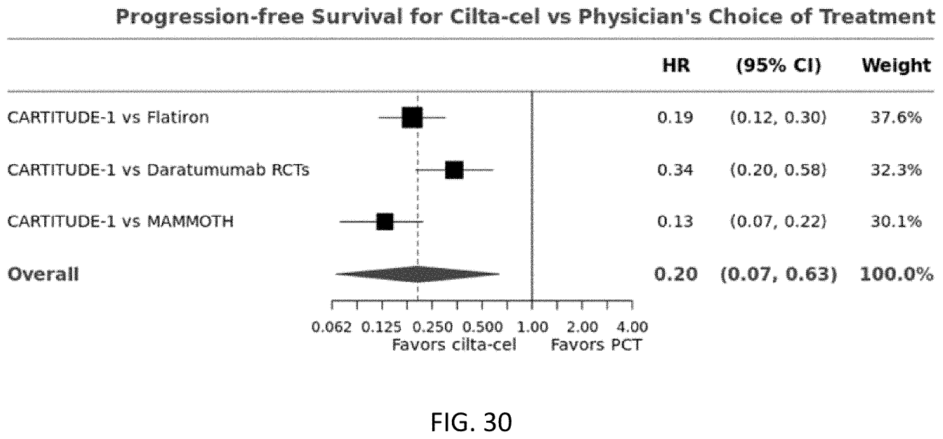

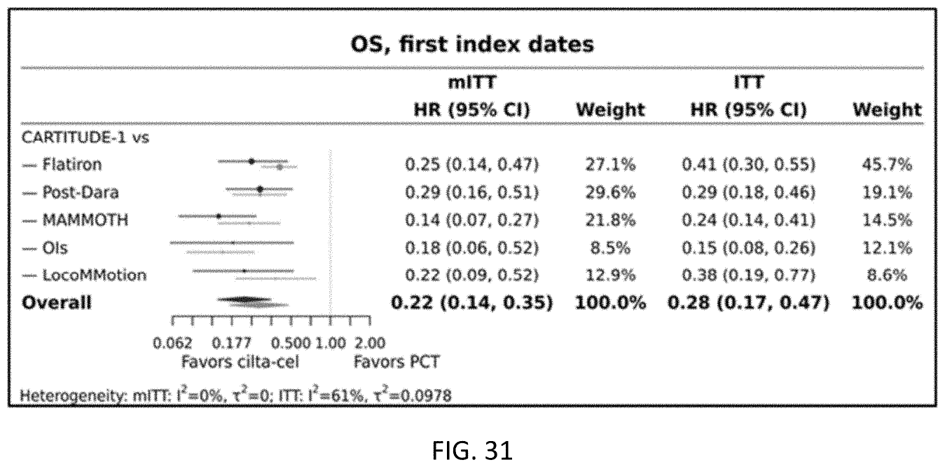

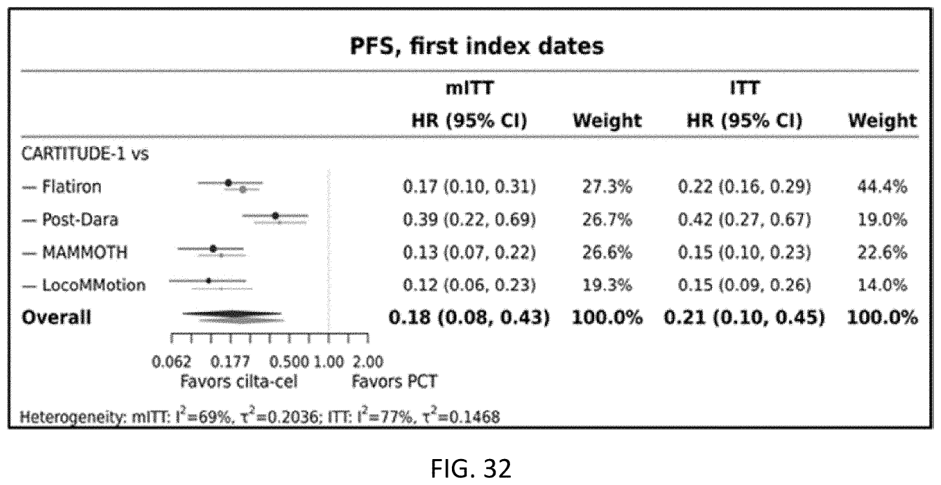

Based on availability of data, the main meta-analyses included four ITCs for OS and three ITCs for PFS. Sensitivity analyses including all enrolled participants confirmed the results of the main meta-analyses. FIGS. 29-32 show the meta-analysis comparisons, using either all index dates or first index dates, of the overall survival (OS) or progression-free survival (PFS) of patients enrolled/treated in the cilta-cel trial and patients treated with physician's choice of treatment.

Cilta-cel demonstrated a significant advantage over PCT in terms of OS, PFS, TTNT, and ORR, highlighting its potential as an effective therapy in patients with triple-class exposed RRMM. Conclusions were consistent across populations (mITT versus ITT) and available index dates.

In conclusion, the meta-analyses demonstrated that cilta-cel enjoys a significant advantage over PCT in terms of OS and PFS, highlighting its potential as an effective therapy in patients with triple-class exposed RRMM. In the absence of head-to-head comparisons between cilta-cel and treatments used in real-world clinical practice, this meta-analysis of ITCs suggested that cilta-cel offers substantially more clinical benefit than PCT for patients with triple-class exposed RRMM.

TABLE 1
Summary of Subject Treatment Overview; All Enrolled Analysis Set

| | Phase 1b | Phase 2 | Phase 1b + Phase 2 |
|---|---|---|---|
| Analysis set: all enrolled | 35 | 78 | 113 |
| Subject who underwent apheresis | 35 (100.0%) | 78 (100.0%) | 113 (100.0%) |
| Subjects who received conditioning regimen | 30 (85.7%) | 71 (91.0%) | 101 (89.4%) |
| Subjects who received cilta-cel infusion | 29 (82.9%) | 68 (87.2%) | 97 (85.8%) |
| Subjects received conditioning regimen but did not | 1 (2.9%) | 3 (3.8%) | 4 (3.5%) |
| receive cilta-cel infusion | | | |
| Reasons | | | |
| Adverse event | 1 (2.9%) | 0 | 1 (0.9%) |
| Subject refused further study treatment | 0 | 2 (2.6%) | 2 (1.8%) |
| Death | 0 | 1 (1.3%) | 1 (0.9%) |

TABLE 2
Summary of Study Duration of Follow-up; All Treated Analysis Set

| | Phase 1b | Phase 2 | Phase 1b + Phase 2 |
|---|---|---|---|
| Analysis set: all treated | 29 | 68 | 97 |
| Duration of follow-up (months) | | | |
| N | 29 | 68 | 97 |
| Mean (SD) | 16.67 (3.815) | 10.79 (2.597) | 12.55 (4.033) |
| Median | 16.94 | 11.27 | 12.42 |
| Range | (3.3+; 24.9) | (1.5+; 14.8) | (1.5+; 24.9) |

+Denotes subjects who died.

TABLE 3
Summary of Prior Therapies for Multiple Myeloma; All Treated Analysis Set

| | Phase 1b | | Phase 2 | | Phase 1b + Phase 2 | |
|---|---|---|---|---|---|---|
| Analysis set: all treated | 29 | | 68 | | 97 | |
| Number of lines of prior therapies | | | | | | |
| for multiple myeloma | | | | | | |
| N | 29 | | 68 | | 97 | |
| Category, n (%) | | | | | | |
| 3 | 7 | (24.1%) | 10 | (14.7%) | 17 | (17.5%) |
| 4 | 3 | (10.3%) | 13 | (19.1%) | 16 | (16.5%) |
| 5 | 6 | (20.7%) | 9 | (13.2%) | 15 | (15.5%) |
| >5 | 13 | (44.8%) | 36 | (52.9%) | 49 | (50.5%) |
| Mean (SD) | 6.1 | (3.37) | 6.4 | (3.19) | 6.3 | (3.23) |
| Median | 5.0 | | 6.0 | | 6.0 | |
| Range | (3; 18) | | (3; 18) | | (3; 18) | |
| Prior transplantation | 26 | (89.7%) | 61 | (89.7%) | 87 | (89.7%) |
| Autologous | 26 | (89.7%) | 61 | (89.7%) | 87 | (89.7%) |
| 1 | 19 | (65.5%) | 51 | (75.0%) | 70 | (72.2%) |
| 2 | 7 | (24.1%) | 10 | (14.7%) | 17 | (17.5%) |
| Allogenic | 0 | | 8 | (11.8%) | 8 | (8.2%) |
| Prior radiotherapy | 7 | (24.1%) | 40 | (58.8%) | 47 | (48.5%) |
| Prior cancer-related surgery/ | 2 | (6.9%) | 22 | (32.4%) | 24 | (24.7%) |
| procedure | | | | | | |
| Prior PI | 29 | (100.0%) | 68 | (100.0%) | 97 | (100.0%) |
| Bortezomib | 25 | (86.2%) | 67 | (98.5%) | 92 | (94.8%) |
| Carfilzomib | 26 | (89.7%) | 57 | (83.8%) | 83 | (85.6%) |
| Ixazomib | 9 | (31.0%) | 20 | (29.4%) | 29 | (29.9%) |
| Prior IMiD | 29 | (100.0%) | 68 | (100.0%) | 97 | (100.0%) |
| Lenalidomide | 29 | (100.0%) | 67 | (98.5%) | 96 | (99.0%) |
| Pomalidomide | 26 | (89.7%) | 63 | (92.6%) | 89 | (91.8%) |
| Thalidomide | 6 | (20.7%) | 15 | (22.1%) | 21 | (21.6%) |
| Prior PI and Prior IMiD | 29 | (100.0%) | 68 | (100.0%) | 97 | (100.0%) |
| Prior corticosteroids | 29 | (100.0%) | 68 | (100.0%) | 97 | (100.0%) |
| Dexamethasone | 29 | (100.0%) | 68 | (100.0%) | 97 | (100.0%) |
| Prednisone | 3 | (10.3%) | 6 | (8.8%) | 9 | (9.3%) |
| Prior alkylating agents | 28 | (96.6%) | 66 | (97.1%) | 94 | (96.9%) |
| Prior anthracyclines | 9 | (31.0%) | 18 | (26.5%) | 27 | (27.8%) |
| Prior anti-CD38 antibodies | 29 | (100.0%) | 68 | (100.0%) | 97 | (100.0%) |
| Daratumumab | 27 | (93.1%) | 67 | (98.5%) | 94 | (96.9%) |
| Isatuximab | 2 | (6.9%) | 6 | (8.8%) | 8 | (8.2%) |
| TAK-079 | 1 | (3.4%) | 0 | | 1 | (1.0%) |
| Prior Elotuzumab | 4 | (13.8%) | 19 | (27.9%) | 23 | (23.7%) |
| Prior Panobinostat | 5 | (17.2%) | 6 | (8.8%) | 11 | (11.3%) |
| Prior PI + IMiD + ALKY | 28 | (96.6%) | 66 | (97.1%) | 94 | (96.9%) |
| Prior PI + IMiD + anti-CD38 | 29 | (100.0%) | 68 | (100.0%) | 97 | (100.0%) |
| antibodies | | | | | | |
| Prior PI + IMiD + anti-CD38 | 28 | (96.6%) | 66 | (97.1%) | 94 | (96.9%) |
| antibodies + ALKY | | | | | | |
| Prior penta-exposed (at least | 22 | (75.9%) | 59 | (86.8%) | 81 | (83.5%) |
| 2 PIs + at least 2 IMiDs + 1 | | | | | | |

anti-CD38 antibodies)

TABLE 4
Summary of Refractory Status to Prior Multiple Myeloma Therapy; All Treated Analysis Set

| | Phase 1b | | Phase 2 | | Phase 1b + Phase 2 |
|---|---|---|---|---|---|
| Analysis set: all treated | 29 | | 68 | | 97 |
| Refractory at any point to prior therapy | 29 | (100.0%) | 68 | (100.0%) | 97 (100.0%) |
| Refractory Status | | | | | |
| PI + IMiD + anti-CD38 antibody | 25 | (86.2%) | 60 | (88.2%) | 85 (87.6%) |
| Any PI | 25 | (86.2%) | 62 | (91.2%) | 87 (89.7%) |
| Any IMiD | 28 | (96.6%) | 67 | (98.5%) | 95 (97.9%) |
| Any anti-CD38 antibody | 29 | (100.0%) | 67 | (98.5%) | 96 (99.0%) |
| At least 2 PIs + at least 2 IMiDs + 1 anti-CD38 | 9 | (31.0%) | 32 | (47.1%) | 41 (42.3%) |
| antibody | | | | | |
| Refractory to last line of prior therapy | 28 | (96.6%) | 68 | (100.0%) | 96 (99.0%) |
| Refractory to | | | | | |
| Bortezomib | 15 | (51.7%) | 51 | (75.0%) | 66 (68.0%) |
| Carfilzomib | 21 | (72.4%) | 42 | (61.8%) | 63 (64.9%) |
| Ixazomib | 7 | (24.1%) | 20 | (29.4%) | 27 (27.8%) |
| Lenalidomide | 22 | (75.9%) | 57 | (83.8%) | 79 (81.4%) |
| Pomalidomide | 22 | (75.9%) | 59 | (86.8%) | 81 (83.5%) |
| Thalidomide | 1 | (3.4%) | 7 | (10.3%) | 8 (8.2%) |
| Daratumumab | 27 | (93.1%) | 67 | (98.5%) | 94 (96.9%)^{a} |
| Isatuximab | 2 | (6.9%) | 5 | (7.4%) | 7 (7.2%) |
| TAK-079 | 1 | (3.4%) | 0 | | 1 (1.0%) |
| Elotuzumab | 1 | (3.4%) | 18 | (26.5%) | 19 (19.6%) |
| Panobinostat | 3 | (10.3%) | 5 | (7.4%) | 8 (8.2%) |

^{a}Two additional subjects were refractory to other anti-CD38 antibodies

TABLE 5
Pre-infusion Medications

| Medication | Dose | Administration |
|---|---|---|
| Antihistamine | diphenhydramine (50 mg) | Oral - administer 1 hour (±15 minutes) |
| | or equivalent | prior to cilta-cel infusion |
| | | Or |
| | | IV- start infusion 30 minutes (±15 minutes) |
| | | prior to cilta-cel infusion |
| Antipyretic | acetaminophen (650 mg | Oral or IV - administer 30 minutes (±15 minutes) |
| | to 1,000 mg) or | prior to cilta-cel infusion |
| | equivalent | |

TABLE 6
Criteria for Response to Multiple Myeloma Treatment

| Response | Response Criteria |
|---|---|
| Stringent | CR as defined below, plus |
| complete | Normal FLC ratio, and |
| response | Absence of clonal plasma cells (PCs) by immunohistochemistry or 2- to 4-color flow |
| (sCR) | cytometry |
| Complete | Negative immunofixation of serum and urine, and |
| response | Disappearance of any soft tissue plasmacytomas, and |
| (CR)^{a} | <5% PCs in bone marrow |
| | No evidence of initial monoclonal protein isotype(s) on immunofixation of the serum |
| | and urine.^{b} |
| Very good | Serum and urine M-component detectable by immunofixation but not on |
| partial response | electrophoresis, or |
| (VGPR)^{a} | ≥90% reduction in serum M-component plus urine M-component <100 mg/24 hours |
| Partial response | ≥50% reduction of serum M-protein and reduction in 24-hour urinary M-protein by |
| (PR) | ≥90% or to <200 mg/24 hours |
| | If serum and urine M-protein were not measurable, a decrease ≥50% in the difference |
| | between involved and uninvolved FLC levels was required in place of the M-protein |
| | criteria |
| | If serum and urine M-protein were not measurable, and serum FLC assay was also not |
| | measurable, ≥50% reduction in bone marrow PCs was required in place of M-protein, |
| | provided baseline percentage had been ≥30% |
| | In addition to the above criteria, if present at baseline, ≥50% reduction in the size of |
| | soft tissue plasmacytomas was also required. |
| Minimal | ≥25% but ≤49% reduction of serum M-protein and reduction in 24-hour urine |
| response | M-protein by 50% to 89% |
| (MR) | In addition to the above criteria, if present at baseline, ≥50% reduction in the size of |
| | soft tissue plasmacytomas was also required. |
| Stable disease | Not meeting criteria for sCR, CR, VGPR, PR, MR, or progressive disease |
| Progressive | Any one or more of the following criteria: |
| disease^{c} | Increase of 25% from lowest response value in any of the following: |
| | Serum M-component (absolute increase must be ≥0.5 g/dL), and/or |
| | Urine M-component (absolute increase must be ≥200 mg/24 hours), and/or |
| | Only in subjects without measurable serum and urine M-protein levels: the |
| | difference between involved and uninvolved FLC levels (absolute increase must |
| | be >10 mg/dL) |
| | Only in subjects without measurable serum and urine M-protein levels and without |
| | measurable disease by FLC levels, bone marrow PC percentage (absolute increase |
| | must be ≥10%). |
| | Appearance of a new lesion(s), ≥50% increase from nadir in sum of the products of |
| | the maximal perpendicular diameters of measured lesions of >1 lesion, or ≥50% |
| | increase in the longest diameter of a previous lesion >1 cm in short axis |
| | Definite development of new bone lesions or definite increase in the size of existing |
| | bone lesions |
| | ≥50% increase in circulating plasma cells (minimum of 200 cells per uL) if this was |
| | the only measure of disease |

^{a}Clarifications to the criteria for coding CR and VGPR in subjects in whom the only measurable disease is by serum FLC levels: CR in such subjects indicates a normal FLC ratio of 0.26 to 1.65 in addition to CR criteria listed above. VGPR in such subjects requires a ≥90% decrease in the difference between involved and uninvolved FLC levels. For patients achieving very good partial response by other criteria, a soft tissue plasmacytoma must decrease by more than 90% in the sum of the maximal perpendicular diameter (SPD) compared with baseline.
^{b}In some cases it is possible that the original M protein light-chain isotype is still detected on immunofixation but the accompanying heavy-chain component has disappeared; this would not be considered as a CR even though the heavy-chain component is not detectable, since it is possible that the clone evolved to one that secreted only light chains. Thus, if a patient has IgA lambda myeloma, then to qualify as CR there should be no IgA detectable on serum or urine immunofixation; if free lambda is detected without IgA, then it must be accompanied by a different heavy chain isotype (IgG, IgM, etc.).
^{c}Clarifications to the criteria for coding progressive disease: bone marrow criteria for progressive disease are to be used only in subjects without measurable disease by M-protein and by FLC levels; “25% increase” refers to M-protein, and FLC, and does not refer to bone lesions, or soft tissue plasmacytomas and the “lowest response value” does not need to be a confirmed value.
Notes:
All response categories (CR, sCR, VGPR, PR, MR, and progressive disease) require 2 consecutive assessments made at any time before the institution of any new therapy; CR, sCR, VGPR, PR, MR, and stable disease categories also require no known evidence of progressive or new bone lesions if radiographic studies were performed. VGPR and CR categories require serum and urine studies regardless of whether disease at baseline was measurable on serum, urine, both, or neither.
Radiographic studies are not required to satisfy these response requirements. Bone marrow assessments need not be confirmed. For progressive disease, serum M-component increases of ≥1 g/dL are sufficient to define relapse if lowest M-component is ≥5 g/dL.

TABLE 7
Cytokine Release Syndrome ASTCT Consensus Grading System

| Grade | Toxicity |
|---|---|
| Grade 1 | Fever^{a} (Temperature ≥38°) |
| Grade 2 | Fever^{a} (Temperature ≥38°) with either: |
| | Hypotension not requiring vasopressors |
| | And/or^{c} hypoxia requiring low-flow nasal cannula^{b} or blow-by. |
| Grade 3 | Fever^{a} (Temperature ≥38°) with either: |
| | Hypotension requiring a vasopressor with or without vasopressin, |
| | And/or^{c} hypoxia requiring high-flow nasal cannula^{b}, facemask, nonrebreather mask, |
| | or Venturi mask. |
| Grade 4 | Fever^{a} (Temperature ≥38°) with either: |
| | hypotension requiring multiple vasopressors (excluding vasopressin), |
| | And/or^{c} hypoxia requiring positive pressure (eg, CPAP, BiPAP, intubation and |
| | mechanical ventilation). |
| Grade 5 | Death |

^{a}Fever not attributable to any other cause. In patients who have CRS then receive antipyretics or anticytokine therapy such as tocilizumab or steroids, fever is no longer required to grade subsequent CRS severity. In this case, CRS grading is driven by hypotension and/or hypoxia.
^{b}Low-flow nasal cannula is defined as oxygen delivered at ≤6 L/minute or blow-by oxygen delivery. High-flow nasal cannula is defined as oxygen delivered at >6 L/minute.
^{c}CRS grade is determined by the more severe event: hypotension or hypoxia not attributable to any other cause.
Note:
Organ toxicities associated with CRS may be graded according to CTCAE v5.0 but they do not influence CRS grading.

TABLE 8
Immune Effector Cell-associated Neurotoxicity Syndrome
(ICANS) ASTCT Consensus Grading System^{a, b}

| Neurotoxicity | | | | |
|---|---|---|---|---|
| Domain | Grade 1 | Grade 2 | Grade 3 | Grade 4 |
| ICE Score | 7-9 | 3-6 | 0-2 | 0 (patient is |
| | | | | unarousable and |
| | | | | unable to perform |
| | | | | ICE). |
| Depressed Level of | Awakens | Awakens to | Awakens only to tactile | Patient is |
| Consciousness | spontaneously. | voice. | stimulus. | unarousable or |
| | | | | requires vigorous or |
| | | | | repetitive tactile |
| | | | | stimuli to arouse. |
| | | | | Stupor or coma. |
| Seizure | N/A | N/A | Any clinical seizure, | Life-threatening |
| | | | focal or generalized, that | prolonged seizure |
| | | | resolves rapidly; or | (>5 min); or |
| | | | Non-convulsive | Repetitive clinical |
| | | | seizures on EEG that | or electrical seizures |
| | | | resolve with | without return to |
| | | | intervention. | baseline in between. |
| Motor Findings | N/A | N/A | N/A | Deep focal motor |
| | | | | weakness such as |
| | | | | hemiparesis or |
| | | | | paraparesis. |
| Raised | N/A | N/A | Focal/local edema on | Diffuse cerebral |
| Intracranial | | | neuroimaging. | edema on |
| Pressure/ | | | | neuroimaging; or |
| Cerebral Edema | | | | Decerebrate or |
| | | | | decorticate |
| | | | | posturing; or |
| | | | | Cranial nerve VI |
| | | | | palsy; or |
| | | | | Papilledema; or |
| | | | | Cushing's triad. |

^{a}Toxicity grading according to Lee et al 2019
^{b}ICANS grade is determined by the most severe event (ICE score, level of consciousness, seizure, motor findings, raised ICP/cerebral edema) not attributable to any other cause.
Note:
all other neurological adverse events (not associated with ICANS) should continue to be graded with CTCAE Version 5.0 during both phases of the study

TABLE 9
Overall Best Response Based on International Myeloma Working Group (IMWG) Consensus Criteria, as
Assessed by Independent Review Committee (IRC); All Treated Analysis Set at Median Follow-Up
Time of 12.4 Months

| | Phase 1b | | Phase 2 | | Phase 1b + Phase 2 | |
|---|---|---|---|---|---|---|
| | n (%) | 95% exact CI for % | n (%) | 95% exact CI for % | n (%) | 95% exact CI for % |
| Analysis set: all treated | 29 | | 68 | 97 | | |
| Best response | | | | | | |
| Stringent complete response (sCR) | 25 (86.2%) | (68.3%, 96.1%) | 40 (58.8%) | (46.2%, 70.6%) | 65 (67.0%) | (56.7%, 76.2%) |
| Complete response (CR) | 0 | (NE, NE) | 0 | (NE, NE) | 0 | (NE, NE) |
| MRD-negative CR/sCR ª | 14 (48.3%) | (29.4%, 67.5%) | 19 (27.9%) | (17.7%, 40.1%) | 33 (34.0%) | (24.7%, 44.3%) |
| Very good partial response | 3 (10.3%) | (2.2%, 27.4%) | 22 (32.4%) | (21.5%, 44.8%) | 25 (25.8%) | (17.4%, 35.7%) |
| (VGPR) | | | | | | |
| Partial response (PR) | 1 (3.4%) | (0.1%, 17.8%) | 3 (4.4%) | (0.9%, 12.4%) | 4 (4.1%) | (1.1%, 10.2%) |
| Minimal response (MR) | 0 | (NE, NE) | 0 | (NE, NE) | 0 | (NE, NE) |
| Stable disease (SD) | 0 | (NE, NE) | 0 | (NE, NE) | 0 | (NE, NE) |
| Progressive disease (PD) | 0 | (NE, NE) | 1 (1.5%) | (0.0%, 7.9%) | 1 (1.0%) | (0.0%, 5.6%) |
| Not evaluable (NE) | 0 | (NE, NE) | 2 (2.9%) | (0.4%, 10.2%) | 2 (2.1%) | (0.3%, 7.3%) |
| Overall response (sCR + CR + | 29 | (88.1%, 100.0%) | 65 (95.6%) | (87.6%, 99.1%) | 94 (96.9%) | (91.2%, 99.4%) |
| VGPR + PR) | (100.0%) | | | | | |
| P-value (one-sided, exact binomial | | | | | <0.0001 | |
| test for null hypothesis of overall | | | | | | |
| response rate ≤ 30%) | | | | | | |
| Clinical benefit (Overall response + | 29 | (88.1%, 100.0%) | 65 (95.6%) | (87.6%, 99.1%) | 94 (96.9%) | (91.2%, 99.4%) |
| MR) | (100.0%) | | | | | |
| VGPR or better (sCR + CR + VGPR) | 28 (96.6%) | (82.2%, 99.9%) | 62 (91.2%) | (81.8%, 96.7%) | 90 (92.8%) | (85.7%, 97.0%) |
| CR or better (sCR + CR) | 25 (86.2%) | (68.3%, 96.1%) | 40 (58.8%) | (46.2%, 70.6%) | 65 (67.0%) | (56.7%, 76.2%) |

Keys:
CI = confidence interval.
^{a} MRD-negative CR/sCR. Only MRD assessments (10⁻⁵ testing threshold) within 3 months of achieving CR/sCR until death/progression/subsequent therapy (exclusive) were considered.

TABLE 10
Duration of Response Based on Independent Review Committee (IRC) Assessment; Responders
in All Treated Analysis Set at Median Follow-Up Time of 12.4 Months

| | Phase 1b | | Phase 2 | | Phase 1b + Phase 2 | |
|---|---|---|---|---|---|---|
| Analysis set: responders in all treated | 29 | | 65 | | 94 | |
| Duration of response | | | | | | |
| Number of events (%) | 9 | (31.0%) | 15 | (23.1%) | 24 | (25.5%) |
| Number of censored (%) | 20 | (69.0%) | 50 | (76.9%) | 70 | (74.5%) |
| Kaplan-Meier estimate (months) | | | | | | |
| 25% quantile (95% CI) | 12.0 | (6.0, NE) | 10.3 | (4.5, NE) | 11.1 | (6.0, NE) |
| Median (95% CI) | NE | (15.9, NE) | NE | (NE, NE) | NE | (15.9, NE) |
| 75% quantile (95% CI) | NE | (NE, NE) | NE | (NE, NE) | NE | (NE, NE) |
| 6-month event-free rate % (95% CI) | 93.1 | (75.1, 98.2) | 80.7 | (68.5, 88.5) | 84.6 | (75.4, 90.6) |
| 9-month event-free rate % (95% CI) | 86.2 | (67.3, 94.6) | 77.4 | (64.8, 85.9) | 80.2 | (70.4, 87.0) |
| 12-month event-free rate % (95% CI) | 72.1 | (51.8, 85.0) | 71.9 | (54.8, 83.4) | 68.2 | (54.4, 78.6) |

Key: CI = confidence interval, NE = Not estimable.

TABLE 11
Progression-Free Survival Based on Independent Review Committee (IRC) Assessment;
All Treated Analysis Set at Median Follow-Up Time of 12.4 Months

| | Phase 1b | | Phase 2 | | Phase 1b + Phase 2 | |
|---|---|---|---|---|---|---|
| Analysis set: all treated | 29 | | 68 | | 97 | |
| Progression-free survival | | | | | | |
| Number of events (%) | 9 | (31.0%) | 16 | (23.5%) | 25 | (25.8%) |
| Number of censored (%) | 20 | (69.0%) | 52 | (76.5%) | 72 | (74.2%) |
| Kaplan-Meier estimate (months) | | | | | | |
| 25% quantile (95% CI) | 13.73 | (6.93, NE) | 11.17 | (5.42, NE) | 12.02 | (6.97, NE) |
| Median (95% CI) | NE | (16.79, NE) | NE | (NE, NE) | NE | (16.79, NE) |
| 75% quantile (95% CI) | NE | (NE, NE) | NE | (NE, NE) | NE | (NE, NE) |
| 6-month progression-free survival rate % (95% CI) | 93.1 | (75.1, 98.2) | 85.3 | (74.4, 91.8) | 87.6 | (79.2, 92.8) |
| 9-month progression-free survival rate % (95% CI) | 86.2 | (67.3, 94.6) | 77.8 | (65.9, 86.0) | 80.3 | (70.9, 87.0) |
| 12-month progression-free survival rate % (95% CI) | 82.8 | (63.4, 92.4) | 72.6 | (56.5, 83.6) | 76.6 | (66.0, 84.3) |
| 18-month progression-free survival rate % (95% CI) | 57.7 | (25.9, 79.9) | NE | (NE, NE) | 54.2 | (26.4, 75.4) |

Key: CI = confidence interval.

TABLE 12
Overall Survival; All Treated Analysis Set at Median Follow-Up Time of 12.4 Months

| | Phase 1b | | Phase 2 | | Phase 1b + Phase 2 | |
|---|---|---|---|---|---|---|
| Analysis set: all treated | 29 | | 68 | | 97 | |
| Overall survival | | | | | | |
| Number of events (%) | 5 | (17.2%) | 9 | (13.2%) | 14 | (14.4%) |
| Number of censored (%) | 24 | (82.8%) | 59 | (86.8%) | 83 | (85.6%) |
| Kaplan-Meier estimate (months) | | | | | | |
| 25% quantile (95% CI) | 19.12 | (13.73, NE) | NE | (NE, NE) | 19.12 | (19.12, NE) |
| Median (95% CI) | 22.80 | (19.12, NE) | NE | (NE, NE) | 22.80 | (19.12, NE) |
| 75% quantile (95% CI) | NE | (22.80, NE) | NE | (NE, NE) | NE | (22.80, NE) |
| 6-month overall survival rate % (95% CI) | 96.6 | (77.9, 99.5) | 92.6 | (83.2, 96.9) | 93.8 | (86.7, 97.2) |
| 9-month overall survival rate % (95% CI) | 93.1 | (75.1, 98.2) | 89.7 | (79.5, 94.9) | 90.7 | (82.8, 95.0) |
| 12-month overall survival rate % (95% CI) | 93.1 | (75.1, 98.2) | 86.5 | (75.7, 92.7) | 88.5 | (80.2, 93.5) |
| 18-month overall survival rate % (95% CI) | 89.7 | (71.3, 96.5) | NE | (NE, NE) | 85.8 | (75.4, 92.1) |

Key: CI = confidence interval.

TABLE 13
Summary of Overall Minimal Residual Disease (MRD) Negativity Rate
at 10⁻⁵ in Bone Marrow Based on Next-Generation Sequencing (NGS);
All Treated Analysis Set at Median Follow-Up Time of 12.4 Months

| | Phase 1b | Phase 2 | Phase 1b + Phase 2 |
|---|---|---|---|
| Analysis set: all treated | 29 | 68 | 97 |
| MRD negativity rate (10⁻⁵) | 18 (62.1%) | 35 (51.5%) | 53 (54.6%) |
| 95% exact CI of MRD negative rate | (42.3%, 79.3%) | (39.0%, 63.8%) | (44.2%, 64.8%) |

Key: CI = confidence interval.

TABLE 14
Summary of Overall Minimal Residual Disease (MRD) Negativity Rate at 10⁻⁵
in Bone Marrow Based on Next-Generation Sequencing; Subjects with Evaluable Sample
at 10⁻⁵ in All Treated Analysis Set at Median Follow-Up Time of 12.4 Months

| | Phase 1b | Phase 2 | Phase 1b + Phase 2 |
|---|---|---|---|
| Analysis set: subjects with evaluable | 18 | 39 | 57 |
| sample at 10⁻⁵ in all treated | | | |
| MRD negativity rate (10⁻⁵) | 18 (100.0%) | 35 (89.7%) | 53 (93.0%) |
| 95% exact CI of MRD negative rate | (81.5%, 100.0%) | (75.8%, 97.1%) | (83.0%, 98.1%) |

Key: CI = confidence interval.

TABLE 15
Summary of Treatment-emergent Cytokine Release Syndrome (CRS) Events;
All Treated Analysis Set at Median Follow-Up Time of 12.4 Months

| | Phase 1b | | Phase 2 | | Phase 1b + Phase 2 | |
|---|---|---|---|---|---|---|
| Analysis set: all treated | 29 | | 68 | | 97 | |
| Number of subjects with CRS | 27 | (93.1%) | 65 | (95.6%) | 92 | (94.8%) |
| Maximum toxicity grade | | | | | | |
| Grade 1 | 14 | (48.3%) | 35 | (51.5%) | 49 | (50.5%) |
| Grade 2 | 10 | (34.5%) | 28 | (41.2%) | 38 | (39.2%) |
| Grade 3 | 1 | (3.4%) | 2 | (2.9%) | 3 | (3.1%) |
| Grade 4 | 1 | (3.4%) | 0 | | 1 | (1.0%) |
| Grade 5 | 1 | (3.4%) | 0 | | 1 | (1.0%) |
| Time from initial infusion of CAR- | | | | | | |
| T cells to first onset of CRS (days) | | | | | | |
| N | 27 | | 65 | | 92 | |
| Mean (SD) | 7.0 | (2.01) | 6.4 | (2.28) | 6.6 | (2.21) |
| Median | 7.0 | | 7.0 | | 7.0 | |
| Range | (2; 12) | | (1; 10) | | (1; 12) | |
| Duration of CRS (days) | | | | | | |
| N | 27 | | 65 | | 92 | |
| Mean (SD) | 7.0 | (18.04) | 5.2 | (2.68) | 5.7 | (9.94) |
| Median | 3.0 | | 4.0 | | 4.0 | |
| Range | (2; 97) | | (1; 14) | | (1; 97) | |
| Interquartile range | (2.0; 4.0) | | (3.0; 6.0) | | (3.0; 6.0) | |
| Number of subjects with supportive | 26 | (89.7%) | 62 | (91.2%) | 88 | (90.7%) |
| measures to treat CRS^{a} | | | | | | |
| Anti-IL6 receptor Tocilizumab | 23 | (79.3%) | 44 | (64.7%) | 67 | (69.1%) |
| IL-1 receptor antagonist Anakinra | 6 | (20.7%) | 12 | (17.6%) | 18 | (18.6%) |
| Corticosteroids | 6 | (20.7%) | 15 | (22.1%) | 21 | (21.6%) |
| Vasopressor used | 2 | (6.9%) | 2 | (2.9%) | 4 | (4.1%) |
| Oxygen used | 1 | (3.4%) | 5 | (7.4%) | 6 | (6.2%) |
| Blow-by | 0 | | 0 | | 0 | |
| Nasal cannula low flow (≤6L/min) | 1 | (3.4%) | 5 | (7.4%) | 6 | (6.2%) |
| Nasal cannula high flow (>6L/min) | 0 | | 1 | (1.5%) | 1 | (1.0%) |
| Face mask | 0 | | 0 | | 0 | |
| Non-Rebreather mask | 0 | | 0 | | 0 | |
| Venturi mask | 0 | | 0 | | 0 | |
| Other | 0 | | 0 | | 0 | |
| Positive pressure | 1 | (3.4%) | 0 | | 1 | (1.0%) |
| Bilevel Positive Airway Pressure | 1 | (3.4%) | 0 | | 1 | (1.0%) |
| Intubation/Mechanical | 1 | (3.4%) | 0 | | 1 | (1.0%) |
| Ventilation | | | | | | |
| Other | 24 | (82.8%) | 57 | (83.8%) | 81 | (83.5%) |
| Outcome of CRS | | | | | | |
| N | 27 | | 65 | | 92 | |
| Recovered or resolved | 26 | (96.3%) | 65 | (100.0%) | 91 | (98.9%) |

| Not recovered or not resolved | 0 | | 0 | 0 | |
|---|---|---|---|---|---|
| Recovered or resolved with sequelae | 0 | | 0 | 0 | |
| Recovering or resolving | 0 | | 0 | 0 | |
| Fatal | 1 | (3.7%) | 0 | 1 | (1.1%) |

| | | | |
|---|---|---|---|
| Unknown | 0 | 0 | 0 |

TABLE 16
Summary of Immune Effector Cell-Associated Neurotoxicity (ICANS)
With Onset After Ciltacabtagene Autoleucel Infusion; All Treated
Analysis Set at Median Follow-Up Time of 12.4 Months

| | Phase 1b | | Phase 2 | | Phase 1b + Phase 2 | |
|---|---|---|---|---|---|---|
| Analysis set: all treated | 29 | | 68 | | 97 | |
| Number of subjects with ICANS | 3 | (10.3%)^{a} | 13 | (19.1%) | 16 | (16.5%) |
| Maximum toxicity grade | | | | | | |
| Grade 1 | 2 | (6.9%) | 8 | (11.8%) | 10 | (10.3%) |
| Grade 2 | 0 | | 4 | (5.9%) | 4 | (4.1%) |
| Grade 3 | 1 | (3.4%) | 0 | | 1 | (1.0%) |
| Grade 4 | 0 | | 1 | (1.5%) | 1 | (1.0%) |
| Grade 5 | 0 | | 0 | | 0 | |
| Time from initial infusion of cilta-cel to | | | | | | |
| first onset of ICANS | | | | | | |
| N | 3 | | 13 | | 16 | |
| Mean (SD) | 6.3 | (2.89) | 7.5 | (2.22) | 7.3 | (2.29) |
| Median | 8.0 | | 8.0 | | 8.0 | |
| Range | (3; 8) | | (4; 12) | | (3; 12) | |
| Duration of ICANS (days) | | | | | | |
| N | 3 | | 13 | | 16 | |
| Mean (SD) | 3.7 | (2.08) | 5.2 | (3.09) | 4.9 | (2.93) |
| Median | 3.0 | | 4.0 | | 4.0 | |
| Range | (2; 6) | | (1; 12) | | (1; 12) | |
| Number of subjects with treatment of ICANS | 3 | (10.3%) | 13 | (19.1%) | 16 | (16.5%) |
| IL-1 receptor antagonist anakinra | 0 | | 3 | (4.4%) | 3 | (3.1%) |
| Anti-IL6 receptor tocilizumab | 1 | (3.4%) | 2 | (2.9%) | 3 | (3.1%) |
| Corticosteroid | 1 | (3.4%) | 8 | (11.8%) | 9 | (9.3%) |
| Levetiracetam | 0 | | 1 | (1.5%) | 1 | (1.0%) |
| Dexamethasone | 1 | (3.4%) | 8 | (11.8%) | 9 | (9.3%) |
| Methylprednisolone sodium succinate | 0 | | 1 | (1.5%) | 1 | (1.0%) |
| Pethidine | 0 | | 1 | (1.5%) | 1 | (1.0%) |
| Outcome of ICANS | | | | | | |
| N | 3 | | 13 | | 16 | |
| Recovered or resolved | 3 | (100.0%) | 13 | (100.0%) | 16 | (100.0%) |

TABLE 17
Summary of Cytopenias Following Treatment With Ciltacabtagene Autoleucel;
All Treated Analysis Set at Median Follow-Up Time of 12.4 Months

| | Phase 1b + Phase 2 (N = 97) | | |
|---|---|---|---|
| | | Initial Grade 3/4 (%) | Initial Grade 3/4 (%) |
| | Grade 3/4 (%) After Day 1 | Recovered to <= Grade 2 | Recovered to <= Grade 2 |
| | Dosing | by Day 30 | by Day 60 |
| Thrombocytopenia | 60 (61.9%) | 23 (23.7%) | 41 (42.3%) |
| Neutropenia | 95 (97.9%) | 67 (69.1%) | 85 (87.6%) |
| Lymphopenia | 96 (99.0%) | 84 (86.6%) | 88 (90.7%) |

TABLE 18
Overall Best Response Based on International Myeloma Working Group (IMWG) Consensus
Criteria, as Assessed by Independent Review Committee (IRC); All Treated Analysis Set at
Median Follow-Up Time of 18 Months

| | Phase 1b | | Phase 2 | | Phase 1b + Phase 2 | |
|---|---|---|---|---|---|---|
| | n (%) | 95% CI for % | n (%) | 95% CI for % | n(%) | 95% CI for % |
| Analysis set: all treated | 29 | | 68 | | 97 | |
| Best response | | | | | | |
| Stringent complete response (sCR) | 28 (96.6%) | (82.2%, 99.9%) | 52 (76.5%) | (64.6%, 85.9%) | 80 (82.5%) | (73.4%, 89.4%) |
| Complete response (CR) | 0 | (NE, NE) | 0 | (NE, NE) | 0 | (NE, NE) |
| MRD-negative CR/sCRª | 16 (55.2%) | (35.7%, 73.6%) | 26 (38.2%) | (26.7%, 50.8%) | 42 (43.3%) | (33.3%, 53.7%) |
| Very good partial response (VGPR) | 0 | (NE, NE) | 12 (17.6%) | (9.5%, 28.8%) | 12 (12.4%) | (6.6%, 20.6%) |
| Partial response (PR) | 1 (3.4%) | (0.1%, 17.8%) | 2 (2.9%) | (0.4%, 10.2%) | 3 (3.1%) | (0.6%, 8.8%) |
| Minimal response (MR) | 0 | (NE, NE) | 0 | (NE, NE) | 0 | (NE, NE) |
| Stable disease (SD) | 0 (NE, NE) | 0 | (NE, NE) | 0 | (NE, NE) | |
| Progressive disease (PD) | 0 | (NE, NE) | 1 (1.5%) | (0.0%, 7.9%) | 1 (1.0%) | (0.0%, 5.6%) |
| Not evaluable (NE) | 0 | (NE, NE) | 1 (1.5%) | (0.0%, 7.9%) | 1 (1.0%) | (0.0%, 5.6%) |
| Overall response (sCR + CR + | 29 (100.0%) | (88.1%, 100.0%) | 66 (97.1%) | (89.8%, 99.6%) | 95 (97.9%) | (92.7%. 99.7%) |
| VGPR + PR) P-value | | | | | <0.0001 | |
| Clinical benefit (Overall response + | 29 (100.0%) | (88.1%. 100.0%) | 66 (97.1%) | (89.8%, 99.6%) | 95 (97.9%) | (92.7%. 99.7%) |
| MR) | | | | | | |
| VGPR or better (sCR + CR + VGPR) | 28 (96.6%) | (82.2%, 99.9%) | 64 (94.1%) | (85.6%, 98.4%) | 92 (94.8%) | (88.4%, 98.3%) |
| CR or better (sCR + CR) | 28 (96.6%) | (82.2%. 99.9%) | 52 (76.5%) | (64.6%. 85.9%) | 80 (82.5%) | (73.4%. 89.4%) |

Key:
CI = confidence interval;
NE = not estimable.
^{a}MRD-negative CR/sCR. Only MRD assessments (10⁻⁵ testing threshold) within 3 months of achieving CR/sCR until death/progression/subsequent therapy (exclusive) are considered.
Note:
Response was assessed by independent review committee (IRC), based on International Myeloma Working Group (IMWG) consensus criteria (2016).
Note:
Percentages are calculated with the number of subjects in the all treated analysis set as denominator.
Note:
Exact 95% confidence intervals are provided.
Note:
One-sided p-value from exact binomial test for the null hypothesis of overall response rate < 30% is presented.

TABLE 19
Duration of Response Based on Independent Review Committee (IRC) Assessment;
Responders in All Treated Analysis Set at Median Follow-Up Time of 18 Months

| | Phase 1b | | Phase 2 | | Phase 1b + Phase 2 | |
|---|---|---|---|---|---|---|
| Analysis set: responders in all treated | 29 | | 66 | | 95 | |
| Duration of response | | | | | | |
| Number of events(%) | 12 | (41.4%) | 23 | (34.8%) | 35 | (36.8%) |
| Number of censored (%) | 17 | (58.6%) | 43 | (65.2%) | 60 | (63.2%) |
| Kaplan-Meier estimate (months) | | | | | | |
| 25% quantile (95% CI) | 12.0 | (6.0, 24.3) | 10.3 | (5.1, 20.2) | 11.9 | (6.0, 20.2) |
| Median (95% CI) | NE | (15.9, NE) | NE | (20.2, NE) | NE | (21.8, NE) |
| 75% quantile (95% CI) | NE | (NE, NE) | NE | (NE, NE) | NE | (NE, NE) |
| 6-month event-free rate % (95% CI) | 93.1 | (75.1, 98.2) | 81.8 | (70.2, 89.2) | 85.3 | (76.4, 91.0) |
| 12-month event-free rate % (95% CI) | 72.4 | (52.3, 85.1) | 74.2 | (61.9, 83.1) | 73.5 | (63.3, 81.2) |
| 18-month event-free rate % (95% CI) | 69.0 | (48.8, 82.5) | 65.8 | (52.8, 76.0) | 66.7 | (56.1, 75.3) |
| 21-month event-free rate % (95% CI) | 65.5 | (45.4, 79.7) | 61.4 | (46.3, 73.4) | 62.9 | (51.5, 72.3) |
| 24-month event-free rate % (95% CI) | 61.7 | (41.5, 76.7) | NE | (NE, NE) | 60.2 | (48.0, 70.3) |

Key: CI = confidence interval; NE = not estimable.

TABLE 20
Progression-Free Survival Based on Independent Review Committee (IRC) Assessment;
All Treated Analysis Set at Median Follow-Up Time of 18 Months

| | Phase 1b | | Phase 2 | | Phase 1b + Phase 2 | |
|---|---|---|---|---|---|---|
| Analysis set: all treated | 29 | | 68 | | 97 | |
| Progression-free survival | | | | | | |
| Number of events (%) | 12 | (41.4%) | 24 | (35.3%) | 36 | (37.1%) |
| Number of censored (%) | 17 | (58.6%) | 44 | (64.7%) | 61 | (62.9%) |
| Kaplan-Meier estimate (months) | | | | | | |
| 25% quantile (95% CI) | 13.73 | (6.93, 25.23) | 11.01 | (5.42, 21.09) | 12.85 | (6.97, 21.09) |
| Median (95% CI) | NE | (16.79, NE) | NE | (21.09, NE) | NE | (22.80, NE) |
| 75% quantile (95% CI) | NE | (NE, NE) | NE | (NE, NE) | NE | (NE, NE) |
| 6-month progression-free survival rate % (95% CI) | 93.1 | (75.1, 98.2) | 85.3 | (74.4, 91.8) | 87.6 | (79.2, 92.8) |
| 12-month progression-free survival rate % (95% CI) | 82.8 | (63.4, 92.4) | 73.5 | (61.3, 82.4) | 76.3 | (66.5, 83.6) |
| 18-month progression-free survival rate % (95% CI) | 69.0 | (48.8, 82.5) | 66.0 | (53.4, 75.9) | 66.9 | (56.5, 75.3) |
| 21-month progression-free survival rate % (95% CI) | 69.0 | (48.8, 82.5) | 66.0 | (53.4, 75.9) | 66.9 | (56.5, 75.3) |
| 24-month progression-free survival rate % (95% CI) | 61.9 | (41.8, 76.8) | 61.9 | (47.3, 73.5) | 60.5 | (48.5, 70.4) |

Key: CI = confidence interval; NE = not estimable.

TABLE 21
Overall Survival; All Treated Analysis Set at Median Follow-Up Time of 18 Months

| | Phase 1b | | Phase 2 | | Phase 1b + Phase 2 | |
|---|---|---|---|---|---|---|
| Analysis set all treated | 29 | | 68 | | 97 | |
| Overall survival | | | | | | |
| Number of events(%) | 7 | (24.1%) | 16 | (23.5%) | 23 | (23.7%) |
| Number of censored (%) | 22 | (75.9%) | 52 | (76.5%) | 74 | (76.3%) |
| Kaplan-Meier estimate (months) | | | | | | |
| 25% quantile (95% CI) | 27.24 | (13.73, NE) | NE | (11.01, NE) | 23.59 | (14.62, NE) |
| Median (95% CI) | NE | (27.24, NE) | NE | (NE, NE) | NE | (27.24, NE) |
| 75% quantile (95% CI) | NE | (NE, NE) | NE | (NE, NE) | NE | (NE, NE) |
| 6-month overall survival rate % (95% CI) | 96.6 | (77.9, 99.5) | 92.6 | (83.2, 96.9) | 93.8 | (86.7, 97.2) |
| 12-month overall survival rate % (95% CI) | 93.1 | (75.1, 98.2) | 85.3 | (74.4, 91.8) | 87.6 | (79.2. 92.8) |
| 18-month overall survival rate % (95% CI) | 89.7 | (71.3, 96.5) | 77.9 | (66.1, 86.0) | 81.4 | (72.2, 87.9) |
| 21-month overall survival rate % (95% CI) | 86.2 | (67.3, 94.6) | 76.2 | (64.1, 84.7) | 79.2 | (69.6, 86.1) |
| 24-month overall survival rate % (95% CI) | 78.7 | (58.5, 89.8) | 76.2 | (64.1, 84.7) | 74.0 | (61.9, 82.7) |

Key: CI = confidence interval; NE = not estimable.

TABLE 22
Summary of Overall MRD Negativity Rate in Bone Marrow; All
Treated Analysis Set at Median Follow-Up Time of 18 Months

| | Phase 1b | Phase 2 | Phase 1b + Phase 2 |
|---|---|---|---|
| Analysis set: all treated | 29 | 68 | 97 |
| MRD negativity rate (10⁻⁴) | 23 (79.3%) | 42 (61.8%) | 65 (67.0%) |
| 95% CI^{a} of MRD negative rate | (60.3%, 92.0%) | (49.2%, 73.3%) | (56.7%, 76.2%) |
| MRD negativity rate (10⁻⁵) | 19 (65.5%) | 37 (54.4%) | 56 (57.7%) |
| 95% CI^{a} of MRD negative rate | (45.7%, 82.1%) | (41.9%, 66.5%) | (47.3%, 67.7%) |
| MRD negativity rate (10⁻⁶) | 17 (58.6%) | 21 (30.9%) | 38 (39.2%) |
| 95% CI^{a} of MRD negative rate | (38.9%, 76.5%) | (20.2%, 43.3%) | (29.4%, 49.6%) |

Key: CI = confidence interval.
^{a}Exact 95% confidence interval.
Note:
MRD status result based on next-generation sequencing (NGS).

TABLE 23
Summary of Overall MRD Negativity Rate at 10−5 in Bone Marrow; Subjects with Evaluable
Samples at 10⁻⁵ in All Treated Analysis Set at Median Follow-Up Time of 18 Months

| | Phase 1b | Phase 2 | Phase 1b + Phase 2 |
|---|---|---|---|
| Analysis set subjects with evaluable | 19 | 42 | 61 |
| sample at 10⁻⁵ in all treated | | | |
| MRD negativity rate (10⁻⁵) | 19 (100.0%) | 37 (88.1%) | 56 (91.8%) |
| 95% CI^{a} of MRD negative rate | (82.4%, 100.0%) | (74.4%, 96.0%) | (8 1.9%, 97.3%) |

Key: CI = confidence interval, MRD = minimal residual disease.
^{a}Exact 95% confidence interval.
Note:
Evaluable samples are those pass calibration and QC, and include sufficient cells for evaluation at the respective testing threshold.
Note:
MRD status result based on next-generation sequencing (NGS).

TABLE 24
Summary of Treatment-emergent Cytokine Release Syndrome (CRS)
Events by Qualitative Interview Participation Status (Yes/No);
All Treated Analysis Set at Median Follow-Up Time of 18 Months

| | Phase 1b + Phase 2 | | | | | |
|---|---|---|---|---|---|---|
| | Yes | | No | | Total | |
| Analysis set: all treated | 30 | | 67 | | 97 | |
| Number of subjects with CRS | 29 | (96.7%) | 63 | (94.0%) | 92 | (94.8%) |
| Maximum toxicity grade | | | | | | |
| Grade 1 | 18 | (60.0%) | 31 | (46.3%) | 49 | (50.5%) |
| Grade 2 | 9 | (30.0%) | 29 | (43.3%) | 38 | (39.2%) |
| Grade 3 | 2 | (6.7%) | 1 | (1.5%) | 3 | (3.1%) |
| Grade 4 | 0 | | 1 | (1.5%) | 1 | (1.0%) |
| Grade 5 | 0 | | 1 | (1.5%) | 1 | (1.0%) |
| Time from initial infusion of CAR-T | | | | | | |
| cells to first onset of CRS (days) | | | | | | |
| N | 29 | | 63 | | 92 | |
| Mean (SD) | 6.8 | (2.22) | 6.4 | (2.20) | 6.6 | (2.21) |
| Median | 7.0 | | 7.0 | | 7.0 | |
| Range | (2; 10) | | (1; 12) | | (1; 12) | |
| Duration of CRS (days) | | | | | | |
| N | 29 | | 63 | | 92 | |
| Mean (SD) | 4.6 | (2.76) | 6.2 | (11.86) | 5.7 | (9.94) |
| Median | 4.0 | | 4.0 | | 4.0 | |
| Range | (1; 12) | | (2; 97) | | (1; 97) | |
| Interquartile range | (3.0; 6.0) | | (3.0, 6.0) | | (3.0; 6.0) | |
| <=7 days | 24 | (82.8%) | 57 | (90.5%) | 81 | (88.0%) |
| Number of subjects with supportive | 26 | (86.7%) | 62 | (92.5%) | 88 | (90.7%) |
| measures to treat CRS^{a} | | | | | | |
| Anti-IL6 receptor Tocilizumab | 16 | (53.3%) | 52 | (77.6%) | 68 | (70.1%) |
| IL-1 receptor antagonist Anakinra | 3 | (10.0%) | 15 | (22.4%) | 18 | (18.6%) |
| Corticosteroids | 5 | (16.7%) | 16 | (23.9%) | 21 | (21.6%) |
| IV fluids | 9 | (30.0%) | 20 | (29.9%) | 29 | (29.9%) |
| Vasopressor used | 2 | (6.7%) | 2 | (3.0%) | 4 | (4.1%) |
| Oxygen used | 2 | (6.7%) | 4 | (6.0%) | 6 | (6.2%) |
| Blow-by | 0 | | 0 | | 0 | |
| Nasal cannula low flow (≤6 L/min) | 2 | (6.7%) | 4 | (6.0%) | 6 | (6.2%) |
| Nasal cannula high flow (>6 L min) | 1 | (3.3%) | 0 | | 1 | (1.0%) |
| Face mask | 0 | | 0 | | 0 | |
| Non-Rebreather mask | 0 | | 0 | | 0 | |
| Venturi mask | 0 | | 0 | | 0 | |
| Other | 0 | | 0 | | 0 | |
| Positive pressure | 0 | | 1 | (1.5%) | 1 | (1.0%) |
| Bilevel Positive Airway Pressure | 0 | | 1 | (1.5%) | 1 | (1.0%) |
| Intubation/Mechanical Ventilation | 0 | | 1 | (1.5%) | 1 | (1.0%) |
| Other | 0 | | 0 | | 0 | |
| Analgesics/Antiinflammatory | 21 | (70.0%) | 51 | (76. 1%) | 72 | (74.2%) |
| Antiinfectives | 15 | (50.0%) | 33 | (49.3%) | 48 | (49.5%) |
| Antiiepileptics | 0 | | 1 | (1.5%) | 1 | (1.0%) |
| Other | 1 | (3.3%) | 9 | (13.4%) | 10 | (10.3%) |
| Outcome of CRS | | | | | | |
| N | 29 | | 63 | | 12 | |
| Recovered or resolved | 29 | (100.0%) | 62 | (98.4%) | 91 | (98.9%) |

| | | | | | |
|---|---|---|---|---|---|
| Fatal | 0 | 1 | (1.6%) | 1 | (1.1%) |

Key: CRS = Cytokine Release Syndrome.
^{a}Supportive measures to treat CRS and CRS symptoms are included.
Note:
Percentages calculated with the number of subjects in the all treated analysis set as denominator, except for the outcome of CRS and duration of CRS for which percentages are calculated with the number of subjects with CRS in the all treated analysis set as denominator.
Note:
CRS was originally graded by Lee criteria (Lee et al 2014) in Phase 1b and by ASTCT consensus grading system (Lee et al 2019) in Phase 2, with conversion of grade in Phase 1b to ASTCT based on data in eCRF. Toxicity grade by ASTCT is presented in this table, for both Phase 1b and Phase 2.
Note:
Time from initial infusion of CAR-T cells to first onset of CRS is calculated as first onset dare of CRS − initial infusion date of CAR-T cells + 1.

TABLE 25
Summary of Immune Effector Cell-Associated Neurotoxicity (ICANS) With Onset After
Ciltacabtagene Autoleucel Infusion by Qualitative Interview Participation Status
(Yes/No); All Treated Analysis Set at Median Follow-Up Time of 18 Months

| | Phase 1b + Phase 2 | | | | | |
|---|---|---|---|---|---|---|
| | Yes | | No | | Total | |
| Analysis set all treated | 30 | | 67 | | 97 | |
| Number of subjects with ICANS | 7 | (23.3%) | 9 | (13.4%) | 16 | (16.5%) |
| Maximum toxicity grade | | | | | | |
| Grade 1 | 6 | (20.0%) | 4 | (6.0%) | 10 | (10.3%) |
| Grade 2 | 1 | (3.3%) | 3 | (4.5%) | 4 | (4.1%) |
| Grade 3 | 0 | | 1 | (1.5%) | 1 | (1.0%) |
| Grade 4 | 0 | | 1 | (1.5%) | 1 | (1.0%) |
| Grade 5 | 0 | | 0 | | 0 | |
| Time from initial infusion of JNJ- | | | | | | |
| 68284528 to first onset of ICANS | | | | | | |
| N | 7 | | 9 | | 16 | |
| Mean (SD) | 8.0 | (2.52) | 6.7 | (2.06) | 7.3 | (2.29) |
| Median | 8.0 | | 8.0 | | 8.0 | |
| Range | (4; 12) | | (3; 9) | | (3; 12) | |
| Duration of ICANS (days) | | | | | | |
| N | 7 | | 9 | | 16 | |
| Mean (SD) | 3.6 | (2.23) | 6.0 | (3.08) | 4.9 | (2.93) |
| Median | 3.0 | | 6.0 | | 4.0 | |
| Range | (1; 8) | | (2; 12) | | (1; 12) | |
| Number of subjects with treatment for ICANS | 4 | (13.3%) | 6 | (9.0%) | 10 | (10.3%) |
| IL-1 receptor antagonist anakinra | 0 | | 3 | (4.5%) | 3 | (3.1%) |
| Anti-IL6 receptor tocilizumab | 1 | (3.3%) | 3 | (4.5%) | 4 | (4.1%) |
| Corticosteroid | 3 | (10.0%) | 6 | (9.0%) | 9 | (9.3%) |
| Dexamethasone | 3 | (10.0%) | 6 | (9.0%) | 9 | (9.3%) |
| Methylprednisolone sodium succinate | 0 | | 1 | (1.5%) | 1 | (1.0%) |
| Levetiracetam | 1 | (3.3%) | 1 | (1.5%) | 2 | (2.1%) |
| Pethidine | 1 | (3.3%) | 0 | | 1 | (1.0%) |
| Outcome of ICANS | | | | | | |
| Recovered or resolved | 7 | (23.3%) | 9 | (13.4%) | 16 | (16.5%) |
| Concurrent CRS | | | | | | |
| Yes | 6 | (85.7%) | 9 | (100.0%) | 15 | (93.8%) |

| No | 1 | (14.3%) | 0 | 1 | (6.3%) |
|---|---|---|---|---|---|
| ICANS prior to CRS | 0 | | 0 | 0 | |
| ICANS following CRS | 1 | (3.3%) | 0 | 1 | (1.0%) |

Key: CRS = Cytokine Release Syndrome, ICANS = Immune Effector Cell-Associated Neurotoxicity.
^{a}For 2 subjects in Phase 1b, the reported term is CAR-T cell Related Encephalopathy Syndrome (CRES). These events were reported prior to publication of the ASTCT consensus grading system and graded according to NCI-CTCAE version 5.0. For these 2 subjects, the maximum toxicity grade was Grade 1 and Grade 3, respectively according to NCI-CTCAE version 5.0
Note:
ICANS evaluated according to the ASTCT consensus grading system (Lee el al 2019) or NCI-CTCAE version 5.0.
Note:
Percentages are calculated with the number of subjects in the all treated analysis set as denominator, except for concurrent CRS for which percentages are calculated with the number of subjects with ICANS in the all treated analysis set as denominator.
Note:
Treatments for ICANS include treatments administered for ICANS and symptoms of ICANS.
Note:
ICANS and CRS are considered to be concurrent if there is an overlap in the duration of these respective events.

TABLE 26
Incidences of Prolonged Cytopenias Following Treatment With Ciltacabtagene
Autoleucel; All Treated Analysis Set at Median Follow-Up Time of 18 Months

| | Phase 1b + Phase 2 (N = 97) | | | |
|---|---|---|---|---|
| | | | | Occurrence of Grade |
| | | | | 3/4 (%) > Day 60 |
| | | Initial Grade 3/4 (%) | Initial Grade 3/4 (%) | (After Initial |
| | Grade 3/4 (%) After | Not Recovered^{a} to <= | Not Recovered^{a} to <= | Recovery^{a} of Grade |
| | Day 1 Dosing | Grade 2 by Day 30 | Grade 2 by Day 60 | 3/4) |
| Thrombocytopenia | 60 (61.9%) | 40 (41.2%) | 25 (25.8%) | 6 (6.2%) |
| Neutropenia | 95 (97.9%) | 29 (29.9%) | 10 (10.3%) | 12 (12.4%) |
| Lymphopenia | 96 (99.0%) | 12 (12.4%) | 8 (8.2%) | 30 (30.9%) |
| Anemia | 68 (70.1%) | 1 (1.0%) | 1 (1.0%) | 10 (10.3%) |

^{a}The lab result with the worst toxicity grade will be used for a calendar day. Recovery definition: must have 2 consecutive Grade <=2 results from separate days if recovery period <=10 days.
Notes:
Lab results assessed after Day 1 until Day 100 are included in the analysis.
Notes:
Thrombocytopenia: Grade 3/4 - Platelets count <50.000 cells/μL.
Notes:
Neutropenia: Grade 3/4 - Neutrophil count <1000 cells/μL.
Notes:
Lymphopenia: Grade 3/4 - Lymphocytes count <0.5 × 10⁹/L.
Note:
Anemia: Grade 3 - hemoglobin <8 g/dL. Grade 4 not defined by laboratory count per NCI-CTCAE v5.
Notes:
Percentages are based on the number of treated subjects.

TABLE 27
Select Demographics and Baseline Disease Characteristics; All Treated Analysis Set at Median
Follow-Up Time of 18 Months

| | | Black/African | Three Lines of | >=4 Lines of | Triple Class | Penta-Drug | Cytogenetic | Cytogenetic |
|---|---|---|---|---|---|---|---|---|
| | >−65 Years | American | Prior Therapy | Prior Therapy | Refractory | Refractory | High Risk | Standard Risk |
| Analysis set: all treated | 35 | 17 | 17 | 80 | 85 | 41 | 23 | 68 |
| Age, years | | | | | | | | |
| N | 35 | 17 | 17 | 80 | 85 | 41 | 23 | 68 |
| Category, n (%) | | | | | | | | |
| <65 | 0 | 11 (64.7%) | 13 (76.5%) | 49 (61.3%) | 55 (64.7%) | 26 (63.4%) | 16 (69.6%) | 43 (63.2%) |
| 65-75 | 27 (77.1%) | 4 (23.5%) | 3 (17.6%) | 24 (30.0%) | 23 (27.1%) | 10 (24.4%) | 7 (30.4%) | 18 (26.5%) |
| >75 | 8 (22.9%) | 2 (11.8%) | 1 (5.9%) | 7 (8.8%) | 7 (8.2%) | 5 (12.2%) | 0 | 7 (10.3%) |
| Mean (SD) | 71.2 (4.14) | 60.9 (9.56) | 60.9 (7.59) | 62.2 (8.56) | 61.7 (8.33) | 61.4 (8.92) | 61.1 (7.67) | 62.1 (8.46) |
| Median | 70.0 | 61.0 | 59.0 | 62.0 | 60.0 | 60.0 | 61.0 | 60.5 |
| Range | (65; 78) | (46; 78) | (49; 76) | (43; 78) | (43; 78) | (43; 77) | (49; 75) | (43; 78) |
| Time since initial MM: | | | | | | | | |
| diagnosis to enrollment, years | | | | | | | | |
| N | 35 | 17 | 17 | 80 | 85 | 41 | 23 | 68 |
| Mean (SD) | 7.62 (3.619) | 6.38 (4.195) | 4.72 (2.003) | 7.27 (3.732) | 6.77 (3.705) | 7.16(3.849) | 7.18 (3.873) | 6.64 (3.592) |
| Median | 6.77 | 5.33 | 4.57 | 6.71 | 5.88 | 6.64 | 6.31 | 5.91 |
| Range | (2.4; 18.2) | (2.0; 18.2) | (1.6; 8.4) | (1.6; 18.2) | (1.6; 18.2) | (1.7; 15.0) | (2.5; 16.3) | (1.6; 18.2) |
| Number of lines of prior | | | | | | | | |
| therapies for multiple myeloma | | | | | | | | |
| N | 35 | 17 | 17 | 80 | 85 | 41 | 23 | 68 |
| Mean (SD) | 6.7 (3.36) | 5.4 (2.18) | 3.0 (0.00) | 7.1 (3.12) | 6.4 (3.22) | 7.2 (2.85) | 6.1 (3.40) | 6.5 (3.21) |
| Median | 6.0 | 6.0 | 3.0 | 6.0 | 6.0 | 7.0 | 5.0 | 6.0 |
| Range | (3; 18) | (3; 11) | (3; 3) | (4; 18) | (3; 18) | (3; 14) | (3; 18) | (3; 18) |

Key:
BM = bone marrow;
PC = plasma cell.

TABLE 28
Efficacy outcomes in various subgroups of patients; All Treated Analysis Set at Median Follow-Up
Time of 18 Months

| | | | MRD | | | |
|---|---|---|---|---|---|---|
| | | Median | negativity* | Median | 18-month | 18-month |
| | ORR | DOR | 10⁻⁵ | PFS | PFS | OS |
| | % | months | n | months | % | % |
| Patients, n (%) | (95% CI) | (95% CI) | (%) | (95% CI) | (95% CD) | (95% CI) |
| Overall, 97 (100%) | 97.9% | 21.8 | 56 | 22.8 | 66 | 80.9 |
| | (92.7-99.7) | (21.8-NE) | (91.8) | (22.8-NE) | (54.9-75.0) | (71.4-87.6) |
| ≥65 years, 35 (36%) | 97.1 | NE | 21 | NR | 72.7 | 82.9 |
| | (85.1-99.9) | | (91.3) | | (53.6-84.9) | (65.8-91.9) |
| Black/African American, 17 (18%) | 100.0 | NE | 10 | NR | 56.6 | 80.9 |
| | (80.5-100) | (6.8-NE) | (83.3) | | (29.3-76.8) | (51.3-93.5) |
| 3 Prior LOT, 17 (18%) | 100.0 | 21.8 | 8 | 22.8 | 75.6 | 88.2 |
| | (80.5-100) | (12.9-NE) | (80.0) | (13.8-NE) | (47.3-90.1) | (60.6-96.9) |
| ≥4 Prior LOT, 80 (82%) | 97.5 | NE | 48 | NR | 63.6 | 79.4 |
| | (91.3-99.7) | (15.9-NE) | (94.1) | | (50.9-73.9) | (68.4-86.9) |
| Triple-class refractory, 85 (88%) | 97.6 | NE | 50 | NR | 64.6 | 79.4 |
| | (91.8-99.7) | | (92.6) | | (52.4-74.4) | (68.8-86.7) |
| Penta-drug refractory, 41 (42%) | 95.1 | NE | 17 | NR | 66.7 | 74.7 |
| | (83.5-99.4) | (14.4-NE) | (85.0) | | (49.4-79.3) | (57.9-85.6) |
| Cytogenetic risk | | | | | | |
| Standard risk, 68 (70%) | 97.1 | 21.8 | 40 | 22.8 | 69.5 | 81.8 |
| | (89.8-99.6) | (21.8-NE) | (95.2) | (22.8-NE) | (55.7-79.7) | (70.0-89.3) |
| High risk, 23 (24%) | 100.0 | NE | 14 | NR | 56.5 | 78.0 |
| | (85.2-100) | | (82.4) | | (34.3-73.8) | (55.5-90.2) |
| ISS Stage III, 14 (14%) | 100.0 | 13.8 | 6 | 14.6 | 34.3 | 48.2 |
| | (76.8-100) | (5.1-NE) | (100.0) | (6.1-NE) | (9.4-61.6) | (20.8-71.2) |
| Bone marrow plasma cells | | | | | | |
| ≤30%, 58 (60%) | 98.3 | 21.8 | 28 | 22.8 | 69.6 | 82.6 |
| | (90.8-100) | (21.8-NE) | (96.6) | (22.8-NE) | (55.5-80.0) | (70.0-90.2) |
| >30 to <60%, 17 (18%) | 100.0 | NE | 14 | NR | 75.6 | 91.7 |
| | (80.5-100) | (15.9-NE) | (87.5) | | (37.8-92.3) | (53.9-98.8) |
| ≥60%, 21 (22%) | 95.2 | NE | 14 | NR | 50.6 | 71.4 |
| | (76.2-99.9) | (5.5-NE) | (87.5) | | (27.5-69.9) | (47.2-86.0) |
| Baseline BCMA tumor expression | | | | | | |
| ≥median, 31 (32%) | 96.8 | 21.8 | 16 (94.1) | 22.8 | 71.8 | 86.8 |
| | (83.3-99.9) | (15.9-NE) | | (22.8-NE) | (50.4-85.2) | (68.6-94.9) |
| <median, 31 (32%) | 100.0 | NE | 22 (95.7) | NR | 70.8 | 79.2 |
| | (88.8-100) | | | | (51.4-83.6) | (59.0-90.2) |
| Baseline plasmacytomas^{†} 19 (20%) | 100.0 | 6.8 | 10 (90.9) | 13.8 (5.3- | 46.8 | 64.5 |
| | (82.4-100) | (4.0-NE) | | NE) | (23.7-67.0) | (35.6-83.0) |

*In MRD evaluable patients; MRD was assessed in evaluable samples at 10⁻⁵ threshold by next-generation sequencing (clonoSEQ, Adaptive Biotechnologies) in all treated patients at Day 28, and at 6, 12, 18, and 24 months regardless of the status of disease measured in blood or urine.
^{†}Includes bone-based and extramedullary plasmacytomas.
DOR, duration of response;
LOT, lines of therapy,
MRD, minimal residual disease;
NE, not estimable;
NR, not reached;
ORR, overall response rate;
OS, overall survival;
PFS, progression free survival.

TABLE 29A
Overall Survival; All Treated Analysis Set at Median Follow-Up Time of 18 Months; Subgroup
Analysis (Part A)

| | | Black/African | Three Lines of | >=4 Lines of | Triple Class | Penta-Drug | Cytogenetic | Standard |
|---|---|---|---|---|---|---|---|---|
| | >−65 Years | American | Prior Therapy | Prior Therapy | Refractory | Refractory | High Risk | Risk |
| Analysis set: all treated | 35 | 17 | 17 | 80 | 85 | 41 | 23 | 68 |
| Overall survival | | | | | | | | |
| Number of events (%) | 8 (22.9%) | 5 (29.4%) | 4 (23.5%) | 19 (23.8%) | 20 (23.5%) | 11 (26.8%) | 6 (26.1%) | 16 (23.5%) |
| Number of censored (%) | 27 (77.1%) | 12 (70.6%) | 13 (76.5%) | 61 (76.3%) | 65 (76.5%) | 30 (73.2%) | 17 (73.9%) | 52 (76.5%) |
| Kaplan-Meier estimate | | | | | | | | |
| (months) | | | | | | | | |
| 25% quantile | 22.80 (9.26, | 23.59 (3.25, | 27.24 (8.11, | 23.59 (13.73, | 23.59 | 23.59 (8.11, | 19.12 (3.25, | 23.59 (12.48, |
| (95% CI) | NE) | NE) | NE) | NE) | (13.73, NE) | NE) | NE) | NE) |
| Median (95% CI) | NE (22.80, | NE (19.91, | 27.24 (NE, NE) | NE (NE, NE) | NE (NE, | NE (23.59, | NE (NE, | NE (27.24, |
| | NE | NE) | | | NE) | NE) | NE | NE |
| 75% quantile (95% CI) | NE (NE, NE) | NE (23.59, | 27.24 (NE, NE) | NE (NE, NE) | NE (NE, | NE (NE, | NE (NE, | NE (NE, NE) |
| | | NE) | | | NE) | NE) | NE) | |
| 6-month overall survival | 94.3 (79.0, | 88.2 (60.6, | 100.0 (100.0, | 92.5 (84.1, | 94.1 (86.4, | 90.2 (76.1, | 91.3 (69.5, | 95.6 (86.9, |
| rate % (95% CI) | 98.5) | 96.9) | 100.0) | 96.6) | 97.5) | 96.2) | 97.8) | 98.6) |
| 12-month overall survival | 85.7 (69.0, | 88.2 (60.6, | 88.2 (60.6, | 87.5 (78.0, | 87.1 (77.9, | 87.8 (73.2, | 87.0 (64.8, | 88.2 (77.8, |
| rate % (95% CI) | 93.8) | 96.9) | 96.9) | 93.1) | 92.6) | 94.7) | 95.6) | 93.9) |
| 18-month overall survival | 82.9 (65.8, | 82.4 (54.7, | 88.2 (60.6, | 80.0 (69.4, | 80.0 (69.8. | 75.5 (59.3, | 78.3 (55.4, | 82.3 (71.0, |
| rate % (95% CI) | 91.9) | 93.9) | 96.9) | 87.2) | 87.0) | 86.0) | 90.3) | 89.6) |
| 21-month overall survival | 79.8 (62.2, | 76.0 (48.0, | 81.4 (52.6, | 78.7 (68.0, | 78.6 (68.3, | 75.5 (59.3, | 73.7 (50.5, | 80.7 (69.0, |
| rate % (95% CI) | 89.8) | 90.3) | 93.6) | 86.2) | 86.0) | 86.0) | 87.2) | 88.3) |
| 24-month overall survival | 70.9 (45.4, | 57.0 (18.0, | 81.4 (52.6, | 71.9 (57.7, | 72.7 (59.4, | 68.0 (45.9, | 73.7 (50.5 | 73.6 (58.2, |
| rate % (95% CI) | 86.1) | 83.2) | 93.6) | 82.1) | 82.2) | 82.6) | 87.2) | 84.0) |

Key:
CI = confidence interval;
NE = not estimable;
BM = bone marrow;
PC = plasma cell.

TABLE 29B
Overall Survival; All Treated Analysis Set at Median Follow-Up Time of 18 Months; Subgroup
Analysis (Part B)

| | | | | | Baseline Tumor | Baseline Tumor | |
|---|---|---|---|---|---|---|---|
| | | | Baseline BM | | BCMA | BCMA | Presence of |
| | | Baseline BM | PC > 30 | Baseline BM | Expression < | Expression >= | Baseline |
| | ISS Stage III | PC <= 30% | to <60% | PC >= 60% | median | median | Plasmacytoma |
| Analysis set: all treated | 14 | 58 | 17 | 21 | 31 | 31 | 19 |
| Overall survival | | | | | | | |
| Number of events (%) | 7 (50.0%) | 13 (22.4%) | 1 (5.9%) | 8 (38.1%) | 8 (25.8%) | 6 (19.4%) | 8 (42.1%) |
| Number of censored (%) | 7 (50.0%) | 45 (77.6%) | 16 (94.1%) | 13 (61.9%) | 23 (74.2%) | 25 (80.6%) | 11 (57.9%) |
| Kaplan-Meier estimate | | | | | | | |
| (months) | | | | | | | |
| 25% quantile (95% CI) | 9.26 (3.98, 14.62) | 27.24 (13.73, | NE (16.62, NE) | 12.48 (3.25, NE) | 22.80 (10.02, | 27.24 (9.26, NE) | 12.25 (3.91, |
| | | NE) | | | NE) | | 23.59) |
| Median (95% CI) | NE (8.31, NE) | NE (27.24. NE) | NE (NE. NE) | NE (12.48, NE) | NE (22.80, NE) | NE (27.24. NE) | 23.59 (12.25, |
| | | | | | | | NE) |
| 75% quantile (95% CI) | NE (14.62. NE) | NE (27.24, NE) | NE (NE. NE) | NE (22.80, NE) | NE (NE. NE) | NE (NE, NE) | NE (23.59, NE) |
| 6-month overall survival | 92.9 (59.1, 99.0) | 96.6 (86.9. 99.1) | 100.0 (100.0, | 85. 7 (62.0, 952) | 93.5 (76.6, 98.3) | 96.8 (79.2, 99.5) | 89.5 (64.1, 97.3) |
| rate % (95% CI) | | | 100.0) | | | | |
| 12-month overall survival | 64.3 (34.3, 83.3) | 89.7 (78.4, 95.2) | 100.0 (100.0, | 76.2 (5 1.9, 89.3) | 90.3 (72.9, 96.8) | 90.3 (72.9. 96.8) | 78.9 (53.2. 91.5) |
| rate % (95% CI) | | | 100.0) | | | | |
| 18-month overall survival | 50.0 (22.9, 72.2) | 82.8 (70.3, 90.3) | 94.1 (65.0, 99.1) | 71.4 (47.2, 86.0) | 80.6 (61.9. 90.8) | 87.1 (69.2, 95.0) | 68.0 (42.1, 84.2) |
| rate % (95% CI) | | | | | | | |
| 21-month overall survival | 50.0 (22.9, 72.2) | 81.0 (68.3, 89.0) | 94.1 (65.0, 99.1) | 65.5 (40.6, 82.0) | 77.3 (58.2, 88.5) | 87.1 (69.2, 95.0) | 61.8 (36.0, 79.8) |
| rate % (95% CI) | | | | | | | |
| 24-month overall survival | NE (NE, NE) | 75.9 (59.1, 86.5) | 94.1 (65.0, 99.1) | 52.4 (12.4, 75.6) | 67.6 (40.8, 84.3) | 80.9 (58.2, 92.0) | 46.4 (15.8, 72.6) |
| rate % (95% CI) | | | | | | | |

Key:
CI = confidence interval;
NE = not estimable;
BM = bone marrow;
PC = plasma cell.

TABLE 30A
Progression-Free Survival; All Treated Analysis Set at Median Follow-Up Time of 18 Months;
Subgroup Analysis (Part A)

| | | | Three Lines | >=4 Lines of | | | | Cytogenetic |
|---|---|---|---|---|---|---|---|---|
| | | Black/African | of Prior | Prior | Triple Class | Penta-Drug | Cytogenetic | Standard |
| | >−65 Years | American | Therapy | Therapy | Refractory | Refractory | High Risk | Risk |
| Analysis set: all treated | 35 | 17 | 17 | 80 | 85 | 41 | 23 | 68 |
| Progression-free survival | | | | | | | | |
| Number of events (%) | 10 (28.6%) | 7 (41.2%) | 5 (29.4%) | 31 (38.8%) | 31 (36.5%) | 13 (31.7%) | 11 (47.8%) | 23 (33.8%) |
| Number of censored (%) | 25 (71.4%) | 10 (58.8%) | 12 (70.6%) | 49 (61.3%) | 54 (63.5%) | 28 (68.3%) | 12 (52.2%) | 45 (66.2%) |
| Kaplan-Meier estimate (months) | | | | | | | | |
| 25% quantile (95% CI) | 15.28 (4.99, | 10.35 (32.5, | 22.80 (4.21, | 11.01 (6.51, | 12.85 (6.97, | 13.73 (3.98, | 10.35 (3.25, | 14.62 (6.97, |
| | NE) | NE) | NE) | 16.79) | 16.79) | NE) | 13.80) | 25.23) |
| Median (95% CI) | NE (25.23, | NE (7.69, | NE (13.80, | NE (21.45, | NE (25.23, | NE (NE, NE) | 21.09 (10.84, | NE (22.80, |
| | NE) | NE) | NE) | NE) | NE) | | NE) | NE) |
| 75% quantile (95% CI) | NE (25.23, | NE (NE, NE) | NE (NE, NE) | NE (NE, NE) | NE (NE, NE) | NE (NE, NE) | NE (NE, NE) | NE (NE, NE) |
| | NE) | | | | | | | |
| 6-month progression-free | 85.7 (69.0, | 88.2 (60.6, | 94.1 (65.0, | 86.3 (76.5, | 88.2 (79.2, | 87.8 (732, | 82.6 (60.1, | 91.2 (81.4, |
| survival rate % (95% CI) | 93.8) | 96.9) | 99.1) | 92.1) | 93.5) | 94.7) | 93.1) | 95.9) |
| 12-month progression-free | 82.9 (65.8, | 64.7 (37.7, | 88.2 (60.6, | 73.8 (62.6, | 76.5 (65.9, | 75.6 (59.4, | 69.6 (46.6, | 79.4 (67.7, |
| survival rate % (95% CI) | 91.9) | 82.3) | 96.9) | 82.0) | 84.1) | 86.1) | 84.2) | 87.3) |
| 18-month progression-free | 74.0 (55.9, | 58.2 (31.7, | 75.6 (47.3, | 65.0 (53.4, | 65.7 (54.5, | 68.3 (51.7, | 56.5 (34.3, | 70.4 (57.9, |
| survival rate % (95% CI) | 85.5) | 77.5) | 90.1) | 74.3) | 74.7) | 80.2) | 73.8) | 79.8) |
| 21-month progression-free | 74.0 (55.9, | 58.2 (31.7, | 75.6 (47.3, | 65.0 (53.4, | 65.7 (54.5, | 68.3 (51.7, | 56.5 (34.3, | 70.4 (57.9, |
| survival rate % (95% CI) | 85.5) | 77.5) | 90.1) | 74.3) | 74.7) | 80.2) | 73.8) | 79.8) |
| 24-month progression-free | 74.0 (55.9, | 58.2 (31.7, | 66.2 (35.5, | 60.2 (47.7, | 63.5 (51.8, | 68.3 (51.7, | 48.4 (25.1, | 64.1 (49.5, |
| survival rate % (95% CI) | 85.5) | 77.5) | 84.8) | 70.7) | 73.1) | 80.2) | 68.4) | 75.5) |

Key:
CI = confidence interval;
NE = not estimable;
BM = bone marrow;
PC = plasma cell.

TABLE 30B
Progression-Free Survival; All Treated Analysis Set at Median Follow-
Up Time of 18 Months; Subgroup Analysis (Part B)

| | | | | | Baseline | Baseline | |
|---|---|---|---|---|---|---|---|
| | | | Baseline | | Tumor | Tumor | |
| | | Baseline | BM PC > | Baseline | BCMA | BCMA | Presence of |
| | ISS Stage | BM | 30 to | BM | Expression < | Expression >= | Baseline |
| | III | PC <= 30% | <60% | PC >= 60% | median | median | Plasmacytoma |
| Analysis set: all treated | 14 | 58 | 17 | 21 | 3 | 31 | 19 |
| Progression-free survival | | | | | | | |
| Number of events (%) | 8 (57.1%) | 19 (32.8%) | 6 (35.3%) | 10 (47.6%) | 11 (35.5%) | 9 (29.0%) | 10 (52.6%) |
| Number of censored (%) | 6 (42.9%) | 39 (67.2%) | 11 (64.7%) | 11 (52.4%) | 20 (64.5%) | 22 (71.0%) | 9 (47.4%) |
| Kaplan-Meier estimate | | | | | | | |
| (months) | | | | | | | |
| 25% quantile (95% CI) | 6.51 (3.9, 8, | 13.80 (6.93, | 21.09 (6.97. | 6.51 (0.95, | 13.80 (5.42, | 16.79 (4.99, | 5.32 (3.25, |
| | 14.62) | 25.23) | NE) | 12.85) | 25.23 | NE) | 7.69) |
| Median (95% CI) | 14.95 (6.08, | NE (25.23, | NE (16 79, | NE (6.51. | NE (21.45, | NE (22.80, | 13.80 (5.32, |
| | NE) | NE) | NE) | NE) | NE) | NE) | NE) |
| 75% quantile (95% CI) | NE (14.62, | NE (NE. | NE (21.45, | NE (NE, | NE (25.23, | NE (NE, | NE (13.80, |
| | NE) | NE) | NE) | NE) | NE) | NE) | NE) |
| 6-month progression-free | 85.7 (53.9, | 87.9 (76.3, | 100.0 | 81.0 (56.9, | 90.3 (72.9, | 90.3 (729, | 73.7 (47.9, |
| survival rate % (95% CI) | 96.2) | 94.1) | (100.0, | 92.4) | 96.8) | 96.8) | 88.1) |
| | | | 100.0) | | | | |
| 12-month progression-free | 57.1 (28.4, | 77.6 (64.6, | 88.2 (60.6, | 66.7 (42.5, | 80.6 (61.9, | 80.6 (61.9, | 52.6 (28.7, |
| survival rate % (95% CI) | 78.0) | 86.3) | 96.9) | 82.5) | 90.8) | 90.8) | 71.9) |
| 18-month progression-free | 42.9 (17.7, | 70.7 (57.2, | 76.5 (48.8, | 51.6 (28.7, | 71.0 (51.6, | 74.1 (54.7, | 47.4 (24.4, |
| survival rate % (95% CI) | 66.0) | 80.6) | 90.4) | 70.4) | 83.7) | 86.1) | 67.3) |
| 21-month progression-free | 42.9 (17.7, | 70.7 (57.2, | 76.5 (48.8, | 51.6 (28.7, | 71.0 (51.6, | 74.1 (54.7, | 47.4 (24.4, |
| survival rate % (95% CI) | 66.0) | 80.6) | 90.4) | 70.4) | 83.7) | 86.1) | 67.3) |
| 24-month progression-free | NE (NE, | 66.5 (51.1, | 54.6 (23.0, | 51.6 (28.7, | 63.9 (41.2, | 67.3 (44.8, | 47.4 (24.4, |
| survival rate % (95% CI) | NE) | 78.1) | 78.0) | 70.4) | 79.7). | 82.3) | 67.3) |

Key:
CI = confidence interval;
NE = not estimable;
BM = bone marrow;
PC = plasma cell.

TABLE 31
Summary of Treatment-Emergent Cytokine Release Syndrome (CRS) Events; All Treated
Analysis Set at Median Follow-Up Time of 18 Months; Subgroup Analysis

| | | | Three Lines | >=4 Lines of | | | | Cytogenetic |
|---|---|---|---|---|---|---|---|---|
| | | Black/African | of Prior | Prior | Triple Class | Penta-Drug | Cytogenetic | Standard |
| | >−65 Years | American | Therapy | Therapy | Refractory | Refractory | High Risk | Risk |
| Analysis set: all treated | 35 | 17 | 17 | 80 | 85 | 41 | 23 | 68 |
| Number of subjects with CRS | 34 (97.1%) | 16 (94.1%) | 17 (100.0%) | 75 (93.8%) | 81 (95.3%) | 40 (97.6%) | 22 (95.7%) | 64 (94.1%) |
| Maximum toxicity grade | | | | | | | | |
| Grade 1 | 16 (45.7%) | 11 (64.7%) | 9 (52.9%) | 40 (50.0%) | 42 (49.4%) | 21 (51.2%) | 12 (52.2%) | 34 (50.0%) |
| Grade 2 | 16 (45.7%) | 3 (17.6%) | 6 (35.3%) | 32 (40.0%) | 34 (40.0%) | 18 (43.9%) | 8 (34.8%) | 27 (39.7%) |
| Grade 3 | 0 | 1 (5.9%) | 2 (11.8%) | 1 (1.3%) | 3 (3.5%) | 0 | 1 (4.3%) | 2 (2.9%) |
| Grade 4 | 1 (2.9%) | 0 | 0 | 1 (1.3%) | 1 (1.2%) | 0 | 0 | 01 (1.5%) |
| Grade 5 | 1 (2.9%) | 1 (5.9%) | 0 | 1 (1.3%) | 1 (1.2%) | 1 (2.4%) | 1 (4.3%) | 0 |
| Time from initial infusion | | | | | | | | |
| of CAR-T cells to first | | | | | | | | |
| onset of CRS (days) | | | | | | | | |
| N | 34 | 16 | 17 | 75 | 81 | 40 | 22 | 64 |
| Mean (SD) | 6.4 (2.24) | 6.0 (2.28) | 6.9 (2.01) | 6.5 (2.25) | 6.5 (2.19) | 6.6 (2.00) | 6.0 (2.42) | 6.8 (2.04) |
| Median | 5.5 | 6.5 | 7.0 | 7.0 | 7.0 | 7.0 | 7.0 | 7.0 |
| Range | (2; 12) | (2; 10) | (2; 10) | (1; 12) | (1; 12) | (2; 10) | (2; 10) | (1; 12) |
| Duration of CRS (days) | | | | | | | | |
| N | 34 | 16 | 17 | 75 | 81 | 40 | 22 | 64 |
| Mean (SD) | 7.5 (16.03) | 10.3 (23.22) | 4.5 (2.27) | 6.0 (10.95) | 5.9 (10.57) | 7.2 (14.79) | 9.0 (19.17) | 4.6 (2.55) |
| Median | 4.0 | 4.0 | 4.0 | 4.0 | 4.0 | 4.0 | 4.0 | 4.0 |
| Range | (1; 97) | (2; 97) | (2; 10) | (1; 97) | (1; 97) | (1; 97) | (2; 97) | (1; 14) |
| Interquartile range | (3.0; 6.0) | (3.0; 6.5) | (3.0; 5.0) | (3.0; 6.0) | (3.0; 6.0) | (3.0; 6.0) | (4.0; 6.0) | (3.0; 5.5) |
| <+7 days | 30 (88.2%) | 13 (81.3%) | 15 (88.2%) | 66 (88.0%) | 70 (86.4%) | 35 (87.5%) | 18 (81.8%) | 58 (90.6%) |
| Number of subjects with supportive | 33 (94.3%) | 15 (88.2%) | 17 (100.0%) | 71 (88.8%) | 77 (90.6%) | 38 (92.7%) | 22 (95.7%) | 62 (91.2%) |
| measures t treat CRS | | | | | | | | |
| Anti-IL6 receptor Tocilzumab | 29 (82.9%) | 7 (41.2%) | 12 (70.6%) | 56 (70.0%) | 58 (68.2%) | 31 (75.6%) | 17 (73.9%) | 48 (70.6%) |
| IL-1 receptor antagonist Anakinra | 5 (14.3%) | 4 (23.5%) | 1 (5.9%) | 17 (21.3%) | 16 (18.8%) | 6 (14.6%) | 7 (30.4%) | 10 (14.7%) |
| Corticosteroids | 8 (22.90/4) | 3 (17.6%) | 1 (5.90/4) | 20 (25.0%) | 18 (21.2%) | 9 (22.0%) | 10 (43.5%) | 10 (14.7%) |
| IV fluids | 10 (28.6%) | 2 (11.8%) | 3 (17.6%) | 26 (32.5%) | 26 (30.6%) | 14 (34.1%) | 6 (26.1%) | 21 (30.90%) |
| Vasopressor used | 1 (2.90/4) | 1 (5.90/4) | 2 (11.8%) | 2 (2.5%) | 4 (4.7%) | 0 | 1 (4.3%) | 3 (4.4%) |
| Oxygen used | 4 (11.4%) | 2 (11.8%) | 1 (5.90/4) | 5 (6.3%) | 6 (7.1%) | 3 (7.3%) | 2 (8.7%) | 4 (5.9%) |
| Blow-by | 0 | 0 | 0 | 0 | 0 | 0 | 0 | 0 |
| Nasal cannula low flow (≤6 L/min) | 4 (11.4%) | 2 (11.8%) | 1 (5.90/4) | 5 (6.3%) | 6 (7.1%) | 3 (7.3%) | 2 (8.7%) | 4 (5.9%) |
| Nasal cannula low flow (>6 L/min) | 0 | 1 (5.90/4) | 1 (5.90/4) | 0 | 1 (1.2%) | 0 | 1 (4.3%) | 0 |
| Face mask | 0 | 0 | 0 | 0 | 0 | 0 | 0 | 0 |
| Non-Rebreather mask) | 0 | 0 | 0 | 0 | 0 | 0 | 0 | 0 |
| Venturi | 0 | 0 | 0 | 0 | 0 | 0 | 0 | 0 |
| Other | 0 | 0 | 0 | 0 | 0 | 0 | 0 | 0 |
| Positive pressure | 1 (2.9%) | 1 (5.9%) | 0 | 1 (1.3%) | 1 (1.2%) | 1 (2.4%) | 1 (4.3%) | 0 |
| Bilevel Positive Airway Pressure | 1 (2.9%) | 1 (5.9%) | 0 | 1 (1.3%) | 1 (1.2%) | 1 (2.4%) | 1 (4.3%) | 0 |
| Intubation/Mechanical | 1 (2.9%) | 1 (5.9%) | 0 | 1 (1.3%) | 1 (1.2%) | 1 (2.4%) | 1 4.3%) | 0 |
| Ventilation | | | | | | | | |
| Other | 0 | 0 | 0 | 0 | 0 | 0 | 0 | 0 |
| Analgesics/Antiinflammatory | 27 (77.1%) | 13 (76.5%) | 14 (82.4%) | 58 (72.5%) | 62 (72.9%) | 32 (78.0%) | 18 (78.3%) | 50 (73.5%) |
| Antiinfectives | 18 (51.4%) | 8 (47.1%) | 10 (58.8%) | 38 (47.5%) | 41 (48.2%) | 21 (51.2%) | 14 (60.9%) | 30 (44.1%) |
| Antiepileptics | 1 (2.9%) | 0 | 0 | 1 (1.3%) | 1 (1.2%) | 1 (2.4%) | 1 (4.3%) | 0 |
| Other | 6 (17.1%) | 3 (17.6%) | 1 (5.9%) | 9 (11.3%) | 9 (10.6%) | 5 (12.2%) | 4 (17.4%) | 4 (5.9%) |
| Outcome of NRS | | | | | | | | |
| N | 34 | 16 | 17 | 75 | 81 | 40 | 22 | 64 |
| Recovered or resolved | 33 (97.1%) | 15 (93.8%) | 17 (100.0%) | 74 (98.7%) | 80 (98.8%) | 39 (97.5%) | 21 (95.5%) | 64 (100.0%) |
| Fatal | 1 (2.9%) | 1 (6.3%) | 0 | 1 (1.3%) | 1 (1.2%) | 1 (2.5%) | 1 (4.5%) | 0 |

Key:
BM = bone marrow;
CRS = Cytokine Release Syndrome;
PC = plasma cell
^{a} Supportive measures to treat CRS and CRS symptoms are included.
Note:
Percentages calculated with the number of subjects in the all treated analysis set as denominator, except for the outcome of CRS and duration of CRS for which percentages are calculated with the number of subjects with CRS in the all treated analysis set as denominator.
Note:
CRS was originally graded by Lee criteria (Lee et al 2014) in Phase 1b and by ASTCT consensus grading system (Lee et al 2019) in Phase 2, with conversion of grade in Phase 1b to ASTCT based on data in eCRF. Toxicity grade by ASTCT is presented in this table, for both Phase 1b and Phase 2
Note:
Time from initial infusion of CAR-T cells to first onset of CRS is calculated as first onset dare of CRS-initial infusion date of CAR-T cells + 1.

TABLE 32
Summary of Immune Effector Cell-Associated Neurotoxicity (ICANS) With Onset After
Ciltacabtagene Autoleucel Infusion; All Treated Analysis Set at Median Follow-Up Time of 18
Months; Subgroup Analysis

| | | | Three Lines | >=4 Lines of | | | | Cytogenetic |
|---|---|---|---|---|---|---|---|---|
| | | Black/African | of Prior | Prior | Triple Class | Penta-Drug | Cytogenetic | Standard |
| | >−65 Years | American | Therapy | Therapy | Refractory | Refractory | High Risk | Risk |
| Analysis set: all treated | 35 | 17 | 17 | 80 | 85 | 41 | 23 | 68 |
| Number of subjects with ICANS | 10 (28.6%) | 0 | 2 (11.8%) | 14 (17.5%) | 15 (17.6%) | 8 (19.5%) | 2 (8.7%) | 12 (17.6%) |
| Maximum toxicity grade | | | | | | | | |
| Grade 1 | 6 (17.1%) | 0 | 2 (11.8%) | 8 (10.0%) | 9 (10.6%) | 5 (12.2%) | 1 (4.3%) | 8 (11.8%) |
| Grade 2 | 2 (5.7%) | 0 | 0 | 4 (5.0%) | 4 (4.7%) | 2 (4.9%) | 1 (4.3%) | 3 (4.4%) |
| Grade 3 | 1 (2.9%) | 0 | 0 | 1 (1.3%) | 1 (1.2%) | 0 | 0 | 1 (1.5%) |
| Grade 4 | 1 (2.9%) | 0 | 0 | 1 (1.3%) | 1 (1.2%) | 1 (2.4%) | 0 | 0 |
| Grade 5 | 0 | 0 | 0 | 0 | 0 | 0 | 0 | 0 |
| Time from initial infusion of JNJ- | | | | | | | | |
| 68284528 to first onset of ICANS | | | | | | | | |
| N | 10 | 0 | 2 | 14 | 15 | 8 | 2 | 12 |
| Mean (SD) | 7.3 (2.36) | — | 6.0 (2.83) | 7.4 (2.28) | 7.2 (2.37) | 7./9 (1.25) | 6.0 (2.83) | 7.8 (2.17) |
| Median | 7.5 | — | 6.0 | 8.0 | 8.0 | 8.0 | 6.0 | 8.0 |
| Range | (4; 12) | — | (4; 8) | (3; 12) | (3; 12) | (6; 10) | (4; 8) | (3; 12) |
| Duration of ICANS (days) | | | | | | | | |
| N | 10 | 0 | 2 | 14 | 15 | 8 | 2 | 12 |
| Mean (SD) | 4.5 (2.80) | — | 6.0 (2.83) | 4.8 (3.02) | 5.1 (2.92) | 4.5 (2.33) | 5.0 (2.83) | 4.3 (2.87) |
| Median | 3.5 | — | 6.0 | 4.0 | 4.0 | 4.0 | 5.0 | 4.0 |
| Range | (1; 9) | — | (4; 8) | (1; 12) | (1; 12) | (1; 9) | (3; 7) | (1; 12) |
| Number of subjects with | 6 (17.1%) | 0 | 0 | 10 (12.5%) | 10 (11.8%) | 7 (17.1%) | 2 (8.7%) | 7 (10.3%) |
| treatment for ICANS | | | | | | | | |
| IL-1 receptor antagonist anakinra | 2 (5.7%) | 0 | 0 | 3 (3.8%) | 3 (3.5%) | 2 (4.9% | 1 (4.3%) | 1 (1.5%) |
| Anti-IL6 receptor tocilzumab | 2 (5.7%) | 0 | 0 | 4 (5.0%) | 4 (4.7%) | 3 (7.3%) | 1 (4.3%) | 2 (2.9%) |
| Corticosteroids | 6 (17.1%) | 0 | 0 | 9 (11.3%) | 9 (10.6%) | 6 (14.6%) | 2 (8.7%) | 6 (8.8%) |
| Dexamethasone | 6 (17.1%) | 0 | 0 | 9 (11.3%) | 9 (10.6%) | 6 (14.6%) | 2 (8.7%) | 6 (8.8%) |
| Methylprednisolone sodium | 1 (2.9%) | 0 | 0 | 1 (1.3%) | 1 (1.2%) | 1 (2.4%) | 0 | 0 |
| succinate | | | | | | | | |
| Levetiracetam | 1 (2.9%) | 0 | 0 | 2 (2.5%) | 2 (2.4%) | 2 (4.9%) | 1 (4.3%) | 0 |
| Pethidine | 0 | 0 | 0 | 1 (1.3%) | 1 (1.2%) | 1 (2.4%) | 0 | 1 (1.5%) |
| Outcome of ICANS | | | | | | | | |
| Recovered or resolved | 10 (28.6%) | 0 | 2 (11.8%) | 14 (17.5%) | 15 (17.6%) | 8 (19.5%) | 2 (8.7%) | 12 (17.6%) |
| Concurrent CRS | | | | | | | | |
| Yes | 9 (25.7%) | 0 | 2 (11.8%) | 13 (16.3%) | 14 (16.5%) | 8 (19.5%) | 2 (8.7%) | 11 (16.2%) |
| No | 1 (2.9%) | 0 | 0 | 1 (1.3%) | 1 (1.2%) | 0 | 0 | 1 (1.5%) |
| ICANS prior to CRS | 0 | 0 | 0 | 0 | 0 | 0 | 0 | 0 |
| ICANS following CRS | 1 (2.9%) | 0 | 0 | 1 (1.3%) | 1 (1.2%) | 0 | 0 | 1 (1.5%) |

Key:
BM = bone marrow;
CRS = Cytokine Release Syndrome;
ICANS = Immune Effector Cell-Associated Neurotoxicity;
PC = plasma cell,
TE = treatment-emergent
Note:
ICANS evaluated according to the ASTCT consensus grading system (Lee et al 2019) or NCI-CTCAE version 5.0. For 2 subjects in Phase 1b, the reported term CAR-T cell Related Encephalopathy Syndrome (CRES). These events were reported prior to publication of the ASTCT consensus grading system and grading according to NCI-CTCAE version 5.0. For these 2 subjects, the maximum toxicity grade was Grade 1 and Grade 3, respectively according to NCI-CTCAE version 5.0.
Note:
Percentages are calculated with the number of subjects in the all treated analysis set as denominator.
Note:
Treatment of ICANS include treatments administered for ICANS and symptoms of ICANS.
Note:
ICANS and CRS are considered to be concurrent if there is an overlap in the duration of these respective events.

### Example 8: Approved Drug Product Label

An approved drug product label:

Additional information on warnings and precautions are provided below:

Grade 3 or higher events associated with CRS included increased AST and ALT, hyperbilirubinemia, hypotension, pyrexia, hypoxia, respiratory failure, acute kidney injury, disseminated intravascular coagulation and hemorrhage, HLH/MAS, angina pectoris, supraventricular and ventricular tachycardia, malaise, myalgias, increased-C-reactive protein, ferritin, blood alkaline phosphatase and gamma-glutamyl transferase [see Adverse Reactions (6.1)].

One patient with CRS and suspected HLH/MAS developed a fatal retroperitoneal hemorrhage in the setting of thrombocytopenia, coagulopathy and anticoagulation in another ongoing study of CARVYKTI.

Neurologic toxicities, which may be severe, life-threatening or fatal, occurred following treatment with CARVYKTI. Neurologic toxicities included ICANS, neurologic toxicity with signs and symptoms of parkinsonism, Guillain-Barré Syndrome, immune mediated myelitis, peripheral neuropathies and cranial nerve palsies. Counsel patients on the signs and symptoms of these neurologic toxicities, and on the delayed nature of onset of some of these toxicities. Instruct patients to seek immediate medical attention for further assessment and management if signs or symptoms of any of these neurologic toxicities occur at any time [see Patient Counseling Information (17)].

Of the 25 patients in the CARTITUDE-1 study experiencing any neurotoxicity, five male patients had neurologic toxicity with several signs and symptoms of parkinsonism, distinct from ICANS. Neurologic toxicity with parkinsonism has been reported in other ongoing trials of ciltacabtagene autoleucel. Patients had parkinsonian and non-parkinsonian symptoms that included tremor, bradykinesia, involuntary movements, stereotypy, loss of spontaneous movements, masked facies, apathy, flat affect, fatigue, rigidity, psychomotor retardation, micrographia, dysgraphia, apraxia, lethargy, confusion, somnolence, loss of consciousness, delayed reflexes, hyperreflexia, memory loss, difficulty swallowing, bowel incontinence, falls, stooped posture, shuffling gait, muscle weakness and wasting, motor dysfunction, motor and sensory loss, akinetic mutism and frontal lobe release signs. Symptoms did not respond to one or more of the following treatments attempted in one or more patients—systemic chemotherapy, intrathecal chemotherapy and steroids, dopaminergic agents, systemic corticosteroids, plasmapheresis, and intravenous immunoglobulin and dasatinib. One patient experienced partial resolution with residual gait disturbance without treatment for parkinsonism, immunosuppressants or chemotherapy. The median onset of parkinsonism in the 5 patients in CARTITUDE-1 was 43 days (range: 15 to 108 days) from infusion of ciltacabtagene autoleucel. One patient died of neurologic toxicity with parkinsonism 247 days after administration of ciltacabtagene autoleucel; two patients with ongoing parkinsonism died of infectious causes 162 and 119 days after administration of ciltacabtagene autoleucel; in the remaining 2 patients, symptoms of parkinsonism were ongoing up to 530 days after administration of ciltacabtagene autoleucel. Maximum toxicity grade was 2, 3, 4 and 5 in 1, 2, 1 and 1 patient respectively. All 5 patients had a history of prior CRS (n=4 Grade 2; n=1 Grade 3), while 4 of 5 patients had prior ICANS (n=4 Grade 1).

Immune Mediated Myelitis: Grade 3 myelitis has occurred 25 days following treatment with CARVYKTI in another ongoing study. Symptoms reported included hypoesthesia of the lower extremities and the lower abdomen with impaired sphincter control. Symptoms improved with the use of corticosteroids and intravenous immune globulin. Myelitis was ongoing at the time of death from other cause.

One patient with grade 4 HLH/MAS developed fatal intracerebral and gastrointestinal hemorrhage in the setting of coagulopathy and thrombocytopenia 12 days after treatment in another ongoing study of CARVYKTI [see Warnings and Precautions (5.1)]. Patients who develop HLH/MAS have an increased risk of severe bleeding. Monitor hematological parameters in patients with HLH/MAS and transfuse per institutional guidelines.

Grade 5 infections reported in other studies with CARVYKTI include bronchopulmonary aspergillosis, Pneumocystis jirovecii pneumonia, and CMV colitis (with HSV-1 hepatitis). Another patient developed mycotic aneurysm due to cerebral aspergillosis and died of subarachnoid hemorrhage.

In a randomized controlled study of relapsed or refractory multiple myeloma (CARTITUDE-4), patients treated with ciltacabtagene autoleucel had an increased rate of fatal COVID-19 infections compared to the standard therapy arm. Counsel patients on the importance of prevention measures. Follow institutional guidelines for the vaccination and management of immunocompromised patients with COVID 19.

The teachings of all patents, published applications, and references cited herein are incorporated by reference in their entirety.

While example embodiments have been particularly shown and described, it will be understood by those skilled in the art that various changes in form and details may be made therein without departing from the scope of the embodiments encompassed by the appended claims.

SEQUENCES

SEQ ID NO: 1-Ciltacabtagene autoleucel CAR CD8α signal peptide, CD8α SP amino acid
sequence
MALPVTALLLPLALLLHAARP
SEQ ID NO: 2-Ciltacabtagene autoleucel CAR BCMA binding domain, VHH1 amino acid
sequence
QVKLEESGGGLVQAGRSLRLSCAASEHTFSSHVMGWFRQAPGKERESVAVIGWRDISTS
YADSVKGRFTISRDNAKKTLYLQMNSLKPEDTAVYYCAARRIDAADFDSWGQGTQVT
VSS
SEQ ID NO: 3-Ciltacabtagene autoleucel CAR BCMA binding domain, G4S linker amino
acid sequence
GGGGS
SEQ ID NO: 4-Ciltacabtagene autoleucel CAR BCMA binding domain, VHH2 amino acid
sequence
EVQLVESGGGLVQAGGSLRLSCAASGRTFTMGWFRQAPGKEREFVAAISLSPTLAYYAE
SVKGRFTISRDNAKNTVVLQMNSLKPEDTALYYCAADRKSVMSIRPDYWGQGTQVTVS
S
SEQ ID NO: 5-Ciltacabtagene autoleucel CAR CD8α hinge amino acid sequence
TTTPAPRPPTPAPTIASQPLSLRPEACRPAAGGAVHTRGLDFACD
SEQ ID NO: 6-Ciltacabtagene autoleucel CAR CD8α transmembrane amino acid sequence
IYIWAPLAGTCGVLLLSLVITLYC
SEQ ID NO: 7-Ciltacabtagene autoleucel CAR CD137 Cytoplasmic amino acid sequence
KRGRKKLLYIFKQPFMRPVQTTQEEDGCSCRFPEEEEGGCEL
SEQ ID NO: 8-Ciltacabtagene autoleucel CAR CD3z Cytoplasmic amino acid sequence
RVKFSRSADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR
SEQ ID NO: 9-Ciltacabtagene autoleucel CAR CD8α signal peptide CD8α SP nucleic acid
sequence
ATGGCTCTGCCCGTCACCGCTCTGCTGCTGCCTCTGGCTCTGCTGCTGCACGCTGCTC
GCCCT
SEQ ID NO: 10-Ciltacabtagene autoleucel CAR BCMA binding domain, VHH1 nucleic acid
sequence
CAGGTCAAACTGGAAGAATCTGGCGGAGGCCTGGTGCAGGCAGGACGGAGCCTGCG
CCTGAGCTGCGCAGCATCCGAGCACACCTTCAGCTCCCACGTGATGGGCTGGTTTCG
GCAGGCCCCAGGCAAGGAGAGAGAGAGCGTGGCCGTGATCGGCTGGAGGGACATC
TCCACATCTTACGCCGATTCCGTGAAGGGCCGGTTCACCATCAGCCGGGACAACGCC
AAGAAGACACTGTATCTGCAGATGAACAGCCTGAAGCCCGAGGACACCGCCGTGTA
CTATTGCGCAGCAAGGAGAATCGACGCAGCAGACTTTGATTCCTGGGGCCAGGGCA
CCCAGGTGACAGTGTCTAGC
SEQ ID NO: 11-Ciltacabtagene autoleucel CAR BCMA binding domain, G4S linker (SEQ ID
NO: 3) nucleic acid sequence
GGAGGAGGAGGATCT
SEQ ID NO: 12-Ciltacabtagene autoleucel CAR BCMA binding domain, VHH2 nucleic acid
sequence
GAGGTGCAGCTGGTGGAGAGCGGAGGCGGCCTGGTGCAGGCCGGAGGCTCTCTGAG
GCTGAGCTGTGCAGCATCCGGAAGAACCTTCACAATGGGCTGGTTTAGGCAGGCAC
CAGGAAAGGAGAGGGAGTTCGTGGCAGCAATCAGCCTGTCCCCTACCCTGGCCTAC
TATGCCGAGAGCGTGAAGGGCAGGTTTACCATCTCCCGCGATAACGCCAAGAATAC
AGTGGTGCTGCAGATGAACTCCCTGAAACCTGAGGACACAGCCCTGTACTATTGTGC
CGCCGATCGGAAGAGCGTGATGAGCATTAGACCAGACTATTGGGGGCAGGGAACAC
AGGTGACCGTGAGCAGC
SEQ ID NO: 13-Ciltacabtagene autoleucel CAR CD8α hinge nucleic acid sequence
ACCACGACGCCAGCGCCGCGACCACCAACACCGGCGCCCACCATCGCGTCGCAGCC
CCTGTCCCTGCGCCCAGAGGCGTGCCGGCCAGCGGCGGGGGGCGCAGTGCACACGA
GGGGGCTGGACTTCGCCTGTGAT
SEQ ID NO: 14-Ciltacabtagene autoleucel CAR CD8α transmembrane nucleic acid sequence
ATCTACATCTGGGCGCCCTTGGCCGGGACTTGTGGGGTCCTTCTCCTGTCACTGGTTA
TCACCCTTTACTGC
SEQ ID NO: 15-Ciltacabtagene autoleucel CAR CD137 Cytoplasmic nucleic acid sequence
AAACGGGGCAGAAAGAAACTCCTGTATATATTCAAACAACCATTTATGAGACCAGT
ACAAACTACTCAAGAGGAAGATGGCTGTAGCTGCCGATTTCCAGAAGAAGAAGAAG
GAGGATGTGAACTG
SEQ ID NO: 16-Ciltacabtagene autoleucel CAR CD3z Cytoplasmic nucleic acid sequence
AGAGTGAAGTTCAGCAGGAGCGCAGACGCCCCCGCGTACCAGCAGGGCCAGAACC
AGCTCTATAACGAGCTCAATCTAGGACGAAGAGAGGAGTACGATGTTTTGGACAAG
AGACGTGGCCGGGACCCTGAGATGGGGGGAAAGCCGAGAAGGAAGAACCCTCAGG
AAGGCCTGTACAATGAACTGCAGAAAGATAAGATGGCGGAGGCCTACAGTGAGATT
GGGATGAAAGGCGAGCGCCGGAGGGGCAAGGGGCACGATGGCCTTTACCAGGGTCT
CAGTACAGCCACCAAGGACACCTACGACGCCCTTCACATGCAGGCCCTGCCCCCTCG
CTAA
SEQ ID NO: 17-Ciltacabtagene autoleucel CAR amino acid sequence
MALPVTALLLPLALLLHAARPQVKLEESGGGLVQAGRSLRLSCAASEHTFSSHVMGWF
RQAPGKERESVAVIGWRDISTSYADSVKGRFTISRDNAKKTLYLQMNSLKPEDTAVYYC
AARRIDAADFDSWGQGTQVTVSSGGGGSEVQLVESGGGLVQAGGSLRLSCAASGRTFT
MGWFRQAPGKEREFVAAISLSPTLAYYAESVKGRFTISRDNAKNTVVLQMNSLKPEDT
ALYYCAADRKSVMSIRPDYWGQGTQVTVSSTSTTTPAPRPPTPAPTIASQPLSLRPEACR
PAAGGAVHTRGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNLGRREEYDV
LDKRRGRDPEMGGKPRRKNPQEGLYNELQKDKMAEAYSEIGMKGERRRGKGHDGLY
QGLSTATKDTYDALHMQALPPR

What is claimed:

1. A method of treating a subject, comprising administering to the subject a single infusion of a dose of a composition comprising T cells comprising a chimeric antigen receptor (CAR),
wherein the CAR comprises the amino acid sequence of SEQ ID NO: 17;
wherein the dose comprises 0.5×10⁶ to 1.0×10⁶ of the T cells/kg of body weight of the subject; and
wherein the method comprises completing administering to the subject the dose of T cells within about 2.5 hours at a temperature of about 20° C. to 25° C.

2. The method of claim 1, wherein the subject has relapsed or refractory multiple myeloma, who has received multiple prior lines of therapy, and wherein optionally the subject has received three or more prior lines of therapy.

3. The method of claim 1 or 2, wherein the subject has received four or more prior lines of therapy.

4. The method of claim 2 or 3, wherein the prior lines of therapy comprise a proteasome inhibitor, an immunomodulatory agent, and an anti-CD38 monoclonal antibody.

5. The method of any one of claims 1-4, wherein the T cells are autologous T cells.

6. The method of any one of claims 1-5, wherein the method further comprises:
(1) administering to the subject a lymphodepleting chemotherapy regimen prior to administering to the subject the T cells, wherein optionally:
(a) the lymphodepleting chemotherapy regimen comprises administering cyclophosphamide and fludarabine to the subject,
(b) the lymphodepleting chemotherapy regimen comprises administering cyclophosphamide and fludarabine to the subject intravenously,
(c) the lymphodepleting chemotherapy regimen comprises administering to the subject intravenously cyclophosphamide at a dose of about 300 mg/m² and fludarabine at a dose of 30 mg/m² daily,
(d) the lymphodepleting chemotherapy regimen is for about 3 days,
(e) the method comprises administering to the subject the lymphodepleting chemotherapy regimen for at least about 2-4 days prior to administering to the subject the T cells; or
(2) administering to the subject a premedication for up to 60 minutes prior to administering to the subject the T cells and wherein the premedication comprises an antipyretics and an antihistamine, wherein optionally:
(a) the method comprises administering to the subject the premedication for about 30-60 minutes prior to administering to the subject the T cells,
(b) the antipyretics comprises paracetamol or acetaminophen,
(c) the antipyretics comprises acetaminophen at a dose of about 650-1000 mg,
(d) the antihistamine comprises diphenhydramine,
(e) the diphenhydramine is at a dose of about 25-50 mg or equivalent,
(f) the premedication is administered orally or intravenously, or
(g) the premedication does not comprise a systemic corticosteroid.

7. The method of any one of claims 1-6, wherein the method comprises thawing the dose of the T cells prior to administration, wherein the thawing is completed in no more than about 15 minutes, wherein optionally thawing the dose of the T cells is at a temperature of about 37° C.±2° C.

8. The method of any one of claims 1-7, wherein the method further comprises treating the subject for cytokine release syndrome (CRS) after administering the dose of the T cells, wherein optionally treating the subject for CRS comprises administering an anti-cytokine agent or a corticosteroid to the subject, wherein optionally:
(1) the anti-cytokine agent comprises a monoclonal antibody targeting cytokines, wherein optionally the monoclonal antibody targeting cytokines is an IL-6R inhibitor, wherein optionally the IL-6R inhibitor is tocilizumab, wherein optionally the method comprises administering tocilizumab intravenously at a dose of about 8 mg/kg over about 1 hour, wherein optionally the dose of tocilizumab does not exceed about 800 mg, wherein optionally the dose of tocilizumab is no more than 3 doses in 24 hours or no more than 4 doses in total, wherein further optionally the anti-cytokine agent further comprises an anti-cytokine agent other than tocilizumab, wherein further optionally the anti-cytokine agent further comprises a monoclonal antibody targeting cytokines other than tocilizumab;
(2) the corticosteroid comprises dexamethasone or methylprednisolone, wherein optionally the corticosteroid is dexamethasone, wherein further optionally the method comprises administering to the subject a dose of about 10 mg of dexamethasone intravenously about every 12-24 hours, wherein further optionally the method comprises administering to the subject a dose of about 10 mg of dexamethasone intravenously about every 12 hours, wherein further optionally the method comprises administering to the subject a dose of about 20 mg of dexamethasone intravenously about every 6-12 hours, wherein further optionally the method comprises administering to the subject a dose of about 20 mg of dexamethasone intravenously about every 6 hours, wherein optionally the corticosteroid is methylprednisolone, wherein further optionally the method comprises administering to the subject a dose of about 2 mg/kg methylprednisolone intravenously about every 12 hours, and wherein further optionally the method comprises administering to the subject a dose of about 1-2 g of methylprednisolone intravenously about every 24 hours; or
(3) the method comprises administering an immunosuppressant to the subject.

9. The method of claim 8, wherein CRS comprises fever, pyrexia, hypotension, increased aspartate aminotransferase, chills, increased alanine aminotransferase, sinus tachycardia, hyperbilirubinemia, hypoxia, respiratory failure, acute kidney injury, disseminated intravascular coagulation and hemorrhage (e.g., retroperitoneal, intracerebral or gastrointestinal hemorrhage), hemophagocytic lymphohistiocytosis (HLH), macrophage activation syndrome (MAS), angina pectoris, supraventricular and ventricular tachycardia, malaise, myalgias, increased-C-reactive protein, ferritin, blood alkaline phosphatase, gamma-glutamyl transferase, organ toxicity, or any combination thereof.

10. The method of claim 9, wherein CRS comprises hemophagocytic lymphohistiocytosis (HLH) or macrophage activation syndrome (MAS) and wherein symptom of HLH or MAS comprises hypotension, hypoxia with diffuse alveolar damage, coagulopathy, cytopenia, multi-organ dysfunction including renal dysfunction, or any combination thereof, wherein optionally the method comprises administering a treatment to the subject to alleviate HLH or MAS.

11. The method of any one of claims 1-10, wherein the method further comprises treating the subject for neurologic toxicity after administering the dose of the T cells.

12. The method of claim 11, wherein the neurologic toxicity comprises an immune effector cell-associated neurotoxicity syndrome (ICANS), parkinsonism, Guillain-Barré Syndrome, immune mediated myelitis, peripheral neuropathy, cranial nerve palsy or any combination thereof.

13. The method of claim 12, wherein the neurologic toxicity comprises an ICANS and wherein the ICANS comprises encephalopathy, aphasia, headache, depressed level of consciousness, seizure, motor finding, raised intracranial pressure (ICP), celebral edema, or any combination thereof, wherein optionally the ICANS comprises focal or generalized seizure, non-convulsive seizure on electroencephalogram (EEG), life-threatening prolonged seizure, repetitive clinical or electrical seizure, deep focal motor weakness, hemiparesis, paraparesis, focal or local edema on neuroimaging, stupor, coma, diffuse cerebral edema on neuroimaging, decerebrate or decorticate posturing, cranial nerve VI palsy, papilledema, Cushing's triad, or any combination thereof, wherein optionally:
(1) the method comprises administering to the subject a dose of about 10 mg of dexamethasone intravenously about every 12-24 hours for about 2-3 days,
(2) the method comprises administering to the subject a dose of about 10 mg of dexamethasone intravenously about every 12 hours for about 2-3 days or longer,
(3) the method comprises administering to the subject a dose of about 10-20 mg of dexamethasone intravenously about every 6 hours, or
(4) the method comprises administering to the subject a dose of methylprednisolone at about 1-2 g/day about every 24 hours,
wherein further optionally the ICANS comprises celebral edema, wherein further optionally the method comprises administering to the subject hyperventilation and hyperosmolar therapy, wherein further optionally the method comprises administering to the subject a non-sedating anti-seizure medicine, and wherein further optionally the non-sedating anti-seizure medicine is levetiracetam.

14. The method of claim 12, wherein the neurologic toxicity comprises parkinsonism, wherein optionally the parkinsonism comprises a parkinsonian symptom or a non-parkinsonian symptom, wherein optionally the parkinsonian symptom or the non-parkinsonian symptom comprises tremor, bradykinesia, involuntary movements, stereotypy, loss of spontaneous movements, masked facies, apathy, flat affect, fatigue, rigidity, psychomotor retardation, micrographia, dysgraphia, apraxia, lethargy, confusion, somnolence, loss of consciousness, delayed reflexes, hyperreflexia, memory loss, difficulty swallowing, bowel incontinence, falls, stooped posture, shuffling gait, muscle weakness and wasting, motor dysfunction, motor and sensory loss, akinetic mutism, frontal lobe release signs, or any combination thereof, and wherein optionally the method comprises administering a treatment to the subject to alleviate parkinsonism.

15. The method of claim 12, wherein the neurologic toxicity comprises Guillain-Barré Syndrome, wherein optionally Guillain-Barré Syndrome comprises a symptom consistent with Miller-Fisher variant of Guillain-Barré Syndrome, encephalopathy, motor weakness, speech disturbances, polyradiculoneuritis, or any combination thereof, and wherein optionally the method comprises administering a treatment to the subject to alleviate Guillain-Barré Syndrome.

16. The method of claim 12, wherein the neurologic toxicity comprises immune mediated myelitis, wherein optionally a symptom of immune mediated myelitis comprises hypoesthesia of a lower extremity or lower abdomen with impaired sphincter control, wherein optionally the method comprises administering a treatment to the subject to alleviate immune mediated myelitis, wherein optionally the treatment comprises a corticosteroid or an immune globulin, and wherein optionally the method comprises administering the immune globulin intravenously.

17. The method of claim 12, wherein the neurologic toxicity comprises peripheral neuropathy, wherein optionally the peripheral neuropathy comprises sensory, motor, sensorimotor neuropathy, or any combination thereof, and wherein optionally the method comprises administering a treatment to the subject to alleviate peripheral neuropathy.

18. The method of claim 12, wherein the neurologic toxicity comprises cranial nerve palsy, wherein optionally cranial nerve palsy comprises 3^{rd} cranial nerve palsy, 6^{th} cranial nerve palsy, 7^{th} cranial nerve palsy, or bilateral 7^{th} cranial nerve palsy, and wherein optionally the method comprises administering a treatment to the subject to alleviate cranial nerve palsy.

19. The method of any one of claims 1-18, wherein the method further comprises treating the subject for prolonged or recurrent cytopenia after administering to the subject a lymphodepleting chemotherapy regimen prior to administering to the subject the T cells comprising the CAR or after administering the dose of the T cells comprising the CAR, wherein optionally the prolonged recurrent cytopenia comprises prolonged neutropenia, prolonged thrombocytopenia, recurrent neutropenia, thrombocytopenia, lymphopenia, anemia, or any combination thereof.

20. The method of any one of claims 1-19, wherein the method further comprises treating the subject for an infection, wherein optionally the infection is viral, bacterial, fungal, or by an unspecified pathogen, wherein optionally the infection comprises lung abscess, sepsis, pneumonia, bronchopulmonary aspergillosis, Pneumocystis jirovecii pneumonia, CMV colitis (with HSV-1 hepatitis), mycotic aneurysm, cerebral aspergillosis or COVID-19 infection, wherein optionally the infection causes febrile neutropenia or subarachnoid hemorrhage, wherein optionally the method comprises administering to the subject an antimicrobial, wherein optionally the antimicrobial is an antibiotic, wherein further optionally the antibiotic is a broad-spectrum antibiotic, wherein optionally the infection is viral, and wherein further optionally the method comprises administering to the subject an antiviral therapy or a vaccine.

21. The method of any one of claims 1-20, wherein the method further comprises treating the subject for hypogammaglobulinemia, wherein optionally hypogammaglobulinemia comprises a laboratory IgG level below about 500 mg/dL after administering the dose of the T cells comprising the CAR, and wherein optionally the method comprises administering to the subject a dose of intravenous immunoglobulin (IVIG) after administering the dose of the T cells comprising the CAR.

22. The method of any one of claims 1-21, wherein the method further comprises treating the subject for a hypersensitivity reaction, wherein optionally the hypersensitivity reaction comprises flushing, chest discomfort, tachycardia, wheezing, tremor, burning sensation, anaphylaxis, or any combination thereof, and wherein optionally the method comprises administering to the subject a treatment to alleviate the hypersensitivity reaction.

23. The method of any one of claims 1-22, wherein the method further comprises treating the subject for a secondary malignancy.

24. The method of any one of claims 1-23, wherein the composition further comprises an excipient selected from dimethyl sulfoxide or dextran-40, wherein optionally the excipient is dimethyl sulfoxide, wherein optionally the excipient is about 1-10% of dimethyl sulfoxide, and wherein further optionally the excipient is about 5% of dimethyl sulfoxide.

25. A pharmaceutical product comprising a ciltacabtagene autoleucel suspension for intravenous infusion, wherein the pharmaceutical product is packaged, and wherein the package includes a label that identifies the ciltacabtagene autoleucel suspension as an approved drug product for the treatment of adult patients with relapsed or refractory multiple myeloma after four or more prior lines of therapy, including a proteasome inhibitor, an immunomodulatory agent and an anti-CD38 monoclonal antibody.

26. A method for treating relapsed or refractory multiple myeloma in a patient in need thereof, comprising administering an approved drug product comprising a ciltacabtagene autoleucel suspension in an amount and manner that is described in a drug product label for the approved drug product.

27. A method of selling an approved drug product comprising a ciltacabtagene autoleucel suspension, said method comprising selling such drug product, wherein a drug product label for a reference product for such drug product includes instructions for treating adult patients with relapsed or refractory multiple myeloma after four or more prior lines of therapy, including a proteasome inhibitor, an immunomodulatory agent and an anti-CD38 monoclonal antibody.

28. A method of offering for sale a drug product comprising a ciltacabtagene autoleucel suspension, said method comprising offering for sale such drug product, wherein a drug product label for a reference product for such drug product includes instructions for treating adult patients with relapsed or refractory multiple myeloma after four or more prior lines of therapy, including a proteasome inhibitor, an immunomodulatory agent and an anti-CD38 monoclonal antibody.
